(19) United States
(12) Patent Application Publication
(10) Pub. No.: US 2021/0015869 A1
(43) Pub. Date: Jan. 21, 2021

(54) T CELLS EXPRESSING A RECOMBINANT RECEPTOR, RELATED POLYNUCLEOTIDES AND METHODS

(71) Applicants: Juno Therapeutics, Inc., Seattle, WA (US); Editas Medicine, Inc., Cambridge, MA (US)

(72) Inventors: Stephen Michael BURLEIGH, Seattle, WA (US); Christopher BORGES, Cambridge, MA (US); Christopher Heath NYE, Seattle, WA (US); Blythe D. SATHER, Seattle, WA (US); Queenie VONG, Seattle, WA (US); Gordon Grant WELSTEAD, Cambridge, MA (US)

(73) Assignees: Juno Therapeutics, Inc., Seattle, WA (US); Editas Medicine, Inc., Cambridge, MA (US)

(21) Appl. No.: 17/044,279

(22) PCT Filed: Apr. 3, 2019
(86) PCT No.: PCT/US2019/025681

§ 371 Date: Sep. 30, 2020

Related U.S. Application Data
(60) Provisional application No. 62/653,553, filed on Apr. 5, 2018.

(51) Int. Cl. A61K 35/17; C12N 5/0783; C12N 15/90; C12N 15/86; C07K 14/725; C12N 9/22; C12N 15/113

(52) U.S. Cl. CPC A61K 35/17; C12N 5/0637; C12N 15/907; C12N 15/86; C12N 2310/20; C12N 9/22; C12N 15/113; C12N 2506/45; C12N 2750/14143; C07K 14/7051

(57) ABSTRACT

Provided herein are methods for engineering immune cells, cell compositions containing engineered immune cells, kits and articles of manufacture for targeting nucleic acid sequence encoding a portion of a recombinant receptor, e.g., a recombinant T cell receptor (TCR), to a particular genomic locus and/or for modulating expression of the gene at the genomic locus, and applications thereof in connection with cancer immunotherapy, such as adoptive transfer of engineered T cells. In some aspects, the nucleic acid sequence integrates in-frame into the locus of a receptor encoding gene, and in some aspects, results in expression of the whole recombinant receptor.

### CROSS-REFERENCE TO RELATED APPLICATIONS

This application claims priority from U.S. provisional application No. 62/653,553, filed Apr. 5, 2018, entitled “T CELLS EXPRESSING A RECOMBINANT RECEPTOR, RELATED POLYNUCLEOTIDES AND METHODS,” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 735042015540SeqList.txt, created Apr. 3, 2019 which is 181 kilobytes in size. The information in the electronic format of the Sequence Listing is incorporated by reference in its entirety.

### FIELD

The present disclosure relates to methods for engineering immune cells, cell compositions containing engineered immune cells, kits and articles of manufacture for targeting nucleic acid sequence encoding a portion of a recombinant receptor to a particular genomic locus and/or for modulating expression of the gene at the genomic locus, and applications thereof in connection with cancer immunotherapy comprising adoptive transfer of engineered T cells. In some aspects, the nucleic acid sequence integrates in-frame into the locus of a receptor encoding gene, and in some aspects, results in expression of the whole recombinant receptor.

### BACKGROUND

Adoptive cell therapies that utilize recombinantly expressed T cell receptors (TCRs) or other antigen receptors (e.g. chimeric antigen receptors (CARs)) to recognize tumor antigens represent an attractive therapeutic modality for the treatment of cancers and other diseases. Expression and function of recombinant TCRs or other antigen receptors can be limited and/or heterogeneous in a population of cells. Improved strategies are needed to achieve high and/or homogenous expression levels and function of the recombinant receptors. These strategies can facilitate generation of cells exhibiting desired expression levels and/or properties for use in adoptive immunotherapy, e.g., in treating cancer, infectious diseases and autoimmune diseases.

### SUMMARY

Provided herein are genetically engineered immune cells expressing a recombinant receptor such as a recombinant T cell receptor (TCR), that are engineered by targeted integration of nucleic acid sequences encoding the recombinant receptor. In some aspects, the engineered cells comprise a modified T cell receptor alpha constant (TRAC) locus and/or a modified T cell receptor beta constant (TRBC) locus. In some aspects, the modified TRAC and/or TRBC locus contains nucleic acid sequences encoding a recombinant TCR or a portion or a chain thereof. In some aspects, the provided genetically engineered cells contain modified TRAC and/or TRBC locus that contains a fusion of a transgene sequence encoding a portion of a recombinant TCR, and the endogenous open reading frame of the gene encoding a constant domain of the TCR. In some aspects, also provided are methods of generating an engineered T cell, related cell compositions, nucleic acids and kits for use in generating the engineered cells described herein.

Provided herein is a genetically engineered T cell, containing a modified T cell receptor alpha constant (TRAC) locus. In some embodiments, provided herein is a genetically engineered T cell, containing a modified T cell receptor alpha constant (TRAC) locus, said modified TRAC locus containing a nucleic acid encoding a recombinant TCR or portion thereof, said recombinant TCR or portion thereof containing: (i) a TCR alpha (TCRα) chain comprising a variable alpha (Vα) domain and a constant alpha (Cα) domain, and (ii) a TCR beta (TCRβ) chain comprising a variable beta (Vβ) domain and a constant beta (Cβ) domain, wherein the nucleic acid sequence comprises of (a) a transgene sequence encoding the TCRβ and the Vα domain, and (b) an open reading frame of the endogenous TRAC locus or a partial sequence thereof, wherein the open reading frame encodes at least a portion of the Cα domain of the recombinant TCR.

Provided herein are genetically engineered T cells that contains a modified T cell receptor alpha constant (TRAC) locus. In some aspects, the modified TRAC locus includes a nucleic acid encoding a recombinant TCR or portion thereof, said recombinant TCR or portion thereof comprising: (i) a TCR alpha (TCRα) chain comprising a variable alpha (Vα) domain and a constant alpha (Cα) domain, and (ii) a TCR beta (TCRβ) chain comprising a variable beta (Vβ) domain and a constant beta (Cβ) domain, wherein the nucleic acid sequence comprises of (a) a transgene sequence encoding the TCRβ and the Vα domain, and (b) an open reading frame of the endogenous TRAC locus or a partial sequence thereof, wherein the open reading frame encodes at least a portion of the Cα domain of the recombinant TCR. In some of any such embodiments, a portion of the Cα is encoded by the open reading frame of the endogenous TRAC locus or a partial sequence thereof, and a further portion of the Cα is encoded by the transgene sequence, wherein said further portion of Cα is less than the full length of a native Ca; and the further portion of the Cα and/or the Cβ region encoded by the nucleic acid sequence of (a) comprises one or more modifications compared to a native Cα region and/or a native Cβ region, said one or more modifications introduces one or more cysteine residues that are capable of forming one or more non-native disulfide bridges between the alpha chain and beta chain and/or wherein the Cα and/or the Cβ of the recombinant TCR comprises one or more non-native cysteines.

Also provided are genetically engineered T cells that contain a modified T cell receptor alpha constant (TRAC) locus that includes a nucleic acid encoding a recombinant TCR or portion thereof, said recombinant TCR or portion thereof comprising: (i) a TCR alpha (TCRα) chain comprising a variable alpha (Vα) domain and a constant alpha (Cα) domain, and (ii) a TCR beta (TCRβ) chain comprising a variable beta (Vβ) domain and a constant beta (Cβ) domain, wherein the nucleic acid sequence comprises of (a) a transgene sequence encoding the TCRβ and the Vα domain, and (b) an open reading frame of the endogenous TRAC locus or a partial sequence thereof, wherein the open reading frame encodes at least a portion of the Ca domain of the recombinant TCR, wherein transgene sequence comprises one or more heterologous or regulatory control element(s) comprising a heterologous promoter, operably linked to control expression of the TCR when expressed from a cell introduced with the genetically engineered T cell.

In some embodiments, the transgene sequence is or has been integrated via homology directed repair (HDR). In some embodiments, the modified TRAC locus contains an in-frame fusion of (i) a transgene sequence and (ii) an open reading frame or a partial sequence thereof of the endogenous TRAC locus. In some of any such embodiments, the transgene sequence is in-frame with one or more exons of the open reading frame or partial sequence thereof of the endogenous TRAC locus.

In some embodiments, the transgene sequence does not contain a sequence encoding a 3′ untranslated region (3′ UTR) or an intron. In some embodiments, the open reading frame or a partial sequence thereof contains a 3′ UTR of the endogenous TRAC locus. In some embodiments, a portion of the Cα is encoded by the open reading frame of the endogenous TRAC locus or a partial sequence thereof, and a further portion of the Cα is encoded by the transgene sequence, wherein said further portion of Cα is less than the full length of a native Ca.

In some embodiments, the open reading frame or the partial sequence thereof contains at least one intron and at least one exon of the endogenous TRAC locus. In some embodiments, the transgene sequence is in-frame with one or more exons of the open reading frame or partial sequence thereof of the endogenous TRAC locus.

In some of any such embodiments, the further portion of the Cα is encoded by a sequence of nucleotides that comprises less than four exons, less than three exons, less than two exons, one exon, or less than one full exon the open reading frame of the TRAC locus. In some of any such embodiments, the further portion of the Cα is encoded by a sequence of nucleotides that is less than 400, less than 300, less than 250, less than 200, or less than 150 base pairs in length. In some of any such embodiments, the further portion of the Cα is encoded by a portion of exon 1 of the TRAC locus, wherein the portion of exon 1 is less than the full length of exon 1 the open reading frame of the TRAC locus.

In some embodiments, the transgene sequence is or has been integrated downstream of the most 5′ nucleotide of exon 1 and upstream of the most 3′ nucleotide of exon 1 of the open reading frame of the endogenous TRAC locus. In some embodiments, the at least a portion of Ca is encoded by at least exons 2-4 of the open reading frame of the endogenous TRAC locus or at least a portion of exon 1 and exons 2-4 of the open reading frame of the endogenous TRAC locus. In some embodiments, the at least a portion Cα is encoded by less than the full length of exon 1 of the open reading frame of the endogenous TRAC locus.

In some embodiments, the encoded TCRα chain is capable of dimerizing with a TCRβ chain.

In some embodiments, the encoded Cα contains the sequence selected from any one of SEQ ID NOS: 14, 15, 19, or 24, or a sequence that exhibits at least 85%, 86%, 87%, 88%, 89%, 90%, 91%, 92%, 93%, 94%, 95%, 96%, 97%, 98%, 99% or more sequence identity to any one of SEQ ID NOS: 14, 15, 19, or 24, or a partial sequence thereof.

In some of any of the embodiments provided herein, the encoded Cα comprises the sequence selected from any one of SEQ ID NOS: 19, 24 and 243-252, or a sequence that exhibits at least 85%, 86%, 87%, 88%, 89%, 90%, 91%, 92%, 93%, 94%, 95%, 96%, 97%, 98%, 99% or more sequence identity to any one of SEQ ID NOS: 19, 24 and 243-252, or a partial sequence thereof.

In some embodiments, wherein a further portion of the Cα is encoded by the transgene sequence. In some embodiments, the further portion of the Cα and/or the at least a portion of the Cα is encoded by a sequence of nucleotides starting from residue 3 and up to residue 3155 of the sequence set forth in SEQ ID NO:1 or one or more exons thereof or a sequence that exhibits at least 85%, 86%, 87%, 88%, 89%, 90%, 91%, 92%, 93%, 94%, 95%, 96%, 97%, 98%, 99% or more sequence identity to a sequence of nucleotides starting from residue 3 and up to residue 3155 of the sequence set forth in SEQ ID NO:1 or one or more exons thereof, or a partial sequence thereof. In some embodiments, the further portion of the Cα is encoded by a sequence of nucleotides that is less than 400, less than 300, less than 250, less than 200, or less than 150 base pairs in length. In some embodiments, the further portion of the Cα is encoded by a sequence of nucleotides that contains less than four exons, less than three exons, less than two exons, one exon, or less than one full exon the open reading frame of the TRAC locus. In some embodiments, the further portion of the Cα is encoded by a portion of exon 1 of the TRAC locus, wherein the portion of exon 1 is less than the full length of exon 1 the open reading frame of the TRAC locus. In some embodiments, the further portion of the Cα is encoded by a portion of exon 1 of the TRAC locus, wherein the portion of exon 1 contains a 5′ portion of exon 1. In some embodiments, the further portion of the Cα contains a sequence set forth in SEQ ID NO:142, or a sequence that exhibits at least 85%, 86%, 87%, 88%, 89%, 90%, 91%, 92%, 93%, 94%, 95%, 96%, 97%, 98%, 99% or more sequence identity to SEQ ID NO:142, or a partial sequence thereof.

In some embodiments, the further portion of the Cα and/or the Cβ region encoded by the nucleic acid sequence of (a) contains one or more modifications, optionally a replacement, deletion, or insertion of one or more amino acids compared to a native Cα region and/or a native Cβ region, optionally said one or more modifications introduces one or more cysteine residues that are capable of forming one or more non-native disulfide bridges between the alpha chain and beta chain.

In some of any embodiments, the Cα and/or the Cβ of the recombinant TCR comprises one or more non-native cysteines. In some of any embodiments, the introduction of one or more cysteine residues comprises replacement of a non-cysteine residue with a cysteine residue. In some of any embodiments, the encoded Cα region comprises a cysteine at a position corresponding to position 48 with numbering as set forth in any of SEQ ID NO: 24; and/or the encoded Cβ region comprises a cysteine at a position corresponding to position 57 with numbering as set forth in SEQ ID NO: 20. In some of any embodiments, the encoded Cα comprises the sequence selected from any one of SEQ ID NOS: 248-252, or a partial sequence thereof.

In any of the provided embodiments, the engineered T cell further can contain a genetic disruption at a TRBC locus. In some embodiments, the engineered T cell further contains a genetic disruption at a TRBC1 locus and/or a TRBC2 locus.

In some embodiments, the further portion of the Cα and/or the Cβ region encoded by the nucleic acid sequence of (a) contains one or more modifications, optionally a replacement, deletion, or insertion of one or more amino acids compared to a native Cα region and/or a native Cβ region, optionally said one or more modifications introduces one or more cysteine residues that are capable of forming one or more non-native disulfide bridges between the alpha chain and beta chain.

Provided herein is a genetically engineered T cell, containing a modified T cell receptor beta constant (TRBC) locus. In some embodiments, provided herein is a genetically engineered T cell, containing a modified T cell receptor beta constant (TRBC) locus, said modified TRBC locus containing a nucleic acid encoding a recombinant TCR or portion thereof, said recombinant TCR or portion thereof containing: (i) a TCR beta (TCRβ) chain comprising a variable beta (Vβ) domain and a constant beta (Cβ) domain, and (ii) a TCR alpha (TCRα) chain comprising a variable alpha (Vα) domain and a constant alpha (Cα) domain, wherein the nucleic acid sequence comprises of (a) a transgene sequence encoding the TCRα and the Vβ domain, and (b) an open reading frame of the endogenous TRBC locus or a partial sequence thereof, wherein the open reading frame encodes at least a portion of the Cβ domain of the recombinant TCR.

Also provided herein are genetically engineered T cells that contain a modified T cell receptor beta constant (TRBC) locus. In some of any such embodiments, the modified TRBC locus comprising a nucleic acid encoding a recombinant TCR or portion thereof, said recombinant TCR or portion thereof comprising: (i) a TCR beta (TCRβ) chain comprising a variable beta (Vβ) domain and a constant beta (Cβ) domain, and (ii) a TCR alpha (TCRα) chain comprising a variable alpha (Vα) domain and a constant alpha (Cα) domain, wherein the nucleic acid sequence comprises of (a) a transgene sequence encoding the TCRα and the Vβ domain, and (b) an open reading frame of the endogenous TRBC locus or a partial sequence thereof, wherein the open reading frame encodes at least a portion of the Cβ domain of the recombinant TCR. In some of any such embodiments, a portion of the Cβ is encoded by the open reading frame of the endogenous TRBC locus or a partial sequence thereof, and a further portion of the Cβ is encoded by the transgene sequence, wherein said further portion of Cβ is less than the full length of a native Cβ; and the further portion of the Cβ and/or the Cα region encoded by the nucleic acid sequence of (a) comprises one or more modifications, in some cases a replacement, deletion, or insertion of one or more amino acids compared to a native Cβ region and/or a native Ca region, in some cases said one or more modifications introduces one or more cysteine residues that are capable of forming one or more non-native disulfide bridges between the alpha chain and beta chain.

In some of such embodiments, the transgene sequence is or has been integrated via homology directed repair (HDR). In some embodiments, the TRBC locus is a TRBC1 locus and/or a TRBC2 locus. In some embodiments, the modified TRBC locus contains an in-frame fusion of (i) a transgene sequence and (ii) an open reading frame or a partial sequence thereof of the endogenous TRBC locus. In some embodiments, the transgene sequence is in-frame with one or more exons of the open reading frame or partial sequence thereof of the endogenous TRBC locus. In some embodiments, the transgene sequence does not contain a sequence encoding a 3′ untranslated region (3′ UTR) or an intron. In some embodiments, the open reading frame or a partial sequence thereof contains a 3′ UTR of the endogenous TRBC locus.

In some embodiments, a portion of the Cβ is encoded by the open reading frame of the endogenous TRBC locus or a partial sequence thereof, and a further portion of the Cβ is encoded by the transgene sequence, wherein said further portion of Cβ is less than the full length of a native Cβ.

In some embodiments, the open reading frame or the partial sequence thereof contains at least one intron and at least one exon of the endogenous TRBC locus. In some embodiments, the transgene sequence is in-frame with one or more exons of the open reading frame or partial sequence thereof of the endogenous TRBC locus. In some embodiments, the transgene sequence is or has been integrated downstream of the most 5′ nucleotide of exon 1 and upstream of the most 3′ nucleotide of exon 1 of the open reading frame of the endogenous TRBC locus.

In some embodiments, the at least a portion of Cβ is encoded by at least exons 2-4 of the open reading frame of the endogenous TRBC locus. In some embodiments, the at least a portion of Cβ is encoded by at least a portion of exon 1 and exons 2-4 of the open reading frame of the endogenous TRBC locus. In some embodiments, the at least a portion of Cβ is encoded by less than the full length of exon 1 of the open reading frame of the endogenous TRBC locus.

In some of any such embodiments, the further portion of the Cβ is encoded by a sequence of nucleotides that comprises less than four exons, less than three exons, less than two exons, one exon, or less than one full exon the open reading frame of a TRBC locus. In some of any such embodiments, the further portion of the Cβ is encoded by a sequence of nucleotides that is less than 400, less than 300, less than 250, less than 200, or less than 150 base pairs in length. In some of any such embodiments, the further portion of the Cβ is encoded by a portion of exon 1 of a TRBC locus, wherein the portion of exon 1 is less than the full length of exon 1 the open reading frame of the TRBC locus.

In some embodiments, the encoded TCRβ chain is capable of dimerizing with a TCRα chain.

In some embodiments, the encoded Cβ contains the sequence selected from any one of SEQ ID NO: 16, 17, 20, 21, and 25, or a sequence that exhibits at least 85%, 86%, 87%, 88%, 89%, 90%, 91%, 92%, 93%, 94%, 95%, 96%, 97%, 98%, 99% or more sequence identity to any one of SEQ ID NO: 16, 17, 20, 21, and 25, or a partial sequence thereof.

In some embodiments, the encoded Cβ comprises the sequence selected from any one of SEQ ID NO: 20, 21, 25 and 253-258 or a sequence that exhibits at least 85%, 86%, 87%, 88%, 89%, 90%, 91%, 92%, 93%, 94%, 95%, 96%, 97%, 98%, 99% or more sequence identity to any one of SEQ ID NO: 20, 21, 25 and 253-258, or a partial sequence thereof.

In some embodiments, the further portion of the Cβ and/or the at least a portion of Cβ is encoded by a sequence of nucleotides starting from residue 3 and up to residue 1445 of the sequence set forth in SEQ ID NO:2 or one or more exons thereof or a sequence that exhibits at least 85%, 86%, 87%, 88%, 89%, 90%, 91%, 92%, 93%, 94%, 95%, 96%, 97%, 98%, 99% or more sequence identity to a sequence of nucleotides starting from residue 3 and up to residue 1445 of the sequence set forth in SEQ ID NO:2 or one or more exons thereof, or a partial sequence thereof; or a sequence of nucleotides starting from residue 3 and up to residue 1486 of the sequence set forth in SEQ ID NO:3 or one or more exons thereof or a sequence that exhibits at least 85%, 86%, 87%, 88%, 89%, 90%, 91%, 92%, 93%, 94%, 95%, 96%, 97%, 98%, 99% or more sequence identity to a sequence of nucleotides starting from residue 3 and up to residue 1486 of the sequence set forth in SEQ ID NO:3 or one or more exons thereof, or a partial sequence thereof. In some embodiments, the further portion of the Cα is encoded by a sequence of nucleotides that is less than 400, less than 300, less than 250, less than 200, or less than 150 base pairs in length.

In some embodiments, the further portion of the Cα is encoded by a sequence of nucleotides that contains less than four exons, less than three exons, less than two exons, one exon, or less than one full exon the open reading frame of a TRBC locus. In some embodiments, the further portion of the Cα is encoded by a portion of exon 1 of a TRBC locus, wherein the portion of exon 1 is less than the full length of exon 1 the open reading frame of the TRBC locus or the portion of exon 1 contains a 5′ portion of exon 1.

In some embodiments, the further portion of the Cβ and/or the Cα region encoded by the nucleic acid sequence of (a) contains one or more modifications, optionally a replacement, deletion, or insertion of one or more amino acids compared to a native Cβ region and/or a native Cα region, optionally said one or more modifications introduces one or more cysteine residues that are capable of forming one or more non-native disulfide bridges between the alpha chain and beta chain.

In some of any embodiments, the introduction of one or more cysteine residues comprises replacement of a non-cysteine residue with a cysteine residue. In some of any embodiments, the encoded Cα region comprises a cysteine at a position corresponding to position 48 with numbering as set forth in any of SEQ ID NO: 24; and/or the encoded Cβ region comprises a cysteine at a position corresponding to position 57 with numbering as set forth in SEQ ID NO: 20. In some of any embodiments, the encoded Cβ comprises the sequence selected from any one of SEQ ID NOS: 253 and 256-258, or a partial sequence thereof.

In some embodiments, the engineered T cell further contains a genetic disruption at a TRAC locus.

In some embodiments, the transgene sequence contains one or more multicistronic element(s). In some of such embodiments, the multicistronic element(s) is positioned between the nucleic acid sequence encoding the TCRα or a portion thereof and the nucleic acid sequence encoding the TCRβ or a portion thereof. In some embodiments, the one or more multicistronic element(s) are upstream of the nucleic acid sequence encoding the TCR or a portion of the TCR or the nucleic acid molecule encoding the TCR. In some embodiments, the multicistronic element is or contains a ribosome skip sequence, optionally T2A, P2A, E2A, or F2A.

In some embodiments, the transgene sequence contains one or more heterologous regulatory or control element(s). In some embodiments, the transgene sequence contains one or more heterologous or regulatory control element(s) operably linked to control expression of the TCR when expressed from a cell introduced with the genetically engineered T cell. In some embodiments, the one or more heterologous regulatory or control element contains a promoter, an enhancer, an intron, a polyadenylation signal, a Kozak consensus sequence, a splice acceptor sequence and/or a splice donor sequence.

In some embodiments, the heterologous regulatory or control element contains a heterologous promoter, such as a heterologous promoter selected from among a constitutive promoter, an inducible promoter, a repressible promoter, and/or a tissue-specific promoter. In some embodiments, the heterologous promoter is or contains a human elongation factor 1 alpha (EF1α) promoter or an MND promoter or a variant thereof.

In some embodiments, the Cα and/or the Cβ of the recombinant TCR contain(s) one or more non-native cysteine(s).

In any of the provided embodiments, the recombinant TCR can be capable of binding to an antigen that is associated with, specific to, and/or expressed on a cell or tissue that is associated with a disease, disorder, or condition, such as an infectious disease or disorder, an autoimmune disease, an inflammatory disease, a tumor, or a cancer.

In some embodiments, the antigen is a tumor antigen or a pathogenic antigen, such as a bacterial antigen or viral antigen. In some embodiments, wherein the antigen is a viral antigen, the viral antigen can optionally be from hepatitis A. hepatitis B. hepatitis C virus (HCV), human papilloma virus (HPV), hepatitis viral infections, Epstein-Barr virus (EBV), human herpes virus 8 (HHV-8), human T-cell leukemia virus-1 (HTLV-1), human T-cell leukemia virus-2 (HTLV-2), or a cytomegalovirus (CMV).

In some embodiments, the viral antigen is an antigen from an HPV selected from among HPV-16, HPV-18, HPV-31, HPV-33 and HPV-35, such as an HPV-16 antigen that is an HPV-16 E6 or HPV-16 E7 antigen. In some embodiments, the viral antigen is an EBV antigen selected from among Epstein-Barr nuclear antigen (EBNA)-1, EBNA-2, EBNA-3A, EBNA-3B, EBNA-3C, EBNA-leader protein (EBNA-LP), latent membrane proteins LMP-1, LMP-2A and LMP-2B, EBV-EA, EBV-MA and EBV-VCA. In some embodiments, the viral antigen is an HTLV-antigen that is TAX. In some embodiments, the viral antigen is an HBV antigen that is a hepatitis B core antigen or a hepatitis B envelope antigen.

In some embodiments, antigen is a tumor antigen. In some embodiments, the antigen is selected from among glioma-associated antigen, 3-human chorionic gonadotropin, alpha fetoprotein (AFP), lectin-reactive AFP, thyroglobulin, RAGE-1, MN-CA IX, human telomerase reverse transcriptase, RUl, RU2 (AS), intestinal carboxyl esterase, muthsp70-2, M-CSF, Melanin-A/MART-1, WT-1, S-100, MBP, CD63, MUC1 (e.g. MUC1-8), p53, Ras, cyclin B1, HER-2/neu, carcinoembryonic antigen (CEA), gp100, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A11, MAGE-B1, MAGE-B2, MAGE-B3, MAGE-B4, MAGE-C1, BAGE, GAGE-1, GAGE-2, p15, tyrosinase (e.g. tyrosinase-related protein 1 (TRP-1) or tyrosinase-related protein 2 (TRP-2)), β-catenin, NY-ESO-1, LAGE-1a, PP1, MDM2, MDM4, EGVFvIII, Tax, SSX2, telomerase, TARP, pp65, CDK4, vimentin, S100, eIF-4A1, IFN-inducible p78, melanotransferrin (p97), Uroplakin II, prostate specific antigen (PSA), human kallikrein (huK2), prostate specific membrane antigen (PSM), and prostatic acid phosphatase (PAP), neutrophil elastase, ephrin B2, BA-46, Bcr-abl, E2A-PRL, H4-RET, IGH-IGK, MYL-RAR, Caspase 8, FRa, CD24, CD44, CD133, CD 166, epCAM, CA-125, HE4, Oval, estrogen receptor, progesterone receptor, uPA, PAI-1, CD19, CD20, CD22, ROR1, CD33/IL3Ra, c-Met, PSMA, Glycolipid F77, GD-2, insulin growth factor (IGF)-I, IGF-II, IGF-I receptor and mesothelin.

In any of the provided embodiments, the T cell can be a primary T cell derived from a subject, optionally wherein the subject is a human. In some embodiments, the T cell is a CD8+ T cell or subtypes thereof or a CD4+ T cell or subtypes thereof. In some embodiments, the T cell is derived from a multipotent or pluripotent cell, which optionally is an iPSC.

Also provided herein are compositions containing a plurality of genetically engineered T cells, such as a plurality of any of the engineered cells provided herein. In some embodiments, provided are compositions that contain a plurality of genetically engineered T cells comprising a modified T cell receptor alpha constant (TRAC) locus, the modified TRAC locus comprising a nucleic acid encoding a recombinant TCR or portion thereof, said recombinant TCR or portion thereof comprising: (i) a TCR alpha (TCRα) chain comprising a variable alpha (Vα) domain and a constant alpha (Cα) domain, and (ii) a TCR beta (TCRβ) chain comprising a variable beta (Vβ) domain and a constant beta (Cβ) domain, wherein the nucleic acid sequence comprises of (a) a transgene sequence encoding the TCRβ and the Vα domain, and (b) an open reading frame of the endogenous TRAC locus or a partial sequence thereof, wherein the open reading frame encodes at least a portion of the Cα domain of the recombinant TCR, and the recombinant TCR is capable of binding to an antigen that is associated with, specific to, and/or expressed on a cell or tissue that is associated with a disease, disorder, or condition. In some of any such embodiments, at least 70%, 75%, 80%, 90%, 91%, 92%, 93%, 94%, 95%, 96%, 97%, 98%, or 99% of the engineered cells in the composition comprise a genetic disruption in or of an endogenous T cell receptor alpha constant region (TRAC) gene and/or a T cell receptor beta constant region (TRBC) gene; at least 80%, 90%, 91%, 92%, 93%, 94%, 95%, 96%, 97%, 98%, or 99% of the engineered cells in the composition do not express or do not express detectable levels of a gene product of an endogenous TRAC or TRBC gene. In some of any such embodiments, at least or greater than 35%, 40%, 45%, 50%, 55%, 60%, 65%, 70%, 75%, 80%, or 90% of the cells in the composition express the recombinant TCR and/or exhibits binding to the antigen.

Also provided herein are compositions containing a plurality of genetically engineered T cells comprising a modified T cell receptor alpha constant (TRAC) locus, said modified TRAC locus comprising a nucleic acid encoding a recombinant TCR or portion thereof, said recombinant TCR or portion thereof comprising: (i) a TCR alpha (TCRα) chain comprising a variable alpha (Vα) domain and a constant alpha (Cα) domain, and (ii) a TCR beta (TCRβ) chain comprising a variable beta (Vβ) domain and a constant beta (Cβ) domain, wherein the nucleic acid sequence comprises of (a) a transgene sequence encoding the TCRβ and the Vα domain, and (b) an open reading frame of the endogenous TRAC locus or a partial sequence thereof, wherein the open reading frame encodes at least a portion of the Cα domain of the recombinant TCR. In some of any such embodiments, a portion of the Cα is encoded by the open reading frame of the endogenous TRAC locus or a partial sequence thereof, and a further portion of the Cα is encoded by the transgene sequence, wherein said further portion of Cα is less than the full length of a native Ca. In some of any such embodiments, the further portion of the Cα and/or the Cβ region encoded by the nucleic acid sequence of (a) comprises one or more modifications compared to a native Cα region and/or a native Cβ region, said one or more modifications introduces one or more cysteine residues that are capable of forming one or more non-native disulfide bridges between the alpha chain and beta chain.

Also provided herein are compositions containing a plurality of genetically engineered T cells comprising a modified T cell receptor beta constant (TRBC) locus, said modified TRBC locus comprising a nucleic acid encoding a recombinant TCR or portion thereof, said recombinant TCR or portion thereof comprising: (i) a TCR beta (TCRβ) chain comprising a variable beta (Vβ) domain and a constant beta (Cβ) domain, and (ii) a TCR alpha (TCRα) chain comprising a variable alpha (Vα) domain and a constant alpha (Cα) domain, wherein the nucleic acid sequence comprises of (a) a transgene sequence encoding the TCRα and the Vβ domain, and (b) an open reading frame of the endogenous TRBC locus or a partial sequence thereof, wherein the open reading frame encodes at least a portion of the Cβ domain of the recombinant TCR, and said recombinant TCR is capable of binding to an antigen that is associated with, specific to, and/or expressed on a cell or tissue that is associated with a disease, disorder, or condition. In some of any such embodiments, at least 70%, 75%, 80%, 90%, 91%, 92%, 93%, 94%, 95%, 96%, 97%, 98%, or 99% of the engineered cells in the composition comprise a genetic disruption in or of an endogenous T cell receptor alpha constant region (TRAC) gene and/or a T cell receptor beta constant region (TRBC) gene. In some of any such embodiments, at least 80%, 90%, 91%, 92%, 93%, 94%, 95%, 96%, 97%, 98%, or 99% of the engineered cells in the composition do not express or do not express detectable levels of a gene product of an endogenous TRAC or TRBC gene. In some of any such embodiments, at least or greater than 35%, 40%, 45%, 50%, 55%, 60%, 65%, 70%, 75%, 80%, or 90% of the cells in the composition express the recombinant TCR and/or exhibits binding to the antigen.

Also provided herein are compositions containing a plurality of genetically engineered T cells comprising a modified T cell receptor beta constant (TRBC) locus, said modified TRBC locus comprising a nucleic acid encoding a recombinant TCR or portion thereof, said recombinant TCR or portion thereof comprising: (i) a TCR beta (TCRβ) chain comprising a variable beta (Vβ) domain and a constant beta (Cβ) domain, and (ii) a TCR alpha (TCRα) chain comprising a variable alpha (Vα) domain and a constant alpha (Cα) domain, wherein the nucleic acid sequence comprises of (a) a transgene sequence encoding the TCRα and the Vβ domain, and (b) an open reading frame of the endogenous TRBC locus or a partial sequence thereof, wherein the open reading frame encodes at least a portion of the Cβ domain of the recombinant TCR. In some of any such embodiments, a portion of the Cβ is encoded by the open reading frame of the endogenous TRBC locus or a partial sequence thereof, and a further portion of the Cβ is encoded by the transgene sequence, wherein said further portion of Cβ is less than the full length of a native Cβ. In some of any such embodiments, the further portion of the Cβ and/or the Cα region encoded by the nucleic acid sequence of (a) comprises one or more modifications, optionally a replacement, deletion, or insertion of one or more amino acids compared to a native Cβ region and/or a native Cα region. In some of any such embodiments, the one or more modifications introduces one or more cysteine residues that are capable of forming one or more non-native disulfide bridges between the alpha chain and beta chain.

In some of any embodiments, the recombinant TCR is capable of binding to an antigen that is associated with, specific to, and/or expressed on a cell or tissue that is associated with a disease, disorder, or condition.

In some embodiments, provided herein is a composition containing a plurality of any of the genetically engineered T cells provided herein. In some of any of the embodiments, the composition contains CD4+ and/or CD8+ T cells. In some embodiments, the composition contains CD4+ and CD8+ T cells and the ratio of CD4+ to CD8+ T cells is from or from about 1:3 to 3:1, optionally 1:1.

In some embodiments, the composition contains at least 70%, 75%, 80%, 90%, 91%, 92%, 93%, 94%, 95%, 96%, 97%, 98%, or 99% of the engineered cells in the composition contain a genetic disruption in or of an endogenous T cell receptor alpha constant region (TRAC) gene and/or a T cell receptor beta constant region (TRBC) gene; and/or at least 80%, 90%, 91%, 92%, 93%, 94%, 95%, 96%, 97%, 98%, or 99% of the engineered cells in the composition do not express or do not express detectable levels of a gene product of an endogenous TRAC or TRBC gene. In some embodiments, at least or greater than 35%, 40%, 45%, 50%, 55%, 60%, 65%, 70%, 75%, 80%, or 90% of the cells in the composition express the recombinant TCR and/or exhibit antigen binding (e.g., exhibits binding to the antigen).

In some embodiments, provided are polynucleotides, such as polynucleotides for use in engineering cells. In some embodiments, provided here in are polynucleotides, containing (a) a nucleic acid sequence encoding a portion of a recombinant T cell receptor (TCR), said nucleic acid sequence encoding (i) a T cell receptor beta (TCRβ) chain containing a variable beta (Vβ) domain and a constant beta (Cβ) domain; and (ii) a portion of a T cell receptor alpha (TCRα) chain, wherein the portion of the TCRα chain is less than a full-length native TCRα chain, and (b) one or more homology arms linked to the nucleic acid sequence, wherein the one or more homology arms contain a sequence homologous to one or more region(s) of an open reading frame of a TRAC locus. In some embodiments, the polynucleotide is comprised in a viral vector.

Provided herein are polynulceotides that contain (a) a nucleic acid sequence encoding a portion of a recombinant T cell receptor (TCR), said nucleic acid sequence encoding (i) a T cell receptor beta (TCRβ) chain comprising a variable beta (Vβ) domain and a constant beta (Cβ) domain; and (ii) a portion of a T cell receptor alpha (TCRα) chain, wherein the portion of the TCRα chain is less than a full-length of a native TCRα chain, and (b) one or more homology arms linked to the nucleic acid sequence, wherein the one or more homology arms comprise a sequence homologous to one or more region(s) of an open reading frame of a TRAC locus; wherein, when the TCR or antigen-binding fragment thereof is expressed from a cell introduced with the polynucleotide: a portion of the Cα is encoded by the open reading frame of the endogenous TRAC locus or a partial sequence thereof, and a further portion of the Cα is encoded by the transgene sequence, wherein said further portion of Cα is less than the full length of a native Ca; and the further portion of the Cα and/or the Cβ region encoded by the nucleic acid sequence of (a) comprises one or more modifications compared to a native Cα region and/or a native Cβ region, said one or more modifications introduces one or more cysteine residues that are capable of forming one or more non-native disulfide bridges between the alpha chain and beta chain. In some aspects, and/or wherein the Cα and/or the Cβ of the recombinant TCR comprises one or more non-native cysteines.

Provided herein are polynulceotides that contain (a) a nucleic acid sequence encoding a portion of a recombinant T cell receptor (TCR), said nucleic acid sequence encoding (i) a T cell receptor beta (TCRβ) chain comprising a variable beta (Vβ) domain and a constant beta (Cβ) domain; and (ii) a portion of a T cell receptor alpha (TCRα) chain, wherein the portion of the TCRα chain is less than a full-length of a native TCRα chain, and (b) one or more homology arms linked to the nucleic acid sequence, wherein the one or more homology arms comprise a sequence homologous to one or more region(s) of an open reading frame of a TRAC locus; wherein the transgene sequence comprises one or more heterologous or regulatory control element(s) comprising a heterologous promoter, operably linked to control expression of the TCR when expressed from a cell introduced with the genetically engineered T cell.

In some embodiments, the TCRα chain contains a constant alpha region (Ca), wherein at least a portion of said Cα is encoded by the open reading frame of the endogenous TRAC locus or a partial sequence thereof when the TCR or antigen-binding fragment thereof is expressed from a cell introduced with the polynucleotide.

In some embodiments, wherein the nucleic acid sequence of (a) and the one of the one or more homology arms together contain a sequence of nucleotides encoding the Cα that is less than the full length of a native Ca, wherein at least a portion of the Cα is encoded by the open reading frame of the endogenous TRAC locus or a partial sequence thereof when the TCR or antigen-binding fragment thereof is expressed from a cell introduced with the polynucleotide.

In some embodiments, the nucleic acid sequence encoding the TCRβ chain is upstream of the nucleic acid sequence encoding the portion of the TCRα chain. In some embodiments, the nucleic acid sequence of (a) does not contain an intron. In some embodiments, the nucleic acid sequence of (a) does not contain a sequence encoding a 3′ untranslated region (3′ UTR). In some embodiments, the nucleic acid sequence of (a) is a sequence that is exogenous or heterologous to an open reading frame of an endogenous genomic TRAC locus of a T cell, optionally a human T cell. In some embodiments, the nucleic acid sequence of (a) is in-frame with one or more exons or a partial sequence thereof of the open reading frame of the TRAC locus contained in the one or more homology arm(s). In some embodiments, the one or more exons or a partial sequence thereof of the open reading frame contains a sequence within exon 1 of the open reading frame of the TRAC locus.

In some embodiments, the TCRα chain is capable of dimerizing with a TCRβ chain, when produced from a cell introduced with the polynucleotide. In some embodiments, the TCRα chain contains a variable alpha (Vα) domain.

In some embodiments, a portion of the Cα is encoded by the open reading frame of the endogenous TRAC locus or a partial sequence thereof, and a further portion of the Cα is encoded by the nucleic acid sequence of (a), wherein said further portion of Cα is less than the full length of a native Cα. In some of any embodiments, the open reading frame or the partial sequence thereof comprises at least one intron and at least one exon of the endogenous TRAC locus. In some embodiments, the further portion of the Cα is encoded by a sequence of nucleotides starting from residue 3 and up to residue 3155 of the sequence set forth in SEQ ID NO:1 or one or more exons thereof or a sequence that exhibits at least 85%, 86%, 87%, 88%, 89%, 90%, 91%, 92%, 93%, 94%, 95%, 96%, 97%, 98%, 99% or more sequence identity to a sequence of nucleotides starting from residue 3 and up to residue 3155 of the sequence set forth in SEQ ID NO:1 or one or more exons thereof, or a partial sequence thereof. In some embodiments, the Cα is encoded by a sequence of nucleotides that is less than 400, less than 300, less than 250, less than 200, or less than 150 base pairs in length.

In some embodiments, the further portion of the Cα is encoded by a sequence of nucleotides that contains less than four exons, less than three exons, less than two exons, one exon, or less than one full exon the open reading frame of the TRAC locus. In some embodiments, the further portion of the Cα is encoded by a portion of exon 1 of the TRAC locus, wherein the portion of exon 1 is less than the full length of exon 1 the open reading frame of the TRAC locus, such as wherein the portion of exon 1 contains a 5′ portion of exon 1.

In some of any such embodiments, the encoded Cα comprises the sequence selected from any one of SEQ ID NOS: 19, 24 and 243-252, or a sequence that exhibits at least 85%, 86%, 87%, 88%, 89%, 90%, 91%, 92%, 93%, 94%, 95%, 96%, 97%, 98%, 99% or more sequence identity to any one of SEQ ID NOS: 19, 24 and 243-252, or a partial sequence thereof, when produced from a cell introduced with the polynucleotide. In some of any such embodiments, the at least a portion of Cα is encoded by a sequence of nucleotides starting from residue 3 and up to residue 3155 of the sequence set forth in SEQ ID NO:1 or one or more exons thereof or a sequence that exhibits at least 85%, 86%, 87%, 88%, 89%, 90%, 91%, 92%, 93%, 94%, 95%, 96%, 97%, 98%, 99% or more sequence identity to a sequence of nucleotides starting from residue 3 and up to residue 3155 of the sequence set forth in SEQ ID NO:1 or one or more exons thereof, or a partial sequence thereof.

In some embodiments, the further portion of the Cα contains a sequence set forth in SEQ ID NO:142, or a sequence that exhibits at least 85%, 86%, 87%, 88%, 89%, 90%, 91%, 92%, 93%, 94%, 95%, 96%, 97%, 98%, 99% or more sequence identity to SEQ ID NO:142, or a partial sequence thereof.

In some embodiments, the further portion of the Cα and/or the Cβ region encoded by the nucleic acid sequence of (a) contains one or more modifications, optionally a replacement, deletion, or insertion of one or more amino acids compared to a native Cα region and/or a native Cβ region, optionally said one or more modifications introduces one or more cysteine residues that are capable of forming one or more non-native disulfide bridges between the alpha chain and beta chain.

In some of any embodiments, the introduction of one or more cysteine residues comprises replacement of a non-cysteine residue with a cysteine residue. In some of any embodiments, the encoded Cα region comprises a cysteine at a position corresponding to position 48 with numbering as set forth in any of SEQ ID NO: 24; and/or the encoded Cβ region comprises a cysteine at a position corresponding to position 57 with numbering as set forth in SEQ ID NO: 20, when produced from a cell introduced with the polynucleotide.

In some of any embodiments, the encoded Cα comprises the sequence selected from any one of SEQ ID NOS: 248-252, or a partial sequence thereof, when produced from a cell introduced with the polynucleotide. In some of any embodiments, the portion of the TCRα chain comprises a variable alpha (Vα) domain.

In some embodiments, the one or more homology arm contains a 5′ homology arm and/or a 3′ homology arm. In some embodiments, the 5′ homology arm and 3′ homology arm contains nucleic acid sequences homologous to nucleic acid sequences surrounding a target site, wherein the target site is within the TRAC locus, such as within exon 1 of the TRAC locus.

In some embodiments, the 5′ homology arm contains: a) a sequence containing at or at least at or at least 150, 200, 250, 300, 350, 400, 450, 500, 550, or 600 contiguous nucleotides to a sequence that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 124; b) a sequence containing at or at least at or at least 150, 200, 250, 300, 350, 400, 450, 500, 550, or 600 contiguous nucleotides of the sequence set forth in SEQ ID NO:124; or c) the sequence set forth in SEQ ID NO: 124.

In some embodiments, the 3′ homology arm contains: a) a sequence containing at or at least at or at least 150, 200, 250, 300, 350, 400, 450, 500, 550, or 600 contiguous nucleotides to a sequence that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 125; b) a sequence containing at or at least at or at least 150, 200, 250, 300, 350, 400, 450, 500, 550, or 600 contiguous nucleotides of the sequence set forth in SEQ ID NO:125; or c) the sequence set forth in SEQ ID NO: 125.

In some embodiments, provided herein is a polynucleotide, containing: (a) a nucleic acid sequence encoding a portion of a recombinant T cell receptor (TCR), said nucleic acid sequence encoding (i) a T cell receptor alpha (TCRα) chain containing a variable alpha (Vα) domain and a constant alpha (Cα) domain; and (ii) a portion of a T cell receptor beta (TCRβ) chain, wherein the portion of the TCRβ chain is less than a full-length native TCRβ chain, and (b) one or more homology arms linked to the nucleic acid sequence, wherein the one or more homology arms contain a sequence homologous to one or more region(s) of an open reading frame of a TRBC locus. In some of such embodiments, the TCRβ chain contains a constant beta (Cβ), wherein at least a portion of said C is encoded by the open reading frame of the endogenous TRAC locus or a partial sequence thereof when the TCR or antigen-binding fragment thereof is expressed from a cell introduced with the polynucleotide. In some embodiments, the nucleic acid sequence of (a) and the one of the one or more homology arms together contain a sequence of nucleotides encoding the Cβ that is less than the full length of a native C, wherein at least a portion of the Cβ is encoded by the open reading frame of the endogenous TRBC locus or a partial sequence thereof when the TCR or antigen-binding fragment thereof is expressed from a cell introduced with the polynucleotide.

In some embodiments, the TRBC locus is one or more of TRBC1 or TRBC2.

In some embodiments, the nucleic acid sequence encoding the TCRα chain is upstream of nucleic acid sequence encoding the portion of the TCRβ chain. In some embodiments, the nucleic acid sequence of (a) does not contain an intron. In some embodiments, the nucleic acid sequence of (a) does not contain a sequence encoding a 3′ untranslated region (3′ UTR). In some embodiments, the nucleic acid sequence of (a) is a sequence that is exogenous or heterologous to an open reading frame of an endogenous genomic TRBC locus of a T cell, optionally a human T cell.

In some embodiments, the nucleic acid sequence of (a) is in-frame with one or more exons or a partial sequence thereof of the open reading frame of the TRAC locus contained in the one or more homology arm(s). In some embodiments, the one or more exons or a partial sequence thereof of the open reading frame is or contains a sequence within exon 1 of the open reading frame of the TRBC locus.

In some embodiments, the TCRβ chain is capable of dimerizing with a TCRα chain, when produced from a cell introduced with the polynucleotide. In some of any embodiments, the encoded Cβ comprises the sequence selected from any one of SEQ ID NO: 20, 21, 25 and 253-258 or a sequence that exhibits at least 85%, 86%, 87%, 88%, 89%, 90%, 91%, 92%, 93%, 94%, 95%, 96%, 97%, 98%, 99% or more sequence identity to any one of SEQ ID NO: 20, 21, 25 and 253-258, or a partial sequence thereof, when produced from a cell introduced with the polynucleotide.

In some embodiments, the portion of the TCRβ chain contains a variable beta (V) domain. In some embodiments, a portion of the Cβ is encoded by the open reading frame of the endogenous TRBC locus or a partial sequence thereof, and a further portion of the Cβ is encoded by the nucleic acid sequence of (a), wherein said further portion of Cβ is less than the full length of a native Cβ. In some of any embodiments, the open reading frame or the partial sequence thereof comprises at least one intron and at least one exon of the endogenous TRBC locus. In some of such embodiments, the further portion of the Cβ is encoded by: a sequence of nucleotides starting from residue 3 and up to residue 1445 of the sequence set forth in SEQ ID NO:2 or one or more exons thereof or a sequence that exhibits at least 85%, 86%, 87%, 88%, 89%, 90%, 91%, 92%, 93%, 94%, 95%, 96%, 97%, 98%, 99% or more sequence identity to a sequence of nucleotides starting from residue 3 and up to residue 1445 of the sequence set forth in SEQ ID NO:2 or one or more exons thereof, or a partial sequence thereof; or a sequence of nucleotides starting from residue 3 and up to residue 1486 of the sequence set forth in SEQ ID NO:3 or one or more exons thereof or a sequence that exhibits at least 85%, 86%, 87%, 88%, 89%, 90%, 91%, 92%, 93%, 94%, 95%, 96%, 97%, 98%, 99% or more sequence identity to a sequence of nucleotides starting from residue 3 and up to residue 1486 of the sequence set forth in SEQ ID NO:3 or one or more exons thereof, or a partial sequence thereof. In some of such embodiments,

the further portion of the Cβ is encoded by a sequence of nucleotides that is less than 400, less than 300, less than 250, less than 200, or less than 150 base pairs in length.

In some embodiments, the further portion of the Cβ is encoded by a sequence of nucleotides that encodes less than four exons, less than three exons, less than two exons, one exon, or less than one full exon of the open reading frame of the TRBC locus. In some embodiments, the further portion of the Cβ is encoded by a portion of exon 1 of a TRBC locus, wherein the portion of exon 1 is less than the full length of exon 1 of the open reading frame of the TRBC locus. In some embodiments, the further portion of the TCRα constant domain is encoded by a portion of exon 1 of the TRAC locus, wherein the portion of exon 1 contains a 5′ portion of exon 1.

In some embodiments, the further portion of the Cβ and/or the Cα region encoded by the nucleic acid sequence of (a) contains one or more modifications, optionally a replacement, deletion, or insertion of one or more amino acids compared to a native Cβ region and/or a native Cα region, optionally said one or more modifications introduces one or more cysteine residues that are capable of forming one or more non-native disulfide bridges between the alpha chain and beta chain.

In some of any embodiments, the introduction of one or more cysteine residues comprises replacement of a non-cysteine residue with a cysteine residue. In some of any embodiments, the encoded Cα region comprises a cysteine at a position corresponding to position 48 with numbering as set forth in any of SEQ ID NO: 24; and/or the encoded Cβ region comprises a cysteine at a position corresponding to position 57 with numbering as set forth in SEQ ID NO: 20. In some of any embodiments, the encoded Cβ comprises the sequence selected from any one of SEQ ID NOS: 253 and 256-258, or a partial sequence thereof. In some of any embodiments, the portion of the TCRβ chain comprises a variable beta (V) domain.

In some embodiments, the one or more homology arm contains a 5′ homology arm and/or a 3′ homology arm. In some embodiments, the 5′ homology arm and 3′ homology arm contains nucleic acid sequences homologous to nucleic acid sequences surrounding a target site, wherein the target site is within the open reading frame of the TRBC locus. In some embodiments, the target site is within exon 1 of the open reading frame of the TRBC locus.

In some embodiments, the 5′ homology arm and 3′ homology arm independently are at least or at least about 10, 20, 30, 40, 50, 100, 200, 300, 400, 500, 600, 700, 800, 900, 1000, 1500, or 2000 nucleotides, or less than or less than about 10, 20, 30, 40, 50, 100, 200, 300, 400, 500, 600, 700, 800, 900, 1000, 1500, or 2000 nucleotides. In some of such embodiments, the 5′ homology arm and 3′ homology arm independently from or from about 100 to 1000 nucleotides, 100 to 750 nucleotides, 100 to 600 nucleotides, 100 to 400 nucleotides, 100 to 300 nucleotides, 100 to 200 nucleotides, 200 to 1000 nucleotides, 200 to 750 nucleotides, 200 to 600 nucleotides, 200 to 400 nucleotides, 200 to 300 nucleotides, 300 to 1000 nucleotides, 300 to 750 nucleotides, 300 to 600 nucleotides, 300 to 400 nucleotides, 400 to 1000 nucleotides, 400 to 750 nucleotides, 400 to 600 nucleotides, 600 to 1000 nucleotides, 600 to 750 nucleotides or 750 to 1000 nucleotides in length. In some embodiments, the 5′ homology arm and 3′ homology arm independently are, are about, or are less than about 600 nucleotides in length.

In some of any embodiments, the nucleic acid sequence of (a) is a sequence that is exogenous or heterologous to an open reading frame of an endogenous genomic TRAC locus of a T cell, optionally a human T cell. In some of any embodiments, the 5′ homology arm and 3′ homology arm independently are at least or at least about 10, 20, 30, 40, 50, 100, 200, 300, 400, 500, 600, 700, 800, 900, 1000, 1500, or 2000 nucleotides, or less than or less than about 10, 20, 30, 40, 50, 100, 200, 300, 400, 500, 600, 700, 800, 900, 1000, 1500, or 2000 nucleotides. In some of any embodiments, the 5′ homology arm and 3′ homology arm independently are between at or about 50 and at or about 100 nucleotides in length, at or about 100 and at or about 250 nucleotides in length, at or about 250 and at or about 500 nucleotides in length, at or about 500 and at or about 750 nucleotides in length, at or about 750 and at or about 1000 nucleotides in length, or at or about 1000 and at or about 2000 nucleotides in length. In some of any embodiments, the 5′ homology arm and 3′ homology arm independently are at or about 200, 300, 400, 500, 600, 700 or 800 nucleotides in length, or any value between any of the foregoing.

In some of any embodiments, the 5′ homology arm and 3′ homology arm independently are greater than at or about 300 nucleotides in length, optionally wherein the 5′ homology arm and 3′ homology arm independently are at or about 400, 500 or 600 nucleotides in length or any value between any of the foregoing. In some of any such embodiments, the 5′ homology arm and 3′ homology arm independently are greater than at or about 300 nucleotides in length.

In some embodiments, the nucleic acid sequence of (a) contains one or more multicistronic element(s). In some embodiments, the multicistronic element(s) is positioned between the nucleic acid sequence encoding the TCRα or a portion thereof and the nucleic acid sequence encoding the TCRβ or a portion thereof. In some embodiments, the one or more multicistronic element(s) are upstream of the nucleic acid sequence encoding the TCR or a portion of the TCR or the nucleic acid molecule encoding the TCR. In some embodiments, the multicistronic element is or contains a ribosome skip sequence, optionally T2A, P2A, E2A, or F2A.

In some embodiments, the provided polynucleotide further contains one or more heterologous regulatory or control element(s). In some embodiments, the nucleic acid sequence of (a) contains one or more heterologous or regulatory control element(s) operably linked to control expression of the TCR when expressed from a cell introduced with the polynucleotide, such as a promoter, an enhancer, an intron, a polyadenylation signal, a Kozak consensus sequence, a splice acceptor sequence and/or a splice donor sequence. In some embodiments, the heterologous regulatory or control element contains a heterologous promoter, such as a heterologous promoter is selected from among a constitutive promoter, an inducible promoter, a repressible promoter, and/or a tissue-specific promoter. In some embodiments, the heterologous promoter is or contains a human elongation factor 1 alpha (EF1α) promoter or an MND promoter or a variant thereof.

Any of the provided polynucleotides can be contained in a viral vector. In some embodiments, the viral vector is an AAV vector, such as an AAV vector selected from among AAV1, AAV2, AAV3, AAV4, AAV5, AAV6, AAV7 or AAV8 vector. In some embodiments, the AAV vector is an AAV2 or AAV6 vector.

In some embodiments, the viral vector is a retroviral vector or a lentiviral vector.

In some embodiments, the polynucleotide is a linear polynucleotide. In some embodiments, the linear polynucleotide is a double-stranded polynucleotide or a single-stranded polynucleotide.

In some embodiments, the polynucleotide contains the structure: [5′ homology arm]-[nucleic acid sequence of (a)]-[3′ homology arm]. In some embodiments, the polynucleotide contains the structure: [5′ homology arm]-[multicistronic element]-[nucleic acid sequence of (a)]-[3′ homology arm]. In some embodiments, the polynucleotide contains the structure: [5′ homology arm]-[promoter]-[nucleic acid sequence of (a)]-[3′ homology arm].

In some embodiments, the recombinant TCR encoded by the polynucleotide is capable of binding to an antigen that is associated with, specific to, and/or expressed on a cell or tissue that is associated with a disease, disorder, or condition, such as an infectious disease or disorder, an autoimmune disease, an inflammatory disease, a tumor, or a cancer.

In some embodiments, the antigen is a tumor antigen or a pathogenic antigen. In some of such embodiments, the pathogenic antigen is a bacterial antigen or viral antigen. In some embodiments, the antigen is a viral antigen, optionally a viral antigen from hepatitis A, hepatitis B, hepatitis C virus (HCV), human papilloma virus (HPV), hepatitis viral infections, Epstein-Barr virus (EBV), human herpes virus 8 (HHV-8), human T-cell leukemia virus-1 (HTLV-1), human T-cell leukemia virus-2 (HTLV-2), or a cytomegalovirus (CMV). In some embodiments, the antigen is an antigen from an HPV selected from among HPV-16, HPV-18, HPV-31, HPV-33 and HPV-35, such as an HPV-16 antigen that is an HPV-16 E6 or HPV-16 E7 antigen.

In some embodiments, the viral antigen is an EBV antigen selected from among Epstein-Barr nuclear antigen (EBNA)-1, EBNA-2, EBNA-3A, EBNA-3B, EBNA-3C, EBNA-leader protein (EBNA-LP), latent membrane proteins LMP-1, LMP-2A and LMP-2B, EBV-EA, EBV-MA and EBV-VCA. In some embodiments, the viral antigen is an HTLV-antigen that is TAX.

In some embodiments, the viral antigen is an HBV antigen that is a hepatitis B core antigen or a hepatitis B envelope antigen.

In some embodiments, the antigen is a tumor antigen, such as an antigen is selected from among glioma-associated antigen, 3-human chorionic gonadotropin, alpha fetoprotein (AFP), lectin-reactive AFP, thyroglobulin, RAGE-1, MN-CA IX, human telomerase reverse transcriptase, RUl, RU2 (AS), intestinal carboxyl esterase, mut hsp70-2, M-CSF, Melanin-A/MART-1, WT-1, S-100, MBP, CD63, MUC1 (e.g. MUC1-8), p53, Ras, cyclin B1, HER-2/neu, carcinoembryonic antigen (CEA), gp100, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A11, MAGE-B1, MAGE-B2, MAGE-B3, MAGE-B4, MAGE-C1, BAGE, GAGE-1, GAGE-2, p15, tyrosinase (e.g. tyrosinase-related protein 1 (TRP-1) or tyrosinase-related protein 2 (TRP-2)), β-catenin, NY-ESO-1, LAGE-1a, PP1, MDM2, MDM4, EGVFvIII, Tax, SSX2, telomerase, TARP, pp65, CDK4, vimentin, S100, eIF-4A1, IFN-inducible p78, melanotransferrin (p97), Uroplakin II, prostate specific antigen (PSA), human kallikrein (huK2), prostate specific membrane antigen (PSM), and prostatic acid phosphatase (PAP), neutrophil elastase, ephrin B2, BA-46, Bcr-abl, E2A-PRL, H4-RET, IGH-IGK, MYL-RAR, Caspase 8, FRa, CD24, CD44, CD133, CD 166, epCAM, CA-125, HE4, Oval, estrogen receptor, progesterone receptor, uPA, PAI-1, CD19, CD20, CD22, ROR1, CD33/IL3Ra, c-Met, PSMA, Glycolipid F77, GD-2, insulin growth factor (IGF)-I, IGF-II, IGF-I receptor and mesothelin.

In some of any embodiments, the provided polynucleotide is at least at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length, or any value between any of the foregoing. In some of any embodiments, the polynucleotide is between at or about 2500 and at or about 5000 nucleotides, at or about 3500 and at or about 4500 nucleotides, or at or about 3750 nucleotides and at or about 4250 nucleotides in length.

In some embodiments, provided herein is a method of producing a genetically engineered T cell containing a modified TRAC locus, containing introducing any of the provided nucleotides into a T cell containing a genetic disruption at a TRAC locus.

In some embodiments, provided herein is a method of producing a genetically engineered T cell containing a modified T cell receptor alpha constant (TRAC) locus, the method including: (a) introducing, into a T cell, one or more agent(s) capable of inducing a genetic disruption at a target site within an endogenous TRAC locus of the T cell; and (b) introducing into the T cell a polynucleotide containing a transgene encoding a T cell receptor beta (TCRβ) chain and a portion of a T cell receptor alpha (TCRα) chain, wherein the portion is less than a full-length native TCRα chain, and wherein the transgene is targeted for integration within the endogenous TRAC locus via homology directed repair (HDR), thereby producing a genetically engineered cell containing a modified TRAC locus.

In some embodiments, the introduction of the template polynucleotide is performed after the introduction of the one or more agent(s) capable of inducing a genetic disruption.

Also provided herein are methods, such as methods of producing a genetically engineered T cell. In some embodiments, provided are methods of producing a genetically engineered T cell comprising a modified T cell receptor alpha constant (TRAC) locus, the method comprising: (a) introducing into a T cell, one or more agent(s) capable of inducing a genetic disruption at a target site within an endogenous TRAC locus of the T cell; and (b) introducing into the T cell a polynucleotide comprising a transgene sequence encoding a T cell receptor beta (TCRβ) chain and a portion of a T cell receptor alpha (TCRα) chain, wherein the portion is less than a full-length native TCRα chain; and the transgene is targeted for integration within the endogenous TRAC locus via homology directed repair (HDR); thereby producing a genetically engineered cell comprising a modified TRAC locus. In some of any such embodiments, upon targeted integration of the transgene: a portion of the Cα is encoded by the open reading frame of the endogenous TRAC locus or a partial sequence thereof, and a further portion of the Cα is encoded by the transgene sequence, wherein said further portion of Cα is less than the full length of a native Cα; and the further portion of the Cα and/or the Cβ region encoded by the nucleic acid sequence of (a) comprises one or more modifications compared to a native Cα region and/or a native Cβ region, said one or more modifications introduces one or more cysteine residues that are capable of forming one or more non-native disulfide bridges between the alpha chain and beta chain. In some embodiments, the Cα and/or the Cβ of the recombinant TCR comprises one or more non-native cysteines.

Also provided are methods of producing a genetically engineered T cell comprising a modified T cell receptor alpha constant (TRAC) locus, the method comprising introducing, into a T cell, a polynucleotide comprising a transgene sequence encoding a T cell receptor beta (TCRβ) chain and a portion of a T cell receptor alpha (TCRα) chain, said T cell having a genetic disruption within the endogenous TRAC locus of the T cell; and the transgene is targeted for integration within the endogenous TRAC locus via homology directed repair (HDR); thereby producing a genetically engineered cell comprising a modified TRAC locus, wherein, upon targeted integration of the transgene: a portion of the Cα is encoded by the open reading frame of the endogenous TRAC locus or a partial sequence thereof, and a further portion of the Cα is encoded by the transgene sequence, wherein said further portion of Cα is less than the full length of a native Cα; and the further portion of the Cα and/or the Cβ region encoded by the nucleic acid sequence of (a) comprises one or more modifications compared to a native Cα region and/or a native Cβ region, said one or more modifications introduces one or more cysteine residues that are capable of forming one or more non-native disulfide bridges between the alpha chain and beta chain and/or wherein the Cα and/or the Cβ of the recombinant TCR comprises one or more non-native cysteines.

In some embodiments, provided herein is a method of producing a genetically engineered T cell containing a modified T cell receptor alpha constant (TRAC) locus, the method containing introducing, into a T cell, a polynucleotide containing a transgene encoding a T cell receptor beta (TCRβ) chain and a portion of a T cell receptor alpha (TCRα) chain, said T cell having a genetic disruption within the endogenous TRAC locus of the T cell, wherein the transgene is targeted for integration within the endogenous TRAC locus via homology directed repair (HDR), thereby producing a genetically engineered cell containing a modified TRAC locus. In some of such embodiments, the genetic disruption has been induced by one or more agent(s) capable of inducing a genetic disruption of one or more target site within the endogenous TRAC locus.

In some embodiments of the provided methods, the polynucleotide is from any of the polynucleotide provided herein.

In some embodiments of the provided methods, the modified TRAC locus contains a nucleic acid sequence encoding a recombinant TCR or portion thereof, wherein the nucleic acid sequence contains an in-frame fusion of (i) a transgene sequence and (ii) an open reading frame or a partial sequence thereof of the endogenous TRAC locus. In some embodiments, the transgene sequence does not contain a sequence encoding a 3′ untranslated region (3′ UTR) or an intron. In some embodiments, the open reading frame or a partial sequence thereof contains a 3′ UTR of the endogenous TRAC locus. In some embodiments, the transgene sequence is in-frame with one or more exons of the open reading frame or partial sequence thereof of the endogenous TRAC locus.

In some embodiments, a portion of the Cα is encoded by the open reading frame of the endogenous TRAC locus or a partial sequence thereof, and a further portion of the Cα is encoded by the transgene sequence, wherein said further portion of Cα is less than the full length of a native Cα. In some embodiments, the open reading frame or the partial sequence thereof contains at least one intron and at least one exon of the endogenous TRAC locus. In some embodiments, the transgene sequence is in-frame with one or more exons of the open reading frame or partial sequence thereof of the endogenous TRAC locus. In some embodiments, the transgene sequence is or has been integrated downstream of the most 5′ nucleotide of exon 1 and upstream of the most 3′ nucleotide of exon 1 of the open reading frame of the endogenous TRAC locus. In some embodiments, the at least a portion of Cα is encoded by at least exons 2-4 of the open reading frame of the endogenous TRAC locus.

In some embodiments of the provide methods, the encoded Cα contains the sequence selected from any one of SEQ ID NOS: 14, 15, 19, or 24, or a sequence that exhibits at least 85%, 86%, 87%, 88%, 89%, 90%, 91%, 92%, 93%, 94%, 95%, 96%, 97%, 98%, 99% or more sequence identity to any one of SEQ ID NOS: 14, 15, 19, or 24, or a partial sequence thereof. In some embodiments, the further portion of the Cα contains a sequence set forth in SEQ ID NO:142, or a sequence that exhibits at least 85%, 86%, 87%, 88%, 89%, 90%, 91%, 92%, 93%, 94%, 95%, 96%, 97%, 98%, 99% or more sequence identity to SEQ ID NO:142, or a partial sequence thereof.

In some embodiments of the provided methods, the engineered T cell further contains inducing a genetic disruption at a TRBC locus. In some embodiments, the engineered T cell contains a genetic disruption at a TRBC1 locus and/or a TRBC2 locus.

In some embodiments, provided herein is a method of producing a genetically engineered T cell containing a modified TRBC locus, containing introducing any of the polynucleotides provided herein into a T cell containing a genetic disruption at a TRBC locus.

In some embodiments, provided herein is a method of producing a genetically engineered T cell containing a modified T cell receptor beta constant (TRBC) locus, the method including: (a) introducing, into a T cell, one or more agent(s) capable of inducing a genetic disruption at a target site within an endogenous TRBC locus of the T cell; and (b) introducing into the T cell a polynucleotide containing a transgene encoding a T cell receptor alpha (TCRα) chain and a portion of a T cell receptor beta (TCRβ) chain, wherein the portion is less than a full-length native TCRβ chain, and wherein the transgene is targeted for integration within an endogenous TRBC locus via homology directed repair (HDR), thereby producing a genetically engineered cell containing a modified TRBC locus.

In some embodiments, provided herein is a method of producing a genetically engineered T cell containing a modified T cell receptor beta constant (TRBC) locus, the method containing introducing, into a T cell, a polynucleotide containing a transgene encoding a T cell receptor alpha (TCRα) chain and a portion of a T cell receptor beta (TCRβ) chain, said T cell having a genetic disruption within an endogenous TRBC locus of the T cell, wherein the transgene is targeted for integration within the endogenous TRBC locus via homology directed repair (HDR), thereby producing a genetically engineered cell containing a modified TRBC locus. In some of such embodiments, the genetic disruption has been induced by one or more agent(s) capable of inducing a genetic disruption of one or more target site within the endogenous TRBC locus. In some embodiments, the transgene sequence is in-frame with one or more exons of the open reading frame or partial sequence thereof of the endogenous TRBC locus. In some embodiments, the TRBC locus is a TRBC1 locus and/or a TRBC2 locus.

In some embodiments, the polynucleotide is any of the polynucleotides provided herein.

In some embodiments of the provided method, the modified TRBC locus contains a nucleic acid sequence encoding a recombinant TCR or portion thereof, wherein the nucleic acid sequence contains an in-frame fusion of (i) a transgene sequence and (ii) an open reading frame or a partial sequence thereof of the endogenous TRBC locus. In some embodiments, the transgene sequence does not contain a sequence encoding a 3′ untranslated region (3′ UTR) or an intron. In some embodiments, the open reading frame or a partial sequence thereof contains a 3′ UTR of the endogenous TRBC locus. In some embodiments, a portion of the Cβ is encoded by the open reading frame of the endogenous TRBC locus or a partial sequence thereof, and a further portion of the Cβ is encoded by the transgene sequence, wherein said further portion of Cβ is less than the full length of a native Cβ.

In some embodiments, the open reading frame or the partial sequence thereof contains at least one intron and at least one exon of the endogenous TRBC locus. In some embodiments, the transgene sequence is in-frame with one or more exons of the open reading frame or partial sequence thereof of the endogenous TRBC locus. In some embodiments, the transgene sequence is or has been integrated downstream of the most 5′ nucleotide of exon 1 and upstream of the most 3′ nucleotide of exon 1 of the open reading frame of the endogenous TRBC locus.

In some embodiments of the provided methods, the at least a portion of Cβ is encoded by at least exons 2-4 of the open reading frame of the endogenous TRBC locus. In some embodiments, the encoded Cβ contains the sequence selected from any one of SEQ ID NO: 16, 17, 20, 21, or 25, or a sequence that exhibits at least 85%, 86%, 87%, 88%, 89%, 90%, 91%, 92%, 93%, 94%, 95%, 96%, 97%, 98%, 99% or more sequence identity to SEQ ID NO: 16, 17, 20, 21, or 25, or a partial sequence thereof.

In some embodiments of the provided methods, the engineered T cell further contains inducing a genetic disruption at a TRAC locus.

In some embodiments of the provided methods, the one or more agent(s) capable of inducing a genetic disruption contains a DNA binding protein or DNA-binding nucleic acid that specifically binds to or hybridizes to the target site. In some embodiments, the one or more agent capable of inducing a genetic disruption contains (a) a fusion protein containing a DNA-targeting protein and a nuclease or (b) an RNA-guided nuclease. In some of such embodiments, the DNA-targeting protein or RNA-guided nuclease contains a zinc finger protein (ZFP), a TAL protein, or a clustered regularly interspaced short palindromic nucleic acid (CRISPR)-associated nuclease (Cas) specific for the target site. In some embodiments, the one or more agent contains a zinc finger nuclease (ZFN), a TAL-effector nuclease (TALEN), or and a CRISPR-Cas9 combination that specifically binds to, recognizes, or hybridizes to the target site.

In some embodiments, the each of the one or more agent contains a guide RNA (gRNA) having a targeting domain that is complementary to the at least one target site. In some embodiments, the one or more agent is introduced as a ribonucleoprotein (RNP) complex containing the gRNA and a Cas9 protein. In some embodiments, the RNP is introduced via electroporation, particle gun, calcium phosphate transfection, cell compression or squeezing.

In some embodiments, the one or more agent is introduced as one or more polynucleotide encoding the gRNA and/or a Cas9 protein. In some embodiments, the gRNA has a targeting domain that is complementary to a target site in a TRAC locus and contains a sequence selected from UCUCUCAGCUGGUACACGGC (SEQ ID NO:28), UGGAUUUAGAGUCUCUCAGC (SEQ ID NO:29), ACACGGCAGGGUCAGGGUUC (SEQ ID NO:30), GAGAAUCAAAAUCGGUGAAU (SEQ ID NO:31), GCUGGUACACGGCAGGGUCA (SEQ ID NO:32), CUCAGCUGGUACACGGC (SEQ ID NO:33), UGGUACACGGCAGGGUC (SEQ ID NO:34), GCUAGACAUGAGGUCUA (SEQ ID NO:35), GUCAGAUUUGUUGCUCC (SEQ ID NO:36), UCAGCUGGUACACGGCA (SEQ ID NO:37), GCAGACAGACUUGUCAC (SEQ ID NO:38), GGUACACGGCAGGGUCA (SEQ ID NO:39), CUUCAAGAGCAACAGUGCUG (SEQ ID NO:40), AGAGCAACAGUGCUGUGGCC (SEQ ID NO:41), AAAGUCAGAUUUGUUGCUCC (SEQ ID NO:42), ACAAAACUGUGCUAGACAUG (SEQ ID NO:43), AAACUGUGCUAGACAUG (SEQ ID NO:44), UGUGCUAGACAUGAGGUCUA (SEQ ID NO:45), GGCUGGGGAAGAAGGUGUCUUC (SEQ ID NO:46), GCUGGGGAAGAAGGUGUCUUC (SEQ ID NO:47), GGGGAAGAAGGUGUCUUC (SEQ ID NO:48), GUUUUGUCUGUGAUAUACACAU (SEQ ID NO:49), GGCAGACAGACUUGUCACUGGAUU (SEQ ID NO:50), GCAGACAGACUUGUCACUGGAUU (SEQ ID NO:51), GACAGACUUGUCACUGGAUU (SEQ ID NO:52), GUGAAUAGGCAGACAGACUUGUCA (SEQ ID NO:53), GAAUAGGCAGACAGACUUGUCA (SEQ ID NO:54), GAGUCUCUCAGCUGGUACACGG (SEQ ID NO:55), GUCUCUCAGCUGGUACACGG (SEQ ID NO:56), GGUACACGGCAGGGUCAGGGUU (SEQ ID NO:57) and GUACACGGCAGGGUCAGGGUU (SEQ ID NO:58). In some embodiments, the gRNA has a targeting domain containing the sequence GAGAAUCAAAAUCGGUGAAU (SEQ ID NO:31).

In some embodiments, the gRNA has a targeting domain that is complementary to a target site in one or both of a TRBC1 and a TRBC2 gene and contains a sequence selected from CACCCAGAUCGUCAGCGCCG (SEQ ID NO:59), CAAACACAGCGACCUCGGGU (SEQ ID NO:60), UGACGAGUGGACCCAGGAUA (SEQ ID NO:61), GGCUCUCGGAGAAUGACGAG (SEQ ID NO:62), GGCCUCGGCGCUGACGAUCU (SEQ ID NO:63), GAAAAACGUGUUCCCACCCG (SEQ ID NO:64), AUGACGAGUGGACCCAGGAU (SEQ ID NO:65), AGUCCAGUUCUACGGGCUCU (SEQ ID NO:66), CGCUGUCAAGUCCAGUUCUA (SEQ ID NO:67), AUCGUCAGCGCCGAGGCCUG (SEQ ID NO:68), UCAAACACAGCGACCUCGGG (SEQ ID NO:69), CGUAGAACUGGACUUGACAG (SEQ ID NO:70), AGGCCUCGGCGCUGACGAUC (SEQ ID NO:71), UGACAGCGGAAGUGGUUGCG (SEQ ID NO:72), UUGACAGCGGAAGUGGUUGC (SEQ ID NO:73), UCUCCGAGAGCCCGUAGAAC (SEQ ID NO:74), CGGGUGGGAACACGUUUUUC (SEQ ID NO:75), GACAGGUUUGGCCCUAUCCU (SEQ ID NO:76), GAUCGUCAGCGCCGAGGCCU (SEQ ID NO:77), GGCUCAAACACAGCGACCUC (SEQ ID NO:78), UGAGGGUCUCGGCCACCUUC (SEQ ID NO:79), AGGCUUCUACCCCGACCACG (SEQ ID NO:80), CCGACCACGUGGAGCUGAGC (SEQ ID NO:81), UGACAGGUUUGGCCCUAUCC (SEQ ID NO:82), CUUGACAGCGGAAGUGGUUG (SEQ ID NO:83), AGAUCGUCAGCGCCGAGGCC (SEQ ID NO:84), GCGCUGACGAUCUGGGUGAC (SEQ ID NO:85), UGAGGGCGGGCUGCUCCUUG (SEQ ID NO:86), GUUGCGGGGGUUCUGCCAGA (SEQ ID NO:87), AGCUCAGCUCCACGUGGUCG (SEQ ID NO:88), GCGGCUGCUCAGGCAGUAUC (SEQ ID NO:89), GCGGGGGUUCUGCCAGAAGG (SEQ ID NO:90), UGGCUCAAACACAGCGACCU (SEQ ID NO:91), ACUGGACUUGACAGCGGAAG (SEQ ID NO:92), GACAGCGGAAGUGGUUGCGG (SEQ ID NO:93), GCUGUCAAGUCCAGUUCUAC (SEQ ID NO:94), GUAUCUGGAGUCAUUGAGGG (SEQ ID NO:95), CUCGGCGCUGACGAUCU (SEQ ID NO:96), CCUCGGCGCUGACGAUC (SEQ ID NO:97), CCGAGAGCCCGUAGAAC (SEQ ID NO:98), CCAGAUCGUCAGCGCCG (SEQ ID NO:99), GAAUGACGAGUGGACCC (SEQ ID NO:100), GGGUGACAGGUUUGGCCCUAUC (SEQ ID NO:101), GGUGACAGGUUUGGCCCUAUC (SEQ ID NO:102), GUGACAGGUUUGGCCCUAUC (SEQ ID NO:103), GACAGGUUUGGCCCUAUC (SEQ ID NO:104), GAUACUGCCUGAGCAGCCGCCU (SEQ ID NO:105), GACCACGUGGAGCUGAGCUGGUGG (SEQ ID NO:106), GUGGAGCUGAGCUGGUGG (SEQ ID NO:107), GGGCGGGCUGCUCCUUGAGGGGCU (SEQ ID NO:108), GGCGGGCUGCUCCUUGAGGGGCU (SEQ ID NO:109), GCGGGCUGCUCCUUGAGGGGCU (SEQ ID NO:110), GGGCUGCUCCUUGAGGGGCU (SEQ ID NO:111), GGCUGCUCCUUGAGGGGCU (SEQ ID NO:112), GCUGCUCCUUGAGGGGCU (SEQ ID NO:113), GGUGAAUGGGAAGGAGGUGCACAG (SEQ ID NO:114), GUGAAUGGGAAGGAGGUGCACAG (SEQ ID NO:115) and GAAUGGGAAGGAGGUGCACAG (SEQ ID NO:116). In some embodiments, the gRNA has a targeting domain containing the sequence GGCCUCGGCGCUGACGAUCU (SEQ ID NO:63).

In some of any embodiments, the T cell is a primary T cell from a subject. In some of any embodiments, the subject has or is suspected of having the disease, or disorder condition. In some of any embodiments, the subject is or is suspected of being healthy.

In some embodiments of the method, the T cell is a CD8+ T cell, or a subtype thereof, or is a CD4+ T cell, or a subtype thereof. In some embodiments, the T cell is derived from a multipotent or pluripotent cell, which optionally is an iPSC. In some embodiments of the method, the T cell is autologous to the subject, or allogeneic to the subject.

In some embodiments of the method, the polynucleotide and/or the one or more polynucleotide encoding the gRNA and/or a Cas9 protein is contained in one or more vector(s), which optionally are viral vector(s), such as an AAV vector. In some embodiments, the AAV vector is selected from among AAV1, AAV2, AAV3, AAV4, AAV5, AAV6, AAV7 or AAV8 vector, such as an AAV2 or AAV6 vector. In some embodiments, the viral vector is a retroviral vector or a lentiviral vector.

In some embodiments of the method, the polynucleotide is a linear polynucleotide, such a as a double-stranded polynucleotide or a single-stranded polynucleotide.

In some embodiments of the provided methods, the introduction of the one or more agent capable of inducing a genetic disruption and the introduction of the template polynucleotide are performed simultaneously or sequentially, in any order. In some embodiments, the introduction of the template polynucleotide is performed after the introduction of the one or more agent capable of inducing a genetic disruption. In some embodiments, the template polynucleotide is introduced immediately after, or within about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after the introduction of one or more agents capable of inducing a genetic disruption.

In some embodiments, provided herein is an engineered T cell or a plurality of engineered T cells generated using any of the methods provided herein.

In some embodiments, provided herein is a composition, containing the engineered T cell or plurality of engineered cells provided herein. In some embodiments, the provided composition contains CD4+ and/or CD8+ T cells. In some embodiments, the composition contains CD4+ and CD8+ T cells and the ratio of CD4+ to CD8+ T cells is from or from about 1:3 to 3:1, optionally 1:1.

In some embodiments of the provided compositions, at least 70%, 75%, 80%, 90%, 91%, 92%, 93%, 94%, 95%, 96%, 97%, 98%, or 99% of the engineered cells in the composition contain a genetic disruption in or of an endogenous T cell receptor alpha constant region (TRAC) gene and/or a T cell receptor beta constant region (TRBC) gene; and/or at least 80%, 90%, 91%, 92%, 93%, 94%, 95%, 96%, 97%, 98%, or 99% of the engineered cells in the composition do not express or do not express detectable levels of a gene product of an endogenous TRAC or TRBC gene.

In some embodiments of the provided compositions, at least or greater than 35%, 40%, 45%, 50%, 55%, 60%, 65%, 70%, 75%, 80%, or 90% of the cells in the composition express the recombinant TCR and/or exhibit antigen binding (e.g., exhibits binding to the antigen).

In some embodiments, provided herein is a method of treatment containing administering any of the engineered cells, plurality of engineered cells or compositions provided here in to a subject.

In some embodiments, provided herein is a use of any of the engineered cells, plurality of engineered cells or compositions provided herein for the treatment of a disease or disorder.

In some embodiments, provided herein is a use of any of the engineered cells, plurality of engineered cells or compositions of provided herein in the manufacture of a medicament for treating a disease or disorder.

In some embodiments, provided herein is an engineered cell, plurality of engineered cells or composition of any of the engineered cells, plurality of engineered cells, or compositions provided herein for use in treating a disease or disorder.

In some embodiments, provided herein is an article of manufacture or a kit containing a polynucleotide provided herein, and one or more agent(s) capable of inducing a genetic disruption at a target site within a TRAC locus.

In some embodiments, provided herein is an article of manufacture or a kit containing a polynucleotide containing (a) a nucleic acid sequence encoding a T cell receptor beta (TCRβ) chain and a portion of a T cell receptor alpha (TCRα) chain, wherein the portion is less than a full-length native TCRα chain and (b) one or more homology arm(s) linked to the nucleic acid sequence, wherein the one or more homology arm(s) contain a sequence homologous to one or more region(s) of an open reading frame of a TRAC locus, said open reading frame encoding a TCRα chain; and one or more agent(s) capable of inducing a genetic disruption at a target site within a TRAC locus. In some of such embodiments, the polynucleotide is a polynucleotide provided herein.

In some embodiments, provided herein is an article of manufacture containing a polynucleotide provided herein, and one or more agent(s) capable of inducing a genetic disruption at a target site within a TRBC locus.

In some embodiments, provided herein is an article of manufacture containing a polynucleotide containing (a) a nucleic acid sequence encoding a T cell receptor alpha (TCRα) chain and a portion of a T cell receptor beta (TCRβ) chain, wherein the portion is less than a full-length native TCRβ chain and (b) one or more homology arm(s) linked to the nucleic acid sequence, wherein the one or more homology arm(s) contain(s) a sequence homologous to one or more region(s) of an open reading frame of a TRBC locus, said open reading frame encoding a TCRβ chain; and one or more agent(s) capable of inducing a genetic disruption at a target site within a TRAC locus. In some embodiments, the TRBC locus is TRBC1 and/or TRBC2. In some embodiments, the polynucleotide is selected from among the polynucleotides provided herein.

In some embodiments, the one or more agent(s) capable of inducing a genetic disruption contains a DNA binding protein or DNA-binding nucleic acid that specifically binds to or hybridizes to the target site. In some embodiments, the one or more agent capable of inducing a genetic disruption contains (a) a fusion protein containing a DNA-targeting protein and a nuclease or (b) an RNA-guided nuclease. In some of such embodiments, the DNA-targeting protein or RNA-guided nuclease contains a zinc finger protein (ZFP), a TAL protein, or a clustered regularly interspaced short palindromic nucleic acid (CRISPR)-associated nuclease (Cas) specific for the target site. In some embodiments, the one or more agent contains a zinc finger nuclease (ZFN), a TAL-effector nuclease (TALEN), or and a CRISPR-Cas9 combination that specifically binds to, recognizes, or hybridizes to the target site.

In some embodiments, the each of the one or more agent contains a guide RNA (gRNA) having a targeting domain that is complementary to the at least one target site. In some embodiments, the one or more agent is introduced as a ribonucleoprotein (RNP) complex containing the gRNA and a Cas9 protein. In some embodiments, the RNP is introduced via electroporation, particle gun, calcium phosphate transfection, cell compression or squeezing.

In some embodiments, the one or more agent is introduced as one or more polynucleotide encoding the gRNA and/or a Cas9 protein. In some embodiments, the gRNA has a targeting domain that is complementary to a target site in a TRAC locus and contains a sequence selected from UCUCUCAGCUGGUACACGGC (SEQ ID NO:28), UGGAUUUAGAGUCUCUCAGC (SEQ ID NO:29), ACACGGCAGGGUCAGGGUUC (SEQ ID NO:30), GAGAAUCAAAAUCGGUGAAU (SEQ ID NO:31), GCUGGUACACGGCAGGGUCA (SEQ ID NO:32), CUCAGCUGGUACACGGC (SEQ ID NO:33), UGGUACACGGCAGGGUC (SEQ ID NO:34), GCUAGACAUGAGGUCUA (SEQ ID NO:35), GUCAGAUUUGUUGCUCC (SEQ ID NO:36), UCAGCUGGUACACGGCA (SEQ ID NO:37), GCAGACAGACUUGUCAC (SEQ ID NO:38), GGUACACGGCAGGGUCA (SEQ ID NO:39), CUUCAAGAGCAACAGUGCUG (SEQ ID NO:40), AGAGCAACAGUGCUGUGGCC (SEQ ID NO:41), AAAGUCAGAUUUGUUGCUCC (SEQ ID NO:42), ACAAAACUGUGCUAGACAUG (SEQ ID NO:43), AAACUGUGCUAGACAUG (SEQ ID NO:44), UGUGCUAGACAUGAGGUCUA (SEQ ID NO:45), GGCUGGGGAAGAAGGUGUCUUC (SEQ ID NO:46), GCUGGGGAAGAAGGUGUCUUC (SEQ ID NO:47), GGGGAAGAAGGUGUCUUC (SEQ ID NO:48), GUUUUGUCUGUGAUAUACACAU (SEQ ID NO:49), GGCAGACAGACUUGUCACUGGAUU (SEQ ID NO:50), GCAGACAGACUUGUCACUGGAUU (SEQ ID NO:51), GACAGACUUGUCACUGGAUU (SEQ ID NO:52), GUGAAUAGGCAGACAGACUUGUCA (SEQ ID NO:53), GAAUAGGCAGACAGACUUGUCA (SEQ ID NO:54), GAGUCUCUCAGCUGGUACACGG (SEQ ID NO:55), GUCUCUCAGCUGGUACACGG (SEQ ID NO:56), GGUACACGGCAGGGUCAGGGUU (SEQ ID NO:57) and GUACACGGCAGGGUCAGGGUU (SEQ ID NO:58). In some embodiments, the gRNA has a targeting domain containing the sequence GAGAAUCAAAAUCGGUGAAU (SEQ ID NO:31).

In some embodiments, the gRNA has a targeting domain that is complementary to a target site in one or both of a TRBC1 and a TRBC2 gene and contains a sequence selected from CACCCAGAUCGUCAGCGCCG (SEQ ID NO:59), CAAACACAGCGACCUCGGGU (SEQ ID NO:60), UGACGAGUGGACCCAGGAUA (SEQ ID NO:61), GGCUCUCGGAGAAUGACGAG (SEQ ID NO:62), GGCCUCGGCGCUGACGAUCU (SEQ ID NO:63), GAAAAACGUGUUCCCACCCG (SEQ ID NO:64), AUGACGAGUGGACCCAGGAU (SEQ ID NO:65), AGUCCAGUUCUACGGGCUCU (SEQ ID NO:66), CGCUGUCAAGUCCAGUUCUA (SEQ ID NO:67), AUCGUCAGCGCCGAGGCCUG (SEQ ID NO:68), UCAAACACAGCGACCUCGGG (SEQ ID NO:69), CGUAGAACUGGACUUGACAG (SEQ ID NO:70), AGGCCUCGGCGCUGACGAUC (SEQ ID NO:71), UGACAGCGGAAGUGGUUGCG (SEQ ID NO:72), UUGACAGCGGAAGUGGUUGC (SEQ ID NO:73), UCUCCGAGAGCCCGUAGAAC (SEQ ID NO:74), CGGGUGGGAACACGUUUUUC (SEQ ID NO:75), GACAGGUUUGGCCCUAUCCU (SEQ ID NO:76), GAUCGUCAGCGCCGAGGCCU (SEQ ID NO:77), GGCUCAAACACAGCGACCUC (SEQ ID NO:78), UGAGGGUCUCGGCCACCUUC (SEQ ID NO:79), AGGCUUCUACCCCGACCACG (SEQ ID NO:80), CCGACCACGUGGAGCUGAGC (SEQ ID NO:81), UGACAGGUUUGGCCCUAUCC (SEQ ID NO:82), CUUGACAGCGGAAGUGGUUG (SEQ ID NO:83), AGAUCGUCAGCGCCGAGGCC (SEQ ID NO:84), GCGCUGACGAUCUGGGUGAC (SEQ ID NO:85), UGAGGGCGGGCUGCUCCUUG (SEQ ID NO:86), GUUGCGGGGGUUCUGCCAGA (SEQ ID NO:87), AGCUCAGCUCCACGUGGUCG (SEQ ID NO:88), GCGGCUGCUCAGGCAGUAUC (SEQ ID NO:89), GCGGGGGUUCUGCCAGAAGG (SEQ ID NO:90), UGGCUCAAACACAGCGACCU (SEQ ID NO:91), ACUGGACUUGACAGCGGAAG (SEQ ID NO:92), GACAGCGGAAGUGGUUGCGG (SEQ ID NO:93), GCUGUCAAGUCCAGUUCUAC (SEQ ID NO:94), GUAUCUGGAGUCAUUGAGGG (SEQ ID NO:95), CUCGGCGCUGACGAUCU (SEQ ID NO:96), CCUCGGCGCUGACGAUC (SEQ ID NO:97), CCGAGAGCCCGUAGAAC (SEQ ID NO:98), CCAGAUCGUCAGCGCCG (SEQ ID NO:99), GAAUGACGAGUGGACCC (SEQ ID NO:100), GGGUGACAGGUUUGGCCCUAUC (SEQ ID NO:101), GGUGACAGGUUUGGCCCUAUC (SEQ ID NO:102), GUGACAGGUUUGGCCCUAUC (SEQ ID NO:103), GACAGGUUUGGCCCUAUC (SEQ ID NO:104), GAUACUGCCUGAGCAGCCGCCU (SEQ ID NO:105), GACCACGUGGAGCUGAGCUGGUGG (SEQ ID NO:106), GUGGAGCUGAGCUGGUGG (SEQ ID NO:107), GGGCGGGCUGCUCCUUGAGGGGCU (SEQ ID NO:108), GGCGGGCUGCUCCUUGAGGGGCU (SEQ ID NO:109), GCGGGCUGCUCCUUGAGGGGCU (SEQ ID NO:110), GGGCUGCUCCUUGAGGGGCU (SEQ ID NO:111), GGCUGCUCCUUGAGGGGCU (SEQ ID NO:112), GCUGCUCCUUGAGGGGCU (SEQ ID NO:113), GGUGAAUGGGAAGGAGGUGCACAG (SEQ ID NO:114), GUGAAUGGGAAGGAGGUGCACAG (SEQ ID NO:115) and GAAUGGGAAGGAGGUGCACAG (SEQ ID NO:116). In some embodiments, the gRNA has a targeting domain containing the sequence GGCCUCGGCGCUGACGAUCU (SEQ ID NO:63).

In some embodiments, provided herein is a kit containing an article of manufacture provided herein, and instructions for use. In some of such embodiments, the instructions specify that the one or more agent(s) and the polynucleotide are introduced into the cell. In some embodiments, the instructions specify that the one or more agent(s) and the polynucleotide are introduced simultaneously or sequentially, in any order. In some embodiments, the instructions specify that the introduction of the polynucleotide is performed after the introduction of the one or more agent(s). In some of such embodiments, the instructions specify that the polynucleotide is introduced immediately after, or within about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after the introduction of one or more agents capable of inducing a genetic disruption.

In some of any such embodiments, the polynucleotide is introduced at or about 2 hours after the introduction of the one or more agents.

In some of any embodiments, the method is performed in a plurality of T cells. In some of any embodiments, the plurality T cells comprise CD4+ T cells, CD8+ T cells or CD4+ and CD8+ T cells. In some of any embodiments, the plurality of T cells comprise CD4+ and CD8+ T cells and the ratio of CD4+ to CD8+ T cells is at or about 1:3 to at or about 3:1, optionally at or about 1:2 to at or about 2:1, optionally at or about 1:1.

In some of any embodiments, prior to the introducing of the one or more agent, the method comprises incubating the cells, in vitro with a stimulatory agent(s) under conditions to stimulate or activate the one or more T cells. In some of any embodiments, the stimulatory agent (s) comprises and anti-CD3 and/or anti-CD28 antibodies, optionally anti-CD3/anti-CD28 beads, optionally wherein the bead to cell ratio is or is about 1:1. In some of any embodiments, the methods include removing the stimulatory agent(s) from the one or more immune cells prior to the introducing with the one or more agents.

In some of any embodiments, the method further comprises incubating the cells prior to, during or subsequent to the introducing of the one or more agents and/or the introducing of the template polynucleotide with one or more recombinant cytokines, optionally wherein the one or more recombinant cytokines are selected from the group consisting of IL-2, IL-7, and IL-15.

In some of any embodiments, the one or more recombinant cytokine is added at a concentration selected from a concentration of IL-2 from at or about 10 U/mL to at or about 200 U/mL, optionally at or about 50 IU/mL to at or about 100 U/mL; IL-7 at a concentration of 0.5 ng/mL to 50 ng/mL, optionally at or about 5 ng/mL to at or about 10 ng/mL and/or IL-15 at a concentration of 0.1 ng/mL to 20 ng/mL, optionally at or about 0.5 ng/mL to at or about 5 ng/mL.

In some of any embodiments, the incubation is carried out subsequent to the introducing of the one or more agents and the introducing of the template polynucleotide for up to or approximately 24 hours, 36 hours, 48 hours, 3, 4, 5, 6, 7, 8, 9, 10, 11, 12, 13, 14, 15, 16, 17, 18, 19, 20 or 21 days, optionally up to or about 7 days.

In some of any embodiments, the polynucleotide is at least at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length, or any value between any of the foregoing.

In some of any embodiments, the polynucleotide is between at or about 2500 and at or about 5000 nucleotides, at or about 3500 and at or about 4500 nucleotides, or at or about 3750 nucleotides and at or about 4250 nucleotides in length.

Also provided are cells generated using any of the methods described herein. In some aspects, also provided are compositions comprising such cells. Also provided are kits and articles of manufacture, for use in carrying out any of the methods provided herein. Also provided are methods of treatment, and therapeutic uses, such as therapeutic uses in treating a disease or disorder that involves administration of any of the cells or compositions containing cells described herein.

### BRIEF DESCRIPTION OF THE DRAWINGS

FIGS. 1A and 1B show histograms displaying cell surface staining of CD8, Vbeta22 (recombinant TCR-specific staining; FIG. 1A) and peptide-MHC tetramer complexed with antigen (HPV 16 E7(11-19) peptide; designated “HPV E7(11)”) recognized by the recombinant TCR (FIG. 1B), as assessed by flow cytometry, in T cells subject to knockout of endogenous TCR encoding genes (TRAC and TRBC) and engineered to express an exemplary recombinant anti-HPV 16 E7 T cell receptor (TCR) using various constructs for expression and targeted integration by HDR at the TRAC locus: polynucleotides encoding full sequences of the recombinant TCRα and TCRβ chains linked to the EF1α(“SV40 pA EF1αE7 TCR”) or MND promoter (“SV40 pA MND E7 TCR”) or encoding the full sequence of the recombinant TCRβ chain and partial sequence of the recombinant TCRα chain for in-frame integration with the exon 1 sequence of the endogenous TRAC gene, linked to the MND promoter (“MND E7 TCR 3′ arm”) or the endogenous TRAC promoter via an upstream P2A ribosome skip sequence (“P2A E7 TCR 3′arm”), integrated into the TRAC locus. T cells subjected to mock transfection (“Mock”) were assessed as control.

FIG. 2 shows a graph depicting the results of a cytolytic assay of T cells expressing an exemplary recombinant TCR co-cultured with target cells at an effector to target (E:T) ratio of 5:1, as assessed by lysis of target cells labeled with NucLight Red over time, in T cells subject to knockout of endogenous TCR encoding genes (TRAC and TRBC) and engineered to express an exemplary recombinant anti-HPV 16 E7 T cell receptor (TCR) using various constructs for expression and targeted integration by HDR at the TRAC locus: polynucleotides encoding full sequences of the recombinant TCRα and TCRβ chains linked to the EF1α(“SV40 pA EF1α E7 TCR”) or MND promoter (“SV40 pA MND E7 TCR”) or encoding the full sequence of the recombinant TCRβ chain and partial sequence of the recombinant TCRα chain for in-frame integration with the exon 1 sequence of the endogenous TRAC gene, linked to the MND promoter (“MND E7 TCR 3′ arm”) or the endogenous TRAC promoter via an upstream P2A ribosome skip sequence (“P2A E7 TCR 3′arm”), integrated into the TRAC locus. T cells subjected to mock transfection (“Mock”) and cells expressing a reference TCR (“E7 Ref Mouse”) were used as controls.

FIGS. 3A-3B depict results for the integration at various time points for the various homology arm lengths tested, as assessed by changes in GFP patterns at 24, 48 and 72 hours (FIG. 3A), and at 96 hours or 7 days (FIG. 3B) after transduction with AAV preparations containing the HDR template polynucleotides.

FIGS. 4A-4B depict the change in integration ratio for HDR using the various homology arm lengths, at 24, 48, 72 and 96 hours or 7 days for four different donors, Donor 1 and 2 (FIG. 4A) and Donor 3 and 4 (FIG. 4B).

### DETAILED DESCRIPTION

Provided herein are genetically engineered immune cells expressing a recombinant receptor such as a recombinant T cell receptor (TCR). In some embodiments, the provided genetically engineered immune cells are T cells that comprise a modified T cell receptor alpha constant (TRAC) locus or a modified T cell receptor beta constant (TRBC) locus comprising a nucleic acid encoding a recombinant TCR or portion thereof. In some embodiments, the nucleic acid sequence is or includes a fusion of a transgene and an open reading frame, or a partial sequence thereof, of an endogenous TRAC locus encoding a T cell receptor alpha (TCRα) constant domain. In certain embodiments, the transgene encodes a T cell receptor beta (TCRβ) chain and a portion of a T cell receptor alpha (TCRα) chain, wherein the portion is less than the full-length TCRα chain, e.g., a full-length native TCRα chain. In particular embodiments, the nucleic acid sequence is or includes a fusion of a transgene and an open reading frame, or a partial sequence thereof, of an endogenous TRBC locus encoding a T cell receptor beta (TCRβ) constant domain. In certain embodiments, the transgene encodes a TCRα chain and a portion of a TCRβ chain, and a portion of a TCRβ chain, wherein the portion is less than a full-length TCRβ chain, e.g., a full length TCRβ chain. Also provided are related cell compositions, methods for generating or producing the engineered cells, nucleic acids and kits for use in generating the engineered cells described herein.

T cell-based therapies, such as adoptive T cell therapies (including those involving the administration of engineered cells expressing recombinant TCRs specific for a disease or disorder of interest) can be effective in the treatment of cancer and other diseases and disorders. In certain contexts, available approaches for generating engineered cells for adoptive cell therapy may not always be entirely satisfactory. In some contexts, optimal efficacy can depend on the ability of the administered cells to express the recombinant TCR, and for uniform, homogenous and/or consistent expression of the receptors among cells, such as a population of immune cells and/or cells in a therapeutic cell composition.

In certain aspects, currently available methods, e.g., viral transduction, are not entirely satisfactory. For example, in some aspects, the efficiency of the expression of the recombinant TCR is limited among certain cells or certain cell populations that are engineered using currently available methods. In some cases, the recombinant TCR is only expressed in certain cells among a population of cells, and the level of expression of the recombinant TCR can vary widely among cells in the population. In particular aspects, the level of expression of the recombinant TCR may be difficult to predict, control and/or regulate. In some aspects, random integration of a nucleic acid sequence encoding the recombinant TCR into the genome of the cell may, in some cases, result in adverse and/or unwanted effects due to integration of the nucleic acid sequence into an undesired location in the genome, e.g., into an essential gene or a gene critical in regulating the activity of the cell. In some cases, random or semi-random integration of a nucleic acid sequence encoding the receptor can result in variegated, unregulated, uncontrolled and/or suboptimal expression or antigen binding, oncogenic transformation and transcriptional silencing of the nucleic acid sequence, depending on the site of integration and/or nucleic acid sequence copy number. In some aspects, the insertion of the recombinant TCR into the T cell genome can result in mispairings between recombinant TCR chains and the native TCR chains, thereby reducing the amount of function recombinant TCRs expressed by the cells.

In some aspects, variable integration of the sequences encoding the recombinant receptor can result in inconsistent expression, variable copy number of the nucleic acids, possible insertional mutagenesis and/or variability of receptor expression and/or genetic disruption within the cell composition, such as a therapeutic cell composition. In some aspects, use of particular random integration vectors, such as certain lentiviral vectors, requires the performance of replication competent lentivirus (RCL) assay.

In some embodiments, targeted genetic disruption of one or more of the endogenous TCR gene loci can lead to a reduced risk or chance of mispairing between chains of the engineered or recombinant TCR and the endogenous TCR. Mispaired TCRs can, in some aspects, create a new TCR that could potentially result in a higher risk of undesired or unintended antigen recognition and/or side effects, and/or could reduce expression levels of the desired engineered or recombinant TCR. In some aspects, reducing or preventing endogenous TCR expression can increase expression of the engineered or recombinant TCR in the T cells or T cell compositions as compared to cells in which expression of the TCR is not reduced or prevented. In some embodiments, recombinant TCR expression can be increased by 1.5-fold, 2-fold, 3-fold, 4-fold, 5-fold or more. For example, in some cases, suboptimal expression of an engineered or recombinant TCR can occur due to competition with an endogenous TCR and/or with TCRs having mispaired chains, for signaling domains such as the invariant CD3 signaling molecules that are involved in permitting expression of the complex on the cell surface. In some aspects, currently available methods for delivery of transgenes, e.g., encoding recombinant receptors, such as recombinant TCRs, may show inefficient integration and/or reduced expression of the recombinant receptors. In some aspects, the efficiency of integration and/or expression of the recombinant receptor within a population may be low and/or varied.

In some aspects, development of a humanized and/or fully human recombinant TCR presents technical challenges. For example, in some aspects, a humanized and/or a fully human recombinant TCR receptor competes with endogenous TCR complexes and can form mispairings with endogenous TCRα or TCRβ chains, which may, in certain aspects, reduce recombinant TCR signaling, activity, and/or expression. One method to address these challenges has been to design recombinant TCRs with mouse constant domains to prevent mispairings with endogenous human TCRα or TCRβ chains. However, use of recombinant TCRs with mouse sequences may, in some aspects, present a risk for immune response. The provided polynucleotides, reagents, articles of manufacture, kits, and methods address these challenges by inserting a portion of a recombinant TCR in-frame within an endogenous TCR gene, resulting in a modified locus that encodes a full recombinant TCR. In particular aspects, this insertion serves to disrupt the endogenous TCR gene expression while allowing for the expression of a full humanized and/or human recombinant TCR, reducing the likelihood of competition from or mispairings with endogenous TCR chains. α

In some aspects, the provided embodiments also permit the use of a smaller nucleic acid sequence fragments for engineering compared to existing methods, by utilizing a portion or all of the open reading frame sequences of the endogenous gene encoding a TCRα or TCRβ constant domain, to encode the TCRα or TCRβ chain, or portion thereof, of the recombinant TCR. In some aspects, the provided embodiments may allow accommodation of larger homology arms compared to conventional embodiments that require the entire length of the recombinant TCR in the introduced polynucleotide, and/or allow accommodation of nucleic acid sequences encoding additional molecules, as the length requirement for nucleic acid sequences encoding the recombinant TCR or a portion thereof is reduced. In some aspects, generation, delivery of the nucleic acid sequences, e.g., transgene sequences, and/or targeting efficiency by homology-directed repair (HDR), may be facilitated or improved using the provided embodiments.

In some embodiments, provided herein are methods of generating or producing genetically engineered cells comprising a modified TRAC or TRBC locus in which the modified TRAC or TRBC locus includes nucleic acid sequences encoding a recombinant TCR. In some aspects, the modified TRAC or TRBC locus in the genetically engineered cell comprises a transgene sequence (also referred to herein as exogenous or heterologous nucleic acid sequences) encoding a portion of a recombinant TCR, integrated into an endogenous TRAC or TRBC locus, which normally encodes a TCRα or TCRβ constant domain. In some embodiments, the methods involve inducing a targeted genetic disruption and homology-dependent repair (HDR), using template polynucleotides containing the transgene encoding a portion of the recombinant TCR, thereby targeting integration of the transgene at the TRAC or TRBC locus. Also provided are cells and cell compositions generated by the methods.

In some embodiments, the provided polynucleotides, transgenes, and/or vectors, when delivered into immune cells, result in the expression of recombinant TCRs that can modulate T cell activity, and, in some cases, can modulate T cell differentiation or homeostasis. The resulting genetically engineered cells or cell compositions can be used in adoptive cell therapy methods.

Thus, the provided embodiments can facilitate the production of engineered cells that exhibit improved expression, function and uniformity of expression and/or other desired feature or properties, and ultimately higher efficacy. The provided embodiments can also reduce the length of polynucleotides, transgenes, and/or vectors required to deliver the recombinant TCR to cells, e.g., to allow for sufficient space to package additional elements and/or transgenes within the same vector, e.g., viral vector.

Also provided are methods for engineering, preparing, and producing the engineered cells, and kits and devices for generating or producing the engineered cells. Provided are polynucleotides, e.g., viral vectors that contain a nucleic acid sequence encoding a portion of the chimeric receptor, and methods for introducing such polynucleotides into the cells, such as by transduction or by physical delivery, such as electroporation. Also provided are compositions containing the engineered cells, methods, kits, and devices for administering the cells and compositions to subjects, such as for adoptive cell therapy. In some aspects, the cells are isolated from a subject, engineered, and administered to the same subject. In other aspects, they are isolated from one subject, engineered, and administered to another subject.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. METHODS FOR PRODUCING CELLS EXPRESSING A RECOMBINANT RECEPTOR BY HOMOLOGY-DIRECTED REPAIR (HDR)

Provided herein are methods of producing a genetically engineered immune cell, e.g., a genetically engineered T cell for adoptive cell therapy, related compositions, methods, uses, and kits and articles of manufacture used for performing the methods. Also provided are genetically engineered immune cells expressing a recombinant receptor, such as a recombinant T cell receptor (TCR) and compositions containing such cells, including genetically engineered immune cells produced by any of the provided methods. The immune cells are generally engineered to express a recombinant molecule such as a recombinant receptor, e.g., a recombinant T cell receptor (TCR). In some aspects, the provided embodiments involve specifically targeting nucleic acid sequences encoding a portion of the recombinant receptor, e.g., a TCR, to a particular locus, e.g., at one or more target sites within of the endogenous TCR gene loci. In some embodiments, the nucleic acid sequences are integrated in-frame within the TCR gene loci to produce a modified gene locus that encodes the full recombinant TCR.

In some embodiments, provided are methods of producing a genetically engineered immune cell, e.g., a genetically engineered T cell for adoptive cell therapy. In some embodiments, the provided methods involve introducing into an immune cell one or more agent(s) capable of inducing a genetic disruption of one or more target site(s) (also known as “target position,” “target DNA sequence” or “target location”) within a gene encoding a domain or region of a T cell receptor alpha (TCRα) chain and/or one or more gene(s) encoding a domain or region of a T cell receptor beta (TCRβ) chain (also referred to throughout as “one or more agents” or “agent(s) with reference to aspects of the provided methods); and introducing into the immune cell a polynucleotide, e.g., a template polynucleotide, comprising a transgene encoding a recombinant receptor or a chain thereof, wherein the transgene encoding the recombinant receptor or a chain thereof is targeted at or near one of the at least one target site(s) via homology directed repair (HDR).

In some embodiments, provided herein are methods of generating or producing genetically engineered cells comprising a modified TRAC or TRBC locus in which the modified TRAC or TRBC locus includes nucleic acid sequences encoding a recombinant TCR. In some aspects, the modified TRAC or TRBC locus in the genetically engineered cell comprises a transgene sequence (also referred to herein as exogenous or heterologous nucleic acid sequences) encoding a portion of a recombinant TCR, integrated into an endogenous TRAC or TRBC locus, which normally encodes a TCRα or TCRβ constant domain. In some embodiments, the methods involve inducing a targeted genetic disruption and homology-dependent repair (HDR), using template polynucleotides containing the transgene encoding a portion of the recombinant TCR, thereby targeting integration of the transgene at the TRAC or TRBC locus. Also provided are cells and cell compositions generated by the methods.

In some embodiments, the transgene sequence encoding a portion of the recombinant TCR contains a sequence of nucleotides encoding a TCRβ chain and a portion of a TCRα chain.

In some embodiments, the portion of the TCRα chain encoded by the transgene sequences comprises less than a full length of the TCRα chain. In particular embodiments, the portion of the TCRα chain contains a TCRα variable domain and a portion of a TCRα constant domain that is less than a full length TCR constant domain, e.g., a full length native TCRα constant domain, or does not contain a sequence encoding the TCRα constant domain. In some aspects, upon integration of the transgene sequence into the endogenous TRAC locus, the resulting modified TRAC locus encodes a recombinant TCR receptor, encoded by a fusion of the transgene, targeted by HDR, and an open reading frame or a partial sequence thereof of an endogenous TRAC locus. In some embodiments, the encoded recombinant TCR contains a TCRα chain, e.g., a functional TCRα chain that is capable of binding to a TCRβ chain.

In particular embodiments, the transgene sequence encoding a portion of the recombinant TCR contains a sequence of nucleotides encoding a TCRα chain and/or a portion of a TCRβ chain. In some embodiments, the portion of the TCRβ chain encoded by the transgene sequences is or includes less than a full length of the TCRβ chain. In particular embodiments, the portion of the TCRβ chain contains a TCRβ variable domain and a portion of a TCRβ constant domain that is less than a full length TCR constant domain, e.g., a full length native TCRα constant domain, or does not contain a sequence encoding the TCRβ constant domain. In some aspects, upon integration of the transgene sequence into the endogenous TRBC locus, e.g., a TRBC1 and/or TRBC2 locus, the resulting modified TRBC locus encodes a recombinant TCR receptor, encoded by a fusion of the transgene, targeted by HDR, and an open reading frame or a partial sequence thereof of an endogenous TRBC locus. In some embodiments, the encoded recombinant TCR contains a TCRβ chain, e.g., a functional TCRβ chain that is capable of binding to a TCRα chain.

In some embodiments, the polynucleotide, e.g., the template polynucleotide, comprises a nucleic acid sequence encoding a fraction and/or a portion of a recombinant receptor or chain thereof, e.g., a recombinant TCR or a chain thereof. In certain embodiments, the nucleic acid sequence is targeted at a target site(s) that is within a gene locus that encodes an endogenous receptor, e.g., an endogenous TCR gene. In certain embodiments, the nucleic acid sequence is targeted for in-frame integration within the endogenous gene locus. In particular embodiments, the in-frame integration results in a coding sequence for the recombinant receptor that contains the nucleic acid sequence encoding the portion and/or fragment of the recombinant receptor in frame with the portion and/or fragment of the gene locus that encodes the remaining portion and/or fragment of the receptor, such as to integrate exogenous and endogenous nucleic acid sequences to arrive at a coding sequence encoding a complete, whole, and/or full length recombinant receptor. In certain embodiments, the integration genetically disrupts expression of the endogenous receptor encoded by gene at the target site. In particular embodiments, the transgene encoding the portion of the recombinant receptor is targeted within the gene locus via HDR.

In particular embodiments, the recombinant receptor is a recombinant TCR or chain thereof that contains one or more variable domains and one or more constant domains. In particular embodiments, the transgene encodes the portion and/or fraction of the recombinant TCR that does not include a TCR constant domain, and the transgene is integrated in-frame with the sequence, e.g., genomic DNA sequence, encoding the endogenous TCR constant domain. In certain embodiments, the integration results in a coding sequence that encodes the complete, whole, and/or full length recombinant TCR or chain thereof. In some embodiments, the coding sequence contains the transgene sequence encoding the portion or fragment of the TCR or chain thereof and an endogenous sequence encoding the endogenous TCR constant domain.

In some embodiments, the transgene encodes a portion and/or a fragment of the recombinant receptor that includes a portion and/or a fraction of a constant domain, e.g., a portion or fragment of the constant domain that is completely or partially identical to an endogenous TCR constant domain. In some embodiments, the transgene encodes is integrated in-frame with the sequence, e.g., genomic DNA sequence, encoding the portion and/or fragment of the endogenous TCR constant domain that is not encoded by the transgene. In particular embodiments, the integration results in a coding sequence that encodes the complete, whole, and/or full length recombinant TCR or chain thereof and contains the transgene sequence and the endogenous sequence encoding the endogenous portion or fragment of the TCR constant domain.

In some embodiments, the provided methods involve introducing into an immune cell one or more agent, wherein each of the one or more agent is independently capable of inducing a genetic disruption of a target site within a T cell receptor alpha constant (TRAC) gene and/or a T cell receptor beta constant (TRBC) gene, thereby inducing a genetic disruption of at least one target site; and introducing into the immune cell a template polynucleotide comprising a transgene encoding a recombinant T cell receptor (TCR) or an antigen-binding fragment thereof or a chain thereof, wherein the transgene encoding the recombinant TCR or antigen-binding fragment or chain thereof is targeted for integration at or near one of the at least one target site via homology directed repair (HDR). In particular embodiments, the integration at or near the target site is in frame with a portion of coding sequence of the TRAC or TRBC gene, such as, for example, a portion of the coding sequence downstream, e.g., immediately downstream and/or 3′ adjacent, to the target site.

In some embodiments, one of the at least one the target site(s) is in a T cell receptor alpha constant (TRAC) gene. In some embodiments, one of the at least one the target site(s) is in a T cell receptor beta constant 1 (TRBC1) or T cell receptor beta constant 2 (TRBC2) gene. In some embodiments, the one or more target site(s) is in a TRAC gene and one or both of a TRBC1 and a TRBC2 gene.

In some embodiments, the provided methods involve introducing into an immune cell having a genetic disruption of one or more target site(s) within a gene encoding a domain or region of a T cell receptor alpha (TCRα) chain and/or one or more gene(s) encoding a domain or region of a T cell receptor beta (TCRβ) chain a template polynucleotide comprising a transgene encoding a recombinant receptor, wherein the transgene encoding the recombinant receptor or a chain thereof is targeted at or near one of the at least one target site(s) via HDR.

In provided embodiments, the term “introducing” encompasses a variety of methods of introducing DNA into a cell, either in vitro or in vivo, such methods including transformation, transduction, transfection (e.g. electroporation), and infection. Vectors are useful for introducing DNA encoding molecules into cells. Possible vectors include plasmid vectors and viral vectors.

Viral vectors include retroviral vectors, lentiviral vectors, or other vectors such as adenoviral vectors or adeno-associated vectors. Methods, such as electroporation, also can be used to introduce or deliver protein or ribonucleoprotein (RNP), e.g. containing the Cas9 protein in complex with a targeting gRNA, to cells of interest.

In some cases, the embodiments provided herein involve targeted genetic disruption, e.g., DNA break, at one or more of the endogenous TCR gene loci (such as the endogenous genes encoding the TCRα and/or the TCRβ chains) by gene editing techniques, combined with targeted knock-in of nucleic acids encoding the recombinant receptor (such as a recombinant TCR or a CAR) by homology-directed repair (HDR). In some embodiments, the HDR step requires a break, e.g., a double-stranded break, in the DNA at the target genomic location. In some embodiments, the DNA break occurs as a result of a step in gene editing, for example, DNA breaks generated by targeted nucleases used in gene editing.

In some embodiments, the embodiments involve generating a targeted DNA break using gene editing methods and/or targeted nucleases, followed by HDR based on one or more template polynucleotide(s), e.g., template polynucleotide(s) that contains homology sequences and one or more transgenes, e.g., nucleic acids encoding a recombinant receptor or a chain thereof and/or other exogenous or recombinant nucleic acids, to specifically target and integrate the nucleic acid sequences encoding the recombinant receptor or a chain thereof and/or other exogenous or recombinant nucleic acids at or near the DNA break.

In some embodiments, the targeted genetic disruption and targeted integration of the recombinant receptor-encoding nucleic acids by HDR occurs at one or more target site(s) (also known as “target position,” “target DNA sequence” or “target location”) the endogenous genes that encode one or more domains, regions and/or chains of the endogenous T cell receptor (TCR). In some embodiments, the targeted genetic disruption is induced at the TCRα gene. In some embodiments, the targeted genetic disruption is induced at the TCRβ gene. In some embodiments, the targeted genetic disruption is induced at the endogenous TCRα gene and the endogenous TCRβ gene. Endogenous TCR genes can include one or more of the gene encoding TCRα constant domain (encoded by TRAC in humans) and/or TCRβ constant domain (encoded by TRBC1 or TRBC2 in humans). In particular embodiments, the polynucleotide, e.g., template polynucleotide, contains a nucleic acid sequence that encodes a portion and/or fragment of a recombinant TCR containing a portion and/or a fragment of a TCRα chain. In some embodiments, the portion and/or fragment of the TCRα chain contains a complete, whole, and/or full length TCRα variable domain and a portion and/or a fraction of a TCRα constant domain.

In certain embodiments, the nucleic acid sequence is integrated in-frame into a target site within a TRAC gene. In some embodiments, the in-frame integration results in a coding sequence encoding a whole, complete, and/or full-length recombinant TCR or chain thereof. In particular embodiments, the whole, complete, and/or full length recombinant receptor contains a whole, complete, and/or full length TCRα chain. In some embodiments, the whole, complete, and/or full length recombinant receptor contains a whole, complete, and/or full length TCRα constant domain.

In certain embodiments, the polynucleotide, e.g., template polynucleotide, encodes a portion and/or fragment of a recombinant TCR or chain thereof that contains a portion or a fragment of a TCRβ chain. In some embodiments, the portion and/or fragment of the TCRβ chain contains a portion and/or a fragment of a TCRβ constant domain. In certain embodiments, the transgene is integrated in-frame into a target site within a TRBC gene, e.g., TRBC1 and/or TRBC2. In some embodiments, the in-frame integration results in a coding sequence encoding a whole, complete, and/or full-length recombinant TCR or chain thereof. In particular embodiments, the whole, complete, and/or full length recombinant receptor contains a whole, complete, and/or full length TCRβ chain. In some embodiments, the whole, complete, and/or full length recombinant receptor contains a whole, complete, and/or full length TCRβ constant domain.

In some embodiments, a template polynucleotide is introduced into the engineered cell, prior to, simultaneously with, or subsequent to introduction of agent(s) capable of inducing a targeted genetic disruption. In the presence of a targeted genetic disruption, e.g., DNA break, the template polynucleotide can be used as a DNA repair template, to effectively copy and integrate the transgene, e.g., nucleic acid sequences encoding the recombinant receptor, at or near the site of the targeted genetic disruption by HDR, based on homology between the endogenous gene sequence surrounding the target site and the 5′ and/or 3′ homology arms included in the template polynucleotide.

In some embodiments, the gene editing and HDR steps are performed simultaneously and/or in one experimental reaction. In some embodiments, the gene editing and HDR steps are performed consecutively or sequentially, in one or consecutive experimental reaction(s). In some embodiments, the gene editing and HDR steps are performed in separate experimental reactions, simultaneously or at different times.

The immune cells can include a population of cells containing T cells. Such cells can be cells that have been obtained from a subject, such as obtained from a peripheral blood mononuclear cells (PBMC) sample, an unfractionated T cell sample, a lymphocyte sample, a white blood cell sample, an apheresis product, or a leukapheresis product. In some embodiments, T cells can be separated or selected to enrich T cells in the population using positive or negative selection and enrichment methods. In some embodiments, the population contains CD4+, CD8+ or CD4+ and CD8+ T cells. In some embodiments, the step of introducing the polynucleotide template and the step of introducing the agent (e.g. Cas9/gRNA RNP) can occur simultaneously or sequentially in any order. In particular embodiments, the polynucleotide template is introduced into the immune cells after inducing the genetic disruption by the step of introducing the agent(s) (e.g. Cas9/gRNA RNP). In some embodiments, prior to, during and/or subsequent to introduction of the polynucleotide template and one or more agents (e.g. Cas9/gRNA RNP), the cells are cultured or incubated under conditions to stimulate expansion and/or proliferation of cells.

In particular embodiments of the provided methods, the introduction of the template polynucleotide is performed after the introduction of the one or more agent capable of inducing a genetic disruption. Any method for introducing the one or more agent(s) can be employed as described, depending on the particular agent(s) used for inducing the genetic disruption. In some aspects, the disruption is carried out by gene editing, such as using an RNA-guided nuclease such as a clustered regularly interspersed short palindromic nucleic acid (CRISPR)-Cas system, such as CRISPR-Cas9 system, specific for the TRAC or TRBC locus being disrupted. In some embodiments, an agent containing a Cas9 and a guide RNA (gRNA) containing a targeting domain, which targets a region of the TRAC or TRBC locus, is introduced into the cell. In some embodiments, the agent is or comprises a ribonucleoprotein (RNP) complex of Cas9 and gRNA containing the TRAC/TRBC-targeted targeting domain (Cas9/gRNA RNP). In some embodiment, the introduction includes contacting the agent or portion thereof with the cells, in vitro, which can include cultivating or incubating the cell and agent for up to 24, 36 or 48 hours or 3, 4, 5, 6, 7, or 8 days. In some embodiments, the introduction further can include effecting delivery of the agent into the cells. In various embodiments, the methods, compositions and cells according to the present disclosure utilize direct delivery of ribonucleoprotein (RNP) complexes of Cas9 and gRNA to cells, for example by electroporation. In some embodiments, the RNP complexes include a gRNA that has been modified to include a 3′ poly-A tail and a 5′ Anti-Reverse Cap Analog (ARCA) cap. In some cases, electroporation of the cells to be modified includes cold-shocking the cells, e.g. at 32° C. following electroporation of the cells and prior to plating.

In such aspects of the provided methods, a template polynucleotide is introduced into the cells after introduction with the one or more agent(s), such as Cas9/gRNA RNP, e.g. that has been introduced via electroporation. In some embodiments, the template polynucleotide is introduced immediately after the introduction of the one or more agents capable of inducing a genetic disruption. In some embodiments, the template polynucleotide is introduced into the cells within at or about 30 seconds, within at or about 1 minute, within at or about 2 minutes, within at or about 3 minutes, within at or about 4 minutes, within at or about 5 minutes, within at or about 6 minutes, within at or about 6 minutes, within at or about 8 minutes, within at or about 9 minutes, within at or about 10 minutes, within at or about 15 minutes, within at or about 20 minutes, within at or about 30 minutes, within at or about 40 minutes, within at or about 50 minutes, within at or about 60 minutes, within at or about 90 minutes, within at or about 2 hours, within at or about 3 hours or within at or about 4 hours after the introduction of one or more agents capable of inducing a genetic disruption. In some embodiments, the template polynucleotide is introduced into cells at time between at or about 15 minutes and at or about 4 hours after introducing the one or more agent(s), such as between at or about 15 minutes and at or about 3 hours, between at and about 15 minutes and at or about 2 hours, between at or about 15 minutes and at or about 1 hour, between at or about 15 minutes and at or about 30 minutes, between at or about 30 minutes and at or about 4 hours, between at or about 30 minutes and at or about 3 hours, between at or about 30 minutes and at or about 2 hours, between at or about 30 minutes and at or about 1 hour, between at or about 1 hour and at or about 4 hours, between at or about 1 hour and at or about 3 hours, between at or about 1 hour and at or about 2 hours, between at or about 2 hours and at or about 4 hours, between at or about 2 hours and at or about 3 hours or between at or about 3 hours and at or about 4 hours. In some embodiments, the template polynucleotide is introduced into cells at or about 2 hours after the introduction of the one or more agents. such as Cas9/gRNA RNP, e.g. that has been introduced via electroporation.

Any method for introducing the template polynucleotide can be employed as described, depending on the particular methods used for delivery of the template polynucleotide to cells. Exemplary methods include those for transfer of nucleic acids encoding the receptors, including via viral, e.g., retroviral or lentiviral, transduction, transposons, and electroporation. In particular embodiments, viral transduction methods are employed. In some embodiments, template polynucleotides can be transferred or introduced into cells sing recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 November 29(11): 550-557. In particular embodiments, the viral vector is an AAV such as an AAV2 or an AAV6.

In some embodiments, prior to, during or subsequent to contacting the agent with the cells and/or prior to, during or subsequent to effecting delivery (e.g. electroporation), the provided methods include incubating the cells in the presence of a cytokine, a stimulating agent and/or an agent that is capable of inducing proliferation, stimulation or activation of the immune cells (e.g. T cells). In some embodiments, at least a portion of the incubation is in the presence of a stimulating agent that is or comprises an antibody specific for CD3 an antibody specific for CD28 and/or a cytokine, such as anti-CD3/anti-CD28 beads. In some embodiments, at least a portion of the incubation is in the presence of a cytokine, such as one or more of recombinant IL-2, recombinant IL-7 and/or recombinant IL-15. In some embodiments, the incubation is for up to 8 days hours before or after the introduction with the one or more agent(s), such as Cas9/gRNA RNP, e.g. via electroporation, and template polynucleotide, such as up to 24 hours, 36 hours or 48 hours or 3, 4, 5, 6, 7 or 8 days.

In some embodiments, the method includes activating or stimulating cells with a stimulating agent (e.g. anti-CD3/anti-CD28 antibodies) prior to introducing the agent, e.g. Cas9/gRNA RNP, and the polynucleotide template. In some embodiments, the incubation in the presence of a stimulating agent (e.g. anti-CD3/anti-CD28) is for 6 hours to 96 hours, such as 24-48 hours or 24-36 hours prior to the introduction with the one or more agent(s), such as Cas9/gRNA RNP, e.g. via electroporation. In some embodiments, the incubation with the stimulating agents can further include the presence of a cytokine, such as one or more of recombinant IL-2, recombinant IL-7 and/or recombinant IL-15. In some embodiments, the incubation is carried out in the presence of a recombinant cytokine, such as IL-2 (e.g. 1 U/mL to 500 U/mL, such as 10 U/mL to 200 U/mL, for example at least or about 50 U/mL or 100 U/mL), IL-7 (e.g. 0.5 ng/mL to 50 ng/mL, such as 1 ng/mL to 20 ng/mL, for example, at least or about 5 ng/mL or 10 ng/mL) or IL-15 (e.g. 0.1 ng/mL to 50 ng/mL, such as 0.5 ng/mL to 25 ng/mL, for example, at least or about 1 ng/mL or 5 ng/mL). In some embodiments the stimulating agent(s) (e.g. anti-CD3/anti-CD28 antibodies) is washed or removed from the cells prior to introducing or delivering into the cells the agent(s) capable of inducing a genetic disruption Cas9/gRNA RNP and/or the polynucleotide template. In some embodiments, prior to the introducing of the agent(s), the cells are rested, e.g. by removal of any stimulating or activating agent. In some embodiments, prior to introducing the agent(s), the stimulating or activating agent and/or cytokines are not removed.

In some embodiments, subsequent to the introduction of the agent(s), e.g.

Cas9/gRNA, and/or the polynucleotide template the cells are incubated, cultivated or cultured in the presence of a recombinant cytokine, such as one or more of recombinant IL-2, recombinant IL-7 and/or recombinant IL-15. In some embodiments, the incubation is carried out in the presence of a recombinant cytokine, such as IL-2 (e.g. 1 U/mL to 500 U/mL, such as 10 U/mL to 200 U/mL, for example at least or about 50 U/mL or 100 U/mL), IL-7 (e.g. 0.5 ng/mL to 50 ng/mL, such as 1 ng/mL to 20 ng/mL, for example, at least or about 5 ng/mL or 10 ng/mL) or IL-15 (e.g. 0.1 ng/mL to 50 ng/mL, such as 0.5 ng/mL to 25 ng/mL, for example, at least or about 1 ng/mL or 5 ng/mL). The cells can be incubated or cultivated under conditions to induce proliferation or expansion of the cells. In some embodiments, the cells can be incubated or cultivated until a threshold number of cells is achieved for harvest, e.g. a therapeutically effective dose.

In some embodiments, the incubation during any portion of the process or all of the process can be at a temperature of 30° C.±2° C. to 39° C.±2° C., such as at least or about at least 30° C.±2° C., 32° C.±2° C., 34° C.±2° C. or 37° C.±2° C. In some embodiments, at least a portion of the incubation is at 30° C.±2° C. and at least a portion of the incubation is at 37° C.±2° C.

In some embodiments, upon targeted integration, the nucleic acid sequence present at the modified TRAC or TRBC locus comprises a fusion of a transgene, targeted by HDR, and an open reading frame or a partial sequence thereof of an endogenous TRAC or TRBC locus. In some aspects, the nucleic acid sequence present at the modified TRAC or TRBC locus comprises a transgene that is integrated at an endogenous TRAC or TRBC locus containing an open reading frame encoding a TCRα or TCRβ constant domain. In some aspects, and upon targeted integration or fusion, e.g., in-frame fusion, a portion of the exogenous sequence and a portion of the open reading frame at the endogenous TRAC or TRBC locus together encodes a recombinant TCRα or TCRβ chain. Thus, the provided embodiments utilizes a portion or all of the open reading frame sequences of endogenous TRAC or TRBC loci to encode the full TCRα or TCRβ chain of the recombinant TCR.

In particular embodiments, the nucleic acid sequence present at the modified TRAC locus is or includes a fusion of a transgene, targeted by HDR, and an open reading frame or a partial sequence thereof of an endogenous TRAC locus. In certain embodiments, the nucleic acid sequence present at the modified TRAC locus comprises a transgene that is integrated at an endogenous TRAC locus containing an open reading frame encoding a TCRα constant domain.

In particular embodiments, upon targeted integration or fusion, e.g., in-frame fusion, a portion of the exogenous sequence and a portion of the open reading frame at the endogenous TRAC locus together encodes a recombinant TCRα chain, and/or a protein that contains a TCRα constant domain. In particular embodiments, a TCRα chain and/or a protein containing a TCRα constant domain contains a functional TCRα constant domain, e.g., a TCRα constant domain that is capable of binding to a TCRβ chain.

In some embodiments, the nucleic acid sequence present at the modified TRBC locus, e.g., a modified TRBC1 and/or TRBC2 locus, is or includes a fusion of a transgene, targeted by HDR, and an open reading frame or a partial sequence thereof of an endogenous TRBC locus. In certain embodiments, the nucleic acid sequence present at the modified TRBC locus comprises a transgene that is integrated at an endogenous TRAC locus containing an open reading frame encoding a TCRβ constant domain. In particular embodiments, upon targeted integration or fusion, e.g., in-frame fusion, a portion of the exogenous sequence and a portion of the open reading frame at the endogenous TRBC locus together encodes a recombinant TCRβ chain, and/or a protein that contains a TCRβ constant domain. In particular embodiments, a TCRβ chain and/or a protein containing a TCRβ constant domain contains a functional TCRβ constant domain, e.g., a TCRβ constant domain that is capable of binding to a TCRβ chain.

A. Genetic Disruption

In some embodiments, one or more targeted genetic disruption(s) is induced at the endogenous TCRα gene and/or the endogenous TCRβ gene. In some embodiments, the targeted genetic disruption is induced at one or more of the gene encoding TCRα constant domain (also known as TCRα constant region; encoded by TRAC in humans) and/or TCRβ constant domain (also known as TCRβ constant region; encoded by TRBC1 or TRBC2 in humans). In some embodiments, targeted genetic disruption is induced at the TRAC, TRBC1 and TRBC2 loci. In some embodiments, the targeted genetic disruption is induced in an intron, e.g., a TRAC, TRBC1 or TRBC2 intron. In some embodiments, the targeted genetic disruption is induced in an exon, e.g., a TRAC, TRBC1 or TRBC2 exon.

In some embodiments, targeted genetic disruption results in a DNA break or a nick. In some embodiments, at the site of the DNA break, action of cellular DNA repair mechanisms can result in knock-out, insertion, missense or frameshift mutation, such as a biallelic frameshift mutation, deletion of all or part of the gene. In some embodiments, the genetic disruption can be targeted to one or more exon of a gene or portion thereof, such as within the first or second exon. In some embodiments, a DNA binding protein or DNA-binding nucleic acid, which specifically binds to or hybridizes to the sequences at a region near one of the at least one target site(s), is used for targeted disruption. In some aspects, in the absence of exogenous template polynucleotides for HDR the disruption, the targeted genetic disruption results in a deletion, mutation and or insertion within an exon of the gene. In some embodiments, template polynucleotides, e.g., template polynucleotides that include nucleic acid sequences encoding a recombinant receptor and homology sequences, can be introduced for targeted integration of the recombinant receptor-encoding sequences at or near the site of the genetic disruption by HDR, such as any integration described herein e.g., in Section I.B.

In some embodiments, the genetic disruption is carried by introducing one or more agent(s) capable of inducing a genetic disruption. In some embodiments, such agents comprise a DNA binding protein or DNA-binding nucleic acid that specifically binds to or hybridizes to the gene. In some embodiments, the agent comprises various components, such as a fusion protein comprising a DNA-targeting protein and a nuclease or an RNA-guided nuclease. In some embodiments, the agents can target one or more target locations, e.g., at a TRAC gene and one or both of a TRBC1 and a TRBC2 gene.

In some embodiments, the genetic disruption occurs at a target site (also referred to and/or known as “target position,” “target DNA sequence,” or “target location”). In some embodiments, target site is or includes a site on a target DNA (e.g., genomic DNA) that is modified by the one or more agent(s) capable of inducing a genetic disruption, e.g., a Cas9 molecule complexed with a gRNA that specifies the target site. For example, in some embodiments, the target site may include locations in the DNA, e.g., at an endogenous TRAC or TRBC locus, where cleavage or DNA breaks occur. In some aspects, integration of nucleic acid sequences by HDR can occur at or near the target site or target sequence. In some embodiments, a target site can be a site between two nucleotides, e.g., adjacent nucleotides, on the DNA into which one or more nucleotides is added. The target site may comprise one or more nucleotides that are altered by a template polynucleotide. In some embodiments, the target site is within a target sequence (e.g., the sequence to which the gRNA binds). In some embodiments, a target site is upstream or downstream of a target sequence.

1. Target sites at Endogenous T Cell Receptor (TCR) Encoding Genes

In some embodiments, the targeted genetic disruption occurs at the endogenous genes that encode one or more domains, regions and/or chains of the endogenous T cell receptor (TCR). In some embodiments, the genetic disruption is targeted at the endogenous gene loci that encode TCRα and/or the TCRβ In some embodiments, the genetic disruption is targeted at the gene encoding TCRα constant domain (TRAC in humans) and/or TCRβ constant domain (TRBC1 or TRBC2 in humans).

In some embodiments, a “T cell receptor” or “TCR,” including the endogenous TCRs, is a molecule that contains a variable α and β chains (also known as TCRα and TCRβ, respectively) or a variable γ and δ chains (also known as TCRγ and TCRδ, respectively), or antigen-binding portions thereof, and which is capable of specifically binding to a peptide bound to an MHC molecule. In some embodiments, the TCR is in the a form. Typically, TCRs that exist in αβ and γδ forms are generally structurally similar, but T cells expressing them may have distinct anatomical locations or functions. Typically, one T cell expresses one type of TCR. A TCR can be found on the surface of a cell or in soluble form. Generally, a TCR is found on the surface of T cells (or T lymphocytes) where it is generally responsible for recognizing antigens bound to major histocompatibility complex (MHC) molecules.

In some embodiments, a TCR can contain a variable domain and a constant domain (also known as a constant region), a transmembrane domain and/or a short cytoplasmic tail (see, e.g., Janeway et al., Immunobiology: The Immune System in Health and Disease, 3 Ed., Current Biology Publications, p. 4:33, 1997). In some embodiments, a TCR chain contains one or more constant domain. For example, the extracellular portion of a given TCR chain (e.g., TCRα chain or TCRβ chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) and a constant domain (e.g., a chain constant domain or TCR Ca, typically positions 117 to 259 of the chain based on Kabat numbering or β chain constant domain or TCR Cβ, typically positions 117 to 295 of the chain based on Kabat) adjacent to the cell membrane. For example, in some cases, the extracellular portion of the TCR formed by the two chains contains two membrane-proximal constant domains, and two membrane-distal variable domains.

In some embodiments, the endogenous TCR Cα is encoded by the TRAC gene (IMGT nomenclature). An exemplary nucleotide sequence of the human T cell receptor alpha constant chain (TRAC) gene locus is set forth in SEQ ID NO:1 (NCBI Reference Sequence: NG_001332.3, TRAC). In some embodiments, the encoded endogenous Cα comprises the sequence of amino acids set forth in SEQ ID NO: 19 or 24 (UniProtKB Accession No. P01848 or Genbank Accession No. CAA26636.1).

In humans, an exemplary genomic locus of TRAC comprises an open reading frame that contains 4 exons and 3 introns. An exemplary mRNA transcript of TRAC can span the sequence corresponding to coordinates Chromosome 14: 22, 547, 506-22, 552, 154, on the forward strand, with reference to human genome version GRCh38 (UCSC Genome Browser on Human December 2013 (GRCh38/hg38) Assembly). Table 1 sets forth the coordinates of the exons and introns of the open reading frames and the untranslated regions of the transcript of an exemplary human TRAC locus.

TABLE 1
Coordinates of exons and introns of exemplary human
TRAC locus (GRCh38, Chromosome 14, forward strand).

| | Start (GrCh38) | End (GrCh38) | Length |
|---|---|---|---|
| 5′ UTR and Exon 1 | 22,547,506 | 22,547,778 | 273 |
| Intron 1-2 | 22,547,779 | 22,549,637 | 1,859 |
| Exon 2 | 22,549,638 | 22,549,682 | 45 |
| Intron 2-3 | 22,549,683 | 22,550,556 | 874 |
| Exon 3 | 22,550,557 | 22,550,664 | 108 |
| Intron 3-4 | 22,550,665 | 22,551,604 | 940 |
| Exon 4 and 3′ UTR | 22,551,605 | 22,552,154 | 550 |

In certain embodiments, a genetic disruption is targeted at, near, or within a TRAC locus. In particular embodiments, the genetic disruption is targeted at, near, or within an open reading frame of the TRAC locus. In certain embodiments, the genetic disruption is targeted at, near, or within an open reading frame that encodes a TCRα constant domain. In some embodiments, the genetic disruption is targeted at, near, or within a locus having the nucleic acid sequence set forth in SEQ ID NO: 1, or a sequence having at or at least 70%, 75%, 80%, 85%, 90%, 95%, 97%, 98%, 99%, 99.5%, or 99.9% sequence identity to all or a portion, e.g., at or at least 500, 1,000, 1,500, 2,000, 2,500, 3,000, 3,500, or 4,000 contiguous nucleotides, of the nucleic acid sequence set forth in SEQ ID NO: 1.

In humans, an exemplary genomic locus of TRBC1 comprises an open reading frame that contains 4 exons and 3 introns. An exemplary mRNA transcript of TRBC1 can span the sequence corresponding to coordinates Chromosome 7: 142, 791, 694-142, 793, 368, on the forward strand, with reference to human genome version GRCh38 (UCSC Genome Browser on Human December 2013 (GRCh38/hg38) Assembly). Table 2 sets forth the coordinates of the exons and introns of the open reading frames and the untranslated regions of the transcript of an exemplary human TRBC1 locus.

TABLE 2
Coordinates of exons and introns of exemplary human
TRBC1 locus (GRCh38, Chromosome 7, forward strand).

| | Start (GrCh38) | End (GrCh38) | Length |
|---|---|---|---|
| 5′ UTR and Exon 1 | 142,791,694 | 142,792,080 | 387 |
| Intron 1-2 | 142,792,081 | 142,792,521 | 441 |
| Exon 2 | 142,792,522 | 142,792,539 | 18 |
| Intron 2-3 | 142,792,540 | 142,792,691 | 152 |
| Exon 3 | 142,792,692 | 142,792,798 | 107 |
| Intron 3-4 | 142,792,799 | 142,793,120 | 322 |
| Exon 4 and 3′ UTR | 142,793,121 | 142,793,368 | 248 |

In humans, an exemplary genomic locus of TRBC2 comprises an open reading frame that contains 4 exons and 3 introns. An exemplary mRNA transcript of TRBC2 can span the sequence corresponding to coordinates Chromosome 7: 142, 801, 041-142, 802, 748, on the forward strand, with reference to human genome version GRCh38 (UCSC Genome Browser on Human December 2013 (GRCh38/hg38) Assembly). Table 3 sets forth the coordinates of the exons and introns of the open reading frames and the untranslated regions of the transcript of an exemplary human TRBC2 locus.

TABLE 3
Coordinates of exons and introns of exemplary human
TRBC2 locus (GRCh38, Chromosome 7, forward strand).

| | Start (GrCh38) | End (GrCh38) | Length |
|---|---|---|---|
| 5′ UTR and Exon 1 | 142,801,041 | 142,801,427 | 387 |
| Intron 1-2 | 142,801,428 | 142,801,943 | 516 |
| Exon 2 | 142,801,944 | 142,801,961 | 18 |
| Intron 2-3 | 142,801,962 | 142,802,104 | 143 |
| Exon 3 | 142,802,105 | 142,802,211 | 107 |
| Intron 3-4 | 142,802,212 | 142,802,502 | 291 |
| Exon 4 and 3′ UTR | 142,802,503 | 142,802,748 | 246 |

In some aspects, the transgene (e.g., exogenous nucleic acid sequences) within the template polynucleotide can be used to guide the location of target sites and/or homology arms. In some aspects, the target site of genetic disruption can be used as a guide to design template polynucleotides and/or homology arms used for HDR. In some embodiments, the genetic disruption can be targeted near a desired site of targeted integration of transgene sequences (e.g., encoding a recombinant TCR or a portion thereof). In some aspects, the target site is within an exon of the open reading frame of the TRAC, TRBC1 and/or TRBC2 locus. In some aspects, the target site is within an intron of the open reading frame of the TRAC, TRBC1 and/or TRBC2 locus.

In some embodiments, the endogenous TCR Cβ is encoded by TRBC1 or TRBC2 genes (IMGT nomenclature). An exemplary nucleotide sequence of the human T cell receptor beta constant chain 1 (TRBC1) gene locus is set forth in SEQ ID NO:2 (NCBI Reference Sequence: NG_001333.2, TRBC1); and an exemplary nucleotide sequence of the human T cell receptor beta constant chain 2 (TRBC2) gene locus is set forth in SEQ ID NO:3 (NCBI Reference Sequence: NG_001333.2, TRBC2). In some embodiments, the encoded Cβ has or comprises the sequence of amino acids set forth in SEQ ID NO:20, 21 or 25 (Uniprot Accession No. P01850, A0A5B9 or A0A0G2JNG9). In some embodiments, a genetic disruption is targeted at, near, or within the TRBC1 gene locus. In particular embodiments, the genetic disruption is targeted at, near, or within an open reading frame of the TRBC1 locus. In certain embodiments, the genetic disruption is targeted at, near, or within an open reading frame that encodes a TCRβ constant domain. In some embodiments, the genetic disruption is targeted at, near, or within a locus having the nucleic acid sequence set forth in SEQ ID NO: 2, or a sequence having at or at least 70%, 75%, 80%, 85%, 90%, 95%, 97%, 98%, 99%, 99.5%, or 99.9% sequence identity to all or a portion, e.g., at or at least 500, 1,000, 1,500, 2,000, 2,500, 3,000, 3,500, or 4,000 contiguous nucleotides, of the nucleic acid sequence set forth in SEQ ID NO: 2.

In particular embodiments, a genetic disruption is targeted at, near, or within the TRBC2 locus. In particular embodiments, the genetic disruption is targeted at, near, or within an open reading frame of the TRBC2 locus. In certain embodiments, the genetic disruption is targeted at, near, or within an open reading frame that encodes a TCRβ constant domain. In some embodiments, the genetic disruption is targeted at, near, or within a locus having the nucleic acid sequence set forth in SEQ ID NO: 3, or a sequence having at or at least 70%, 75%, 80%, 85%, 90%, 95%, 97%, 98%, 99%, 99.5%, or 99.9% sequence identity to all or a portion, e.g., at or at least 500, 1,000, 1,500, 2,000, 2,500, 3,000, 3,500, or 4,000 contiguous nucleotides, of the nucleic acid sequence set forth in SEQ ID NO: 3.

In some embodiments, the genetic disruption, e.g., DNA break, is targeted at or in close proximity to the beginning of the coding region (e.g., the early coding region, e.g., within 500 bp from the start codon or the remaining coding sequence, e.g., downstream of the first 500 bp from the start codon). In some embodiments, the genetic disruption, e.g., DNA break, is targeted at early coding region of a gene of interest, e.g., TRAC, TRBC1 and/or TRBC2, including sequence immediately following a transcription start site, within a first exon of the coding sequence, or within 500 bp of the transcription start site (e.g., less than 500, 450, 400, 350, 300, 250, 200, 150, 100 or 50 bp), or within 500 bp of the start codon (e.g., less than 500, 450, 400, 350, 300, 250, 200, 150, 100 or 50 bp).

In some embodiments, the target site is within an exon of the endogenous TRAC locus. In certain embodiments, the target site is within an intron of the endogenous TRAC locus. In some aspects, the target site is within a regulatory or control element, e.g., a promoter, 5′ untranslated region (UTR) or 3′ UTR, of the TRAC locus. In certain embodiments, the target site is within an open reading frame of an endogenous TRAC locus. In particular embodiments, the target site is within an exon within the open reading frame of the TRAC locus.

In particular embodiments, the genetic disruption, e.g., DNA break, is targeted at or within an open reading frame of a gene or locus of interest, e.g., TRAC, TRBC1, and/or TRBC2.

In some embodiments, the genetic disruption is targeted at or within an intron within the open reading frame of a gene or locus of interest. In some embodiments, the genetic disruption is targeted within an exon within the open reading frame of the gene or locus of interest.

In particular embodiments, a genetic disruption, e.g., DNA break, is targeted at or within an intron. In certain embodiments, a genetic disruption, e.g., DNA break, is targeted at or within an exon. In some embodiments, a genetic disruption, e.g., DNA break, is targeted at or within an exon of a gene of interest, e.g., TRAC, TRBC1 and/or TRBC2.

In particular embodiments, a genetic disruption, e.g., DNA break, is targeted within an exon of a TRBC gene, open reading frame, or locus, e.g., TRBC1 and/or the TRBC2. In certain embodiments, the genetic disruption is within the first exon, second exon, third exon, or fourth exon of the TRBC1 and/or the TRBC2 gene, open reading frame, or locus. In some embodiments, the genetic disruption is within the first exon of the TRBC1 and/or the TRBC2 gene, open reading frame, or locus. In certain embodiments, the genetic disruption is within the first exon, second exon, third exon, or fourth exon of the TRBC1 and/or the TRBC2 gene, open reading frame, or locus. In some embodiments, the genetic disruption is between the most 5′ nucleotide of exon 1 and upstream of the most 3′ nucleotide of exon 1. In particular embodiments, the genetic disruption is within the first exon of the TRBC gene, open reading frame, or locus. In some embodiments, the genetic disruption is within 400 bp, 350 bp, 300 bp, 250 bp, 200 bp, 150 bp, 100 bp, or 50 bp downstream from the 5′ end of the first exon in a TRBC1 and/or the TRBC2 gene, open reading frame, or locus. In particular embodiments, the genetic disruption is between 1 bp and 400 bp, between 50 and 300 bp, between 100 bp and 200 bp, or between 100 bp and 150 bp downstream from the 5′ end of the first exon in the TRBC1 and/or the TRBC2 gene, open reading frame, or locus, each inclusive. In certain embodiments, the genetic disruption is between 100 bp and 150 bp downstream from the 5′ end of the first exon in the TRBC1 and/or the TRBC2 gene, open reading frame, or locus, inclusive.

In particular embodiments, a genetic disruption, e.g., DNA break, is targeted within an exon of a TRBC gene, open reading frame, or locus, e.g., TRBC1 and/or the TRBC2. In certain embodiments, the genetic disruption is within the first exon, second exon, third exon, or fourth exon of the TRBC gene, open reading frame, or locus. In some embodiments, the genetic disruption is within the first exon of the TRBC gene, open reading frame, or locus. In certain embodiments, the genetic disruption is within the first exon, second exon, third exon, or fourth exon of the TRBC gene, open reading frame, or locus. In some embodiments, the genetic disruption is between the 5′ nucleotide of exon 1 and upstream of the 3′ nucleotide of exon 1. In particular embodiments, the genetic disruption is within the first exon of the TRBC gene, open reading frame, or locus. In some embodiments, the genetic disruption is within 400 bp, 350 bp, 300 bp, 250 bp, 200 bp, 150 bp, 100 bp, or 50 bp downstream from the 5′ end of the first exon in a TRBC gene, open reading frame, or locus. In particular embodiments, the genetic disruption is between 1 bp and 400 bp, between 50 and 300 bp, between 100 bp and 200 bp, or between 100 bp and 150 bp downstream from the 5′ end of the first exon in the TRBC gene, open reading frame, or locus, each inclusive. In certain embodiments, the genetic disruption is between 100 bp and 150 bp downstream from the 5′ end of the first exon in the TRBC gene, open reading frame, or locus, inclusive.

2. Methods of Genetic Disruption

Methods for generating a genetic disruption, including those described herein, can involve the use of one or more agent(s) capable of inducing a genetic disruption, such as engineered systems to induce a genetic disruption, a cleavage and/or a double strand break (DSB) or a nick in a target site or target position in the endogenous DNA such that repair of the break by an error born process such as non-homologous end joining (NHEJ) or repair using a repair template HDR can result in the knock out of a gene and/or the insertion of a sequence of interest (e.g., exogenous nucleic acid sequences or transgene encoding a portion of a chimeric receptor) at or near the target site or position. Also provided are one or more agent(s) capable of inducing a genetic disruption, for use in the methods provided herein. In some aspects, the one or more agent(s) can be used in combination with the template nucleotides provided herein, for homology directed repair (HDR) mediated targeted integration of the transgene sequences (e.g., described herein in Section I.B).

In some embodiments, the one or more agent(s) capable of inducing a genetic disruption comprises a DNA binding protein or DNA-binding nucleic acid that specifically binds to or hybridizes to a particular site or position in the genome, e.g., a target site or target position. In some aspects, the targeted genetic disruption, e.g., DNA break or cleavage, of the endogenous genes encoding TCR is achieved using a protein or a nucleic acid is coupled to or complexed with a gene editing nuclease, such as in a chimeric or fusion protein. In some embodiments, the one or more agent(s) capable of inducing a genetic disruption comprises an RNA-guided nuclease or a fusion protein comprising a DNA-targeting protein and a nuclease.

In some embodiments, the agent comprises various components, such as an RNA-guided nuclease, or a fusion protein comprising a DNA-targeting protein and a nuclease. In some embodiments, the targeted genetic disruption is carried out using a DNA-targeting molecule that includes a DNA-binding protein such as one or more zinc finger protein (ZFP) or transcription activator-like effectors (TALEs), fused to a nuclease, such as an endonuclease. In some embodiments, the targeted genetic disruption is carried out using RNA-guided nucleases such as a clustered regularly interspaced short palindromic nucleic acid (CRISPR)-associated nuclease (Cas) system (including Cas and/or Cfp1). In some embodiments, the targeted genetic disruption is carried using agents capable of inducing a genetic disruption, such as sequence-specific or targeted nucleases, including DNA-binding targeted nucleases and gene editing nucleases such as zinc finger nucleases (ZFN) and transcription activator-like effector nucleases (TALENs), and RNA-guided nucleases such as a CRISPR-associated nuclease (Cas) system, specifically designed to be targeted to the at least one target site(s), sequence of a gene or a portion thereof. Exemplary ZFNs, TALEs, and TALENs are described in, e.g., Lloyd et al., Frontiers in Immunology, 4(221): 1-7 (2013).

Zinc finger proteins (ZFPs), transcription activator-like effectors (TALEs), and CRISPR system binding domains can be “engineered” to bind to a predetermined nucleotide sequence, for example via engineering (altering one or more amino acids) of the recognition helix region of a naturally occurring ZFP or TALE protein. Engineered DNA binding proteins (ZFPs or TALEs) are proteins that are non-naturally occurring. Rational criteria for design include application of substitution rules and computerized algorithms for processing information in a database storing information of existing ZFP and/or TALE designs and binding data. See, e.g., U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some embodiments, the one or more agent(s) specifically targets the at least one target site(s), e.g., at or near a gene of interest, e.g., TRAC, TRBC1 and/or TRBC2. In some embodiments, the agent comprises a ZFN, TALEN or a CRISPR/Cas9 combination that specifically binds to, recognizes, or hybridizes to the target site(s). In some embodiments, the CRISPR/Cas9 system includes an engineered crRNA/tracr RNA (“single guide RNA”) to guide specific cleavage. In some embodiments, the agent comprises nucleases based on the Argonaute system (e.g., from T. thermophilus, known as ‘TtAgo’, (Swarts et al., (2014) Nature 507(7491): 258-261). Targeted cleavage using any of the nuclease systems described herein can be exploited to insert the sequences of a transgene, e.g., nucleic acid sequences encoding a recombinant receptor, into a specific target location, e.g., at endogenous TCR genes, using either HDR or NHEJ-mediated processes.

In some embodiments, a “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3, and 6) on a zinc finger recognition helix. Thus, for example, the ZFP or ZFP-containing molecule is non-naturally occurring, e.g., is engineered to bind to a target site of choice.

In some cases, the DNA-targeting molecule is or comprises a zinc-finger DNA binding domain fused to a DNA cleavage domain to form a zinc-finger nuclease (ZFN). For example, fusion proteins comprise the cleavage domain (or cleavage half-domain) from at least one Type IIS restriction enzyme and one or more zinc finger binding domains, which may or may not be engineered. In some cases, the cleavage domain is from the Type IIS restriction endonuclease FokI, which generally catalyzes double-stranded cleavage of DNA, at 9 nucleotides from its recognition site on one strand and 13 nucleotides from its recognition site on the other. See, e.g., U.S. Pat. Nos. 5,356,802; 5,436,150 and 5,487,994; Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269: 978-982. Some gene-specific engineered zinc fingers are available commercially. For example, a platform called CompoZr, for zinc-finger construction is available that provides specifically targeted zinc fingers for thousands of targets. See, e.g., Gaj et al., Trends in Biotechnology, 2013, 31(7), 397-405. In some cases, commercially available zinc fingers are used or are custom designed.

In some embodiments, the one or more target site(s), e.g., within TRAC, TRBC1 and/or TRBC2 genes can be targeted for genetic disruption by engineered ZFNs. Exemplary ZFN that target endogenous T cell receptor (TCR) genes include those described in, e.g., US 2015/0164954, US 2011/0158957, US 2015/0056705, U.S. Pat. No. 8,956,828 and Torikawa et al. (2012) Blood 119:5697-5705, the disclosures of which are incorporated by reference in their entireties, or those set forth in any of SEQ ID NOS:213-224 (TRAC) or SEQ ID NOS: 225 and 226 (TRBC).

Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from different bacterial species. The new modular proteins have the advantage of displaying more sequence variability than TAL repeats. In some embodiments, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In some embodiments, critical amino acids 12 and 13 can be mutated towards other amino acid residues in order to modulate their specificity towards nucleotides A, T, C and G and in particular to enhance this specificity.

In some embodiments, a “TALE DNA binding domain” or “TALE” is a polypeptide comprising one or more TALE repeat domains/units. The repeat domains, each comprising a repeat variable diresidue (RVD), are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. TALE proteins may be designed to bind to a target site using canonical or non-canonical RVDs within the repeat units. See, e.g., U.S. Pat. Nos. 8,586,526 and 9,458,205.

In some embodiments, a “TALE-nuclease” (TALEN) is a fusion protein comprising a nucleic acid binding domain typically derived from a Transcription Activator Like Effector (TALE) and a nuclease catalytic domain that cleaves a nucleic acid target sequence. The catalytic domain comprises a nuclease domain or a domain having endonuclease activity, like for instance I-TevI, ColE7, NucA and Fok-I. In a particular embodiment, the TALE domain can be fused to a meganuclease like for instance I-CreI and I-OnuI or functional variant thereof. In some embodiments, the TALEN is a monomeric TALEN. A monomeric TALEN is a TALEN that does not require dimerization for specific recognition and cleavage, such as the fusions of engineered TAL repeats with the catalytic domain of I-TevI described in WO2012138927. TALENs have been described and used for gene targeting and gene modifications (see, e.g., Boch et al. (2009) Science 326(5959): 1509-12.; Moscou and Bogdanove (2009) Science 326(5959): 1501; Christian et al. (2010) Genetics 186(2): 757-61; Li et al. (2011) Nucleic Acids Res 39(1): 359-72).

In some embodiments, the TRAC, TRBC1 and/or TRBC2 genes can be targeted for genetic disruption by engineered TALENs. Exemplary TALEN that target endogenous T cell receptor (TCR) genes include those described in, e.g., WO 2017/070429, WO 2015/136001, US20170016025 and US20150203817, the disclosures of which are incorporated by reference in their entireties.

In some embodiments, a “TtAgo” is a prokaryotic Argonaute protein thought to be involved in gene silencing. TtAgo is derived from the bacteria Thermus thermophilus. See, e.g. Swarts et al (2014) Nature 507(7491): 258-261; G. Sheng et al., (2013) Proc. Natl. Acad. Sci. U.S.A. 111, 652. A “TtAgo system” is all the components required including e.g. guide DNAs for cleavage by a TtAgo enzyme.

In some embodiments, an engineered zinc finger protein, TALE protein or CRISPR/Cas system is not found in nature and whose production results primarily from an empirical process such as phage display, interaction trap or hybrid selection. See e.g., U.S. Pat. Nos. 5,789,538; 5,925,523; 6,007,988; 6,013,453; 6,200,759; WO 95/19431; WO 96/06166; WO 98/53057; WO 98/54311; WO 00/27878; WO 01/60970; WO 01/88197 and WO 02/099084.

Zinc finger and TALE DNA-binding domains can be engineered to bind to a predetermined nucleotide sequence, for example via engineering (altering one or more amino acids) of the recognition helix region of a naturally occurring zinc finger protein or by engineering of the amino acids involved in DNA binding (the repeat variable diresidue or RVD region). Therefore, engineered zinc finger proteins or TALE proteins are proteins that are non-naturally occurring. Non-limiting examples of methods for engineering zinc finger proteins and TALEs are design and selection. A designed protein is a protein not occurring in nature whose design/composition results principally from rational criteria. Rational criteria for design include application of substitution rules and computerized algorithms for processing information in a database storing information of existing ZFP or TALE designs (canonical and non-canonical RVDs) and binding data. See, for example, U.S. Pat. Nos. 9,458,205; 8,586,526; 6,140,081; 6,453,242; and 6,534,261; see also WO 98/53058; WO 98/53059; WO 98/53060; WO 02/016536 and WO 03/016496.

Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, e.g., U.S. Pat. Nos. 9,255,250; 9,200,266; 9,045,763; 9,005,973; 9,150,847; 8,956,828; 8,945,868; 8,703,489; 8,586,526; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publications 20030232410; 20050208489; 20050026157; 20050064474; 20060063231; 20080159996; 201000218264; 20120017290; 20110265198; 20130137104; 20130122591; 20130177983; 20130196373; 20140120622; 20150056705; 20150335708; 20160030477 and 20160024474, the disclosures of which are incorporated by reference in their entireties. Also provided are one or more agents capable of introducing a genetic disruption. Also provided are polynucleotides (e.g., nucleic acid molecules) encoding one or more components of the one or more agent(s) capable of inducing a genetic disruption.

a. Crispr/Cas9

In some embodiments, the targeted genetic disruption, e.g., DNA break, of the endogenous genes encoding TCR, such as TRAC and TRBC1 or TRBC2 in humans is carried out using clustered regularly interspaced short palindromic repeats (CRISPR) and CRISPR-associated (Cas) proteins. See Sander and Joung, (2014) Nature Biotechnology, 32(4): 347-355.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

In some aspects, the CRISPR/Cas nuclease or CRISPR/Cas nuclease system includes a non-coding guide RNA (gRNA), which sequence-specifically binds to DNA, and a Cas protein (e.g., Cas9), with nuclease functionality.

1) Guide RNA (gRNA)

In some embodiments, the one or more agent(s) comprises at least one of: a guide RNA (gRNA) having a targeting domain that is complementary with a target site of a TRAC gene; a gRNA having a targeting domain that is complementary with a target site of one or both of a TRBC1 and a TRBC2 gene; or at least one nucleic acid encoding the gRNA.

In some aspects, a “gRNA molecule” is to a nucleic acid that promotes the specific targeting or homing of a gRNA molecule/Cas9 molecule complex to a target nucleic acid, such as a locus on the genomic DNA of a cell. gRNA molecules can be unimolecular (having a single RNA molecule), sometimes referred to herein as “chimeric” gRNAs, or modular (comprising more than one, and typically two, separate RNA molecules). In general, a guide sequence, e.g., guide RNA, is any polynucleotide sequences comprising at least a sequence portion that has sufficient complementarity with a target polynucleotide sequence, such as the TRAC, TRBC1 and/or TRBC2 genes in humans, to hybridize with the target sequence at the target site and direct sequence-specific binding of the CRISPR complex to the target sequence. In some embodiments, in the context of formation of a CRISPR complex, “target sequence” generally refers to a sequence to which a guide sequence is designed to have complementarity, where hybridization between the target sequence and a domain, e.g., targeting domain, of the guide RNA promotes the formation of a CRISPR complex. Full complementarity is not necessarily required, provided there is sufficient complementarity to cause hybridization and promote formation of a CRISPR complex. Generally, a guide sequence is selected to reduce the degree of secondary structure within the guide sequence. Secondary structure may be determined by any suitable polynucleotide folding algorithm.

In some embodiments, a guide RNA (gRNA) specific to a target locus of interest (e.g. at the TRAC, TRBC1 and/or TRBC2 loci in humans) is used to RNA-guided nucleases, e.g., Cas, to induce a DNA break at the target site or target position. Methods for designing gRNAs and exemplary targeting domains can include those described in, e.g., International PCT Pub. Nos. WO2015/161276, WO2017/193107 and WO2017/093969 US2016/272999 and US2015/056705.

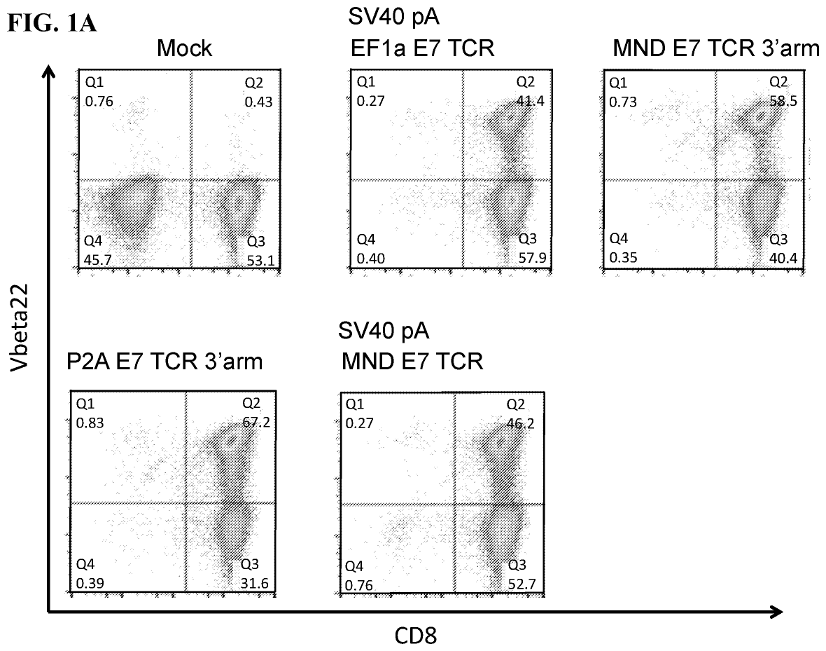

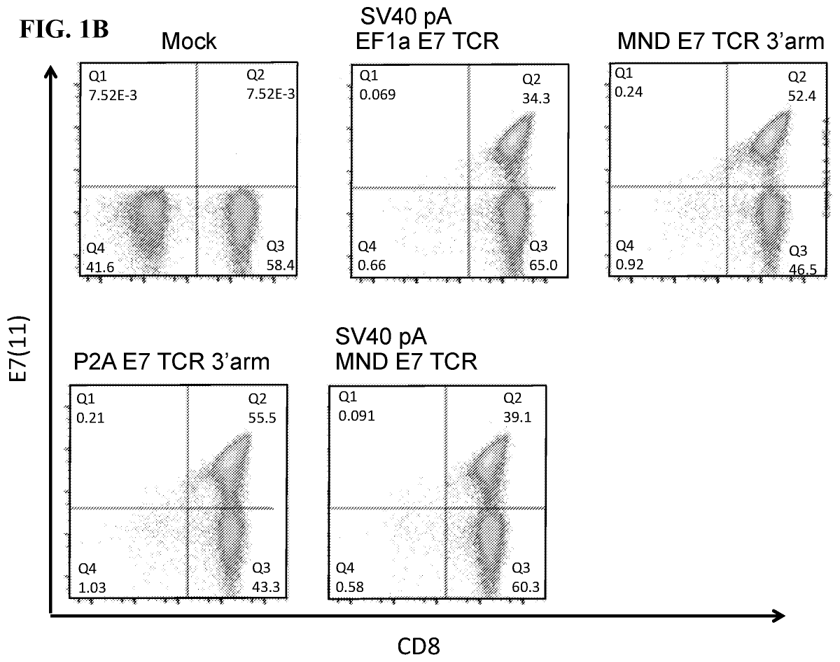

Several exemplary gRNA structures, with domains indicated thereon, are described in WO2015/161276, e.g., in FIGS. 1A-1G therein. While not wishing to be bound by theory, with regard to the three dimensional form, or intra- or inter-strand interactions of an active form of a gRNA, regions of high complementarity are sometimes shown as duplexes in WO2015/161276, e.g., in FIGS. 1A-1G therein and other depictions provided herein.

In some cases, the gRNA is a unimolecular or chimeric gRNA comprising, from 5′ to 3′: a targeting domain which targets a target site or position, such within as a sequence from the TRAC locus (exemplary nucleotide sequence of the human TRAC gene locus set forth in SEQ ID NO:1; NCBI Reference Sequence: NG_001332.3, TRAC; exemplary genomic sequence described in Table 1 herein); a first complementarity domain; a linking domain; a second complementarity domain (which is complementary to the first complementarity domain); a proximal domain; and optionally, a tail domain. In some cases, the gRNA is a unimolecular or chimeric gRNA comprising, from 5′ to 3′: a targeting domain which targets a target site or position, such as within a sequence from the TRBC1 or TRBC2 locus (exemplary nucleotide sequence of the human TRBC1 gene locus set forth in SEQ ID NO:2; NCBI Reference Sequence: NG_001333.2, TRBC1; exemplary genomic sequence described in Table 2 herein; exemplary nucleotide sequence of the human TRBC2 gene locus set forth in SEQ ID NO:3; NCBI Reference Sequence: NG_001333.2, TRBC2; exemplary genomic sequence described in Table 3 herein); a first complementarity domain; a linking domain; a second complementarity domain (which is complementary to the first complementarity domain); a proximal domain; and optionally, a tail domain.

In other cases, the gRNA is a modular gRNA comprising first and second strands. In these cases, the first strand preferably includes, from 5′ to 3′: a targeting domain (which targets a target site or position, such as within a sequence from TRAC locus (exemplary nucleotide sequence of the human TRAC gene locus set forth in SEQ ID NO:1; NCBI Reference Sequence: NG_001332.3, TRAC; exemplary genomic sequence described in Table 1 herein) or TRBC1 or TRBC2 locus (exemplary nucleotide sequence of the human TRBC1 gene locus set forth in SEQ ID NO:2; NCBI Reference Sequence: NG_001333.2, TRBC11; exemplary genomic sequence described in Table 2 herein; exemplary nucleotide sequence of the human TRBC2 gene locus set forth in SEQ ID NO:3; NCBI Reference Sequence: NG_001333.2, TRBC2); and a first complementarity domain. The second strand generally includes, from 5′ to 3′: optionally, a 5′ extension domain; a second complementarity domain; a proximal domain; and optionally, a tail domain.

A) Targeting Domain

Examples of the placement of targeting domains include those described in WO2015/161276, e.g., in FIGS. 1A-1G therein. The targeting domain comprises a nucleotide sequence that is complementary, e.g., at least 80, 85, 90, 95, 98 or 99% complementary, e.g., fully complementary, to the target sequence on the target nucleic acid. The strand of the target nucleic acid comprising the target sequence is referred to herein as the “complementary strand” of the target nucleic acid. Guidance on the selection of targeting domains can be found, e.g., in Fu Y et al., Nat Biotechnol 2014 (doi: 10.1038/nbt.2808) and Sternberg S H et al., Nature 2014 (doi: 10.1038/nature13011).

The targeting domain is part of an RNA molecule and will therefore comprise the base uracil (U), while any DNA encoding the gRNA molecule will comprise the base thymine (T). While not wishing to be bound by theory, In some embodiments, it is believed that the complementarity of the targeting domain with the target sequence contributes to specificity of the interaction of the gRNA molecule/Cas9 molecule complex with a target nucleic acid. It is understood that in a targeting domain and target sequence pair, the uracil bases in the targeting domain will pair with the adenine bases in the target sequence. In some embodiments, the target domain itself comprises in the 5′ to 3′ direction, an optional secondary domain, and a core domain. In some embodiments, the core domain is fully complementary with the target sequence. In some embodiments, the targeting domain is 5 to 50 nucleotides in length. The strand of the target nucleic acid with which the targeting domain is complementary is referred to herein as the complementary strand. Some or all of the nucleotides of the domain can have a modification, e.g., to render it less susceptible to degradation, improve bio-compatibility, etc.

By way of non-limiting example, the backbone of the target domain can be modified with a phosphorothioate, or other modification(s). In some cases, a nucleotide of the targeting domain can comprise a 2′ modification, e.g., a 2-acetylation, e.g., a 2′ methylation, or other modification(s).

In various embodiments, the targeting domain is 16-26 nucleotides in length (i.e. it is 16 nucleocides in length, or 17 nucleotides in length, or 18, 19, 20, 21, 22, 23, 24, 25 or 26 nucleotides in length.

B) Exemplary Targeting Domains

Exemplary targeting domains contained within the gRNA for targeting the genetic disruption of the human TRAC, TRBC1 or TRBC2 include those described in, e.g., WO2015/161276, WO2017/193107, WO2017/093969, US2016/272999 and US2015/056705 or a targeting domain that can bind to the targeting sequences described in the foregoing. Exemplary targeting domains contained within the gRNA for targeting the genetic disruption of the human TRAC locus using S. pyogenes or S. aureus Cas9 can include any of those set forth in Table-4.

TABLE 4
Exemplary TRAC gRNA targeting domain sequences

| | | | SEQ |
|---|---|---|---|
| | | | ID |
| gRNA Name | Targeting Domain | Cas9 species | NO: |
| TRAC-10 | UCUCUCAGCUGGUACACGGC | S. pyogenes | 28 |
| TRAC-110 | UGGAUUUAGAGUCUCUCAGC | S. pyogenes | 29 |
| TRAC-116 | ACACGGCAGGGUCAGGGUUC | S. pyogenes | 30 |
| TRAC-16 | GAGAAUCAAAAUCGGUGAAU | S. pyogenes | 31 |
| TRAC-4 | GCUGGUACACGGCAGGGUCA | S. pyogenes | 32 |
| TRAC-49 | CUCAGCUGGUACACGGC | S. pyogenes | 33 |
| TRAC-2 | UGGUACACGGCAGGGUC | S. pyogenes | 34 |
| TRAC-30 | GCUAGACAUGAGGUCUA | S. pyogenes | 35 |
| TRAC-43 | GUCAGAUUUGUUGCUCC | S. pyogenes | 36 |
| TRAC-23 | UCAGCUGGUACACGGCA | S. pyogenes | 37 |
| TRAC-34 | GCAGACAGACUUGUCAC | S. pyogenes | 38 |
| TRAC-25 | GGUACACGGCAGGGUCA | S. pyogenes | 39 |
| TRAC-128 | CUUCAAGAGCAACAGUGCUG | S. pyogenes | 40 |
| TRAC-105 | AGAGCAACAGUGCUGUGGCC | S. pyogenes | 41 |
| TRAC-106 | AAAGUCAGAUUUGUUGCUCC | S. pyogenes | 42 |
| TRAC-123 | ACAAAACUGUGCUAGACAUG | S. pyogenes | 43 |
| TRAC-64 | AAACUGUGCUAGACAUG | S. pyogenes | 44 |
| TRAC-97 | UGUGCUAGACAUGAGGUCUA | S. pyogenes | 45 |
| TRAC-148 | GGCUGGGGAAGAAGGUGUCUUC | S. aureus | 46 |
| TRAC-147 | GCUGGGGAAGAAGGUGUCUUC | S. aureus | 47 |
| TRAC-234 | GGGGAAGAAGGUGUCUUC | S. aureus | 48 |
| TRAC-167 | GUUUUGUCUGUGAUAUACACAU | S. aureus | 49 |
| TRAC-177 | GGCAGACAGACUUGUCACUGGA | S. aureus | 50 |
| | UU | | |
| TRAC-176 | GCAGACAGACUUGUCACUGGAUU | S. aureus | 51 |
| TRAC-257 | GACAGACUUGUCACUGGAUU | S. aureus | 52 |
| TRAC-233 | GUGAAUAGGCAGACAGACUUGU | S. aureus | 53 |
| | CA | | |
| TRAC-231 | GAAUAGGCAGACAGACUUGUCA | S. aureus | 54 |
| TRAC-163 | GAGUCUCUCAGCUGGUACACGG | S. aureus | 55 |
| TRAC-241 | GUCUCUCAGCUGGUACACGG | S. aureus | 56 |
| TRAC-179 | GGUACACGGCAGGGUCAGGGUU | S. aureus | 57 |
| TRAC-178 | GUACACGGCAGGGUCAGGGUU | S. aureus | 58 |

Exemplary targeting domains contained within the gRNA for targeting the genetic disruption of the human TRBC1 or TRBC2 locus using S. pyogenes or S. aureus Cas9 can include any of those set forth in Table 5

TABLE 5
Exemplary TRBC1 or TRBC2 gRNA targeting
domain sequences

| | | | SEQ |
|---|---|---|---|
| gRNA | | Cas9 | ID |
| Name | Targeting Domain | species | NO: |
| TRBC-40 | CACCCAGAUCGUCAGCGCCG | S. pyogenes | 59 |
| TRBC-52 | CAAACACAGCGACCUCGGGU | S. pyogenes | 60 |
| TRBC-25 | UGACGAGUGGACCCAGGAUA | S. pyogenes | 61 |
| TRBC-35 | GGCUCUCGGAGAAUGACGAG | S. pyogenes | 62 |
| TRBC-50 | GGCCUCGGCGCUGACGAUCU | S. pyogenes | 63 |
| TRBC-39 | GAAAAACGUGUUCCCACCCG | S. pyogenes | 64 |
| TRBC-49 | AUGACGAGUGGACCCAGGAU | S. pyogenes | 65 |
| TRBC-51 | AGUCCAGUUCUACGGGCUCU | S. pyogenes | 66 |
| TRBC-26 | CGCUGUCAAGUCCAGUUCUA | S. pyogenes | 67 |
| TRBC-47 | AUCGUCAGCGCCGAGGCCUG | S. pyogenes | 68 |
| TRBC-45 | UCAAACACAGCGACCUCGGG | S. pyogenes | 69 |
| TRBC-34 | CGUAGAACUGGACUUGACAG | S. pyogenes | 70 |
| TRBC-227 | AGGCCUCGGCGCUGACGAUC | S. pyogenes | 71 |
| TRBC-41 | UGACAGCGGAAGUGGUUGCG | S. pyogenes | 72 |
| TRBC-30 | UUGACAGCGGAAGUGGUUGC | S. pyogenes | 73 |
| TRBC-206 | UCUCCGAGAGCCCGUAGAAC | S. pyogenes | 74 |
| TRBC-32 | CGGGUGGGAACACGUUUUUC | S. pyogenes | 75 |
| TRBC-276 | GACAGGUUUGGCCCUAUCCU | S. pyogenes | 76 |
| TRBC-274 | GAUCGUCAGCGCCGAGGCCU | S. pyogenes | 77 |
| TRBC-230 | GGCUCAAACACAGCGACCUC | S. pyogenes | 78 |
| TRBC-235 | UGAGGGUCUCGGCCACCUUC | S. pyogenes | 79 |
| TRBC-38 | AGGCUUCUACCCCGACCACG | S. pyogenes | 80 |
| TRBC-223 | CCGACCACGUGGAGCUGAGC | S. pyogenes | 81 |
| TRBC-221 | UGACAGGUUUGGCCCUAUCC | S. pyogenes | 82 |
| TRBC-48 | CUUGACAGCGGAAGUGGUUG | S. pyogenes | 83 |
| TRBC-216 | AGAUCGUCAGCGCCGAGGCC | S. pyogenes | 84 |
| TRBC-210 | GCGCUGACGAUCUGGGUGAC | S. pyogenes | 85 |
| TRBC-268 | UGAGGGCGGGCUGCUCCUUG | S. pyogenes | 86 |
| TRBC-193 | GUUGCGGGGGUUCUGCCAGA | S. pyogenes | 87 |
| TRBC-246 | AGCUCAGCUCCACGUGGUCG | S. pyogenes | 88 |
| TRBC-228 | GCGGCUGCUCAGGCAGUAUC | S. pyogenes | 89 |
| TRBC-43 | GCGGGGGUUCUGCCAGAAGG | S. pyogenes | 90 |
| TRBC-272 | UGGCUCAAACACAGCGACCU | S. pyogenes | 91 |
| TRBC-33 | ACUGGACUUGACAGCGGAAG | S. pyogenes | 92 |
| TRBC-44 | GACAGCGGAAGUGGUUGCGG | S. pyogenes | 93 |
| TRBC-211 | GCUGUCAAGUCCAGUUCUAC | S. pyogenes | 94 |
| TRBC-253 | GUAUCUGGAGUCAUUGAGGG | S. pyogenes | 95 |
| TRBC-18 | CUCGGCGCUGACGAUCU | S. pyogenes | 96 |
| TRBC-6 | CCUCGGCGCUGACGAUC | S. pyogenes | 97 |
| TRBC-85 | CCGAGAGCCCGUAGAAC | S. pyogenes | 98 |
| TRBC-129 | CCAGAUCGUCAGCGCCG | S. pyogenes | 99 |
| TRBC-93 | GAAUGACGAGUGGACCC | S. pyogenes | 100 |
| TRBC-415 | GGGUGACAGGUUUGGCCCUA | S. aureus | 101 |
| | UC | | |
| TRBC-414 | GGUGACAGGUUUGGCCCUAU | S. aureus | 102 |
| | C | | |
| TRBC-310 | GUGACAGGUUUGGCCCUAUC | S. aureus | 103 |
| TRBC-308 | GACAGGUUUGGCCCUAUC | S. aureus | 104 |
| TRBC-401 | GAUACUGCCUGAGCAGCCGC | S. aureus | 105 |
| | CU | | |
| TRBC-468 | GACCACGUGGAGCUGAGCUG | S. aureus | 106 |
| | GUGG | | |
| TRBC-462 | GUGGAGCUGAGCUGGUGG | S. aureus | 107 |
| TRBC-424 | GGGCGGGCUGCUCCUUGAGG | S. aureus | 108 |
| | GGCU | | |
| TRBC-423 | GGCGGGCUGCUCCUUGAGGG | S. aureus | 109 |
| | GCU | | |
| TRBC-422 | GCGGGCUGCUCCUUGAGGGG | S. aureus | 110 |
| | CU | | |
| TRBC-420 | GGGCUGCUCCUUGAGGGGCU | S. aureus | 111 |
| TRBC-419 | GGCUGCUCCUUGAGGGGCU | S. aureus | 112 |
| TRBC-418 | GCUGCUCCUUGAGGGGCU | S. aureus | 113 |
| TRBC-445 | GGUGAAUGGGAAGGAGGUGC | S. aureus | 114 |
| | ACAG | | |
| TRBC-444 | GUGAAUGGGAAGGAGGUGCA | S. aureus | 115 |
| | CAG | | |
| TRBC-442 | GAAUGGGAAGGAGGUGCACA | S. aureus | 116 |
| | G | | |

In some embodiments, the gRNA for targeting TRAC, TRBC1 and/or TRBC2 can be any that are described herein, or are described elsewhere e.g., in WO2015/161276, WO2017/193107, WO2017/093969, US2016/272999 and US2015/056705 or a targeting domain that can bind to the targeting sequences described in the foregoing. In some embodiments, the sequence targeted by the CRISPR/Cas9 gRNA in the TRAC gene locus is set forth in SEQ ID NOS: 117, 163 and 165-211, such as GAGAATCAAAATCGGTGAAT (SEQ ID NO:163) or ATTCACCGATTTTGATTCTC (SEQ ID NO:117). In some embodiments, the sequence targeted by the CRISPR/Cas9 gRNA in the TRBC1 and/or TRBC2 gene loci is set forth in SEQ ID NOS: 118, 164 and 212, such as GGCCTCGGCGCTGACGATCT (SEQ ID NO:164) or GATCGTCAGCGCCGAGGCC (SEQ ID NO:118). In some embodiments, the gRNA targeting domain sequence for targeting a target site in the TRAC gene locus is GAGAAUCAAAAUCGGUGAAU (SEQ ID NO:31). In some embodiments, the gRNA targeting domain sequence for targeting a target site in the TRBC1 and/or TRBC2 gene loci is GGCCUCGGCGCUGACGAUCU (SEQ ID NO:63).

In some embodiments, the gRNA for targeting the TRAC gene locus can be obtained by in vitro transcription of the sequence AGCGCTCTCGTACAGAGTTGGCATTATAATACGACTCACTATAGGGGAGAATCAAA ATCGGTGAATGTTTTAGAGCTAGAAATAGCAAGTTAAAATAAGGCTAGTCCGTTAT CAACTTGAAAAAGTGGCACCGAGTCGGTGCTTTTTTT (set forth in SEQ ID NO:26; bold and underlined portion is complementary to the target site in the TRAC locus), or chemically synthesized, where the gRNA had the sequence 5′-GAG AAU CAA AAU CGG UGA AUG UUUUAGAGCUAGAAAUAGCAAGUUAAAAUAAGGCUAGUCCGUUAUCAA CUU GAA AAA GUG GCA CCG AGU CGG UGC UUU U-3′ (set forth in SEQ ID NO:27; see Osborn et al., Mol Ther. 24(3):570-581 (2016)). Other exemplary gRNA sequences to generate a genetic disruption of the endogenous genes encoding TCR domains or regions, e.g., TRAC, TRBC1 and/or TRBC2 are described, e.g., in International PCT Publication No. WO2015/161276. Exemplary methods for gene editing of the endogenous TCR loci include those described in, e.g. U.S. Publication Nos. US2011/0158957, US2014/0301990, US2015/0098954, US2016/0208243; US2016/272999 and US2015/056705; International PCT Publication Nos. WO2014/191128, WO2015/136001, WO2015/161276, WO2016/069283, WO2016/016341; and Osborn et al. (2016) Mol. Ther. 24(3):570-581. Any of the known methods can be used to generate a genetic disruption of the endogenous genes encoding TCR domains or regions can be used in the embodiments provided herein.

In some embodiments, targeting domains include those for introducing a genetic disruption at the TRAC, TRBC1 and/or TRBC2 loci using S. pyogenes Cas9 or using N. meningitidis Cas9.

In some embodiments, targeting domains include those for introducing a genetic disruption at the TRAC, TRBC1 and/or TRBC2 loci using S. pyogenes Cas9. Any of the targeting domains can be used with a S. pyogenes Cas9 molecule that generates a double stranded break (Cas9 nuclease) or a single-stranded break (Cas9 nickase).

In some embodiments, dual targeting is used to create two nicks on opposite DNA strands by using S. pyogenes Cas9 nickases with two targeting domains that are complementary to opposite DNA strands, e.g., a gRNA comprising any minus strand targeting domain may be paired with any gRNA comprising a plus strand targeting domain. In some embodiments, the two gRNAs are oriented on the DNA such that PAMs face outward and the distance between the 5′ ends of the gRNAs is 0-50 bp. In some embodiments, two gRNAs are used to target two Cas9 nucleases or two Cas9 nickases, for example, using a pair of Cas9 molecule/gRNA molecule complex guided by two different gRNA molecules to cleave the target domain with two single stranded breaks on opposing strands of the target domain. In some embodiments, the two Cas9 nickases can include a molecule having HNH activity, e.g., a Cas9 molecule having the RuvC activity inactivated, e.g., a Cas9 molecule having a mutation at D10, e.g., the D10A mutation, a molecule having RuvC activity, e.g., a Cas9 molecule having the HNH activity inactivated, e.g., a Cas9 molecule having a mutation at H840, e.g., a H840A, or a molecule having RuvC activity, e.g., a Cas9 molecule having the HNH activity inactivated, e.g., a Cas9 molecule having a mutation at N863, e.g., N863A. In some embodiments, each of the two gRNAs are complexed with a D10A Cas9 nickase.

In some embodiments, the target sequence (target domain) is at or near the TRAC, TRBC1 and/or TRBC2 locus, such as any part of the TRAC, TRBC1 and/or TRBC2 coding sequence set forth in SEQ ID NO: 1-3 or described in Tables 1-3 herein. In some embodiments, the target nucleic acid complementary to the targeting domain is located at an early coding region of a gene of interest, such as TRAC, TRBC1 and/or TRBC2. Targeting of the early coding region can be used to genetic disruption (i.e., eliminate expression of) the gene of interest. In some embodiments, the early coding region of a gene of interest includes sequence immediately following a start codon (e.g., ATG), or within 500 bp of the start codon (e.g., less than 500, 450, 400, 350, 300, 250, 200, 150, 100, 50 bp, 40 bp, 30 bp, 20 bp, or 10 bp). In particular examples, the target nucleic acid is within 200 bp, 150 bp, 100 bp, 50 bp, 40 bp, 30 bp, 20 bp or 10 bp of the start codon. In some examples, the targeting domain of the gRNA is complementary, e.g., at least 80, 85, 90, 95, 98 or 99% complementary, e.g., fully complementary, to the target sequence on the target nucleic acid, such as the target nucleic acid in the TRAC, TRBC1 and/or TRBC2 locus.

In some aspects, the gRNA can target a site within an exon of the open reading frame of the endogenous TRAC, TRBC1 and/or TRBC2 locus. In some aspects, the gRNA can target a site within an intron of the open reading frame of the TRAC, TRBC1 and/or TRBC2 locus. In some aspects, the gRNA can target a site within a regulatory or control element, e.g., a promoter, of the TRAC, TRBC1 and/or TRBC2 locus. In some aspects, the target site at the TRAC, TRBC1 and/or TRBC2 locus that is targeted by the gRNA can be any target sites described herein, e.g., in Section I.A.1. In some embodiments, the gRNA can target a site within or in close proximity to exons corresponding to early coding region, e.g., exon 1, 2 or 3 of the open reading frame of the endogenous TRAC, TRBC1 and/or TRBC2 locus, or including sequence immediately following a transcription start site, within exon 1, 2, or 3, or within less than 500, 450, 400, 350, 300, 250, 200, 150, 100 or 50 bp of exon 1, 2, or 3. In some embodiments, the gRNA can target a site at or near exon 2 of the endogenous TRAC, TRBC1 and/or TRBC2 locus, or within less than 500, 450, 400, 350, 300, 250, 200, 150, 100 or 50 bp of exon 2.

C) The First Complementarity Domain

Examples of first complementarity domains include those described in WO2015/161276, e.g., in FIGS. 1A-1G therein. The first complementarity domain is complementary with the second complementarity domain described herein, and generally has sufficient complementarity to the second complementarity domain to form a duplexed region under at least some physiological conditions. The first complementarity domain is typically 5 to 30 nucleotides in length, and may be 5 to 25 nucleotides in length, 7 to 25 nucleotides in length, 7 to 22 nucleotides in length, 7 to 18 nucleotides in length, or 7 to 15 nucleotides in length. In various embodiments, the first complementary domain is 5, 6, 7, 8, 9, 10, 11, 12, 13, 14, 15, 16, 17, 18, 19, 20, 21, 22, 23, 24, or 25 nucleotides in length.

Typically, the first complementarity domain does not have exact complementarity with the second complementarity domain target. In some embodiments, the first complementarity domain can have 1, 2, 3, 4 or 5 nucleotides that are not complementary with the corresponding nucleotide of the second complementarity domain. For instance, a segment of 1, 2, 3, 4, 5 or 6, (e.g., 3) nucleotides of the first complementarity domain may not pair in the duplex, and may form a non-duplexed or looped-out region. In some instances, an unpaired, or loop-out, region, e.g., a loop-out of 3 nucleotides, is present on the second complementarity domain. This unpaired region optionally begins 1, 2, 3, 4, 5, or 6, e.g., 4, nucleotides from the 5′ end of the second complementarity domain.

The first complementarity domain can include 3 subdomains, which, in the 5′ to 3′ direction are: a 5′ subdomain, a central subdomain, and a 3′ subdomain. In some embodiments, the 5′ subdomain is 4-9, e.g., 4, 5, 6, 7, 8 or 9 nucleotides in length. In some embodiments, the central subdomain is 1, 2, or 3, e.g., 1, nucleotide in length. In some embodiments, the 3′ subdomain is 3 to 25, e.g., 4-22, 4-18, or 4 to 10, or 3, 4, 5, 6, 7, 8, 9, 10, 11, 12, 13, 14, 15, 16, 17, 18, 19, 20, 21, 22, 23, 24, or 25, nucleotides in length.

In some embodiments, the first and second complementarity domains, when duplexed, comprise 11 paired nucleotides, for example, in the gRNA sequence (one paired strand underlined, one bolded):

(SEQ ID NO: 144)
NNNNNNNNNNNNNNNNNNNNGUUUUAGAGCUAGAAAUAGCAAGUUAAAAU
AAGGCUAGUCCGUUAUCAACUUGAAAAAGUGGCACCGAGUCGGUGC.

In some embodiments, the first and second complementarity domains, when duplexed, comprise 15 paired nucleotides, for example in the gRNA sequence (one paired strand underlined, one bolded):

(SEQ ID NO: 145)
NNNNNNNNNNNNNNNNNNNNGUUUUAGAGCUAUGCUGAAAAGCAUAGCAA
GUUAAAAUAAGGCUAGUCCGUUAUCAACUUGAAAAAGUGGCACCGAGUCG
GUGC.

In some embodiments the first and second complementarity domains, when duplexed, comprise 16 paired nucleotides, for example in the gRNA sequence (one paired strand underlined, one bolded):

(SEQ ID NO: 146)
NNNNNNNNNNNNNNNNNNNNGUUUUAGAGCUAUGCUGGAAACAGCAUAGC
AAGUUAAAAUAAGGCUAGUCCGUUAUCAACUUGAAAAAGUGGCACCGAGU
CGGUGC.

In some embodiments the first and second complementarity domains, when duplexed, comprise 21 paired nucleotides, for example in the gRNA sequence (one paired strand underlined, one bolded):

(SEQ ID NO: 147)
NNNNNNNNNNNNNNNNNNNNGUUUUAGAGCUAUGCUGUUUUGGAAACAAA
ACAGCAUAGCAAGUUAAAAUAAGGCUAGUCCGUUAUCAACUUGAAAAAGU
GGCACCGAGUCGGUGC.

In some embodiments, nucleotides are exchanged to remove poly-U tracts, for example in the gRNA sequences (exchanged nucleotides underlined):

(SEQ ID NO: 148)
NNNNNNNNNNNNNNNNNNNNGUAUUAGAGCUAGAAAUAGCAAGUUAAUAU
AAGGCUAGUCCGUUAUCAACUUGAAAAAGUGGCACCGAGUCGGUGC;
(SEQ ID NO: 149)
NNNNNNNNNNNNNNNNNNNNGUUUAAGAGCUAGAAAUAGCAAGUUUAAAU
AAGGCUAGUCCGUUAUCAACUUGAAAAAGUGGCACCGAGUCGGUGC;
and
(SEQ ID NO: 150)
NNNNNNNNNNNNNNNNNNNNGUAUUAGAGCUAUGCUGUAUUGGAAACAAU
ACAGCAUAGCAAGUUAAUAUAAGGCUAGUCCGUUAUCAACUUGAAAAAGU
GGCACCGAGUCGGUGC.

The first complementarity domain can share homology with, or be derived from, a naturally occurring first complementarity domain. In some embodiments, it has at least 50% homology with a first complementarity domain disclosed herein, e.g., an S. pyogenes, S. aureus, N. meningtidis, or S. thermophilus, first complementarity domain.

It should be noted that one or more, or even all of the nucleotides of the first complementarity domain, can have a modification along the lines discussed herein for the targeting domain.

D) The Linking Domain

Examples of linking domains include those described in WO2015/161276, e.g., in FIGS. 1A-1G therein. In a unimolecular or chimeric gRNA, the linking domain serves to link the first complementarity domain with the second complementarity domain of a unimolecular gRNA. The linking domain can link the first and second complementarity domains covalently or non-covalently. In some embodiments, the linkage is covalent. In some embodiments, the linking domain covalently couples the first and second complementarity domains, see, e.g., WO2015/161276, e.g., in FIGS. 1B-1E therein. In some embodiments, the linking domain is, or comprises, a covalent bond interposed between the first complementarity domain and the second complementarity domain. Typically the linking domain comprises one or more, e.g., 2, 3, 4, 5, 6, 7, 8, 9, or 10 nucleotides, but in various embodiments the linker can be 20, 30, 40, 50 or even 100 nucleotides in length.

In modular gRNA molecules, the two molecules are associated by virtue of the hybridization of the complementarity domains and a linking domain may not be present. See e.g., WO2015/161276, e.g., in FIG. 1A therein.

A wide variety of linking domains are suitable for use in unimolecular gRNA molecules. Linking domains can consist of a covalent bond, or be as short as one or a few nucleotides, e.g., 1, 2, 3, 4, or 5 nucleotides in length. In some embodiments, a linking domain is 2, 3, 4, 5, 6, 7, 8, 9, 10, 15, 20, or 25 or more nucleotides in length. In some embodiments, a linking domain is 2 to 50, 2 to 40, 2 to 30, 2 to 20, 2 to 10, or 2 to 5 nucleotides in length. In some embodiments, a linking domain shares homology with, or is derived from, a naturally occurring sequence, e.g., the sequence of a tracrRNA that is 5′ to the second complementarity domain. In some embodiments, the linking domain has at least 50% homology with a linking domain disclosed herein.

As discussed herein in connection with the first complementarity domain, some or all of the nucleotides of the linking domain can include a modification.

E) The 5′ Extension Domain

In some cases, a modular gRNA can comprise additional sequence, 5′ to the second complementarity domain, referred to herein as the 5′ extension domain, WO2015/161276, e.g., in FIG. 1A therein. In some embodiments, the 5′ extension domain is 2-10, 2-9, 2-8, 2-7, 2-6, 2-5, or 2-4 nucleotides in length. In some embodiments, the 5′ extension domain is 2, 3, 4, 5, 6, 7, 8, 9, or 10 or more nucleotides in length.
F) The Second Complementarity Domain

Examples of second complementarity domains include those described in WO2015/161276, e.g., in FIGS. 1A-1G therein. The second complementarity domain is complementary with the first complementarity domain, and generally has sufficient complementarity to the second complementarity domain to form a duplexed region under at least some physiological conditions. In some cases, e.g., as shown in WO2015/161276, e.g., in FIG. 1A-1B therein, the second complementarity domain can include sequence that lacks complementarity with the first complementarity domain, e.g., sequence that loops out from the duplexed region.

The second complementarity domain may be 5 to 27 nucleotides in length, and in some cases may be longer than the first complementarity region. For instance, the second complementary domain can be 7 to 27 nucleotides in length, 7 to 25 nucleotides in length, 7 to 20 nucleotides in length, or 7 to 17 nucleotides in length. More generally, the complementary domain may be 5, 6, 7, 8, 9, 10, 11, 12, 13, 14, 15, 16, 17, 18, 19, 20, 21, 22, 23, 24, 25, or 26 nucleotides in length.

In some embodiments, the second complementarity domain comprises 3 subdomains, which, in the 5′ to 3′ direction are: a 5′ subdomain, a central subdomain, and a 3′ subdomain. In some embodiments, the 5′ subdomain is 3 to 25, e.g., 4 to 22, 4 tol8, or 4 to 10, or 3, 4, 5, 6, 7, 8, 9, 10, 11, 12, 13, 14, 15, 16, 17, 18, 19, 20, 21, 22, 23, 24, or 25 nucleotides in length. In some embodiments, the central subdomain is 1, 2, 3, 4 or 5, e.g., 3, nucleotides in length. In some embodiments, the 3′ subdomain is 4 to 9, e.g., 4, 5, 6, 7, 8 or 9 nucleotides in length.

In some embodiments, the 5′ subdomain and the 3′ subdomain of the first complementarity domain, are respectively, complementary, e.g., fully complementary, with the 3′ subdomain and the 5′ subdomain of the second complementarity domain.

The second complementarity domain can share homology with or be derived from a naturally occurring second complementarity domain. In some embodiments, it has at least 50% homology with a second complementarity domain disclosed herein, e.g., an S. pyogenes, S. aureus, N. meningtidis, or S. thermophilus, first complementarity domain.

Some or all of the nucleotides of the second complementarity domain can have a modification, e.g., a modification described herein.

G) The Proximal Domain

Examples of proximal domains include those described in WO2015/161276, e.g., in FIGS. 1A-1G therein. In some embodiments, the proximal domain is 5 to 20 nucleotides in length. In some embodiments, the proximal domain can share homology with or be derived from a naturally occurring proximal domain. In some embodiments, it has at least 50% homology with a proximal domain disclosed herein, e.g., an S. pyogenes, S. aureus, N. meningtidis, or S. thermophilus, proximal domain.

Some or all of the nucleotides of the proximal domain can have a modification along the lines described herein.

H) The Tail Domain

Examples of tail domains include those described in WO2015/161276, e.g., in FIGS. 1A-1G therein. As can be seen by inspection of the tail domains in WO2015/161276, e.g., in FIG. 1A and FIGS. 1B-1F therein, a broad spectrum of tail domains are suitable for use in gRNA molecules. In various embodiments, the tail domain is 0 (absent), 1, 2, 3, 4, 5, 6, 7, 8, 9, or 10 nucleotides in length. In certain embodiments, the tail domain nucleotides are from or share homology with sequence from the 5′ end of a naturally occurring tail domain, see e.g., WO2015/161276, e.g., in FIG. 1D or 1E therein. The tail domain also optionally includes sequences that are complementary to each other and which, under at least some physiological conditions, form a duplexed region.

Tail domains can share homology with or be derived from naturally occurring proximal tail domains. By way of non-limiting example, a given tail domain according to various embodiments of the present disclosure may share at least 50% homology with a naturally occurring tail domain disclosed herein, e.g., an S. pyogenes, S. aureus, N. meningtidis, or S. thermophilus, tail domain.

In certain cases, the tail domain includes nucleotides at the 3′ end that are related to the method of in vitro or in vivo transcription. When a T7 promoter is used for in vitro transcription of the gRNA, these nucleotides may be any nucleotides present before the 3′ end of the DNA template. When a U6 promoter is used for in vivo transcription, these nucleotides may be the sequence UUUUUU. When alternate pol-III promoters are used, these nucleotides may be various numbers or uracil bases or may include alternate bases.

As a non-limiting example, in various embodiments the proximal and tail domain, taken together comprise the following sequences:

(SEQ ID NO: 151)
AAGGCUAGUCCGUUAUCAACUUGAAAAAGUGGCACCGAGUCGGUGCU,
(SEQ ID NO: 152)
AAGGCUAGUCCGUUAUCAACUUGAAAAAGUGGCACCGAGUCGGUGGUGC,
(SEQ ID NO: 153)
AAGGCUAGUCCGUUAUCAACUUGAAAAAGUGGCACCGAGUCGGUGCGGAU
C,
(SEQ ID NO: 154)
AAGGCUAGUCCGUUAUCAACUUGAAAAAGUG,
(SEQ ID NO: 155)
AAGGCUAGUCCGUUAUCA,
or
(SEQ ID NO: 156)
AAGGCUAGUCCG.

In some embodiments, the tail domain comprises the 3′ sequence UUUUUU, e.g., if a U6 promoter is used for transcription. In some embodiments, the tail domain comprises the 3′ sequence UUUU, e.g., if an H1 promoter is used for transcription. In some embodiments, tail domain comprises variable numbers of 3′ Us depending, e.g., on the termination signal of the pol-II promoter used. In some embodiments, the tail domain comprises variable 3′ sequence derived from the DNA template if a T7 promoter is used. In some embodiments, the tail domain comprises variable 3′ sequence derived from the DNA template, e.g., if in vitro transcription is used to generate the RNA molecule. In some embodiments, the tail domain comprises variable 3′ sequence derived from the DNA template, e.g., if a pol-II promoter is used to drive transcription.

In some embodiments a gRNA has the following structure: 5′ [targeting domain]-[first complementarity domain]-[linking domain]-[second complementarity domain]-[proximal domain]-[tail domain]-3′ wherein, the targeting domain comprises a core domain and optionally a secondary domain, and is 10 to 50 nucleotides in length; the first complementarity domain is 5 to 25 nucleotides in length and, In some embodiments has at least 50, 60, 70, 80, 85, 90, 95, 98 or 99% homology with a reference first complementarity domain disclosed herein; the linking domain is 1 to 5 nucleotides in length; the proximal domain is 5 to 20 nucleotides in length and, In some embodiments has at least 50, 60, 70, 80, 85, 90, 95, 98 or 99% homology with a reference proximal domain disclosed herein; and the tail domain is absent or a nucleotide sequence is 1 to 50 nucleotides in length and, In some embodiments has at least 50, 60, 70, 80, 85, 90, 95, 98 or 99% homology with a reference tail domain disclosed herein.

I) Exemplary Chimeric gRNAs

In some embodiments, a unimolecular, or chimeric, gRNA comprises, preferably from 5′ to 3′: a targeting domain, e.g., comprising 15, 16, 17, 18, 19, 20, 21, 22, 23, 24, 25, or 26 nucleotides (which is complementary to a target nucleic acid); a first complementarity domain; a linking domain; a second complementarity domain (which is complementary to the first complementarity domain); a proximal domain; and a tail domain, wherein, (a) the proximal and tail domain, when taken together, comprise at least 15, 18, 20, 25, 30, 31, 35, 40, 45, 49, 50, or 53 nucleotides; (b) there are at least 15, 18, 20, 25, 30, 31, 35, 40, 45, 49, 50, or 53 nucleotides 3′ to the last nucleotide of the second complementarity domain; or (c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In some embodiments, the sequence from (a), (b), or (c), has at least 60, 75, 80, 85, 90, 95, or 99% homology with the corresponding sequence of a naturally occurring gRNA, or with a gRNA described herein. In some embodiments, the proximal and tail domain, when taken together, comprise at least 15, 18, 20, 25, 30, 31, 35, 40, 45, 49, 50, or 53 nucleotides. In some embodiments, there are at least 15, 18, 20, 25, 30, 31, 35, 40, 45, 49, 50, or 53 nucleotides 3′ to the last nucleotide of the second complementarity domain. In some embodiments, there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain. In some embodiments, the targeting domain comprises, has, or consists of, 16, 17, 18, 19, 20, 21, 22, 23, 24, 25 or 26 nucleotides (e.g., 16, 17, 18, 19, 20, 21, 22, 23, 24, 25 or 26 consecutive nucleotides) having complementarity with the target domain, e.g., the targeting domain is 16, 17, 18, 19, 20, 21, 22, 23, 24, 25 or 26 nucleotides in length.

In some embodiments, the unimolecular, or chimeric, gRNA molecule (comprising a targeting domain, a first complementary domain, a linking domain, a second complementary domain, a proximal domain and, optionally, a tail domain) comprises the following sequence in which the targeting domain is depicted as 20 Ns but could be any sequence and range in length from 16 to 26 nucleotides and in which the gRNA sequence is followed by 6 Us, which serve as a termination signal for the U6 promoter, but which could be either absent or fewer in number: NNNNNNNNNNNNNNNNNNNNGUUUUAGAGCUAGAAAUAGCAAGUUAAAAUAAG GCUAGUCCGUUAUCAACUUGAAAAAGUGGCACCGAGUCGGUGCUUUUUU (SEQ ID NO:157). In some embodiments, the unimolecular, or chimeric, gRNA molecule is a S. pyogenes gRNA molecule.

In some embodiments, the unimolecular, or chimeric, gRNA molecule (comprising a targeting domain, a first complementary domain, a linking domain, a second complementary domain, a proximal domain and, optionally, a tail domain) comprises the following sequence in which the targeting domain is depicted as 20 Ns but could be any sequence and range in length from 16 to 26 nucleotides and in which the gRNA sequence is followed by 6 Us, which serve as a termination signal for the U6 promoter, but which could be either absent or fewer in number: NNNNNNNNNNNNNNNNNNNNGUUUUAGUACUCUGGAAACAGAAUCUACUAAAAC AAGGCAAAAUGCCGUGUUUAUCUCGUCAACUUGUUGGCGAGAUUUUUU (SEQ ID NO:158). In some embodiments, the unimolecular, or chimeric, gRNA molecule is a S. aureus gRNA molecule. The sequences and structures of exemplary chimeric gRNAs are also shown in WO2015/161276, e.g., in FIGS. 10A-10B therein.

J) Exemplary Modular gRNAs

In some embodiments, a modular gRNA comprises first and second strands. The first strand comprises, preferably from 5′ to 3′; a targeting domain, e.g., comprising 15, 16, 17, 18, 19, 20, 21, 22, 23, 24, 25, or 26 nucleotides; a first complementarity domain. The second strand comprises, preferably from 5′ to 3′: optionally a 5′ extension domain; a second complementarity domain; a proximal domain; and a tail domain, wherein: (a) the proximal and tail domain, when taken together, comprise at least 15, 18, 20, 25, 30, 31, 35, 40, 45, 49, 50, or 53 nucleotides; (b) there are at least 15, 18, 20, 25, 30, 31, 35, 40, 45, 49, 50, or 53 nucleotides 3′ to the last nucleotide of the second complementarity domain; or (c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In some embodiments, the sequence from (a), (b), or (c), has at least 60, 75, 80, 85, 90, 95, or 99% homology with the corresponding sequence of a naturally occurring gRNA, or with a gRNA described herein. In some embodiments, the proximal and tail domain, when taken together, comprise at least 15, 18, 20, 25, 30, 31, 35, 40, 45, 49, 50, or 53 nucleotides. In some embodiments there are at least 15, 18, 20, 25, 30, 31, 35, 40, 45, 49, 50, or 53 nucleotides 3′ to the last nucleotide of the second complementarity domain.

In some embodiments, there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In some embodiments, the targeting domain has, or consists of, 16, 17, 18, 19, 20, 21, 22, 23, 24, 25 or 26 nucleotides (e.g., 16, 17, 18, 19, 20, 21, 22, 23, 24, 25 or 26 consecutive nucleotides) having complementarity with the target domain, e.g., the targeting domain is 16, 17, 18, 19, 20, 21, 22, 23, 24, 25 or 26 nucleotides in length.

K) Methods for Designing gRNAs

Methods for designing gRNAs are described herein, including methods for selecting, designing and validating targeting domains. Exemplary targeting domains are also provided herein. Targeting domains discussed herein can be incorporated into the gRNAs described herein.

In some embodiments, a guide RNA (gRNA) specific to the target gene (e.g. TRAC, TRBC1 and/or TRBC2 in humans) is used to RNA-guided nucleases, e.g., Cas, to induce a DNA break at the target site or target position. Methods for designing gRNAs and exemplary targeting domains can include those described in, e.g., in International PCT Publication No. WO2015/161276. Targeting domains of can be incorporated into the gRNA that is used to target Cas9 nucleases to the target site or target position.

Methods for selection and validation of target sequences as well as off-target analyses are described, e.g., in Mali et al., 2013 Science 339(6121): 823-826; Hsu et al. Nat Biotechnol, 31(9): 827-32; Fu et al., 2014 Nat Biotechnol, doi: 10.1038/nbt.2808. PubMed PMID: 24463574; Heigwer et al., 2014 Nat Methods 11(2):122-3. doi: 10.1038/nmeth.2812.

PubMed PMID: 24481216; Bae et al., 2014 Bioinformatics PubMed PMID: 24463181; Xiao A et al., 2014 Bioinformatics PubMed PMID: 24389662.

In some embodiments, a software tool can be used to optimize the choice of gRNA within a user's target sequence, e.g., to minimize total off-target activity across the genome. Off target activity may be other than cleavage. For example, for each possible gRNA choice using S. pyogenes Cas9, software tools can identify all potential off-target sequences (preceding either NAG or NGG PAMs) across the genome that contain up to a certain number (e.g., 1, 2, 3, 4, 5, 6, 7, 8, 9, or 10) of mismatched base-pairs. The cleavage efficiency at each off-target sequence can be predicted, e.g., using an experimentally-derived weighting scheme. Each possible gRNA can then be ranked according to its total predicted off-target cleavage; the top-ranked gRNAs represent those that are likely to have the greatest on-target and the least off-target cleavage.

Other functions, e.g., automated reagent design for gRNA vector construction, primer design for the on-target Surveyor assay, and primer design for high-throughput detection and quantification of off-target cleavage via next-generation sequencing, can also be included in the tool.

Candidate gRNA molecules can be evaluated by art-known methods or as described herein.

In some embodiments, gRNAs for use with S. pyogenes, S. aureus, and N. meningitidis Cas9s are identified using a DNA sequence searching algorithm, e.g., using a custom gRNA design software based on the public tool cas-offinder (Bae et al. Bioinformatics. 2014; 30(10): 1473-1475). The custom gRNA design software scores guides after calculating their genome-wide off-target propensity. Typically matches ranging from perfect matches to 7 mismatches are considered for guides ranging in length from 17 to 24. In some aspects, once the off-target sites are computationally determined, an aggregate score is calculated for each guide and summarized in a tabular output using a web-interface. In addition to identifying potential gRNA sites adjacent to PAM sequences, the software also can identify all PAM adjacent sequences that differ by 1, 2, 3 or more nucleotides from the selected gRNA sites. In some embodiments, gGenomic DNA sequences for each gene are obtained from the UCSC Genome browser and sequences can be screened for repeat elements using the publicly available RepeatMasker program. RepeatMasker searches input DNA sequences for repeated elements and regions of low complexity. The output is a detailed annotation of the repeats present in a given query sequence.

Following identification, gRNAs can be ranked into tiers based on one or more of their distance to the target site, their orthogonality and presence of a 5′ G (based on identification of close matches in the human genome containing a relevant PAM, e.g., in the case of S. pyogenes, a NGG PAM, in the case of S. aureus, NNGRR (e.g., a NNGRRT or NNGRRV) PAM, and in the case of N. meningtidis, a NNNNGATT or NNNNGCTT PAM).

Orthogonality refers to the number of sequences in the human genome that contain a minimum number of mismatches to the target sequence. A “high level of orthogonality” or “good orthogonality” may, for example, refer to 20-mer targeting domains that have no identical sequences in the human genome besides the intended target, nor any sequences that contain one or two mismatches in the target sequence. Targeting domains with good orthogonality are selected to minimize off-target DNA cleavage. It is to be understood that this is a non-limiting example and that a variety of strategies could be utilized to identify gRNAs for use with S. pyogenes, S. aureus and N. meningitidis or other Cas9 enzymes.

In some embodiments, gRNAs for use with the S. pyogenes Cas9 can be identified using the publicly available web-based ZiFiT server (Fu et al., Improving CRISPR-Cas nuclease specificity using truncated guide RNAs. Nat Biotechnol. 2014 Jan. 26. doi: 10.1038/nbt.2808. PubMed PMID: 24463574, for the original references see Sander et al., 2007, NAR 35:W599-605; Sander et al., 2010, NAR 38: W462-8). In addition to identifying potential gRNA sites adjacent to PAM sequences, the software also identifies all PAM adjacent sequences that differ by 1, 2, 3 or more nucleotides from the selected gRNA sites. In some aspects, genomic DNA sequences for each gene can be obtained from the UCSC Genome browser and sequences can be screened for repeat elements using the publicly available Repeat-Masker program. RepeatMasker searches input DNA sequences for repeated elements and regions of low complexity. The output is a detailed annotation of the repeats present in a given query sequence.

Following identification, gRNAs for use with a S. pyogenes Cas9 can be ranked into tiers, e.g. into 5 tiers. In some embodiments, the targeting domains for first tier gRNA molecules are selected based on their distance to the target site, their orthogonality and presence of a 5′ G (based on the ZiFiT identification of close matches in the human genome containing an NGG PAM). In some embodiments, both 17-mer and 20-mer gRNAs are designed for targets.

In some aspects, gRNAs are also selected both for single-gRNA nuclease cutting and for the dual gRNA nickase strategy. Criteria for selecting gRNAs and the determination for which gRNAs can be used for which strategy can be based on several considerations. In some embodiments, gRNAs for both single-gRNA nuclease cleavage and for a dual-gRNA paired “nickase” strategy are identified. In some embodiments for selecting gRNAs, including the determination for which gRNAs can be used for the dual-gRNA paired “nickase” strategy, gRNA pairs should be oriented on the DNA such that PAMs are facing out and cutting with the D10A Cas9 nickase will result in 5′ overhangs. In some aspects, it can be assumed that cleaving with dual nickase pairs will result in deletion of the entire intervening sequence at a reasonable frequency. However, cleaving with dual nickase pairs can also often result in indel mutations at the site of only one of the gRNAs. Candidate pair members can be tested for how efficiently they remove the entire sequence versus just causing indel mutations at the site of one gRNA.

In some embodiments, the targeting domains for first tier gRNA molecules can be selected based on (1) a reasonable distance to the target position, e.g., within the first 500 bp of coding sequence downstream of start codon, (2) a high level of orthogonality, and (3) the presence of a 5′ G. In some embodiments, for selection of second tier gRNAs, the requirement for a 5′G can be removed, but the distance restriction is required and a high level of orthogonality was required. In some embodiments, third tier selection uses the same distance restriction and the requirement for a 5′G, but removes the requirement of good orthogonality. In some embodiments, fourth tier selection uses the same distance restriction but removes the requirement of good orthogonality and start with a 5′G. In some embodiments, fifth tier selection removes the requirement of good orthogonality and a 5′G, and a longer sequence (e.g., the rest of the coding sequence, e.g., additional 500 bp upstream or downstream to the transcription target site) is scanned. In certain instances, no gRNA is identified based on the criteria of the particular tier.

In some embodiments, gRNAs are identified for single-gRNA nuclease cleavage as well as for a dual-gRNA paired “nickase” strategy.

In some aspects, gRNAs for use with the N. meningitidis and S. aureus Cas9s can be identified manually by scanning genomic DNA sequence for the presence of PAM sequences. These gRNAs can be separated into two tiers. In some embodiments, for first tier gRNAs, targeting domains are selected within the first 500 bp of coding sequence downstream of start codon. In some embodiments, for second tier gRNAs, targeting domains are selected within the remaining coding sequence (downstream of the first 500 bp). In certain instances, no gRNA is identified based on the criteria of the particular tier.

In some embodiments, another strategy for identifying guide RNAs (gRNAs) for use with S. pyogenes, S. aureus and N. meningtidis Cas9s can use a DNA sequence searching algorithm. In some aspects, guide RNA design is carried out using a custom guide RNA design software based on the public tool cas-offinder (Bae et al. Bioinformatics. 2014; 30(10): 1473-1475). Said custom guide RNA design software scores guides after calculating their genome wide off-target propensity. Typically matches ranging from perfect matches to 7 mismatches are considered for guides ranging in length from 17 to 24. Once the off-target sites are computationally determined, an aggregate score is calculated for each guide and summarized in a tabular output using a web-interface. In addition to identifying potential gRNA sites adjacent to PAM sequences, the software also identifies all PAM adjacent sequences that differ by 1, 2, 3 or more nucleotides from the selected gRNA sites. In some embodiments, genomic DNA sequence for each gene is obtained from the UCSC Genome browser and sequences are screened for repeat elements using the publically available RepeatMasker program. RepeatMasker searches input DNA sequences for repeated elements and regions of low complexity. The output is a detailed annotation of the repeats present in a given query sequence.

In some embodiments, following identification, gRNAs are ranked into tiers based on their distance to the target site or their orthogonality (based on identification of close matches in the human genome containing a relevant PAM, e.g., in the case of S. pyogenes, a NGG PAM, in the case of S. aureus, NNGRR (e.g., a NNGRRT or NNGRRV) PAM, and in the case of N. meningtidis, a NNNNGATT or NNNNGCTT PAM. In some aspects, targeting domains with good orthogonality are selected to minimize off-target DNA cleavage.

As an example, for S. pyogenes and N. meningtidis targets, 17-mer, or 20-mer gRNAs can be designed. As another example, for S. aureus targets, 18-mer, 19-mer, 20-mer, 21-mer, 22-mer, 23-mer and 24-mer gRNAs can be designed.

In some embodiments, gRNAs for both single-gRNA nuclease cleavage and for a dual-gRNA paired “nickase” strategy are identified. In some embodiments for selecting gRNAs, including the determination for which gRNAs can be used for the dual-gRNA paired “nickase” strategy, gRNA pairs should be oriented on the DNA such that PAMs are facing out and cutting with the D10A Cas9 nickase will result in 5′ overhangs. In some aspects, it can be assumed that cleaving with dual nickase pairs will result in deletion of the entire intervening sequence at a reasonable frequency. However, cleaving with dual nickase pairs can also often result in indel mutations at the site of only one of the gRNAs. Candidate pair members can be tested for how efficiently they remove the entire sequence versus just causing indel mutations at the site of one gRNA.

For designing strategies for genetic disruption, in some embodiments, the targeting domains for tier 1 gRNA molecules for S. pyogenes are selected based on their distance to the target site and their orthogonality (PAM is NGG). In some cases, the targeting domains for tier 1 gRNA molecules are selected based on (1) a reasonable distance to the target position, e.g., within the first 500 bp of coding sequence downstream of start codon and (2) a high level of orthogonality. In some aspects, for selection of tier 2 gRNAs, a high level of orthogonality is not required. In some cases, tier 3 gRNAs remove the requirement of good orthogonality and a longer sequence (e.g., the rest of the coding sequence) can be scanned. In certain instances, no gRNA is identified based on the criteria of the particular tier.

For designing strategies for genetic disruption, in some embodiments, the targeting domain for tier 1 gRNA molecules for N. meningtidis were selected within the first 500 bp of the coding sequence and had a high level of orthogonality. The targeting domain for tier 2 gRNA molecules for N. meningtidis were selected within the first 500 bp of the coding sequence and did not require high orthogonality. The targeting domain for tier 3 gRNA molecules for N. meningtidis were selected within a remainder of coding sequence downstream of the 500 bp.

Note that tiers are non-inclusive (each gRNA is listed only once). In certain instances, no gRNA was identified based on the criteria of the particular tier.

For designing strategies for genetic disruption, in some embodiments, the targeting domain for tier 1 gRNA molecules for S. aureus is selected within the first 500 bp of the coding sequence, has a high level of orthogonality, and contains a NNGRRT PAM. In some embodiments, the targeting domain for tier 2 gRNA molecules for S. aureus is selected within the first 500 bp of the coding sequence, no level of orthogonality is required, and contains a NNGRRT PAM. In some embodiments, the targeting domain for tier 3 gRNA molecules for S. aureus are selected within the remainder of the coding sequence downstream and contain a NNGRRT PAM. In some embodiments, the targeting domain for tier 4 gRNA molecules for S. aureus are selected within the first 500 bp of the coding sequence and contain a NNGRRV PAM.

In some embodiments, the targeting domain for tier 5 gRNA molecules for S. aureus are selected within the remainder of the coding sequence downstream and contain a NNGRRV PAM. In certain instances, no gRNA is identified based on the criteria of the particular tier. 2) Cas9

Cas9 molecules of a variety of species can be used in the methods and compositions described herein. While the S. pyogenes, S. aureus, N. meningitidis, and S. thermophilus Cas9 molecules are the subject of much of the disclosure herein, Cas9 molecules of, derived from, or based on the Cas9 proteins of other species listed herein can be used as well. In other words, while the much of the description herein uses S. pyogenes, S. aureus, N. meningitidis, and S. thermophilus Cas9 molecules, Cas9 molecules from the other species can replace them. Such species include: Acidovorax avenae, Actinobacillus pleuropneumoniae, Actinobacillus succinogenes, Actinobacillus suis, Actinomyces sp., Cycliphilusdenitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gammaproteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., or Verminephrobacter eiseniae. Examples of Cas9 molecules can include those described in, e.g., WO2015/161276, WO2017/193107, WO2017/093969, US2016/272999 and US2015/056705.

A Cas9 molecule, or Cas9 polypeptide, as that term is used herein, refers to a molecule or polypeptide that can interact with a gRNA molecule and, in concert with the gRNA molecule, homes or localizes to a site which comprises a target domain and PAM sequence. Cas9 molecule and Cas9 polypeptide, as those terms are used herein, refer to naturally occurring Cas9 molecules and to engineered, altered, or modified Cas9 molecules or Cas9 polypeptides that differ, e.g., by at least one amino acid residue, from a reference sequence, e.g., the most similar naturally occurring Cas9 molecule.

Crystal structures have been determined for two different naturally occurring bacterial Cas9 molecules (Jinek et al., Science, 343(6176):1247997, 2014) and for S. pyogenes Cas9 with a guide RNA (e.g., a synthetic fusion of crRNA and tracrRNA) (Nishimasu et al., Cell, 156:935-949, 2014; and Anders et al., Nature, 2014, doi: 10.1038/naturel3579).

A naturally occurring Cas9 molecule comprises two lobes: a recognition (REC) lobe and a nuclease (NUC) lobe; each of which further comprises domains described herein. An exemplary schematic of the organization of important Cas9 domains in the primary structure is described in WO2015/161276, e.g., in FIGS. 8A-8B therein. The domain nomenclature and the numbering of the amino acid residues encompassed by each domain used throughout this disclosure is as described in Nishimasu et al. The numbering of the amino acid residues is with reference to Cas9 from S. pyogenes.

The REC lobe comprises the arginine-rich bridge helix (BH), the REC1 domain, and the REC2 domain. The REC lobe does not share structural similarity with other known proteins, indicating that it is a Cas9-specific functional domain. The BH domain is a long α-helix and arginine rich region and comprises amino acids 60-93 of the sequence of S. pyogenes Cas9. The REC1 domain is important for recognition of the repeat:anti-repeat duplex, e.g., of a gRNA or a tracrRNA, and is therefore critical for Cas9 activity by recognizing the target sequence. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat:anti-repeat duplex. The REC2 domain comprises amino acids 180-307 of the sequence of S. pyogenes Cas9.

The NUC lobe comprises the RuvC domain (also referred to herein as RuvC-like domain), the HNH domain (also referred to herein as HNH-like domain), and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases, and cleaves a single strand, e.g., the complementary strand of the target nucleic acid molecule. The HNH domain lies between the RuvC I-III motifs and comprises amino acids 775-908 of the sequence of S. pyogenes Cas9.

The PI domain interacts with the PAM of the target nucleic acid molecule, and comprises amino acids 1099-1368 of the sequence of S. pyogenes Cas9.

A) a RuvC-Like Domain and an HNH-Like Domain

In some embodiments, a Cas9 molecule or Cas9 polypeptide comprises an HNH-like domain and a RuvC-like domain. In some embodiments, cleavage activity is dependent on a RuvC-like domain and an HNH-like domain. A Cas9 molecule or Cas9 polypeptide, e.g., an eaCas9 molecule or eaCas9 polypeptide, can comprise one or more of the following domains: a RuvC-like domain and an HNH-like domain. In some embodiments, a Cas9 molecule or Cas9 polypeptide is an eaCas9 molecule or eaCas9 polypeptide and the eaCas9 molecule or eaCas9 polypeptide comprises a RuvC-like domain, e.g., a RuvC-like domain described herein, and/or an HNH-like domain, e.g., an HNH-like domain described herein.

B) RuvC-Like Domains

In some embodiments, a RuvC-like domain cleaves, a single strand, e.g., the non-complementary strand of the target nucleic acid molecule. The Cas9 molecule or Cas9 polypeptide can include more than one RuvC-like domain (e.g., one, two, three or more RuvC-like domains). In some embodiments, a RuvC-like domain is at least 5, 6, 7, 8 amino acids in length but not more than 20, 19, 18, 17, 16 or 15 amino acids in length. In some embodiments, the Cas9 molecule or Cas9 polypeptide comprises an N-terminal RuvC-like domain of about 10 to 20 amino acids, e.g., about 15 amino acids in length.

C) N-Terminal RuvC-Like Domains

Some naturally occurring Cas9 molecules comprise more than one RuvC-like domain with cleavage being dependent on the N-terminal RuvC-like domain. Accordingly, Cas9 molecules or Cas9 polypeptide can comprise an N-terminal RuvC-like domain.

In embodiment, the N-terminal RuvC-like domain is cleavage competent.

In embodiment, the N-terminal RuvC-like domain is cleavage incompetent.

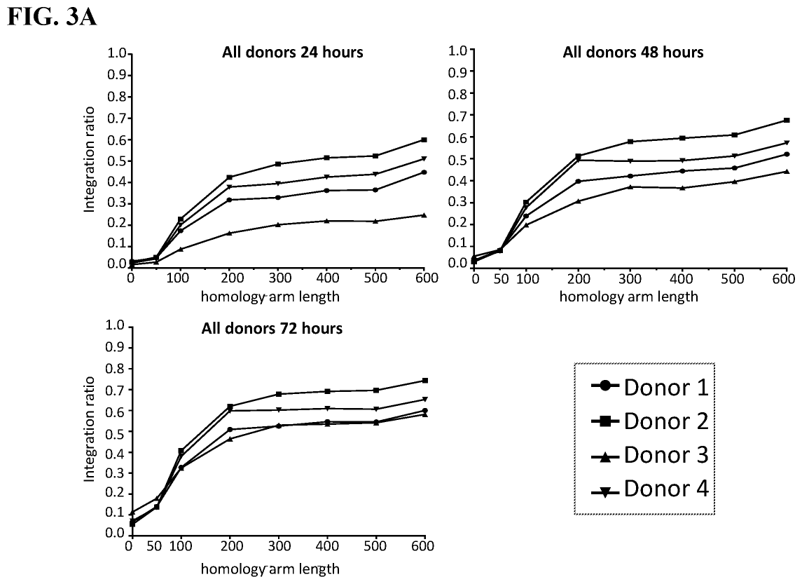

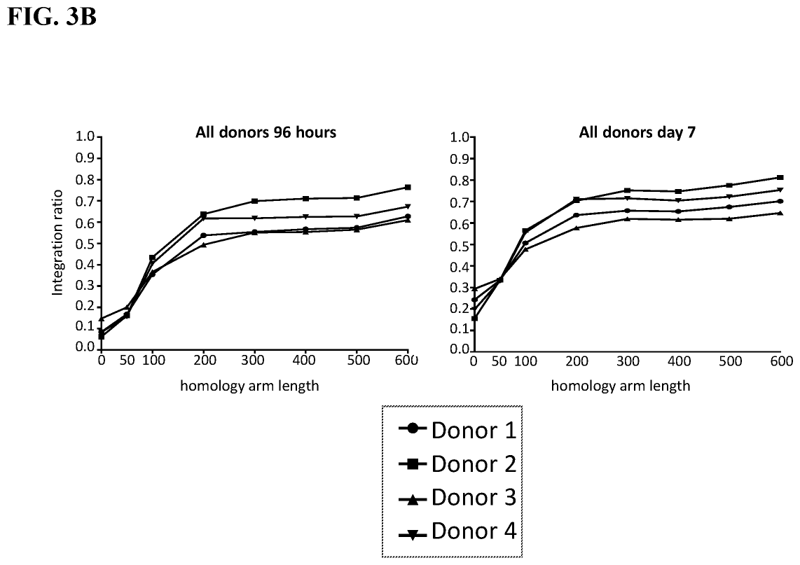

In some embodiments, the N-terminal RuvC-like domain differs from a sequence of an N-terminal RuvC like domain disclosed herein, e.g., in WO2015/161276, e.g., in FIGS. 3A-3B or FIGS. 7A-7B therein, as many as 1 but no more than 2, 3, 4, or 5 residues. In some embodiments, 1, 2, or all 3 of the highly conserved residues identified WO2015/161276, e.g., in FIGS. 3A-3B or FIGS. 7A-7B therein are present.

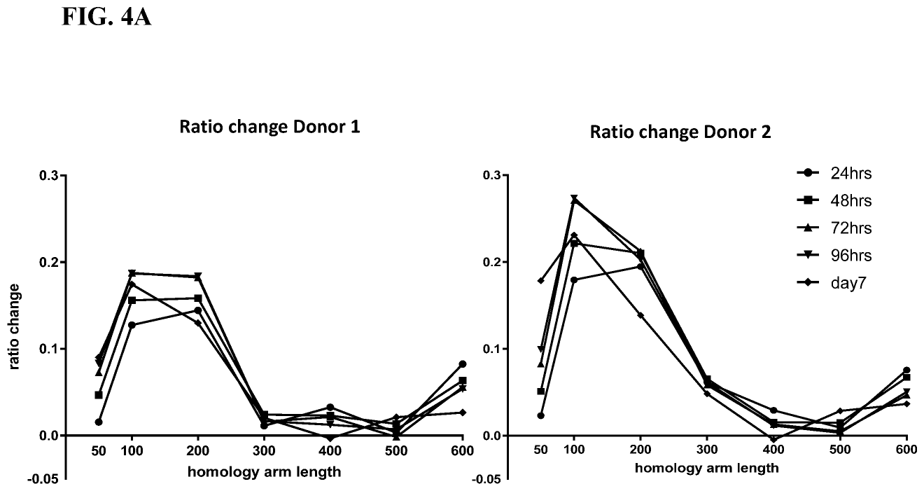

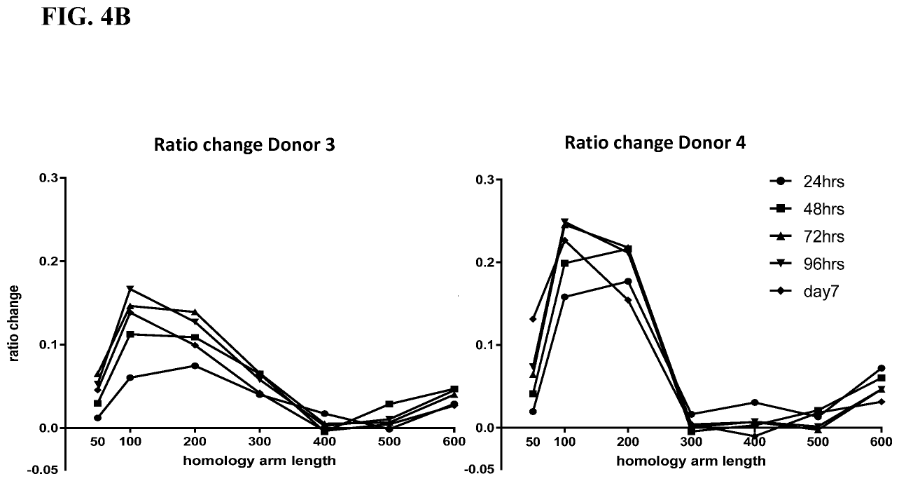

In some embodiments, the N-terminal RuvC-like domain differs from a sequence of an N-terminal RuvC-like domain disclosed herein, e.g., in WO2015/161276, e.g., in FIGS. 4A-4B or FIGS. 7A-7B therein, as many as 1 but no more than 2, 3, 4, or 5 residues. In some embodiments, 1, 2, 3 or all 4 of the highly conserved residues identified in WO2015/161276, e.g., in FIGS. 4A-4B or FIGS. 7A-7B therein are present.

D) Additional RuvC-Like Domains

In addition to the N-terminal RuvC-like domain, the Cas9 molecule or Cas9 polypeptide, e.g., an eaCas9 molecule or eaCas9 polypeptide, can comprise one or more additional RuvC-like domains. In some embodiments, the Cas9 molecule or Cas9 polypeptide can comprise two additional RuvC-like domains. Preferably, the additional RuvC-like domain is at least 5 amino acids in length and, e.g., less than 15 amino acids in length, e.g., 5 to 10 amino acids in length, e.g., 8 amino acids in length.

E) HNH-Like Domains

In some embodiments, an HNH-like domain cleaves a single stranded complementary domain, e.g., a complementary strand of a double stranded nucleic acid molecule. In some embodiments, an HNH-like domain is at least 15, 20, 25 amino acids in length but not more than 40, 35 or 30 amino acids in length, e.g., 20 to 35 amino acids in length, e.g., 25 to 30 amino acids in length. Exemplary HNH-like domains are described herein.

In some embodiments, the HNH-like domain is cleavage competent.

In some embodiments, the HNH-like domain is cleavage incompetent.

In some embodiments, the HNH-like domain differs from a sequence of an HNH-like domain disclosed herein, e.g., in WO2015/161276, e.g., in FIGS. 5A-5C or FIGS. 7A-7B therein, as many as 1 but no more than 2, 3, 4, or 5 residues. In some embodiments, 1 or both of the highly conserved residues identified in WO2015/161276, e.g., in FIGS. 5A-5C or FIGS. 7A-7B therein are present.

In some embodiments, the HNH-like domain differs from a sequence of an HNH-like domain disclosed herein, e.g., in WO2015/161276, e.g., in FIGS. 6A-6B or FIGS. 7A-7B therein, as many as 1 but no more than 2, 3, 4, or 5 residues. In some embodiments, 1, 2, all 3 of the highly conserved residues identified in WO2015/161276, e.g., in FIGS. 6A-6B or FIGS. 7A-7B therein are present.

F) Nuclease and Helicase Activities

In some embodiments, the Cas9 molecule or Cas9 polypeptide is capable of cleaving a target nucleic acid molecule. Typically wild type Cas9 molecules cleave both strands of a target nucleic acid molecule. Cas9 molecules and Cas9 polypeptides can be engineered to alter nuclease cleavage (or other properties), e.g., to provide a Cas9 molecule or Cas9 peolypeptide which is a nickase, or which lacks the ability to cleave target nucleic acid. A Cas9 molecule or Cas9 polypeptide that is capable of cleaving a target nucleic acid molecule is referred to herein as an eaCas9 molecule or eaCas9 polypeptide

In some embodiments, an eaCas9 molecule or eaCas9 polypeptide comprises one or more of the following activities: a nickase activity, i.e., the ability to cleave a single strand, e.g., the non-complementary strand or the complementary strand, of a nucleic acid molecule; a double stranded nuclease activity, i.e., the ability to cleave both strands of a double stranded nucleic acid and create a double stranded break, which In some embodiments is the presence of two nickase activities; an endonuclease activity; an exonuclease activity; and a helicase activity, i.e., the ability to unwind the helical structure of a double stranded nucleic acid.

In some embodiments, an enzymatically active or eaCas9 molecule or eaCas9 polypeptide cleaves both strands and results in a double stranded break. In some embodiments, an eaCas9 molecule cleaves only one strand, e.g., the strand to which the gRNA hybridizes to, or the strand complementary to the strand the gRNA hybridizes with. In some embodiments, an eaCas9 molecule or eaCas9 polypeptide comprises cleavage activity associated with an HNH-like domain. In some embodiments, an eaCas9 molecule or eaCas9 polypeptide comprises cleavage activity associated with an N-terminal RuvC-like domain. In some embodiments, an eaCas9 molecule or eaCas9 polypeptide comprises cleavage activity associated with an HNH-like domain and cleavage activity associated with an N-terminal RuvC-like domain. In some embodiments, an eaCas9 molecule or eaCas9 polypeptide comprises an active, or cleavage competent, HNH-like domain and an inactive, or cleavage incompetent, N-terminal RuvC-like domain. In some embodiments, an eaCas9 molecule or eaCas9 polypeptide comprises an inactive, or cleavage incompetent, HNH-like domain and an active, or cleavage competent, N-terminal RuvC-like domain.

Some Cas9 molecules or Cas9 polypeptides have the ability to interact with a gRNA molecule, and in conjunction with the gRNA molecule localize to a core target domain, but are incapable of cleaving the target nucleic acid, or incapable of cleaving at efficient rates. Cas9 molecules having no, or no substantial, cleavage activity are referred to herein as an eiCas9 molecule or eiCas9 polypeptide. For example, an eiCas9 molecule or eiCas9 polypeptide can lack cleavage activity or have substantially less, e.g., less than 20, 10, 5, 1 or 0.1% of the cleavage activity of a reference Cas9 molecule or eiCas9 polypeptide, as measured by an assay described herein.

G) Targeting and PAMs

A Cas9 molecule or Cas9 polypeptide, is a polypeptide that can interact with a guide RNA (gRNA) molecule and, in concert with the gRNA molecule, localizes to a site which comprises a target domain and a PAM sequence.

In some embodiments, the ability of an eaCas9 molecule or eaCas9 polypeptide to interact with and cleave a target nucleic acid is PAM sequence dependent. A PAM sequence is a sequence in the target nucleic acid. In some embodiments, cleavage of the target nucleic acid occurs upstream from the PAM sequence. EaCas9 molecules from different bacterial species can recognize different sequence motifs (e.g., PAM sequences). In some embodiments, an eaCas9 molecule of S. pyogenes recognizes the sequence motif NGG, NAG, NGA and directs cleavage of a target nucleic acid sequence 1 to 10, e.g., 3 to 5, base pairs upstream from that sequence. See, e.g., Mali et al., Science 2013; 339(6121): 823-826. In some embodiments, an eaCas9 molecule of S. thermophilus recognizes the sequence motif NGGNG and/or NNAGAAW (W=A or T) and directs cleavage of a target nucleic acid sequence 1 to 10, e.g., 3 to 5, base pairs upstream from these sequences. See, e.g., Horvath et al., Science 2010; 327(5962):167-170, and Deveau et al., J Bacteriol 2008; 190(4): 1390-1400. In some embodiments, an eaCas9 molecule of S. mutans recognizes the sequence motif NGG and/or NAAR (R=A or G)) and directs cleavage of a core target nucleic acid sequence 1 to 10, e.g., 3 to 5 base pairs, upstream from this sequence. See, e.g., Deveau et al., J Bacteriol 2008; 190(4): 1390-1400. In some embodiments, an eaCas9 molecule of S. aureus recognizes the sequence motif NNGRR (R=A or G) and directs cleavage of a target nucleic acid sequence 1 to 10, e.g., 3 to 5, base pairs upstream from that sequence. In some embodiments, an eaCas9 molecule of S. aureus recognizes the sequence motif NNGRRT (R=A or G) and directs cleavage of a target nucleic acid sequence 1 to 10, e.g., 3 to 5, base pairs upstream from that sequence. In some embodiments, an eaCas9 molecule of S. aureus recognizes the sequence motif NNGRRV (R=A or G) and directs cleavage of a target nucleic acid sequence 1 to 10, e.g., 3 to 5, base pairs upstream from that sequence. In some embodiments, an eaCas9 molecule of N. meningitidis recognizes the sequence motif NNNNGATT or NNNGCTT (R=A or G, V=A, G or C and directs cleavage of a target nucleic acid sequence 1 to 10, e.g., 3 to 5, base pairs upstream from that sequence. See, e.g., Hou et al., PNAS Early Edition 2013, 1-6. The ability of a Cas9 molecule to recognize a PAM sequence can be determined, e.g., using a transformation assay described in Jinek et al., Science 2012 337:816. In the aforementioned embodiments, N can be any nucleotide residue, e.g., any of A, G, C or T.

As is discussed herein, Cas9 molecules can be engineered to alter the PAM specificity of the Cas9 molecule.

Exemplary naturally occurring Cas9 molecules are described in Chylinski et al., RNA Biology 2013 10:5, 727-737. Such Cas9 molecules include Cas9 molecules of a cluster 1-78 bacterial family.

Exemplary naturally occurring Cas9 molecules include a Cas9 molecule of a cluster 1 bacterial family. Examples include a Cas9 molecule of: S. pyogenes (e.g., strain SF370, MGAS10270, MGAS10750, MGAS2096, MGAS315, MGAS5005, MGAS6180, MGAS9429, NZ131 and SSI-1), S. thermophilus (e.g., strain LMD-9), S. pseudoporcinus (e.g., strain SPIN 20026), S. mutans (e.g., strain UA159, NN2025), S. macacae (e.g., strain NCTC11558), S. gallolyticus (e.g., strain UCN34, ATCC BAA-2069), S. equines (e.g., strain ATCC 9812, MGCS 124), S. dysdalactiae (e.g., strain GGS 124), S. bovis (e.g., strain ATCC 700338), S. anginosus (e.g., strain F0211), S. agalactiae (e.g., strain NEM316, A909), Listeria monocytogenes (e.g., strain F6854), Listeria innocua (L. innocua, e.g., strain Clip11262), Enterococcus italicus (e.g., strain DSM 15952), or Enterococcus faecium (e.g., strain 1,231,408). Another exemplary Cas9 molecule is a Cas9 molecule of Neisseria meningitidis (Hou et al., PNAS Early Edition 2013, 1-6).

In some embodiments, a Cas9 molecule or Cas9 polypeptide, e.g., an eaCas9 molecule or eaCas9 polypeptide, comprises an amino acid sequence: having 60%, 65%, 70%, 75%, 80%, 85%, 90%, 95%, 96%, 97%, 98% or 99% homology with; differs at no more than, 2, 5, 10, 15, 20, 30, or 40% of the amino acid residues when compared with; differs by at least 1, 2, 5, 10 or 20 amino acids but by no more than 100, 80, 70, 60, 50, 40 or 30 amino acids from; or is identical to any Cas9 molecule sequence described herein, or a naturally occurring Cas9 molecule sequence, e.g., a Cas9 molecule from a species listed herein (e.g., SEQ ID NOS:159-162, 227 and 228) or described in Chylinski et al., RNA Biology 2013 10:5, 727-737; Hou et al., PNAS Early Edition 2013, 1-6. In some embodiments, the Cas9 molecule or Cas9 polypeptide comprises one or more of the following activities: a nickase activity; a double stranded cleavage activity (e.g., an endonuclease and/or exonuclease activity); a helicase activity; or the ability, together with a gRNA molecule, to home to a target nucleic acid.

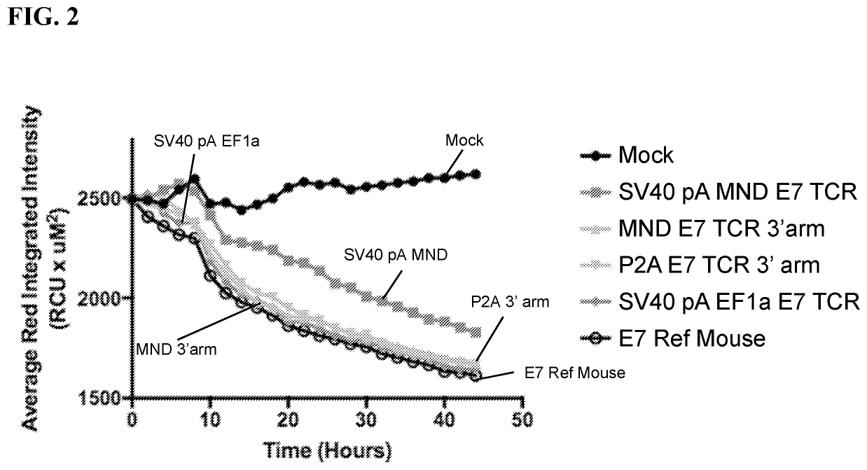

In some embodiments, a Cas9 molecule or Cas9 polypeptide comprises the amino acid sequence of the consensus sequence of WO2015/161276, e.g., in FIGS. 2A-2G therein, wherein “*” indicates any amino acid found in the corresponding position in the amino acid sequence of a Cas9 molecule of S. pyogenes, S. thermophilus, S. mutans and L. innocua, and “-” indicates any amino acid. In some embodiments, a Cas9 molecule or Cas9 polypeptide differs from the sequence of the consensus sequence of SEQ ID NOS: 159-162, 227 and 228 or the consensus sequence disclosed in WO2015/161276, e.g., in FIGS. 2A-2G therein by at least 1, but no more than 2, 3, 4, 5, 6, 7, 8, 9, or 10 amino acid residues. In some embodiments, a Cas9 molecule or Cas9 polypeptide comprises the amino acid sequence of SEQ ID NO:228 or as described in WO2015/161276, e.g., in FIGS. 7A-7B therein, wherein “*” indicates any amino acid found in the corresponding position in the amino acid sequence of a Cas9 molecule of S. pyogenes, or N. meningitidis, “-” indicates any amino acid, and “-” indicates any amino acid or absent. In some embodiments, a Cas9 molecule or Cas9 polypeptide differs from the sequence of SEQ ID NO:227 or 228 or as described in WO2015/161276, e.g., in FIGS. 7A-7B therein by at least 1, but no more than 2, 3, 4, 5, 6, 7, 8, 9, or 10 amino acid residues.

A comparison of the sequence of a number of Cas9 molecules indicate that certain regions are conserved. These are identified herein as: region 1 (residues 1 to 180, or in the case of region 1′residues 120 to 180); region 2 (residues 360 to 480); region 3 (residues 660 to 720); region 4 (residues 817 to 900); and region 5 (residues 900 to 960).

In some embodiments, a Cas9 molecule or Cas9 polypeptide comprises regions 1-5, together with sufficient additional Cas9 molecule sequence to provide a biologically active molecule, e.g., a Cas9 molecule having at least one activity described herein. In some embodiments, each of regions 1-6, independently, have, 50%, 60%, 70%, or 80% homology with the corresponding residues of a Cas9 molecule or Cas9 polypeptide described herein, e.g., set forth in SEQ ID NOS: 159-162, 227 and 228 or a sequence disclosed in WO2015/161276, e.g., from FIGS. 2A-2G or from FIGS. 7A-7B therein.

H) Engineered or Altered Cas9 Molecules and Cas9 Polypeptides

Cas9 molecules and Cas9 polypeptides described herein, e.g., naturally occurring Cas9 molecules, can possess any of a number of properties, including: nickase activity, nuclease activity (e.g., endonuclease and/or exonuclease activity); helicase activity; the ability to associate functionally with a gRNA molecule; and the ability to target (or localize to) a site on a nucleic acid (e.g., PAM recognition and specificity). In some embodiments, a Cas9 molecule or Cas9 polypeptide can include all or a subset of these properties. In typical embodiments, a Cas9 molecule or Cas9 polypeptide has the ability to interact with a gRNA molecule and, in concert with the gRNA molecule, localize to a site in a nucleic acid. Other activities, e.g., PAM specificity, cleavage activity, or helicase activity can vary more widely in Cas9 molecules and Cas9 polypeptides.

Cas9 molecules include engineered Cas9 molecules and engineered Cas9 polypeptides (“engineered,” as used in this context, means merely that the Cas9 molecule or Cas9 polypeptide differs from a reference sequences, and implies no process or origin limitation). An engineered Cas9 molecule or Cas9 polypeptide can comprise altered enzymatic properties, e.g., altered nuclease activity, (as compared with a naturally occurring or other reference Cas9 molecule) or altered helicase activity. As discussed herein, an engineered Cas9 molecule or Cas9 polypeptide can have nickase activity (as opposed to double strand nuclease activity). In some embodiments an engineered Cas9 molecule or Cas9 polypeptide can have an alteration that alters its size, e.g., a deletion of amino acid sequence that reduces its size, e.g., without significant effect on one or more, or any Cas9 activity. In some embodiments, an engineered Cas9 molecule or Cas9 polypeptide can comprise an alteration that affects PAM recognition. E.g., an engineered Cas9 molecule can be altered to recognize a PAM sequence other than that recognized by the endogenous wild-type PI domain. In some embodiments a Cas9 molecule or Cas9 polypeptide can differ in sequence from a naturally occurring Cas9 molecule but not have significant alteration in one or more Cas9 activities.

Cas9 molecules or Cas9 polypeptides with desired properties can be made in a number of ways, e.g., by alteration of a parental, e.g., naturally occurring, Cas9 molecules or Cas9 polypeptides, to provide an altered Cas9 molecule or Cas9 polypeptide having a desired property. For example, one or more mutations or differences relative to a parental Cas9 molecule, e.g., a naturally occurring or engineered Cas9 molecule, can be introduced. Such mutations and differences comprise: substitutions (e.g., conservative substitutions or substitutions of non-essential amino acids); insertions; or deletions. In some embodiments, a Cas9 molecule or Cas9 polypeptide can comprises one or more mutations or differences, e.g., at least 1, 2, 3, 4, 5, 10, 15, 20, 30, 40 or 50 mutations but less than 200, 100, or 80 mutations relative to a reference, e.g., a parental, Cas9 molecule.

In some embodiments, a mutation or mutations do not have a substantial effect on a Cas9 activity, e.g. a Cas9 activity described herein. In some embodiments, a mutation or mutations have a substantial effect on a Cas9 activity, e.g. a Cas9 activity described herein.

I) Non-Cleaving and Modified-Cleavage Cas9 Molecules and Cas9 Polypeptides

In some embodiments, a Cas9 molecule or Cas9 polypeptide comprises a cleavage property that differs from naturally occurring Cas9 molecules, e.g., that differs from the naturally occurring Cas9 molecule having the closest homology. For example, a Cas9 molecule or Cas9 polypeptide can differ from naturally occurring Cas9 molecules, e.g., a Cas9 molecule of S. pyogenes, as follows: its ability to modulate, e.g., decreased or increased, cleavage of a double stranded nucleic acid (endonuclease and/or exonuclease activity), e.g., as compared to a naturally occurring Cas9 molecule (e.g., a Cas9 molecule of S. pyogenes); its ability to modulate, e.g., decreased or increased, cleavage of a single strand of a nucleic acid, e.g., a non-complementary strand of a nucleic acid molecule or a complementary strand of a nucleic acid molecule (nickase activity), e.g., as compared to a naturally occurring Cas9 molecule (e.g., a Cas9 molecule of S. pyogenes); or the ability to cleave a nucleic acid molecule, e.g., a double stranded or single stranded nucleic acid molecule, can be eliminated.

J) Modified Cleavage eaCas9 Molecules and eaCas9 Polypeptides

In some embodiments, an eaCas9 molecule or eaCas9 polypeptide comprises one or more of the following activities: cleavage activity associated with an N-terminal RuvC-like domain; cleavage activity associated with an HNH-like domain; cleavage activity associated with an HNH-like domain and cleavage activity associated with an N-terminal RuvC-like domain.

In some embodiments, an eaCas9 molecule or eaCas9 polypeptide comprises an active, or cleavage competent, HNH-like domain and an inactive, or cleavage incompetent, N-terminal RuvC-like domain. An exemplary inactive, or cleavage incompetent N-terminal RuvC-like domain can have a mutation of an aspartic acid in an N-terminal RuvC-like domain, e.g., an aspartic acid at position 9 of the consensus sequence of SEQ ID NOS: 159-162, 227 and 228 or the consensus sequence disclosed in WO2015/161276, e.g., in FIGS. 2A-2G therein or an aspartic acid at position 10 of SEQ ID NO:228, e.g., can be substituted with an alanine. In some embodiments, the eaCas9 molecule or eaCas9 polypeptide differs from wild type in the N-terminal RuvC-like domain and does not cleave the target nucleic acid, or cleaves with significantly less efficiency, e.g., less than 20, 10, 5, 1 or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, or S. thermophilus. In some embodiments, the reference Cas9 molecule is the naturally occurring Cas9 molecule having the closest sequence identity or homology.

In some embodiments, an eaCas9 molecule or eaCas9 polypeptide comprises an inactive, or cleavage incompetent, HNH domain and an active, or cleavage competent, N-terminal RuvC-like domain. Exemplary inactive, or cleavage incompetent HNH-like domains can have a mutation at one or more of: a histidine in an HNH-like domain, e.g., a histidine shown at position 856 of the consensus sequence of SEQ ID NOS: 159-162, 227 and 228 or the consensus sequence disclosed in WO2015/161276, e.g., in FIGS. 2A-2G therein, e.g., can be substituted with an alanine; and one or more asparagines in an HNH-like domain, e.g., an asparagine shown at position 870 of the consensus sequence of SEQ ID NOS: 159-162, 227 and 228 or the consensus sequence disclosed in WO2015/161276, e.g., in FIGS. 2A-2G therein and/or at position 879 of the consensus sequence of SEQ ID NOS: 159-162, 227 and 228 or the consensus sequence disclosed in WO2015/161276, e.g., in FIGS. 2A-2G therein, e.g., can be substituted with an alanine. In some embodiments, the eaCas9 differs from wild type in the HNH-like domain and does not cleave the target nucleic acid, or cleaves with significantly less efficiency, e.g., less than 20, 10, 5, 1 or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, or S. thermophilus. In some embodiments, the reference Cas9 molecule is the naturally occurring Cas9 molecule having the closest sequence identity or homology.

In some embodiments, an eaCas9 molecule or eaCas9 polypeptide comprises an inactive, or cleavage incompetent, HNH domain and an active, or cleavage competent, N-terminal RuvC-like domain. Exemplary inactive, or cleavage incompetent HNH-like domains can have a mutation at one or more of: a histidine in an HNH-like domain, e.g., a histidine shown at position 856 of the consensus sequence of SEQ ID NOS: 159-162, 227 and 228 or the consensus sequence disclosed in WO2015/161276, e.g., in FIGS. 2A-2G therein, e.g., can be substituted with an alanine; and one or more asparagines in an HNH-like domain, e.g., an asparagine shown at position 870 of the consensus sequence of SEQ ID NOS: 159-162, 227 and 228 or the consensus sequence disclosed in WO2015/161276, e.g., in FIGS. 2A-2G therein and/or at position 879 of the consensus sequence of SEQ ID NOS: 159-162, 227 and 228 or the consensus sequence disclosed in WO2015/161276, e.g., in FIGS. 2A-2G therein, e.g., can be substituted with an alanine. In some embodiments, the eaCas9 differs from wild type in the HNH-like domain and does not cleave the target nucleic acid, or cleaves with significantly less efficiency, e.g., less than 20, 10, 5, 1 or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, or S. thermophilus. In some embodiments, the reference Cas9 molecule is the naturally occurring Cas9 molecule having the closest sequence identity or homology.

K) Alterations in the Ability to Cleave One or Both Strands of a Target Nucleic Acid

In some embodiments, exemplary Cas9 activities comprise one or more of PAM specificity, cleavage activity, and helicase activity. A mutation(s) can be present, e.g., in: one or more RuvC-like domain, e.g., an N-terminal RuvC-like domain; an HNH-like domain; a region outside the RuvC-like domains and the HNH-like domain. In some embodiments, a mutation(s) is present in a RuvC-like domain, e.g., an N-terminal RuvC-like. In some embodiments, a mutation(s) is present in an HNH-like domain. In some embodiments, mutations are present in both a RuvC-like domain, e.g., an N-terminal RuvC-like domain, and an HNH-like domain.

Exemplary mutations that may be made in the RuvC domain or HNH domain with reference to the S. pyogenes sequence include: D10A, E762A, H840A, N854A, N863A and/or D986A.

In some embodiments, a Cas9 molecule or Cas9 polypeptide is an eiCas9 molecule or eiCas9 polypeptide comprising one or more differences in a RuvC domain and/or in an HNH domain as compared to a reference Cas9 molecule, and the eiCas9 molecule or eiCas9 polypeptide does not cleave a nucleic acid, or cleaves with significantly less efficiency than does wildtype, e.g., when compared with wild type in a cleavage assay, e.g., as described herein, cuts with less than 50, 25, 10, or 1% of a reference Cas9 molecule, as measured by an assay described herein.

Whether or not a particular sequence, e.g., a substitution, may affect one or more activity, such as targeting activity, cleavage activity, etc., can be evaluated or predicted, e.g., by evaluating whether the mutation is conservative. In some embodiments, a “non-essential” amino acid residue, as used in the context of a Cas9 molecule, is a residue that can be altered from the wild-type sequence of a Cas9 molecule, e.g., a naturally occurring Cas9 molecule, e.g., an eaCas9 molecule, without abolishing or more preferably, without substantially altering a Cas9 activity (e.g., cleavage activity), whereas changing an “essential” amino acid residue results in a substantial loss of activity (e.g., cleavage activity).

In some embodiments, a Cas9 molecule or Cas9 polypeptide comprises a cleavage property that differs from naturally occurring Cas9 molecules, e.g., that differs from the naturally occurring Cas9 molecule having the closest homology. For example, a Cas9 molecule or Cas9 polypeptide can differ from naturally occurring Cas9 molecules, e.g., a Cas9 molecule of S aureus, S. pyogenes, or C. jejuni as follows: its ability to modulate, e.g., decreased or increased, cleavage of a double stranded break (endonuclease and/or exonuclease activity), e.g., as compared to a naturally occurring Cas9 molecule (e.g., a Cas9 molecule of S aureus, S. pyogenes, or C. jejuni); its ability to modulate, e.g., decreased or increased, cleavage of a single strand of a nucleic acid, e.g., a non-complementary strand of a nucleic acid molecule or a complementary strand of a nucleic acid molecule (nickase activity), e.g., as compared to a naturally occurring Cas9 molecule (e.g., a Cas9 molecule of S aureus, S. pyogenes, or C. jejuni); or the ability to cleave a nucleic acid molecule, e.g., a double stranded or single stranded nucleic acid molecule, can be eliminated.

In some embodiments, the altered Cas9 molecule or Cas9 polypeptide is an eaCas9 molecule or eaCas9 polypeptide comprising one or more of the following activities: cleavage activity associated with a RuvC domain; cleavage activity associated with an HNH domain; cleavage activity associated with an HNH domain and cleavage activity associated with a RuvC domain.

In some embodiments, the altered Cas9 molecule or Cas9 polypeptide is an eiCas9 molecule or eaCas9 polypeptide which does not cleave a nucleic acid molecule (either double stranded or single stranded nucleic acid molecules) or cleaves a nucleic acid molecule with significantly less efficiency, e.g., less than 20, 10, 5, 1 or 0.1% of the cleavage activity of a reference Cas9 molecule, e.g., as measured by an assay described herein. The reference Cas9 molecule can be a naturally occurring unmodified Cas9 molecule, e.g., a naturally occurring Cas9 molecule such as a Cas9 molecule of S. pyogenes, S. thermophilus, S. aureus, C. jejuni or N. meningitidis. In some embodiments, the reference Cas9 molecule is the naturally occurring Cas9 molecule having the closest sequence identity or homology. In some embodiments, the eiCas9 molecule or eiCas9 polypeptide lacks substantial cleavage activity associated with a RuvC domain and cleavage activity associated with an HNH domain.

In some embodiments, the altered Cas9 molecule or Cas9 polypeptide is an eaCas9 molecule or eaCas9 polypeptide comprising the fixed amino acid residues of S. pyogenes shown in the consensus sequence disclosed in WO2015/161276, e.g., in FIGS. 2A-2G therein, and has one or more amino acids that differ from the amino acid sequence of S. pyogenes (e.g., has a substitution) at one or more residue (e.g., 2, 3, 5, 10, 15, 20, 30, 50, 70, 80, 90, 100, 200 amino acid residues) in SEQ ID NO:164 or residue represented by an “-” in the consensus sequence disclosed in WO2015/161276, e.g., in FIGS. 2A-2G therein.

In some embodiments, the altered Cas9 molecule or Cas9 polypeptide, e.g., an eaCas9 molecule, can be a fusion, e.g., of two of more different Cas9 molecules or Cas9 polypeptides, e.g., of two or more naturally occurring Cas9 molecules of different species. For example, a fragment of a naturally occurring Cas9 molecule of one species can be fused to a fragment of a Cas9 molecule of a second species. As an example, a fragment of Cas9 molecule of S. pyogenes comprising an N-terminal RuvC-like domain can be fused to a fragment of Cas9 molecule of a species other than S. pyogenes (e.g., S. thermophilus) comprising an HNH-like domain.

L) Cas9 Molecules With Altered PAM Recognition Or No PAM Recognition

Naturally occurring Cas9 molecules can recognize specific PAM sequences, for example the PAM recognition sequences described herein for, e.g., S. pyogenes, S. thermophilus, S. mutans, S. aureus and N. meningitidis.

In some embodiments, a Cas9 molecule or Cas9 polypeptide has the same PAM specificities as a naturally occurring Cas9 molecule. In other embodiments, a Cas9 molecule or Cas9 polypeptide has a PAM specificity not associated with a naturally occurring Cas9 molecule, or a PAM specificity not associated with the naturally occurring Cas9 molecule to which it has the closest sequence homology. For example, a naturally occurring Cas9 molecule can be altered, e.g., to alter PAM recognition, e.g., to alter the PAM sequence that the Cas9 molecule or Cas9 polypeptide recognizes to decrease off target sites and/or improve specificity; or eliminate a PAM recognition requirement. In some embodiments, a Cas9 molecule can be altered, e.g., to increase length of PAM recognition sequence and/or improve Cas9 specificity to high level of identity, e.g., to decrease off target sites and increase specificity. In some embodiments, the length of the PAM recognition sequence is at least 4, 5, 6, 7, 8, 9, 10 or 15 amino acids in length.

Cas9 molecules or Cas9 polypeptides that recognize different PAM sequences and/or have reduced off-target activity can be generated using directed evolution. Exemplary methods and systems that can be used for directed evolution of Cas9 molecules are described, e.g., in Esvelt et al. Nature 2011, 472(7344): 499-503. Candidate Cas9 molecules can be evaluated, e.g., by methods described herein.

Alterations of the PI domain, which mediates PAM recognition, are discussed herein.

M) Synthetic Cas9 Molecules and Cas9 Polypeptides with Altered PI Domains

Current genome-editing methods are limited in the diversity of target sequences that can be targeted by the PAM sequence that is recognized by the Cas9 molecule utilized. A synthetic Cas9 molecule (or Syn-Cas9 molecule), or synthetic Cas9 polypeptide (or Syn-Cas9 polypeptide), as that term is used herein, refers to a Cas9 molecule or Cas9 polypeptide that comprises a Cas9 core domain from one bacterial species and a functional altered PI domain, i.e., a PI domain other than that naturally associated with the Cas9 core domain, e.g., from a different bacterial species.

In some embodiments, the altered PI domain recognizes a PAM sequence that is different from the PAM sequence recognized by the naturally-occurring Cas9 from which the Cas9 core domain is derived. In some embodiments, the altered PI domain recognizes the same PAM sequence recognized by the naturally-occurring Cas9 from which the Cas9 core domain is derived, but with different affinity or specificity. A Syn-Cas9 molecule or Syn-Cas9 polypeptide can be, respectively, a Syn-eaCas9 molecule or Syn-eaCas9 polypeptide or a Syn-eiCas9 molecule Syn-eiCas9 polypeptide.

An exemplary Syn-Cas9 molecule or Syn-Cas9 polypeptide comprises: a) a Cas9 core domain, e.g., a Cas9 core domain, e.g., a S. aureus, S. pyogenes, or C. jejuni Cas9 core domain; and b) an altered PI domain from a species X Cas9 sequence.

In some embodiments, the RKR motif (the PAM binding motif) of said altered PI domain comprises: differences at 1, 2, or 3 amino acid residues; a difference in amino acid sequence at the first, second, or third position; differences in amino acid sequence at the first and second positions, the first and third positions, or the second and third positions; as compared with the sequence of the RKR motif of the native or endogenous PI domain associated with the Cas9 core domain.

In some embodiments, a Syn-Cas9 molecule or Syn-Cas9 polypeptide may also be size-optimized, e.g., the Syn-Cas9 molecule or Syn-Cas9 polypeptide comprises one or more deletions, and optionally one or more linkers disposed between the amino acid residues flanking the deletions. In some embodiments, a Syn-Cas9 molecule or Syn-Cas9 polypeptide comprises a REC deletion.

N) Size-Optimized Cas9 Molecules and Cas9 Polypeptides

Engineered Cas9 molecules and engineered Cas9 polypeptides described herein include a Cas9 molecule or Cas9 polypeptide comprising a deletion that reduces the size of the molecule while still retaining desired Cas9 properties, e.g., essentially native conformation, Cas9 nuclease activity, and/or target nucleic acid molecule recognition. Provided herein are Cas9 molecules or Cas9 polypeptides comprising one or more deletions and optionally one or more linkers, wherein a linker is disposed between the amino acid residues that flank the deletion. Methods for identifying suitable deletions in a reference Cas9 molecule, methods for generating Cas9 molecules with a deletion and a linker, and methods for using such Cas9 molecules will be apparent upon review of this document.

A Cas9 molecule, e.g., a S. aureus, S. pyogenes, or C. jejuni, Cas9 molecule, having a deletion is smaller, e.g., has reduced number of amino acids, than the corresponding naturally-occurring Cas9 molecule. The smaller size of the Cas9 molecules allows increased flexibility for delivery methods, and thereby increases utility for genome-editing. A Cas9 molecule or Cas9 polypeptide can comprise one or more deletions that do not substantially affect or decrease the activity of the resultant Cas9 molecules or Cas9 polypeptides described herein. Activities that are retained in the Cas9 molecules or Cas9 polypeptides comprising a deletion as described herein include one or more of the following: a nickase activity, i.e., the ability to cleave a single strand, e.g., the non-complementary strand or the complementary strand, of a nucleic acid molecule; a double stranded nuclease activity, i.e., the ability to cleave both strands of a double stranded nucleic acid and create a double stranded break, which In some embodiments is the presence of two nickase activities; an endonuclease activity; an exonuclease activity; a helicase activity, i.e., the ability to unwind the helical structure of a double stranded nucleic acid; and recognition activity of a nucleic acid molecule, e.g., a target nucleic acid or a gRNA.

Activity of the Cas9 molecules or Cas9 polypeptides described herein can be assessed using the activity assays described herein or are known.

O) Identifying regions suitable for deletion

Suitable regions of Cas9 molecules for deletion can be identified by a variety of methods. Naturally-occurring orthologous Cas9 molecules from various bacterial species, can be modeled onto the crystal structure of S. pyogenes Cas9 (Nishimasu et al., Cell, 156:935-949, 2014) to examine the level of conservation across the selected Cas9 orthologs with respect to the three-dimensional conformation of the protein. Less conserved or unconserved regions that are spatially located distant from regions involved in Cas9 activity, e.g., interface with the target nucleic acid molecule and/or gRNA, represent regions or domains are candidates for deletion without substantially affecting or decreasing Cas9 activity.

P) REC-Optimized Cas9 Molecules and Cas9 Polypeptides

A REC-optimized Cas9 molecule, or a REC-optimized Cas9 polypeptide, as that term is used herein, refers to a Cas9 molecule or Cas9 polypeptide that comprises a deletion in one or both of the REC2 domain and the RE1_{CT} domain (collectively a REC deletion), wherein the deletion comprises at least 10% of the amino acid residues in the cognate domain. A REC-optimized Cas9 molecule or Cas9 polypeptide can be an eaCas9 molecule or eaCas9 polypeptide, or an eiCas9 molecule or eiCas9 polypeptide. An exemplary REC-optimized Cas9 molecule or REC-optimized Cas9 polypeptide comprises: a) a deletion selected from: i) a REC2 deletion; ii) a REC1_{CT} deletion; or iii) a REC1_{SUB} deletion.

Optionally, a linker is disposed between the amino acid residues that flank the deletion. In some embodiments a Cas9 molecule or Cas9 polypeptide includes only one deletion, or only two deletions. A Cas9 molecule or Cas9 polypeptide can comprise a REC2 deletion and a REC1_{CT} deletion. A Cas9 molecule or Cas9 polypeptide can comprise a REC2 deletion and a REC1_{SUB} deletion.

Generally, the deletion will contain at least 10% of the amino acids in the cognate domain, e.g., a REC2 deletion will include at least 10% of the amino acids in the REC2 domain.

A deletion can comprise: at least 10, 20, 30, 40, 50, 60, 70, 80, or 90% of the amino acid residues of its cognate domain; all of the amino acid residues of its cognate domain; an amino acid residue outside its cognate domain; a plurality of amino acid residues outside its cognate domain; the amino acid residue immediately N terminal to its cognate domain; the amino acid residue immediately C terminal to its cognate domain; the amino acid residue immediately N terminal to its cognate and the amino acid residue immediately C terminal to its cognate domain; a plurality of, e.g., up to 5, 10, 15, or 20, amino acid residues N terminal to its cognate domain; a plurality of, e.g., up to 5, 10, 15, or 20, amino acid residues C terminal to its cognate domain; a plurality of, e.g., up to 5, 10, 15, or 20, amino acid residues N terminal to its cognate domain and a plurality of e.g., up to 5, 10, 15, or 20, amino acid residues C terminal to its cognate domain.

In some embodiments, a deletion does not extend beyond: its cognate domain; the N terminal amino acid residue of its cognate domain; the Cβ terminal amino acid residue of its cognate domain.

A REC-optimized Cas9 molecule or REC-optimized Cas9 polypeptide can include a linker disposed between the amino acid residues that flank the deletion. Suitable linkers for use between the amino acid resides that flank a REC deletion in a REC-optimized Cas9 molecule is described herein.

In some embodiments, a REC-optimized Cas9 molecule or REC-optimized Cas9 polypeptide comprises an amino acid sequence that, other than any REC deletion and associated linker, has at least 50, 55, 60, 65, 70, 75, 80, 85, 90, 95, 99, or 100% homology with the amino acid sequence of a naturally occurring Cas9, e.g., a S. aureus Cas9 molecule, a S. pyogenes Cas9 molecule, or a C. jejuni Cas9 molecule.

In some embodiments, a REC-optimized Cas9 molecule or REC-optimized Cas9 polypeptide comprises an amino acid sequence that, other than any REC deletion and associated linker, differs by no more than 1, 2, 3, 4, 5, 6, 7, 8, 9, 10, 15, 20, or 25, amino acid residues from the amino acid sequence of a naturally occurring Cas9, e.g., a S. aureus Cas9 molecule, a S. pyogenes Cas9 molecule, or a C. jejuni Cas9 molecule.

In some embodiments, a REC-optimized Cas9 molecule or REC-optimized Cas9 polypeptide comprises an amino acid sequence that, other than any REC deletion and associate linker, differs by no more than 1, 2, 3, 4, 5, 6, 7, 8, 9, 10, 15, 20, or 25% of the, amino acid residues from the amino acid sequence of a naturally occurring Cas9, e.g., a S. aureus Cas9 molecule, a S. pyogenes Cas9 molecule, or a C. jejuni Cas9 molecule.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology).

Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

The percent identity between two amino acid sequences can also be determined using the algorithm of E. Meyers and W. Miller, (1988) Comput. Appl. Biosci. 4:11-17) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (1970) J. Mol. Biol. 48:444-453) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

Sequence information for exemplary REC deletions are provided for 83 naturally-occurring Cas9 orthologs described in, e.g., International PCT Pub. Nos. WO2015/161276, WO2017/193107 and WO2017/093969.

Q) Nucleic Acids Encoding Cas9 Molecules

Nucleic acids encoding the Cas9 molecules or Cas9 polypeptides, e.g., an eaCas9 molecule or eaCas9 polypeptide, can be used in connection with any of the embodiments provided herein.

Exemplary nucleic acids encoding Cas9 molecules or Cas9 polypeptides are described in Cong et al., Science 2013, 399(6121):819-823; Wang et al., Cell 2013, 153(4):910-918; Mali et al., Science 2013, 399(6121):823-826; Jinek et al., Science 2012, 337(6096):816-821, and WO2015/161276, e.g., in FIG. 8 therein.

In some embodiments, a nucleic acid encoding a Cas9 molecule or Cas9 polypeptide can be a synthetic nucleic acid sequence. For example, the synthetic nucleic acid molecule can be chemically modified. In some embodiments, the Cas9 mRNA has one or more (e.g., all of the following properties: it is capped, polyadenylated, substituted with 5-methylcytidine and/or pseudouridine.

In addition, or alternatively, the synthetic nucleic acid sequence can be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein.

In addition, or alternatively, a nucleic acid encoding a Cas9 molecule or Cas9 polypeptide may comprise a nuclear localization sequence (NLS). Nuclear localization sequences are known.

If any Cas9 sequences are fused with a peptide or polypeptide at the C-terminus, it is understood that the stop codon will be removed.

R) Other Cas Molecules and Cas Polypeptides

Various types of Cas molecules or Cas polypeptides can be used to practice the inventions disclosed herein. In some embodiments, Cas molecules of Type II Cas systems are used. In other embodiments, Cas molecules of other Cas systems are used. For example, Type I or Type III Cas molecules may e used. Exemplary Cas molecules (and Cas systems) are described, e.g., in Haft et al., PLoS Computational Biology 2005, 1(6): e60 and Makarova et al., Nature Review Microbiology 2011, 9:467-477, the contents of both references are incorporated herein by reference in their entirety. Exemplary Cas molecules (and Cas systems) are also shown in Table 6.

TABLE 6
Cas Systems

| | | | Structure of | Families (and | |
|---|---|---|---|---|---|
| | | | encoded | superfamily) | |
| Gene | System type | Name from | protein (PDB | of encoded | |
| name^{‡} | or subtype | Haft et al.^{§} | accessions)^{¶} | protein^{#}** | Representatives |
| cas1 | Type I | cas1 | 3GOD, 3LFX | COG1518 | SERP2463, |
| | Type II | | and 2YZS | | SPy1047 and ygbT |
| | Type III | | | | |
| cas2 | Type I | cas2 | 2IVY, 2I8E | COG1343 and | SERP2462, |
| | Type II | | and 3EXC | COG3512 | SPy1048, SPy1723 |
| | Type III | | | | (N-terminal |
| | | | | | domain) and ygbF |
| cas3′ | Type I^{‡‡} | cas3 | NA | COG1203 | APE1232 and ygcB |
| cas3″ | Subtype I-A | NA | NA | COG2254 | APE1231 and |
| | Subtype I-B | | | | BH0336 |
| cas4 | Subtype I-A | cas4 and | NA | COG1468 | APE1239 and |
| | Subtype I-B | csa1 | | | BH0340 |
| | Subtype I-C | | | | |
| | Subtype I-D | | | | |
| | Subtype II-B | | | | |
| cas5 | Subtype I-A | cas5a, | 3KG4 | COG1688 | APE1234, BH0337, |
| | Subtype I-B | cas5d, | | (RAMP) | devS and ygcI |
| | Subtype I-C | cas5e, | | | |
| | Subtype I-E | cas5h, | | | |
| | | cas5p, | | | |
| | | cas5t and | | | |
| | | cmx5 | | | |
| cas6 | Subtype I-A | cas6 and | 3I4H | COG1583 and | PF1131 and slr7014 |
| | Subtype I-B | cmx6 | | COG5551 | |
| | Subtype I-D | | | (RAMP) | |
| | Subtype III-A | | | | |
| | Subtype III-B | | | | |
| cas6e | Subtype I-E | cse3 | 1WJ9 | (RAMP) | ygcH |
| cas6f | Subtype I-F | csy4 | 2XLJ | (RAMP) | y1727 |
| cas7 | Subtype I-A | csa2, csd2, | NA | COG1857 and | devR and ygcJ |
| | Subtype I-B | cse4, csh2, | | COG3649 | |
| | Subtype I-C | csp1 and | | (RAMP) | |
| | Subtype I-E | cst2 | | | |
| csm6 | Subtype III-A | APE2256 | 2WTE | COG1517 | APE2256 and |
| | | and csm6 | | | SSO1445 |
| cmr1 | Subtype III-B | cmr1 | NA | COG1367 | PF1130 |
| | | | | (RAMP) | |
| cmr3 | Subtype III-B | cmr3 | NA | COG1769 | PF1128 |
| | | | | (RAMP) | |
| cmr4 | Subtype III-B | cmr4 | NA | COG1336 | PF1126 |
| | | | | (RAMP) | |
| cmr5 | Subtype III-B^{‡‡} | cmr5 | 2ZOP and | COG3337 | MTH324 and |
| | | | 2OEB | | PF1125 |
| cmr6 | Subtype III-B | cmr6 | NA | COG1604 | PF1124 |
| | | | | (RAMP) | |
| csb1 | Subtype I-U | GSU0053 | NA | (RAMP) | Balac_1306 and |
| | | | | | GSU0053 |
| csb2 | Subtype I-U^{§§} | NA | NA | (RAMP) | Balac_1305 and |
| | | | | | GSU0054 |
| csb3 | Subtype I-U | NA | NA | (RAMP) | Balac_1303^{§§} |
| csx17 | Subtype I-U | NA | NA | NA | Btus_2683 |
| csx14 | Subtype I-U | NA | NA | NA | GSU0052 |
| csx10 | Subtype I-U | csx10 | NA | (RAMP) | Caur_2274 |
| csx16 | Subtype III-U | VVA1548 | NA | NA | VVA1548 |
| csaX | Subtype III-U | csaX | NA | NA | SSO1438 |
| csx3 | Subtype III-U | csx3 | NA | NA | AF1864 |
| csx1 | Subtype III-U | csa3, csx1, | 1XMX and | COG1517 and | MJ1666, NE0113, |
| | | csx2, | 2I71 | COG4006 | PF1127 and |
| | | DXTHG, | | | TM1812 |
| | | NE0113 | | | |
| | | and | | | |
| | | TIGR02710 | | | |
| csx15 | Unknown | NA | NA | TTE2665 | TTE2665 |
| csf1 | Type U | csf1 | NA | NA | AFE_1038 |
| csf2 | Type U | csf2 | NA | (RAMP) | AFE_1039 |
| csf3 | Type U | csf3 | NA | (RAMP) | AFE_1040 |
| csf4 | Type U | csf4 | NA | NA | AFE_1037 |

3) Cpf1

In some embodiments, the guide RNA or gRNA promotes the specific association targeting of an RNA-guided nuclease such as a Cas9 or a Cpf1 to a target sequence such as a genomic or episomal sequence in a cell. In general, gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner et al. (Molecular Cell 56(2), 333-339, Oct. 23, 2014 (Briner), which is incorporated by reference), and in Cotta-Ramusino.

Guide RNAs, whether unimolecular or modular, generally include a targeting domain that is fully or partially complementary to a target, and are typically 10-30 nucleotides in length, and in certain embodiments are 16-24 nucleotides in length (for instance, 16, 17, 18, 19, 20, 21, 22, 23 or 24 nucleotides in length). In some aspects, the targeting domains are at or near the 5′ terminus of the gRNA in the case of a Cas9 gRNA, and at or near the 3′ terminus in the case of a Cpf1 gRNA. While the foregoing description has focused on gRNAs for use with Cas9, it should be appreciated that other RNA-guided nucleases have been (or may in the future be) discovered or invented which utilize gRNAs that differ in some ways from those described to this point. For instance, Cpf1 (“CRISPR from Prevotella and Franciscella 1”) is a recently discovered RNA-guided nuclease that does not require a tracrRNA to function. (Zetsche et al., 2015, Cell 163, 759-771 Oct. 22, 2015 (Zetsche I), incorporated by reference herein). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpfl gRNA).

Although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, in some aspects in this disclosure, gRNAs may be described solely in terms of their targeting domain sequences.

More generally, some aspects of the present disclosure relate to systems, methods and compositions that can be implemented using multiple RNA-guided nucleases. Unless otherwise specified, the term gRNA should be understood to encompass any suitable gRNA that can be used with any RNA-guided nuclease, and not only those gRNAs that are compatible with a particular species of Cas9 or Cpf1. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

RNA-guided nucleases include, but are not limited to, naturally-occurring Class 2 CRISPR nucleases such as Cas9, and Cpf1, as well as other nucleases derived or obtained therefrom. In functional terms, RNA-guided nucleases are defined as those nucleases that: (a) interact with (e.g. complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g. Cas9 vs. Cpf1), species (e.g. S. pyogenes vs. S. aureus) or variation (e.g. full-length vs. truncated or split; naturally-occurring PAM specificity vs. engineered PAM specificity, etc.) of RNA-guided nuclease.

In addition to recognizing specific sequential orientations of PAMs and protospacers, RNA-guided nucleases in some embodiments can also recognize specific PAM sequences. S. aureus Cas9, for instance, generally recognizes a PAM sequence of NNGRRT or NNGRRV, wherein the N residues are immediately 3′ of the region recognized by the gRNA targeting domain. S. pyogenes Cas9 generally recognizes NGG PAM sequences. And F. novicida Cpf1 generally recognizes a TTN PAM sequence.

The crystal structure of Acidaminococcus sp. Cpf1 in complex with crRNA and a double-stranded (ds) DNA target including a TTTN PAM sequence has been solved by Yamano et al. (Cell. 2016 May 5; 165(4): 949-962 (Yamano), incorporated by reference herein). Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, —II and —III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:antirepeat duplex in Cas9 gRNAs.

Nucleic acids encoding RNA-guided nucleases, e.g., Cas9, Cpf1 or functional fragments thereof, are provided herein. Exemplary nucleic acids encoding RNA-guided nucleases have been described previously (see, e.g., Cong 2013; Wang 2013; Mali 2013; Jinek 2012).

3. Delivery of Agents for Genetic Disruption

In some embodiments, the targeted genetic disruption, e.g., DNA break, of the endogenous genes encoding TCR, such as TRAC and TRBC1 or TRBC2 in humans is carried out by delivering or introducing one or more agent(s) capable of inducing a genetic disruption, e.g., Cas9 and/or gRNA components, to a cell, using any of a number of known delivery method or vehicle for introduction or transfer to cells, for example, using viral, e.g., lentiviral, delivery vectors, or any of the known methods or vehicles for delivering Cas9 molecules and gRNAs. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505. In some embodiments, nucleic acid sequences encoding one or more components of one or more agent(s) capable of inducing a genetic disruption, e.g., DNA break, is introduced into the cells, e.g., by any methods for introducing nucleic acids into a cell described herein or known. In some embodiments, a vector encoding components of one or more agent(s) capable of inducing a genetic disruption such as a CRISPR guide RNA and/or a Cas9 enzyme can be delivered into the cell.

In some embodiments, the one or more agent(s) capable of inducing a genetic disruption, e.g., one or more agent(s) that is a Cas9/gRNA, is introduced into the cell as a ribonucleoprotein (RNP) complex. RNP complexes include a sequence of ribonucleotides, such as an RNA or a gRNA molecule, and a protein, such as a Cas9 protein or variant thereof. For example, the Cas9 protein is delivered as RNP complex that comprises a Cas9 protein and a gRNA molecule targeting the target sequence, e.g., using electroporation or other physical delivery method. In some embodiments, the RNP is delivered into the cell via electroporation or other physical means, e.g., particle gun, Calcium Phosphate transfection, cell compression or squeezing. In some embodiments, the RNP can cross the plasma membrane of a cell without the need for additional delivery agents (e.g., small molecule agents, lipids, etc.). In some embodiments, delivery of the one or more agent(s) capable of inducing genetic disruption, e.g., CRISPR/Cas9, as an RNP offers an advantage that the targeted disruption occurs transiently, e.g., in cells to which the RNP is introduced, without propagation of the agent to cell progenies. For example, delivery by RNP minimizes the agent from being inherited to its progenies, thereby reducing the chance of off-target genetic disruption in the progenies. In such cases, the genetic disruption and the targeted knock-in (discussed further in Section I.B) can be inherited by the progeny cells, but without the agent itself, which may further introduce off-target genetic disruptions, being passed on to the progeny cells.

Agent(s) and components capable of inducing a genetic disruption, e.g., a Cas9 molecule and gRNA molecule, can be introduced into target cells in a variety of forms using a variety of delivery methods and formulations, as set forth in Tables 7 and 8, or methods described in, e.g., WO 2015/161276; US 2015/0056705, US 2016/0272999, US 2017/0211075; or US 2017/0016027. As described further herein, the delivery methods and formulations can be used to deliver template polynucleotides and/or other agents to the cell in prior or subsequent steps of the methods described herein.

TABLE 7
Exemplary Delivery Methods

| Elements | | |
|---|---|---|
| Cas9 | gRNA | |
| Mole- | mole- | |
| cule(s) | cule(s) | Comments |
| DNA | DNA | In this embodiment, a Cas9 molecule and a gRNA |
| | | are transcribed from DNA. In this embodiment, |
| | | they are encoded on separate molecules. |
| DNA | | In this embodiment, a Cas9 molecule and a gRNA |
| | | are transcribed from DNA, here from a single |
| | | molecule. |
| DNA | RNA | In this embodiment, a Cas9 molecule is transcribed |
| | | from DNA, and a gRNA is provided as in vitro |
| | | transcribed or synthesized RNA |
| mRNA | RNA | In this embodiment, a Cas9 molecule is translated |
| | | from in vitro transcribed mRNA, and a gRNA is |
| | | provided as in vitro transcribed or synthesized RNA. |
| mRNA | DNA | In this embodiment, a Cas9 molecule is translated |
| | | from in vitro transcribed mRNA, and a gRNA is |
| | | transcribed from DNA. |
| Protein | DNA | In this embodiment, a Cas9 molecule is provided as |
| | | a protein, and a gRNA is transcribed from DNA. |
| Protein | RNA | In this embodiment, a Cas9 molecule is provided as |
| | | a protein, and a gRNA is provided as transcribed or |
| | | synthesized RNA. |

TABLE 8
Comparison of Exemplary Delivery Methods

| | | Delivery | | | Type of |
|---|---|---|---|---|---|
| | | into Non- | Duration of | Genome | Molecule |
| Delivery Vector/Mode | | Dividing Cells | Expression | Integration | Delivered |
| Physical (e.g., electroporation, particle | | YES | Transient | NO | Nucleic |
| gun, Calcium Phosphate transfection, | | | | | Acids and |
| cell compression or squeezing) | | | | | Proteins |
| Viral | Retrovirus | NO | Stable | YES | RNA |
| | Lentivirus | YES | Stable | YES/NO with | RNA |
| | | | | modifications | |
| | Adenovirus | YES | Transient | NO | DNA |
| | Adeno-Associated | YES | Stable | NO | DNA |
| | Virus (AAV) | | | | |
| | Vaccinia Virus | YES | Very | NO | DNA |
| | | | Transient | | |
| | Herpes Simplex Virus | YES | Stable | NO | DNA |
| Non-Viral | Cationic Liposomes | YES | Transient | Depends on | Nucleic |
| | | | | what is | Acids and |
| | | | | delivered | Proteins |
| | Polymeric | YES | Transient | Depends on | Nucleic |
| | Nanoparticles | | | what is | Acids and |
| | | | | delivered | Proteins |
| Biological | Attenuated Bacteria | YES | Transient | NO | Nucleic |
| Non-Viral | | | | | Acids |
| Delivery | Engineered | YES | Transient | NO | Nucleic |
| Vehicles | Bacteriophages | | | | Acids |
| | Mammalian Virus-like | YES | Transient | NO | Nucleic |
| | Particles | | | | Acids |
| | Biological liposomes: | YES | Transient | NO | Nucleic |
| | Erythrocyte Ghosts | | | | Acids |
| | and Exosomes | | | | |

In some embodiments, DNA encoding Cas9 molecules and/or gRNA molecules, or RNP complexes comprising a Cas9 molecule and/or gRNA molecules, can be delivered into cells by known methods or as described herein. For example, Cas9-encoding and/or gRNA-encoding DNA can be delivered, e.g., by vectors (e.g., viral or non-viral vectors), non-vector based methods (e.g., using naked DNA or DNA complexes), or a combination thereof. In some embodiments, the polynucleotide containing the agent(s) and/or components thereof is delivered by a vector (e.g., viral vector/virus or plasmid). The vector may be any described herein.

In some aspects, a CRISPR enzyme (e.g. Cas9 nuclease) in combination with (and optionally complexed with) a guide sequence is delivered to the cell. For example, one or more elements of a CRISPR system is derived from a type I, type II, or type III CRISPR system. For example, one or more elements of a CRISPR system are derived from a particular organism comprising an endogenous CRISPR system, such as Streptococcus pyogenes, Staphylococcus aureus or Neisseria meningitides.

In some embodiments, a Cas9 nuclease (e.g., that encoded by mRNA from Staphylococcus aureus or from Streptococcus pyogenes, e.g. pCW-Cas9, Addgene #50661, Wang et al. (2014) Science, 3:343-80-4; or nuclease or nickase lentiviral vectors available from Applied Biological Materials (ABM; Canada) as Cat. No. K002, K003, K005 or K006) and a guide RNA specific to the target gene (e.g. TRAC, TRBC1 and/or TRBC2 in humans) are introduced into cells. In some embodiments, gRNA sequences that is or comprises a targeting domain sequence targeting the target site in a particular gene, such as the TRAC, TRBC1 and/or TRBC2 genes, designed or identified. A genome-wide gRNA database for CRISPR genome editing is publicly available, which contains exemplary single guide RNA (sgRNA) sequences targeting constitutive exons of genes in the human genome or mouse genome (see e.g., genescript.com/gRNA-database.html; see also, Sanjana et al. (2014) Nat. Methods, 11:783-4).

In some aspects, the gRNA sequence is or comprises a sequence with minimal off-target binding to a non-target site or position.

In some embodiments, the polynucleotide containing the agent(s) and/or components thereof or RNP complex is delivered by a non-vector based method (e.g., using naked DNA or DNA complexes). For example, the DNA or RNA or proteins or combination thereof, e.g., ribonucleoprotein (RNP) complexes, can be delivered, e.g., by organically modified silica or silicate (Ormosil), electroporation, transient cell compression or squeezing (e.g., as described in Lee, et al. (2012) Nano Lett 12: 6322-27, Kollmannsperger et al (2016) Nat Comm 7, 10372 doi:10.1038/ncomms10372), gene gun, sonoporation, magnetofection, lipid-mediated transfection, dendrimers, inorganic nanoparticles, calcium phosphates, or a combination thereof.

In some embodiments, delivery via electroporation comprises mixing the cells with the Cas9- and/or gRNA-encoding DNA or RNP complex in a cartridge, chamber or cuvette and applying one or more electrical impulses of defined duration and amplitude. In some embodiments, delivery via electroporation is performed using a system in which cells are mixed with the Cas9- and/or gRNA-encoding DNA in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, the delivery vehicle is a non-viral vector. In some embodiments, the non-viral vector is an inorganic nanoparticle. Exemplary inorganic nanoparticles include, e.g., magnetic nanoparticles (e.g., Fe₃MnO₂) and silica. The outer surface of the nanoparticle can be conjugated with a positively charged polymer (e.g., polyethylenimine, polylysine, polyserine) which allows for attachment (e.g., conjugation or entrapment) of payload. In some embodiments, the non-viral vector is an organic nanoparticle. Exemplary organic nanoparticles include, e.g., SNALP liposomes that contain cationic lipids together with neutral helper lipids which are coated with polyethylene glycol (PEG), and protamine-nucleic acid complexes coated with lipid. Exemplary lipids and/or polymers are known and can be used in the provided embodiments.

In some embodiments, the vehicle has targeting modifications to increase target cell update of nanoparticles and liposomes, e.g., cell specific antigens, monoclonal antibodies, single chain antibodies, aptamers, polymers, sugars, and cell penetrating peptides (e.g., described in US 2016/0272999). In some embodiments, the vehicle uses fusogenic and endosome-destabilizing peptides/polymers. In some embodiments, the vehicle undergoes acid-triggered conformational changes (e.g., to accelerate endosomal escape of the cargo). In some embodiments, a stimulus-cleavable polymer is used, e.g., for release in a cellular compartment. For example, disulfide-based cationic polymers that are cleaved in the reducing cellular environment can be used.

In some embodiments, the delivery vehicle is a biological non-viral delivery vehicle.

In some embodiments, the vehicle is an attenuated bacterium (e.g., naturally or artificially engineered to be invasive but attenuated to prevent pathogenesis and expressing the transgene (e.g., Listeria monocytogenes, certain Salmonella strains, Bifidobacterium longum, and modified Escherichia coli), bacteria having nutritional and tissue-specific tropism to target specific cells, bacteria having modified surface proteins to alter target cell specificity). In some embodiments, the vehicle is a genetically modified bacteriophage (e.g., engineered phages having large packaging capacity, less immunogenicity, containing mammalian plasmid maintenance sequences and having incorporated targeting ligands). In some embodiments, the vehicle is a mammalian virus-like particle. For example, modified viral particles can be generated (e.g., by purification of the “empty” particles followed by ex vivo assembly of the virus with the desired cargo). The vehicle can also be engineered to incorporate targeting ligands to alter target tissue-specificity. In some embodiments, the vehicle is a biological liposome. For example, the biological liposome is a phospholipid-based particle derived from human cells (e.g., erythrocyte ghosts, which are red blood cells broken down into spherical structures derived from the subject (e.g., tissue targeting can be achieved by attachment of various tissue or cell-specific ligands), or secretory exosomes—subject-derived membrane-bound nanovesicles (30-100 nm) of endocytic origin (e.g., can be produced from various cell types and can therefore be taken up by cells without the need for targeting ligands).

In some embodiments, RNA encoding Cas9 molecules and/or gRNA molecules, can be delivered into cells, e.g., target cells described herein, by known methods or as described herein. For example, Cas9-encoding and/or gRNA-encoding RNA can be delivered, e.g., by microinjection, electroporation, transient cell compression or squeezing (e.g., as described in Lee, et al. (2012) Nano Lett 12: 6322-27), lipid-mediated transfection, peptide-mediated delivery, or a combination thereof.

In some embodiments, delivery via electroporation comprises mixing the cells with the RNA encoding Cas9 molecules and/or gRNA molecules in a cartridge, chamber or cuvette and applying one or more electrical impulses of defined duration and amplitude. In some embodiments, delivery via electroporation is performed using a system in which cells are mixed with the RNA encoding Cas9 molecules and/or gRNA molecules in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, Cas9 molecules can be delivered into cells by known methods or as described herein. For example, Cas9 protein molecules can be delivered, e.g., by microinjection, electroporation, transient cell compression or squeezing (e.g., as described in Lee, et al. (2012) Nano Lett 12: 6322-27), lipid-mediated transfection, peptide-mediated delivery, or a combination thereof. Delivery can be accompanied by DNA encoding a gRNA or by a gRNA.

In some embodiments, delivery via electroporation comprises mixing the cells with the Cas9 molecules with or without gRNA molecules in a cartridge, chamber or cuvette and applying one or more electrical impulses of defined duration and amplitude. In some embodiments, delivery via electroporation is performed using a system in which cells are mixed with the Cas9 molecules with or without gRNA molecules in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, the polynucleotide containing the agent(s) and/or components thereof is delivered by a combination of a vector and a non-vector based method. For example, a virosome comprises a liposome combined with an inactivated virus (e.g., HIV or influenza virus), which can result in more efficient gene transfer than either a viral or a liposomal method alone.

In some embodiments, more than one agent(s) or components thereof are delivered to the cell. For example, in some embodiments, agent(s) capable of inducing a genetic disruption of two or more locations in the genome, e.g., the TRAC, TRBC1 and/or TRBC2 loci, are delivered to the cell. In some embodiments, agent(s) and components thereof are delivered using one method. For example, in some embodiments, agent(s) for inducing a genetic disruption of TRAC, TRBC1 and/or TRBC2 loci are delivered as polynucleotides encoding the components for genetic disruption. In some embodiments, one polynucleotide can encode agents that target the TRAC, TRBC1 and/or TRBC2 loci. In some embodiments, two or more different polynucleotides can encode the agents that target TRAC, TRBC1 and/or TRBC2 loci. In some embodiments, the agents capable of inducing a genetic disruption can be delivered as ribonucleoprotein (RNP) complexes, and two or more different RNP complexes can be delivered together as a mixture, or separately.

In some embodiments, one or more nucleic acid molecules other than the one or more agent(s) capable of inducing a genetic disruption and/or component thereof, e.g., the Cas9 molecule component and/or the gRNA molecule component, such as a template polynucleotide for HDR-directed integration (such as any template polynucleotide described herein, e.g., in Section I-B), are delivered. In some embodiments, the nucleic acid molecule, e.g., template polynucleotide, is delivered at the same time as one or more of the components of the Cas system. In some embodiments, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Cas system are delivered. In some embodiments, the nucleic acid molecule, e.g., template polynucleotide, is delivered by a different means from one or more of the components of the Cas system, e.g., the Cas9 molecule component and/or the gRNA molecule component. The nucleic acid molecule, e.g., template polynucleotide, can be delivered by any of the delivery methods described herein. For example, the nucleic acid molecule, e.g., template polynucleotide, can be delivered by a viral vector, e.g., a retrovirus or a lentivirus, and the Cas9 molecule component and/or the gRNA molecule component can be delivered by electroporation. In some embodiments, the nucleic acid molecule, e.g., template polynucleotide, includes one or more transgenes, e.g., transgenes that encode a recombinant TCR, a recombinant CAR and/or other gene products.

B. Targeted Integration via Homology Directed Repair (HDR)

In some of the embodiments provided herein, homology-directed repair (HDR) can be utilized for targeted integration of a specific portion of the template polynucleotide containing a transgene, e.g., nucleic acid sequence encoding a recombinant receptor, at a particular location in the genome, e.g., the TRAC, TRBC1 and/or TRBC2 locus. In some embodiments, the presence of a genetic disruption (e.g., a DNA break, such as described in Section I.A) and a template polynucleotide containing one or more homology arms (e.g., containing nucleic acid sequences homologous sequences surrounding the genetic disruption) can induce or direct HDR, with homologous sequences acting as a template for DNA repair. Based on homology between the endogenous gene sequence surrounding the genetic disruption and the 5′ and/or 3′ homology arms included in the template polynucleotide, cellular DNA repair machinery can use the template polynucleotide to repair the DNA break and resynthesize genetic information at the site of the genetic disruption, thereby effectively inserting or integrating the transgene sequences in the template polynucleotide at or near the site of the genetic disruption. In some embodiments, the genetic disruption, e.g., TRAC, TRBC1 and/or TRBC2 locus, can be generated by any of the methods for generating a targeted genetic disruption described herein.

Also provided are polynucleotides, e.g., template polynucleotides described herein. In some embodiments, the provided polynucleotides can be employed in the methods described herein, e.g., involving HDR, to target transgene sequences encoding a portion of a recombinant receptor, e.g., recombinant TCR, at the endogenous TRAC, TRBC1 and/or TRBC2 locus.

In some embodiments, the template polynucleotide is or comprises a polynucleotide containing a transgene (exogenous or heterologous nucleic acids sequences) encoding a recombinant receptor or a portion thereof (e.g., one or more chain(s), region(s) or domain(s) of the recombinant receptor), and homology sequences (e.g., homology arms) that are homologous to sequences at or near the endogenous genomic site, e.g., at the endogenous TRAC, TRBC1 and/or TRBC2 locus. In some aspects, the template polynucleotide is introduced as a linear DNA fragment or comprised in a vector. In some aspects, the step for inducing genetic disruption and the step for targeted integration (e.g., by introduction of the template polynucleotide) are performed simultaneously or sequentially.

1. Homology-Directed Repair (HDR)

In some embodiments, homology-directed repair (HDR) can be utilized for targeted integration or insertion of one or more nucleic acid sequences, e.g., transgene sequences, at one or more target site(s) in the genome, e.g., the TRAC, TRBC1 and/or TRBC2 locus. In some embodiments, the nuclease-induced HDR can be used to alter a target sequence, integrate a transgene at a particular target location, and/or to edit or repair a mutation in a particular target gene.

Alteration of nucleic acid sequences at the target site can occur by HDR with an exogenously provided template polynucleotide (also referred to as donor polynucleotide or template sequence). For example, the template polynucleotide provides for alteration of the target sequence, such as insertion of the transgene contained within the template polynucleotide. In some embodiments, a plasmid or a vector can be used as a template for homologous recombination. In some embodiments, a linear DNA fragment can be used as a template for homologous recombination. In some embodiments, a single stranded template polynucleotide can be used as a template for alteration of the target sequence by alternate methods of homology directed repair (e.g., single strand annealing) between the target sequence and the template polynucleotide. Template polynucleotide-effected alteration of a target sequence depends on cleavage by a nuclease, e.g., a targeted nuclease such as CRISPR/Cas9. Cleavage by the nuclease can comprise a double strand break or two single strand breaks.

In some embodiments, “recombination” refers to a process of exchange of genetic information between two polynucleotides. In some embodiments, “homologous recombination (HR)” refers to the specialized form of such exchange that takes place, for example, during repair of double-strand breaks in cells via homology-directed repair mechanisms. This process requires nucleotide sequence homology, uses a template polynucleotide to template repair of a target DNA (i.e., the one that experienced the double-strand break, e.g., target site in the endogenous gene), and is variously known as “non-crossover gene conversion” or “short tract gene conversion,” because it leads to the transfer of genetic information from the template polynucleotide to the target. In some embodiments, such transfer can involve mismatch correction of heteroduplex DNA that forms between the broken target and the template polynucleotide, and/or “synthesis-dependent strand annealing,” in which the template polynucleotide is used to resynthesize genetic information that will become part of the target, and/or related processes. Such specialized HR often results in an alteration of the sequence of the target molecule such that part or all of the sequence of the template polynucleotide is incorporated into the target polynucleotide.

In some embodiments, a template polynucleotide, e.g., polynucleotide containing transgene, is integrated into the genome of a cell via homology-independent mechanisms. The methods comprise creating a double-stranded break (DSB) in the genome of a cell and cleaving the template polynucleotide molecule using a nuclease, such that the template polynucleotide is integrated at the site of the DSB. In some embodiments, the template polynucleotide is integrated via non-homology dependent methods (e.g., NHEJ). Upon in vivo cleavage the template polynucleotides can be integrated in a targeted manner into the genome of a cell at the location of a DSB. The template polynucleotide can include one or more of the same target sites for one or more of the nucleases used to create the DSB. Thus, the template polynucleotide may be cleaved by one or more of the same nucleases used to cleave the endogenous gene into which integration is desired. In some embodiments, the template polynucleotide includes different nuclease target sites from the nucleases used to induce the DSB. As described herein, the genetic disruption of the target site or target position can be created by any mechanisms, such as ZFNs, TALENs, CRISPR/Cas9 system, or TtAgo nucleases.

In some embodiments, DNA repair mechanisms can be induced by a nuclease after (1) a single double-strand break, (2) two single strand breaks, (3) two double stranded breaks with a break occurring on each side of the target site, (4) one double stranded break and two single strand breaks with the double strand break and two single strand breaks occurring on each side of the target site (5) four single stranded breaks with a pair of single stranded breaks occurring on each side of the target site, or (6) one single stranded break. In some embodiments, a single-stranded template polynucleotide is used and the target site can be altered by alternative HDR.

Template polynucleotide-effected alteration of a target site depends on cleavage by a nuclease molecule. Cleavage by the nuclease can comprise a nick, a double strand break, or two single strand breaks, e.g., one on each strand of the DNA at the target site. After introduction of the breaks on the target site, resection occurs at the break ends resulting in single stranded overhanging DNA regions.

In canonical HDR, a double-stranded template polynucleotide is introduced, comprising homologous sequence to the target site that will either be directly incorporated into the target site or used as a template to insert the transgene or correct the sequence of the target site. After resection at the break, repair can progress by different pathways, e.g., by the double Holliday junction model (or double strand break repair, DSBR, pathway) or the synthesis-dependent strand annealing (SDSA) pathway.

In the double Holliday junction model, strand invasion by the two single stranded overhangs of the target site to the homologous sequences in the template polynucleotide occurs, resulting in the formation of an intermediate with two Holliday junctions. The junctions migrate as new DNA is synthesized from the ends of the invading strand to fill the gap resulting from the resection. The end of the newly synthesized DNA is ligated to the resected end, and the junctions are resolved, resulting in the insertion at the target site, e.g., insertion of the transgene in template polynucleotide. Crossover with the template polynucleotide may occur upon resolution of the junctions.

In the SDSA pathway, only one single stranded overhang invades the template polynucleotide and new DNA is synthesized from the end of the invading strand to fill the gap resulting from resection. The newly synthesized DNA then anneals to the remaining single stranded overhang, new DNA is synthesized to fill in the gap, and the strands are ligated to produce the modified DNA duplex.

In alternative HDR, a single strand template polynucleotide, e.g., template polynucleotide, is introduced. A nick, single strand break, or double strand break at the target site, for altering a desired target site, is mediated by a nuclease molecule, and resection at the break occurs to reveal single stranded overhangs. Incorporation of the sequence of the template polynucleotide to correct or alter the target site of the DNA typically occurs by the SDSA pathway, as described herein.

“Alternative HDR”, or alternative homology-directed repair, in some embodiments, refers to the process of repairing DNA damage using a homologous nucleic acid (e.g., an endogenous homologous sequence, e.g., a sister chromatid, or an exogenous nucleic acid, e.g., a template polynucleotide). Alternative HDR is distinct from canonical HDR in that the process utilizes different pathways from canonical HDR, and can be inhibited by the canonical HDR mediators, RAD51 and BRCA2. Also, alternative HDR uses a single-stranded or nicked homologous nucleic acid for repair of the break. “Canonical HDR”, or canonical homology-directed repair, in some embodiments, refers to the process of repairing DNA damage using a homologous nucleic acid (e.g., an endogenous homologous sequence, e.g., a sister chromatid, or an exogenous nucleic acid, e.g., a template nucleic acid). Canonical HDR typically acts when there has been significant resection at the double strand break, forming at least one single stranded portion of DNA In a normal cell, HDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2 and the homologous nucleic acid is typically double-stranded. Unless indicated otherwise, the term “HDR” in some embodiments encompasses canonical HDR and alternative HDR.

In some embodiments, double strand cleavage is effected by a nuclease, e.g., a Cas9 molecule having cleavage activity associated with an HNH-like domain and cleavage activity associated with a RuvC-like domain, e.g., an N-terminal RuvC-like domain, e.g., a wild type Cas9. Such embodiments require only a single gRNA.

In some embodiments, one single strand break, or nick, is effected by a nuclease molecule having nickase activity, e.g., a Cas9 nickase. A nicked DNA at the target site can be a substrate for alternative HDR.

In some embodiments, two single strand breaks, or nicks, are effected by a nuclease, e.g., Cas9 molecule, having nickase activity, e.g., cleavage activity associated with an HNH-like domain or cleavage activity associated with an N-terminal RuvC-like domain. Such embodiments usually require two gRNAs, one for placement of each single strand break. In some embodiments, the Cas9 molecule having nickase activity cleaves the strand to which the gRNA hybridizes, but not the strand that is complementary to the strand to which the gRNA hybridizes. In some embodiments, the Cas9 molecule having nickase activity does not cleave the strand to which the gRNA hybridizes, but rather cleaves the strand that is complementary to the strand to which the gRNA hybridizes. In some embodiments, the nickase has HNH activity, e.g., a Cas9 molecule having the RuvC activity inactivated, e.g., a Cas9 molecule having a mutation at D10, e.g., the D10A mutation. D10A inactivates RuvC; therefore, the Cas9 nickase has (only) HNH activity and will cut on the strand to which the gRNA hybridizes (e.g., the complementary strand, which does not have the NGG PAM on it). In some embodiments, a Cas9 molecule having an H840, e.g., an H840A, mutation can be used as a nickase. H840A inactivates HNH; therefore, the Cas9 nickase has (only) RuvC activity and cuts on the non-complementary strand (e.g., the strand that has the NGG PAM and whose sequence is identical to the gRNA). In some embodiments, the Cas9 molecule is an N-terminal RuvC-like domain nickase, e.g., the Cas9 molecule comprises a mutation at N863, e.g., N863A.

In some embodiments, in which a nickase and two gRNAs are used to position two single strand nicks, one nick is on the + strand and one nick is on the − strand of the target DNA. The PAMs are outwardly facing. The gRNAs can be selected such that the gRNAs are separated by, from about 0-50, 0-100, or 0-200 nucleotides. In some embodiments, there is no overlap between the target sequences that are complementary to the targeting domains of the two gRNAs. In some embodiments, the gRNAs do not overlap and are separated by as much as 50, 100, or 200 nucleotides. In some embodiments, the use of two gRNAs can increase specificity, e.g., by decreasing off-target binding (Ran et al., Cell 2013).

In some embodiments, a single nick can be used to induce HDR, e.g., alternative HDR. It is contemplated herein that a single nick can be used to increase the ratio of HR to NHEJ at a given cleavage site, e.g., target site. In some embodiments, a single strand break is formed in the strand of the DNA at the target site to which the targeting domain of said gRNA is complementary. In another embodiment, a single strand break is formed in the strand of the DNA at the target site other than the strand to which the targeting domain of said gRNA is complementary.

In some embodiments, other DNA repair pathways such as single strand annealing (SSA), single-stranded break repair (SSBR), mismatch repair (MMR), base excision repair (BER), nucleotide excision repair (NER), intrastrand cross-link (ICL), translesion synthesis (TLS), error-free postreplication repair (PRR) can be employed by the cell to repair a double-stranded or single-stranded break created by the nucleases.

2. Placement of the Genetic Disruption (e.g., DNA Strand Breaks)

Targeted integration results in the transgene being integrated into a specific gene or locus in the genome. The transgene may be integrated anywhere at or near one of the at least one target site(s) or site in the genome. In some embodiments, the transgene is integrated at or near one of the at least one target site(s), for example, within 300, 250, 200, 150, 100, 50, 10, 5, 4, 3, 2, 1 or fewer base pairs upstream or downstream of the site of cleavage, such as within 100, 50, 10, 5, 4, 3, 2, 1 base pairs of either side of the target site, such as within 50, 10, 5, 4, 3, 2, 1 base pairs of either side of the target site. In some embodiments, the integrated sequence comprising the transgene does not include any vector sequences (e.g., viral vector sequences). In some embodiments, the integrated sequence includes a portion of the vector sequences (e.g., viral vector sequences).

The double strand break or single strand break in one of the strands should be sufficiently close to the site for targeted integration such that an alteration is produced in the desired region, e.g., insertion of transgene or correction of a mutation occurs. In some embodiments, the distance is not more than 10, 25, 50, 100, 200, 300, 350, 400 or 500 nucleotides. In some embodiments, it is believed that the break should be sufficiently close to the site for targeted integration such that the break is within the region that is subject to exonuclease-mediated removal during end resection. In some embodiments, the targeting domain is configured such that a cleavage event, e.g., a double strand or single strand break, is positioned within 1, 2, 3, 4, 5, 10, 15, 20, 25, 30, 35, 40, 45, 50, 60, 70, 80, 90, 100, 150, 200, 300, 350, 400 or 500 nucleotides of the region desired to be altered, e.g., site for targeted insertion. The break, e.g., a double strand or single strand break, can be positioned upstream or downstream of the region desired to be altered, e.g., site for targeted insertion. In some embodiments, a break is positioned within the region desired to be altered, e.g., within a region defined by at least two mutant nucleotides. In some embodiments, a break is positioned immediately adjacent to the region desired to be altered, e.g., immediately upstream or downstream of site for targeted integration.

In some embodiments, a single strand break is accompanied by an additional single strand break, positioned by a second gRNA molecule. For example, the targeting domains are configured such that a cleavage event, e.g., the two single strand breaks, are positioned within 1, 2, 3, 4, 5, 10, 15, 20, 25, 30, 35, 40, 45, 50, 60, 70, 80, 90, 100, 150, 200, 300, 350, 400 or 500 nucleotides of a site for targeted integration. In some embodiments, the first and second gRNA molecules are configured such, that when guiding a Cas9 nickase, a single strand break will be accompanied by an additional single strand break, positioned by a second gRNA, sufficiently close to one another to result in alteration of the desired region. In some embodiments, the first and second gRNA molecules are configured such that a single strand break positioned by said second gRNA is within 10, 20, 30, 40, or 50 nucleotides of the break positioned by said first gRNA molecule, e.g., when the Cas9 is a nickase. In some embodiments, the two gRNA molecules are configured to position cuts at the same position, or within a few nucleotides of one another, on different strands, e.g., essentially mimicking a double strand break.

In some embodiments, in which a gRNA (unimolecular (or chimeric) or modular gRNA) and Cas9 nuclease induce a double strand break for the purpose of inducing HDR-mediated insertion of transgene or correction, the cleavage site is between 0-200 bp (e.g., 0-175, 0 to 150, 0 to 125, 0 to 100, 0 to 75, 0 to 50, 0 to 25, 25 to 200, 25 to 175, 25 to 150, 25 to 125, 25 to 100, 25 to 75, 25 to 50, 50 to 200, 50 to 175, 50 to 150, 50 to 125, 50 to 100, 50 to 75, 75 to 200, 75 to 175, 75 to 150, 75 to 125, 75 to 100 bp) away from the site for targeted integration.

In some embodiments, the cleavage site is between 0-100 bp (e.g., 0 to 75, 0 to 50, 0 to 25, 25 to 100, 25 to 75, 25 to 50, 50 to 100, 50 to 75 or 75 to 100 bp) away from the site for targeted integration.

In some embodiments, one can promote HDR by using nickases to generate a break with overhangs. In some embodiments, the single stranded nature of the overhangs can enhance the cell's likelihood of repairing the break by HDR as opposed to, e.g., NHEJ.

Specifically, in some embodiments, HDR is promoted by selecting a first gRNA that targets a first nickase to a first target site, and a second gRNA that targets a second nickase to a second target site which is on the opposite DNA strand from the first target site and offset from the first nick. In some embodiments, the targeting domain of a gRNA molecule is configured to position a cleavage event sufficiently far from a preselected nucleotide, e.g., the nucleotide of a coding region, such that the nucleotide is not altered. In some embodiments, the targeting domain of a gRNA molecule is configured to position an intronic cleavage event sufficiently far from an intron/exon border, or naturally occurring splice signal, to avoid alteration of the exonic sequence or unwanted splicing events. In some embodiments, the targeting domain of a gRNA molecule is configured to position in an early exon, to allow deletion or knock-out of the endogenous gene, and/or allow in-frame integration of the transgene at or near one of the at least one target site(s).

In some embodiments, a double strand break can be accompanied by an additional double strand break, positioned by a second gRNA molecule. In some embodiments, a double strand break can be accompanied by two additional single strand breaks, positioned by a second gRNA molecule and a third gRNA molecule.

In some embodiments, two gRNAs, e.g., independently, unimolecular (or chimeric) or modular gRNA, are configured to position a double-strand break on both sides of a site for targeted integration.

3. Template Polynucleotides

A template polynucleotide having homology with sequences at or near one or more target site(s) in the endogenous DNA can be used to alter the structure of a target DNA, e.g., targeted insertion of the transgene. In some embodiments, the template polynucleotide contains homology sequences (e.g., homology arms) flanking the transgene, e.g., nucleic acid sequences encoding a recombinant receptor, for targeted insertion. In some embodiments, the homology sequences target the transgene at one or more of the TRAC, TRBC1 and/or TRBC2 loci. In some embodiments, the template polynucleotide includes additional sequences (coding or non-coding sequences) between the homology arms, such as a regulatory sequences, such as promoters and/or enhancers, splice donor and/or acceptor sites, internal ribosome entry site (IRES), sequences encoding ribosome skipping elements (e.g., 2A peptides), markers and/or SA sites, and/or one or more additional transgenes.

The sequence of interest in the template polynucleotide may comprise one or more sequences encoding a functional polypeptide (e.g., a cDNA), with or without a promoter.

In some embodiments, the transgene contained in the template polynucleotide comprises a sequence encoding a cell surface receptor (e.g., a recombinant receptor) or a chain thereof, an antibody, an antigen, an enzyme, a growth factor, a nuclear receptor, a hormone, a lymphokine, a cytokine, a reporter, functional fragments or functional variants and combinations, e.g., of any of those described herein. The transgene may encode a one or more proteins useful in cancer therapies, for example one or more chimeric antigen receptors (CARs) and/or a recombinant T cell receptor (TCR). In some embodiments, the transgene can encode any of the recombinant receptors described in Section IV or any chains, regions and/or domains thereof. In some embodiments, the transgene encodes a recombinant T cell receptor (TCR) or any chains, regions and/or domains thereof.

In certain embodiments, the polynucleotide, e.g., template polynucleotide contains and/or includes a transgene encoding a fraction and/or a portion of a recombinant receptor, e.g., a recombinant TCR or a chain thereof. In particular embodiments, the transgene is targeted at a target site(s) that is within a gene, locus, or open reading frame that encodes an endogenous receptor, e.g., an endogenous TCR gene. In some embodiments, the transgene is targeted for in-frame integration within the gene locus, such as to result in a coding sequence that encodes a complete, whole, and/or full length recombinant receptor.

In certain embodiments, the template polynucleotide includes or contains a transgene, a portion of a transgene, and/or a nucleic acid encodes recombinant receptor is a recombinant TCR or chain thereof that contains one or more variable domains and one or more constant domains. In certain embodiments, one or more of the recombinant TCR constant domains shares complete, e.g., at or about 100% identity, to an endogenous TCR constant domain. In particular embodiments, the transgene encodes the portion and/or fraction of the recombinant TCR that does not include the constant domain, and the transgene is integrated in-frame with the sequence, e.g., genomic DNA sequence, encoding the endogenous TCR constant domain. In certain embodiments, the integration results in a coding sequence that encodes the complete, whole, and/or full length recombinant TCR or chain thereof. In some embodiments, the coding sequence contains the transgene sequence encoding the portion or fragment of the TCR or chain thereof and an endogenous sequence encoding the endogenous TCR constant domain.

In certain embodiments, the recombinant TCR or chain thereof contains one or more constant domains that shares complete, e.g., at or about 100% identity, to all or a portion and/or fragment of an endogenous TCR constant domain. In some embodiments, the transgene encodes a portion and/or a fragment of the recombinant receptor that includes a portion and/or a fraction of a constant domain, e.g., a portion or fragment of the constant domain that is completely or partially identical to an endogenous TCR constant domain. In some embodiments, the transgene is integrated in-frame with the sequence, e.g., genomic DNA sequence, encoding the portion and/or fragment of the endogenous TCR constant domain that is not encoded by the transgene.

In particular embodiments, the integration results in a coding sequence that encodes the complete, whole, and/or full length recombinant TCR or chain thereof and contains the transgene sequence and the endogenous sequence encoding the endogenous portion or fragment of the TCR constant domain.

In some embodiments, the transgene encodes a portion of a TCR chain, wherein the portion of the TCR chain is less than a full length, native TCR chain. In some embodiments, the transgene encodes a portion of a TCRα chain that is less than a full length native TCRα chain, e.g., a human TCRα chain. In some embodiments, the portion of the TCRα chain is or includes a TCRα variable domain, e.g., a full length TCRα variable domain, and a portion of a TCRα constant domain. In particular embodiments, the transgene is or contains a sequence of nucleotides that encodes a TCR variable domain and a portion of a sequence of nucleotides encoding a TCRα constant domain that that is less than a full length of a native sequence of nucleotides that encodes a TCRα constant domain. In certain embodiments, the transgene contains a sequence of nucleotides encoding a portion of a TCRα chain that is or includes less than 4 exons, 3 full exons, less than 3 exons, 2 full exons, less than 2 exons, 1 exon, or less than one exon of a gene, locus, or open reading frame that encodes a TCRα domain.

In certain embodiments, the transgene contains a sequence of nucleic acids encoding a portion of a TCR chain, e.g., a portion of a TCRα chain or a portion of a TCRβ chain. In some embodiments, the transgene contains a sequence of nucleic acids encoding a portion of a TCR constant domain, e.g., a portion of a TCRα constant domain or a TCRβ constant domain. In particular embodiments, the sequence of nucleotides encoding the TCR constant domain is or is less than 4,600 nucleotides, 4,500 nucleotides, 4,000 nucleotides, 3,500 nucleotides, 3,000 nucleotides, 2,500 nucleotides, 2,000 nucleotides, 1,800 nucleotides, 1,600 nucleotides, 1,500 nucleotides, 1,400 nucleotides, 1,300 nucleotides, 1,200 nucleotides, 1,100 nucleotides, 1,000 nucleotides, 800 nucleotides, 700 nucleotides, 600 nucleotides, 500 nucleotides, 450 nucleotides, 400 nucleotides, 350 nucleotides, 300 nucleotides, 250 nucleotides, 200 nucleotides, 150 nucleotides, 100 nucleotides, or 50 nucleotides in length. In some embodiment, the transgene contains a sequence of nucleic acids encoding portion of TCR constant domain having at, about, or less than 4,600, 4,500, 4,000, 3,500, 3,000, 2,500, 2,000, 1,800, 1,600, 1,500, 1,400, 1,300, 1,200, 1,100, 1,000, 800, 700, 600, 500, 450, 400, 350, 300, 250, 200, 150, 100, or 50 contiguous nucleotides of a sequence having at or at least 70%, 75%, 80%, 85%, 90%, 95%, 97%, 98%, 99%, 99.5%, or 99.9% sequence identity to all or a portion of the nucleic acid sequence set forth in SEQ ID NOS: 1, 2, or 3.

In certain embodiments, the transgene encodes a portion of a TCRβ chain that is less than a full length native TCRβ chain, e.g., a human TCRβ chain. In some embodiments, the portion of the TCRβ chain is or includes a TCRβ variable domain, e.g., a full length TCRβ variable domain, and a portion of a TCRβ constant domain. In particular embodiments, the transgene is or contains a sequence of nucleotides that encodes a TCR variable domain and a portion of a sequence of nucleotides encoding a TCRβ constant domain that that is less than the full length of a native sequence of nucleotides that encodes a TCRβ constant domain. In certain embodiments, the transgene contains a sequence of nucleotides encoding a portion of a TCRβ chain that is or includes less than 4 exons, 3 full exons, less than 3 exons, 2 full exons, less than 2 exons, 1 exon, or less than one exon of a gene, locus, or open reading frame that encodes a TCRβ domain.

In some of embodiments, the transgene contains a sequence encoding a portion of TCRα chain and/or a portion of a TCRα constant domain that has been codon-optimized. In some of embodiments, the transgene contains a sequence encoding a portion of TCRβ chain and/or a portion of a TCRβ constant domain that has been codon-optimized.

In particular embodiments, the transgene does not contain any introns, e.g., TRAC, TRBC1, and/or TRBC2 introns, or portions thereof. In some embodiments, the transgene contains a sequence of nucleotides encoding a portion of a TCRα chain. In particular embodiments, the sequence of nucleotides encoding the portion of the TCRα chain does not contain any introns or portions thereof. In certain embodiments, the transgene contains a sequence of nucleotides encoding a portion of a TCRβ chain. In some embodiments, the sequence of nucleotides encoding the portion of the TCRβ chain does not contain any introns.

In some embodiments, the transgene encodes a portion of a TCRα chain with less than 100% amino acid sequence identity to a corresponding portion of a native or endogenous TCRα chain. In some embodiments, the portion of the TCRα chain contains an amino acid sequence with, with about, or with at least 70%, 75%, 80%, 85%, 90%, 95%, 98%, 99%, or greater than 99% identity but less than 100% identity to a corresponding portion of a native or endogenous TCRα chain. In particular embodiments, the transgene encodes a portion of a TCRα constant domain with less than 100% amino acid sequence identity to a corresponding portion of a native or endogenous TCRα constant domain. In some embodiments, the portion of the TCRα constant domain contains an amino acid sequence with, with about, or with at least 70%, 75%, 80%, 85%, 90%, 95%, 98%, 99%, or greater than 99% identity but less than 100% identity to a corresponding portion of a native or endogenous TCRα chain.

In particular embodiments, the transgene encodes a portion of a TCRβ chain with less than 100% amino acid sequence identity to a corresponding portion of a native or endogenous TCRβ chain. In certain embodiments, the portion of the TCRβ chain contains an amino acid sequence with, with about, or with at least 70%, 75%, 80%, 85%, 90%, 95%, 98%, 99%, or greater than 99% identity but less than 100% identity to a corresponding portion of a native or endogenous TCRα chain. In particular embodiments, the transgene encodes a portion of a TCRβ constant domain with less than 100% amino acid sequence identity to a corresponding portion of a native or endogenous TCRβ constant domain. In some embodiments, the portion of the TCRβ constant domain contains an amino acid sequence with, with about, or with at least 70%, 75%, 80%, 85%, 90%, 95%, 98%, 99%, or greater than 99% identity but less than 100% identity to a corresponding portion of a native or endogenous TCRβ chain.

In certain embodiments, the transgene contains one or more modifications(s) to introduce one or more cysteine residues that are capable of forming one or more non-native disulfide bridges between the TCRα chain and TCRβ chain. In some embodiments, the transgene encodes a portion of a TCRα chain containing a TCRα constant domain containing a cysteine at a position corresponding to position 48 with numbering as set forth in SEQ ID NO: 24. In some embodiments, the portion of the alpha constant domain contains a portion of the TCRα constant domain having an amino acid sequence set forth in any of SEQ ID NOS: 19 or 24, or a sequence of amino acids that has, has about, or has at least 70%, 75%, 80%, 85% 90%, 95%, 97%, 98%, 99% sequence identity thereto containing one or more cysteine residues capable of forming a non-native disulfide bond with a TCRβ chain.

In some embodiments, the transgene encodes a portion of a TCRβ chain containing a portion of a TCRβ constant domain containing a cysteine at a position corresponding to position 57 with numbering as set forth in SEQ ID NO: 20. In some embodiments, the portion of the alpha constant domain contains a portion of the TCRα constant domain having an amino acid sequence set forth in any of SEQ ID NOS: 20, 21 or 25, or a sequence of amino acids that has, has about, or has at least 70%, 75%, 80%, 85% 90%, 95%, 97%, 98%, 99% sequence identity thereto containing one or more cysteine residues capable of forming a non-native disulfide bond with a TCRα chain.

In particular embodiments, the transgene encodes a portion of a TCRα chain and/or a TCRα constant domain containing one or more modifications to introduce one or more disulfide bonds. In some embodiments, the transgene encodes a portion of a TCRα chain and/or a TCRα constant domain with one or more modifications to remove or prevent a native disulfide bond, e.g., between a TCRα and beta chain. In some embodiments, one or more native cysteines that form and/or are capable of forming a native inter-chain disulfide bond are substituted to another residue, e.g., serine or alanine. In some embodiments, the portion of the TCRα chain and/or TCRα constant domain is modified to replace one or more non-cysteine residues to a cysteine. In some embodiments, the one or more non-native cysteine residues are capable for forming non-native disulfide bonds, e.g., with a TCRβ chain. In some embodiments, embodiments, the cysteine is introduced at one or more of residue Thr48, Thr45, Tyr10, Thr45, and Ser15 with reference to numbering of a TCRα constant domain set forth in SEQ ID NO: 24. In certain embodiments, cysteines can be introduced at residue Ser57, Ser77, Serl7, Asp59, of Glu15 of the TCR β chain with reference to numbering of TCRβ chain set forth in SEQ ID NO: 20. Exemplary non-native disulfide bonds of a TCR are described in published International PCT No. WO2006/000830, WO2006/037960, and Kuball et al. (2007) Blood, 109:2331-2338. In some embodiments, the transgene encodes a portion of a TCRα chain and/or a TCRα constant domain containing one or more modifications to introduce one or more disulfide bonds.

In some embodiments, the transgene encodes all or a portion of a TCRα chain and/or a TCRα constant domain with one or more modifications to remove or prevent a native disulfide bond, e.g., between the TCRα chain encoded by the transgene and the endogenous TCRβ chain. In some embodiments, one or more native cysteines that form and/or are capable of forming a native interchain disulfide bond are substituted to another residue, e.g., serine or alanine. In some embodiments, the portion of the TCRα chain and/or TCRα constant domain is modified to replace one or more non-cysteine residues to a cysteine. In some embodiments, the one or more non-native cysteine residues are capable for forming non-native disulfide bonds, e.g., with a TCRβ chain encoded by the transgene. In some embodiments, the transgene encodes all or a portion of a TCRβ chain and/or a TCRβ constant domain with one or more modifications to remove or prevent a native disulfide bond, e.g., between the TCRβ chain encoded by the transgene and the endogenous TCRα chain. In some embodiments, one or more native cysteines that form and/or are capable of forming a native interchain disulfide bond are substituted to another residue, e.g., serine or alanine. In some embodiments, the portion of the TCRβ chain and/or TCRβ constant domain is modified to replace one or more non-cysteine residues to a cysteine. In some embodiments, the one or more non-native cysteine residues are capable for forming non-native disulfide bonds, e.g., with a TCRα chain encoded by the transgene.

In some embodiments, one or more different template polynucleotides are used for targeting integration of the transgene at one or more different target sites. For targeting integration at different target sites, one or more genetic disruptions (e.g., DNA break) are generated at one or more of the target sites; and one or more different homology sequences are used for targeting integration of the transgene into the respective target site. In some embodiments, the transgene inserted at each site is the same or substantially the same. In some embodiments, transgene inserted at each site are different. In some embodiments, two or more different transgenes, encoding two or more different domains or chains of a protein, is inserted at one or more target sites. In some embodiments, the transgene encoding the recombinant TCR or antigen-binding fragment or chain thereof encodes one chain of a recombinant TCR and the second transgene encodes a different chain of the recombinant TCR. In some embodiments, the transgene encoding the recombinant TCR or antigen-binding fragment or chain thereof encodes the alpha (a) chain of the recombinant TCR and the second transgene encodes the beta (0) chain of the recombinant TCR. In some embodiments, two or more transgene encoding different domains of the recombinant receptors are targeted for integration at two or more target sites. For example, in some embodiments, transgene encoding a recombinant TCRα chain is targeted for integration at the TRAC locus, and transgene encoding a recombinant TCRβ chain is targeted for integration at the TRBC1 and/or TRBC2 loci.

In some embodiments, the one or more target sites are at or near one or more of the TRAC, TRBC1 and/or TRBC2 loci. In some embodiments, the first target site is at or near the coding sequence of the TRAC gene locus, and the second target site is at or near the coding sequence of the TRBC1 gene locus. In some embodiments, the first target site is at or near the coding sequence of the TRAC gene locus, and the second target site is at or near the coding sequence of the TRBC2 gene locus. In some embodiments, the first target site is at or near the coding sequence of the TRAC gene locus, and the second target site both the TRBC1 and TRBC2 loci, e.g., at a sequence that is conserved between TRBC1 and TRBC2.

In some embodiments, one or more different DNA sites, e.g., TRAC, TRBC1 and/or TRBC2 loci, are targeted, and one or more transgene are inserted at each site. In some embodiments, the transgene inserted at each site is the same or substantially the same. In some embodiments, transgene inserted at each site are different. In some embodiments, a transgene is only inserted at one of the target sites (e.g., TRAC locus), and another target site is targeted for gene editing (e.g., knock-out).

In some embodiments, any of the lengths and positions of the homology arms and relative position to the target site(s), such as any described herein, can also apply to the one or more second template polynucleotide(s).

In some embodiments, nuclease-induced HDR results in an insertion of a transgene (also called “exogenous sequence” or “transgene sequence”) for expression of a transgene for targeted insertion. The template polynucleotide sequence is typically not identical to the genomic sequence where it is placed. A template polynucleotide sequence can contain a non-homologous sequence flanked by two regions of homology to allow for efficient HDR at the location of interest. Additionally, template polynucleotide sequence can comprise a vector molecule containing sequences that are not homologous to the region of interest in cellular chromatin. A template polynucleotide sequence can contain several, discontinuous regions of homology to cellular chromatin. For example, for targeted insertion of sequences not normally present in a region of interest, said sequences can be present in a transgene and flanked by regions of homology to sequence in the region of interest.

Polynucleotides for insertion can also be referred to as “transgene” or “exogenous sequences” or “donor” polynucleotides or molecules. The template polynucleotide can be DNA, single-stranded and/or double-stranded and can be introduced into a cell in linear or circular form. See also, U.S. Patent Publication Nos. 20100047805 and 20110207221. The template polynucleotide can also be introduced in DNA form, which may be introduced into the cell in circular or linear form. If introduced in linear form, the ends of the template polynucleotide can be protected (e.g., from exonucleolytic degradation) by any known methods. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. If introduced in double-stranded form, the template polynucleotide may include one or more nuclease target site(s), for example, nuclease target sites flanking the transgene to be integrated into the cell's genome. See, e.g., U.S. Patent Publication No. 20130326645.

In some embodiments, the double-stranded template polynucleotide includes sequences (e.g., coding sequences, also referred to as transgene) greater than 1 kb in length, for example between 2 and 200 kb, between 2 and 10 kb (or any value there between). The double-stranded template polynucleotide also includes at least one nuclease target site, for example. In some embodiments, the template polynucleotide includes at least 2 target sites, for example for a pair of ZFNs or TALENs. Typically, the nuclease target sites are outside the transgene sequences, for example, 5′ and/or 3′ to the transgene sequences, for cleavage of the transgene. The nuclease cleavage site(s) may be for any nuclease(s). In some embodiments, the nuclease target site(s) contained in the double-stranded template polynucleotide are for the same nuclease(s) used to cleave the endogenous target into which the cleaved template polynucleotide is integrated via homology-independent methods.

In some embodiments, the nucleic acid template system is double stranded. In some embodiments, the nucleic acid template system is single stranded. In some embodiments, the nucleic acid template system comprises a single stranded portion and a double stranded portion.

In some embodiments, the template polynucleotide contains the transgene, e.g., recombinant receptor-encoding nucleic acid sequences, flanked by homology sequences (also called “homology arms”) on the 5′ and 3′ ends, to allow the DNA repair machinery, e.g., homologous recombination machinery, to use the template polynucleotide as a template for repair, effectively inserting the transgene into the target site of integration in the genome. The homology arm should extend at least as far as the region in which end resection may occur, e.g., in order to allow the resected single stranded overhang to find a complementary region within the template polynucleotide. The overall length could be limited by parameters such as plasmid size or viral packaging limits. In some embodiments, a homology arm does not extend into repeated elements, e.g., ALU repeats or LINE repeats.

Exemplary homology arm lengths include at least or at least about 50, 100, 200, 250, 300, 400, 500, 600, 700, 750, 800, 900, 1000, 2000, 3000, 4000, or 5000 nucleotides. In some embodiments, the homology arm length is 50-100, 100-250, 250-500, 500-750, 750-1000, 1000-2000, 2000-3000, 3000-4000, or 4000-5000 nucleotides.

In some embodiments, one or more of the homology arms contain a sequence of nucleotides that encode a portion of a TCR, e.g., a TCR constant domain such as an alpha or beta TCR. In some embodiments, one or more homology arms are connected or linked in frame with the transgene. Thus, in some embodiments, one or more of the homology arms and the transgene encode a portion of a TCR chain that is larger than the portion of the TCR chain encoded by the transgene alone. In some embodiments, the combination of one or more of the homology arms and the transgene encode a full exon of a gene, locus, or open reading frame that encodes a TCR constant domain, e.g., a TCRα or TCRβ constant domain. In some embodiments, one or more homology arms contain a sequence of nucleotides that encodes all or a portion of an intron, e.g., an TRAC, TRBC1, or TRBC2 intron.

Target site (also known as “target position,” “target DNA sequence” or “target location”), in some embodiments, refers to a site on a target DNA (e.g., the chromosome) that is modified by the one or more agent(s) capable of inducing a genetic disruption, e.g., a Cas9 molecule. For example, the target site can be a modified Cas9 molecule cleavage of the DNA at the target site and template polynucleotide directed modification, e.g., targeted insertion of the transgene, at the target site. In some embodiments, a target site can be a site between two nucleotides, e.g., adjacent nucleotides, on the DNA into which one or more nucleotides is added.

The target site may comprise one or more nucleotides that are altered by a template polynucleotide. In some embodiments, the target site is within a target sequence (e.g., the sequence to which the gRNA binds). In some embodiments, a target site is upstream or downstream of a target sequence (e.g., the sequence to which the gRNA binds). In some aspects, a pair of single stranded breaks (e.g., nicks) on each side of the target site can be generated.

In some embodiments, the template polynucleotide comprises about 500 to 1000, e.g., 600 to 900 or 700 to 800, base pairs of homology on either side of the target site at the endogenous gene. In some embodiments, the template polynucleotide comprises about 500, 600, 700, 800, 900 or 1000 base pairs homology 5′ of the target site, 3′ of the target site, or both 5′ and 3′ of the target site. In certain embodiments, the target site is within the TRAC, TRBC1, and/or TRBC2 gene, locus, or open reading frame.

In some embodiments, the template polynucleotide comprises about 10, 20, 30, 40, 50, 100, 200, 300, 400, 500, 600, 700, 800, 900, 1000, 1500, 2000, 3000, 4000, or 5000 base pairs homology 3′ of the target site. In some embodiments, the template polynucleotide comprises about 100 to 500, 200 to 400 or 250 to 350, base pairs homology 3′ of the transgene and/or target site. In some embodiments, the template polynucleotide comprises less than about 100, 90, 80, 70, 60, 50, 40, 30, 20, 15, or 10 base pairs homology 5′ of the target site. In some embodiments, the target site is within the TRAC, TRBC1, and/or TRBC2 gene, locus, or open reading frame.

In some embodiments, the template polynucleotide comprises about 10, 20, 30, 40, 50, 100, 200, 300, 400, 500, 600, 700, 800, 900, 1000, 1500, 2000, 3000, 4000, or 5000 base pairs homology 5′ of the target site. In some embodiments, the template polynucleotide comprises about 100 to 500, 200 to 400 or 250 to 350, base pairs homology 5′ of the transgene and/or target site. In some embodiments, the template polynucleotide comprises less than about 100, 90, 80, 70, 60, 50, 40, 30, 20, 15, or 10 base pairs homology 3′ of the target site. In particular embodiments, the target site is within the TRAC, TRBC1, and/or TRBC2 gene, locus, or open reading frame.

In some embodiments, a template polynucleotide is to a nucleic acid sequence which can be used in conjunction with one or more agent(s) capable of introducing a genetic disruption to alter the structure of a target site. In some embodiments, the target site is modified to have the some or all of the sequence of the template polynucleotide, typically at or near cleavage site(s). In some embodiments, the template polynucleotide is single stranded. In some embodiments, the template polynucleotide is double stranded. In some embodiments, the template polynucleotide is DNA, e.g., double stranded DNA In some embodiments, the template polynucleotide is single stranded DNA. In some embodiments, the template polynucleotide is encoded on the same vector backbone, e.g. AAV genome, plasmid DNA, as the Cas9 and gRNA. In some embodiments, the template polynucleotide is excised from a vector backbone in vivo, e.g., it is flanked by gRNA recognition sequences. In some embodiments, the template polynucleotide is on a separate polynucleotide molecule as the Cas9 and gRNA. In some embodiments, the Cas9 and the gRNA are introduced in the form of a ribonucleoprotein (RNP) complex, and the template polynucleotide is introduced as a polynucleotide molecule, e.g., in a vector.

In some embodiments, the template polynucleotide alters the structure of the target site, e.g., insertion of transgene, by participating in a homology directed repair event. In some embodiments, the template polynucleotide alters the sequence of the target site.

In some embodiments, the template polynucleotide includes sequence that corresponds to a site on the target sequence that is cleaved by one or more agent(s) capable of introducing a genetic disruption. In some embodiments, the template polynucleotide includes sequence that corresponds to both, a first site on the target sequence that is cleaved in a first agent capable of introducing a genetic disruption, and a second site on the target sequence that is cleaved in a second agent capable of introducing a genetic disruption.

A template polynucleotide typically comprises the following components: [5′ homology arm]-[transgene]-[3′ homology arm]. The homology arms provide for recombination into the chromosome, thus insertion of the transgene into the DNA at or near the cleavage site e.g., target site(s). In some embodiments, the homology arms flank the most distal cleavage sites.

In some embodiments, the 3′ end of the 5′ homology arm is the position next to the 5′ end of the transgene. In some embodiments, the 5′ homology arm can extend at, at about, or at least 10, 20, 30, 40, 50, 100, 200, 300, 400, 500, 600, 700, 800, 900, 1000, 1500, 2000, 3000, 4000, or 5000 nucleotides 5′ from the 5′ end of the transgene.

In some embodiments, the 5′ end of the 3′ homology arm is the position next to the 3′ end of the transgene. In some embodiments, the 3′ homology arm can extend at least 10, 20, 30, 40, 50, 100, 200, 300, 400, 500, 600, 700, 800, 900, 1000, 1500, 2000, 3000, 4000, or 5000 nucleotides 3′ from the 3′ end of the transgene.

In some embodiments, for targeted insertion, the homology arms, e.g., the 5′ and 3′ homology arms, may each comprise about 1000 base pairs (bp) of sequence flanking the most distal gRNAs (e.g., 1000 bp of sequence on either side of the mutation).

In some embodiments, one or more second template polynucleotide comprising one or more second transgene can be introduced. In some embodiments, the one or more second transgene is targeted for integration at or near one of the at least one target site via homology directed repair (HDR).

In some embodiments, the one or more second template polynucleotide comprises the structure [second 5′ homology arm]-[one or more second transgene]-[second 3′ homology arm]. The homology arms provide for recombination into the chromosome, thus insertion of the transgene into the DNA at or near the cleavage site e.g., target site(s). In some embodiments, the homology arms flank the most distal cleavage sites. In some embodiments, the second 5′ homology arm and second 3′ homology arm comprises nucleic acid sequences homologous to nucleic acid sequences surrounding the at least one target site. In some embodiments, the second 5′ homology arm comprises nucleic acid sequences that are homologous to nucleic acid sequences second 5′ of the target site. In some embodiments, the second 3′ homology arm comprises nucleic acid sequences that are homologous to nucleic acid sequences second 3′ of the target site. In some embodiments, the second 5′ homology arm and second 3′ homology arm independently are at least or at least about 10, 20, 30, 40, 50, 100, 200, 300, 400, 500, 600, 700, 800, 900, 1000, 1500, or 2000 base pairs, or less than or less than about 10, 20, 30, 40, 50, 100, 200, 300, 400, 500, 600, 700, 800, 900, 1000, 1500, or 2000 base pairs. In some embodiments, the second 5′ homology arm and second 3′ homology arm independently are between about 50 and 100, 100 and 250, 250 and 500, 500 and 750, 750 and 1000, 1000 and 2000 base pairs. In some embodiments, the second 5′ homology arm and second 3′ homology arm independently are about 10, 20, 30, 40, 50, 100, 200, 300, 400, 500, 600, 700, 800, 900, 1000, 1500, or 2000 base pairs.

In some embodiments, the one or more second transgene is targeted for integration at or near the target site in the TRAC gene. In some embodiments, the one or more second transgene is targeted for integration at or near the target site in the TRBC1 or the TRBC2 gene.

It is contemplated herein that one or both homology arms may be shortened to avoid including certain sequence repeat elements, e.g., Alu repeats or LINE elements. For example, a 5′ homology arm may be shortened to avoid a sequence repeat element. In some embodiments, a 3′ homology arm may be shortened to avoid a sequence repeat element. In some embodiments, both the 5′ and the 3′ homology arms may be shortened to avoid including certain sequence repeat elements. It is contemplated herein that template polynucleotides for targeted insertion may be designed for use as a single-stranded oligonucleotide, e.g., a single-stranded oligodeoxynucleotide (ssODN). When using a ssODN, 5′ and 3′ homology arms may range up to about 200 base pairs (bp) in length, e.g., at least 25, 50, 75, 100, 125, 150, 175, or 200 bp in length. Longer homology arms are also contemplated for ssODNs as improvements in oligonucleotide synthesis continue to be made. In some embodiments, a longer homology arm is made by a method other than chemical synthesis, e.g., by denaturing a long double stranded nucleic acid and purifying one of the strands, e.g., by affinity for a strand-specific sequence anchored to a solid substrate.

In some embodiments, alternative HDR proceeds more efficiently when the template polynucleotide has extended homology 5′ to the target site (i.e., in the 5′ direction of the target site strand). Accordingly, in some embodiments, the template polynucleotide has a longer homology arm and a shorter homology arm, wherein the longer homology arm can anneal 5′ of the target site. In some embodiments, the arm that can anneal 5′ to the target site is at least 25, 50, 75, 100, 125, 150, 175, or 200, 300, 400, 500, 600, 700, 800, 900, 1000, 1500, 2000, 3000, 4000, or 5000 nucleotides from the target site or the 5′ or 3′ end of the transgene. In some embodiments, the arm that can anneal 5′ to the target site is at least 10%, 20%, 30%, 40%, or 50% longer than the arm that can anneal 3′ to the target site. In some embodiments, the arm that can anneal 5′ to the target site is at least 2×, 3×, 4×, or 5× longer than the arm that can anneal 3′ to the target site. Depending on whether a ssDNA template can anneal to the intact strand or the target site strand, the homology arm that anneals 5′ to the target site may be at the 5′ end of the ssDNA template or the 3′ end of the ssDNA template, respectively.

Similarly, in some embodiments, the template polynucleotide has a 5′ homology arm, a transgene, and a 3′ homology arm, such that the template polynucleotide contains extended homology to the 5′ of the target site. For example, the 5′ homology arm and 3′ homology arm may be substantially the same length, but the transgene may extend farther 5′ of the target site than 3′ of the target site. In some embodiments, the homology arm extends at least 10%, 20%, 30%, 40%, 50%, 2×, 3×, 4×, or 5× further to the 5′ end of the target site than the 3′ end of the target site.

In some embodiments alternative HDR proceeds more efficiently when the template polynucleotide is centered on the target site. Accordingly, in some embodiments, the template polynucleotide has two homology arms that are essentially the same size.

For instance, the first homology arm of a template polynucleotide may have a length that is within 10%, 9%, 8%, 7%, 6%, 5%, 4%, 3%, 2%, or 1% of the second homology arm of the template polynucleotide.

Similarly, in some embodiments, the template polynucleotide has a 5′ homology arm, a transgene, and a 3′ homology arm, such that the template polynucleotide extends substantially the same distance on either side of the target site. For example, the homology arms may have different lengths, but the transgene may be selected to compensate for this. For example, the transgene may extend further 5′ from the target site than it does 3′ of the target site, but the homology arm 5′ of the target site is shorter than the homology arm 3′ of the target site, to compensate. The converse is also possible, e.g., that the transgene may extend further 3′ from the target site than it does 5′ of the target site, but the homology arm 3′ of the target site is shorter than the homology arm 5′ of the target site, to compensate.

In some embodiments, the template polynucleotide is a single stranded nucleic acid. In another embodiment, the template polynucleotide is a double stranded nucleic acid. In some embodiments, the template polynucleotide comprises a nucleotide sequence, e.g., of one or more nucleotides, that will be added to or will template a change in the target DNA. In some embodiments, the template polynucleotide comprises a nucleotide sequence that may be used to modify the target site. In some embodiments, the template polynucleotide comprises a nucleotide sequence, e.g., of one or more nucleotides, that corresponds to wild type sequence of the target DNA, e.g., of the target site.

The template polynucleotide may comprise a transgene. In some embodiments, the template polynucleotide comprises a 5′ homology arm. In some embodiments, the template nucleic acid comprises a 3′ homology arm.

In some embodiments, the template polynucleotide is linear double stranded DNA. The length may be, e.g., about 200-5000 base pairs, e.g., about 200, 300, 400, 500, 600, 700, 800, 900, 1000, 1200, 1400, 1600, 1800, 2000, 2500, 3000, 4000 or 5000 base pairs. The length may be, e.g., at least 200, 300, 400, 500, 600, 700, 800, 900, 1000, 1200, 1400, 1600, 1800, 2000, 2500, 3000, 4000 or 5000 base pairs. In some embodiments, the length is no greater than 200, 300, 400, 500, 600, 700, 800, 900, 1000, 1200, 1400, 1600, 1800, 2000, 2500, 3000, 4000 or 5000 base pairs. In some embodiments, a double stranded template polynucleotide has a length of about 160 base pairs, e.g., about 200-4000, 300-3500, 400-3000, 500-2500, 600-2000, 700-1900, 800-1800, 900-1700, 1000-1600, 1100-1500 or 1200-1400 base pairs.

The template polynucleotide can be linear single stranded DNA In some embodiments, the template polynucleotide is (i) linear single stranded DNA that can anneal to the nicked strand of the target DNA, (ii) linear single stranded DNA that can anneal to the intact strand of the target DNA, (iii) linear single stranded DNA that can anneal to the transcribed strand of the target DNA, (iv) linear single stranded DNA that can anneal to the non-transcribed strand of the target DNA, or more than one of the preceding.

The length may be, e.g., about 200-5000 base pairs, e.g., about 200, 300, 400, 500, 600, 700, 800, 900, 1000, 1200, 1400, 1600, 1800, 2000, 2500, 3000, 4000 or 5000 nucleotides.

The length may be, e.g., at least 200, 300, 400, 500, 600, 700, 800, 900, 1000, 1200, 1400, 1600, 1800, 2000, 2500, 3000, 4000 or 5000 nucleotides. In some embodiments, the length is no greater than 200, 300, 400, 500, 600, 700, 800, 900, 1000, 1200, 1400, 1600, 1800, 2000, 2500, 3000, 4000 or 5000 nucleotides. In some embodiments, a single stranded template polynucleotide has a length of about 160 nucleotides, e.g., about 200-4000, 300-3500, 400-3000, 500-2500, 600-2000, 700-1900, 800-1800, 900-1700, 1000-1600, 1100-1500 or 1200-1400 nucleotides.

In some embodiments, the template polynucleotide is circular double stranded DNA, e.g., a plasmid.

In some embodiments, the template polynucleotide comprises about 500 to 1000 base pairs of homology on either side of the transgene and/or the target site. In some embodiments, the template polynucleotide comprises about 10, 20, 30, 40, 50, 100, 200, 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises at least 10, 20, 30, 40, 50, 100, 200, 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises no more than 10, 20, 30, 40, 50, 100, 200, 300, 400, 500, 600, 700, 800, 900, 1000, 1500, or 2000 base pairs of homology 5′ of the target site or transgene, 3′ of the target site or transgene, or both 5′ and 3′ of the target site or transgene.

In some embodiments, the length of any of the polynucleotides, e.g., template polynucleotides, may be, e.g., at or about 200-10000 nucleotides, e.g., at or about 200, 300, 400, 500, 600, 700, 800, 900, 1000, 1200, 1400, 1600, 1800, 2000, 2500, 3000, 4000, 5000, 6000, 7000, 8000, 9000 or 10000 nucleotides, or a value between any of the foregoing. In some embodiments, the length may be, e.g., at least at or about 200, 300, 400, 500, 600, 700, 800, 900, 1000, 1200, 1400, 1600, 1800, 2000, 2500, 3000, 4000, 5000, 6000, 7000, 8000, 9000 or 10000 nucleotides, or a value between any of the foregoing. In some embodiments, the length is no greater than at or about 200, 300, 400, 500, 600, 700, 800, 900, 1000, 1200, 1400, 1600, 1800, 2000, 2500, 3000, 4000, 5000, 6000, 7000, 8000, 9000 or 10000 nucleotides. In some embodiments, the length is at or about 200-4000, 300-3500, 400-3000, 500-2500, 600-2000, 700-1900, 800-1800, 900-1700, 1000-1600, 1100-1500 or 1200-1400 nucleotides. In some embodiments, the polynucleotide is at least at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length, or any value between any of the foregoing. In some embodiments, the polynucleotide is between at or about 2500 and at or about 5000 nucleotides, at or about 3500 and at or about 4500 nucleotides, or at or about 3750 nucleotides and at or about 4250 nucleotides in length. In some embodiments, the polynucleotide is at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length.

In some embodiments, the template polynucleotide contains homology arms for targeting the endogenous TRAC locus (exemplary nucleotide sequence of the human TRAC gene locus set forth in SEQ ID NO:1; NCBI Reference Sequence: NG_001332.3, TRAC). In some embodiments, the genetic disruption of the TRAC locus is introduced at early coding region the gene, including sequence immediately following a transcription start site, within a first exon of the coding sequence, or within 500 bp of the transcription start site (e.g., less than 500, 450, 400, 350, 300, 250, 200, 150, 100 or 50 bp), or within 500 bp of the start codon (e.g., less than 500, 450, 400, 350, 300, 250, 200, 150, 100 or 50 bp). In some embodiments, the genetic disruption is introduced using any of the targeted nucleases and/or gRNAs described herein, e.g., in Section I.A. In some embodiments, the template polynucleotide comprises about 500 to 1000, e.g., 600 to 900 or 700 to 800, base pairs of homology on either side of the genetic disruption introduced by the targeted nucleases and/or gRNAs. In some embodiments, the template polynucleotide comprises about 500, 600, 700, 800, 900 or 1000 base pairs of 5′ homology arm sequences, which is homologous to 500, 600, 700, 800, 900 or 1000 base pairs of sequences 5′ of the genetic disruption (e.g., at TRAC locus), the transgene, and about 500, 600, 700, 800, 900 or 1000 base pairs of 3′ homology arm sequences, which is homologous to 500, 600, 700, 800, 900 or 1000 base pairs of sequences 3′ of the genetic disruption (e.g., at TRAC locus).

In some embodiments, the template polynucleotide contains homology arms for targeting the endogenous TRBC1 or TRBC2 locus (exemplary nucleotide sequence of the human TRBC1 gene locus set forth in SEQ ID NO:2; NCBI Reference Sequence: NG_001333.2, TRBC1; exemplary nucleotide sequence of the human TRBC2 gene locus set forth in SEQ ID NO:3; NCBI Reference Sequence: NG_001333.2, TRBC2). In some embodiments, the genetic disruption of the TRBC1 or TRBC2 locus is introduced at early coding region the gene, including sequence immediately following a transcription start site, within a first exon of the coding sequence, or within 500 bp of the transcription start site (e.g., less than 500, 450, 400, 350, 300, 250, 200, 150, 100 or 50 bp), or within 500 bp of the start codon (e.g., less than 500, 450, 400, 350, 300, 250, 200, 150, 100 or 50 bp). In some embodiments, the genetic disruption is introduced using any of the targeted nucleases and/or gRNAs described herein, e.g., in Section I.A. In some embodiments, the template polynucleotide comprises about 500 to 1000, e.g., 600 to 900 or 700 to 800, base pairs of homology on either side of the genetic disruption introduced by the targeted nucleases and/or gRNAs. In some embodiments, the template polynucleotide comprises about 500, 600, 700, 800, 900 or 1000 base pairs of 5′ homology arm sequences, which is homologous to 500, 600, 700, 800, 900 or 1000 base pairs of sequences 5′ of the genetic disruption (e.g., at TRBC1 or TRBC2 locus), the transgene, and about 500, 600, 700, 800, 900 or 1000 base pairs of 3′ homology arm sequences, which is homologous to 500, 600, 700, 800, 900 or 1000 base pairs of sequences 3′ of the genetic disruption (e.g., at TRBC1 or TRBC2 locus).

In some embodiments, any of the lengths and positions of the homology arms and relative position to the target site(s), such as any described herein, can also apply to the one or more second template polynucleotide(s).

In some instances, the template polynucleotide comprises a promoter, e.g., a promoter that is exogenous and/or not present at or near the target locus. In some embodiments, the promoter drives expression only in a specific cell type (e.g., a T cell or B cell or NK cell specific promoter). In some embodiments in which the functional polypeptide encoding sequences are promoterless, expression of the integrated transgene is then ensured by transcription driven by an endogenous promoter or other control element in the region of interest.

The transgene, including the transgene encoding the recombinant receptor or antigen-binding portion thereof or a chain thereof and/or the one or more second transgene, can be inserted so that its expression is driven by the endogenous promoter at the integration site, namely the promoter that drives expression of the endogenous gene into which the transgene is inserted (e.g., TRAC, TRBC1 and/or TRBC2). For example, the coding sequences in the transgene can be inserted without a promoter, but in-frame with the coding sequence of the endogenous target gene, such that expression of the integrated transgene is controlled by the transcription of the endogenous promoter at the integration site. In some embodiments, the transgene encoding the recombinant TCR or antigen-binding fragment or chain thereof and/or the one or more second transgene independently is operably linked to the endogenous promoter of the gene at the target site. In some embodiments, a ribosome skipping element/self-cleavage element, such as a 2A element, is placed upstream of the transgene coding sequence, such that the ribosome skipping element/self-cleavage element is placed in-frame with the endogenous gene.

In some embodiments, the transgene encodes a portion of a TCR having a TCRα chain or portion thereof, and a TCRβ chain or portion thereof. In some embodiments, the encoded TCRα chain and TCRβ chain are separated by a linker or a spacer region. In some embodiments, a linker sequence is included that links the TCRα and TCRβ chains to form the single polypeptide strand. In some embodiments, the linker is of sufficient length to span the distance between the C terminus of the a chain and the N terminus of the β chain, or vice versa, while also ensuring that the linker length is not so long so that it blocks or reduces bonding to a target peptide-MHC complex. In some embodiments, the linker may be any linker capable of forming a single polypeptide strand, while retaining TCR binding specificity. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)n-P-, wherein n is 5 or 6 and P is proline, G is glycine and S is serine (SEQ ID NO: 22). In some embodiments, the linker has the sequence GSADDAKKDAAKKDGKS (SEQ ID NO: 23). In some embodiments, the linker or spacer between the TCRα chain or portion thereof and the TCRβ chain or portion thereof that is recognized by and/or is capable of being cleaved by a protease. In certain embodiments, the linker or spacer between the TCRα chain or portion thereof and the TCRβ chain or portion thereof contains a ribosome skipping element or a self-cleaving element.

In some embodiments, the transgene is or include a sequence of nucleotides that is or includes the structure [TCRβ chain]-[linker]-[portion of TCRα chain]. In particular embodiments, the transgene is or include a sequence of nucleotides that is or includes the structure [TCRβ chain]-[self-cleaving element]-[portion of TCRα chain]. In certain embodiments, the transgene is or include a sequence of nucleotides that is or includes the structure [TCRβ chain]-[ribosome skipping sequence]-[portion of TCRα chain]. In some embodiments, the transgene is or include a sequence of nucleotides that is or includes the structure [TCRα chain]-[linker]-[portion of TCRβ chain]. In particular embodiments, the transgene is or include a sequence of nucleotides that is or includes the structure [TCRα chain]-[self-cleaving element]-[portion of TCRβ chain]. In certain embodiments, the transgene is or include a sequence of nucleotides that is or includes the structure [TCRα chain]-[ribosome skipping sequence]-[portion of TCRβ chain]. In some embodiments, the structures are encoded by a polynucleotide strand of a single or double stranded polynucleotide, in a 5′ to 3′ orientation.

In some cases, the ribosome skipping element/self-cleavage element, such as a T2A, can cause the ribosome to skip (ribosome skipping) synthesis of a peptide bond at the C-terminus of a 2A element, leading to separation between the end of the 2A sequence and the next peptide downstream (see, for example, de Felipe, Genetic Vaccines and Ther. 2:13 (2004) and de Felipe et al. Traffic 5:616-626 (2004)). This allows the inserted transgene to be controlled by the transcription of the endogenous promoter at the integration site, e.g., TRAC, TRBC1 and/or TRBC2 promoter. Exemplary ribosome skipping element/self-cleavage element include 2A sequences from the foot-and-mouth disease virus (F2A, e.g., SEQ ID NO: 11), equine rhinitis A virus (E2A, e.g., SEQ ID NO: 10), Thosea asigna virus (T2A, e.g., SEQ ID NO: 6 or 7), and porcine teschovirus-1 (P2A, e.g., SEQ ID NO: 8 or 9) as described in U.S. Patent Publication No. 20070116690. In some embodiments, the template polynucleotide includes a P2A ribosome skipping element (sequence set forth in SEQ ID NO: 8 or 9) upstream of the transgene, e.g., recombinant receptor encoding nucleic acids.

In some embodiments, transgene may comprise a promoter and/or enhancer, for example a constitutive promoter or an inducible or tissue-specific promoter. In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1α promoter (EF1α), mouse phosphoglycerate kinase 1 promoter (PGK), and chicken 3-Actin promoter coupled with CMV early enhancer (CAGG). In some embodiments, the constitutive promoter is a synthetic or modified promoter. In some embodiments, the promoter is or comprises an MND promoter, a synthetic promoter that contains the U3 region of a modified MoMuLV LTR with myeloproliferative sarcoma virus enhancer (sequence set forth in SEQ ID NO:18 or 126; see Challita et al. (1995) J. Virol. 69(2):748-755). In some embodiments, the promoter is a tissue-specific promoter. In another embodiment, the promoter is a viral promoter.

In another embodiment, the promoter is a non-viral promoter. In some cases, the promoter is selected from among human elongation factor 1 alpha (EF1α) promoter (sequence set forth in SEQ ID NO:4 or 5) or a modified form thereof (EF1α promoter with HTLV1 enhancer; sequence set forth in SEQ ID NO: 127) or the MND promoter (sequence set forth in SEQ ID NO:18 or 126). In some embodiments, the transgene does not include a regulatory element, e.g. promoter.

In some embodiments, a “tandem” cassette is integrated into the selected site. In some embodiments, one or more of the “tandem” cassettes encode one or more polypeptide or factors, each independently controlled by a regulatory element or all controlled as a multi-cistronic expression system. In some embodiments, such as those where the polynucleotide contains a first and second nucleic acid sequence, the coding sequences encoding each of the different polypeptide chains can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see e.g., U.S. Pat. No. 6,060,273). In some embodiments, transcription units can be engineered as a bicistronic unit containing an IRES (internal ribosome entry site), which allows coexpression of gene products by a message from a single promoter. Alternatively, in some cases, a single promoter may direct expression of an RNA that contains, in a single open reading frame (ORF), two or three polypeptides separated from one another by sequences encoding a self-cleavage peptide (e.g., 2A sequences) or a protease recognition site (e.g., furin), as described herein. The ORF thus encodes a single polypeptide, which, either during (in the case of 2A) or after translation, is processed into the individual proteins. In some embodiments, the “tandem cassette” includes the first component of the cassette comprising a promoterless sequence, followed by a transcription termination sequence, and a second sequence, encoding an autonomous expression cassette or a multi-cistronic expression sequence. In some embodiments, the tandem cassette encodes two or more different polypeptides or factors, e.g., two or more chains or domains of a recombinant receptor. In some embodiments, nucleic acid sequences encoding two or more chains or domains of the recombinant receptor are introduced as tandem expression cassettes or bi- or multi-cistronic cassettes, into one target DNA integration site.

The transgene may be inserted into an endogenous gene such that all, some or none of the endogenous gene is expressed. In some embodiments, the transgene (e.g., with or without peptide-encoding sequences) is integrated into any endogenous locus. In some embodiments, the transgene is integrated into the TRAC, TRBC1 and/or TRBC2 gene loci.

In some embodiments, exogenous sequences may also include transcriptional or translational regulatory sequences, for example, promoters, enhancers, insulators, internal ribosome entry sites, sequences encoding 2A peptides and/or polyadenylation signals. Further, the control elements of the genes of interest can be operably linked to reporter genes to create chimeric genes (e.g., reporter expression cassettes). Additionally, splice acceptor sequences may be included. Exemplary known splice acceptor site sequences include, e.g., CTGACCTCTTCTCTTCCTCCCACAG, (SEQ ID NO:119) (from the human HBB gene) and TTTCTCTCCACAG (SEQ ID NO:120) (from the human Immunoglobulin-gamma gene).

In an exemplary embodiment, the template polynucleotide includes homology arms for targeting at the TRAC locus, regulatory sequences, e.g., promoter, and nucleic acid sequences encoding a recombinant receptor, e.g., TCR. In an exemplary embodiment, an additional template polynucleotide is employed, that includes homology arms for targeting at TRBC1 and/or TRBC2 loci, regulatory sequences, e.g., promoter, and nucleic acid sequences encoding another factor.

In some embodiments, exemplary template polynucleotides contain transgene encoding a recombinant T cell receptor under the operable control of the human elongation factor 1 alpha (EF1α) promoter with HTLV1 enhancer (sequence set forth in SEQ ID NO:127) or the MND promoter (sequence set forth in SEQ ID NO:126) or linked to nucleic acid sequences encoding a P2A ribosome skipping element (sequence set forth in SEQ ID NO:8) to drive expression of the recombinant TCR from the endogenous target gene locus (e.g., TRAC), 5′ homology arm sequence of approximately 800 bp (e.g., set forth in SEQ ID NO:124), 3′ homology arm sequence of approximately 800 bp (e.g., set forth in SEQ ID NO:125) that are homologous to sequences surrounding the target integration site in exon 1 of the human TCR α constant region (TRAC) gene. In some embodiments, the template polynucleotide further contains other nucleic acid sequences, e.g., nucleic acid sequences encoding a marker, e.g., a surface marker or a selection marker. In some embodiments, the template polynucleotide further contains viral vector sequences, e.g., adeno-associated virus (AAV) vector sequences.

The transgene contained on the template polynucleotide described herein may be isolated from plasmids, cells or other sources using known standard techniques such as PCR. Template polynucleotide for use can include varying types of topology, including circular supercoiled, circular relaxed, linear and the like. Alternatively, they may be chemically synthesized using standard oligonucleotide synthesis techniques. In addition, template polynucleotides may be methylated or lack methylation. Template polynucleotides may be in the form of bacterial or yeast artificial chromosomes (BACs or YACs).

A polynucleotide can be introduced into a cell as part of a vector molecule having additional sequences such as, for example, replication origins, promoters and genes encoding antibiotic resistance. Moreover, template polynucleotides can be introduced as naked nucleic acid, as nucleic acid complexed with materials such as a liposome, nanoparticle or poloxamer, or can be delivered by viruses (e.g., adenovirus, AAV, herpesvirus, retrovirus, lentivirus and integrase defective lentivirus (IDLV)).

In other aspects, the template polynucleotide is delivered by viral and/or non-viral gene transfer methods. In preferred embodiments, the template polynucleotide is delivered to the cell via an adeno associated virus (AAV). Any AAV vector can be used, including, but not limited to, AAV1, AAV2, AAV3, AAV4, AAV5, AAV6, AAV7, AAV8 and combinations thereof. In some instances, the AAV comprises LTRs that are of a heterologous serotype in comparison with the capsid serotype (e.g., AAV2 ITRs with AAV5, AAV6, or AAV8 capsids).

The template polynucleotide may be delivered using the same gene transfer system as used to deliver the nuclease (including on the same vector) or may be delivered using a different delivery system that is used for the nuclease. In some embodiments, the template polynucleotide is delivered using a viral vector (e.g., AAV) and the nuclease(s) is(are) delivered in mRNA form. The cell may also be treated with one or more molecules that inhibit binding of the viral vector to a cell surface receptor as described herein prior to, simultaneously and/or after delivery of the viral vector (e.g., carrying the nuclease(s) and/or template polynucleotide).

In some embodiments, the template polynucleotide is comprised in a viral vector, and is at least at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length, or any value between any of the foregoing. In some embodiments, the polynucleotide is comprised in a viral vector, and is between at or about 2500 and at or about 5000 nucleotides, at or about 3500 and at or about 4500 nucleotides, or at or about 3750 nucleotides and at or about 4250 nucleotides in length. In some embodiments, the polynucleotide is comprised in a viral vector, and is at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length.

In some embodiments, the template polynucleotide is an adenovirus vector, e.g., an AAV vector, e.g., a ssDNA molecule of a length and sequence that allows it to be packaged in an AAV capsid. The vector may be, e.g., less than 5 kb and may contain an ITR sequence that promotes packaging into the capsid. The vector may be integration-deficient. In some embodiments, the template polynucleotide comprises about 150 to 1000 nucleotides of homology on either side of the transgene and/or the target site. In some embodiments, the template polynucleotide comprises about 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene. In some embodiments, the template polynucleotide comprises at least 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene. In some embodiments, the template polynucleotide comprises at most 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene.

In some embodiments, the template polynucleotide is a lentiviral vector, e.g., an IDLV (integration deficiency lentivirus). In some embodiments, the template polynucleotide comprises about 500 to 1000 base pairs of homology on either side of the transgene and/or the target site. In some embodiments, the template polynucleotide comprises about 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises at least 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises no more than 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises one or more mutations, e.g., silent mutations that prevent Cas9 from recognizing and cleaving the template polynucleotide. The template polynucleotide may comprise, e.g., at least 1, 2, 3, 4, 5, 10, 20, or 30 silent mutations relative to the corresponding sequence in the genome of the cell to be altered. In some embodiments, the template polynucleotide comprises at most 2, 3, 4, 5, 10, 20, 30, or 50 silent mutations relative to the corresponding sequence in the genome of the cell to be altered. In some embodiments, the cDNA comprises one or more mutations, e.g., silent mutations that prevent Cas9 from recognizing and cleaving the template polynucleotide. The template polynucleotide may comprise, e.g., at least 1, 2, 3, 4, 5, 10, 20, or 30 silent mutations relative to the corresponding sequence in the genome of the cell to be altered. In some embodiments, the template polynucleotide comprises at most 2, 3, 4, 5, 10, 20, 30, or 50 silent mutations relative to the corresponding sequence in the genome of the cell to be altered.

The double-stranded template polynucleotides described herein may include one or more non-natural bases and/or backbones. In particular, insertion of a template polynucleotide with methylated cytosines may be carried out using the methods described herein to achieve a state of transcriptional quiescence in a region of interest.

The template polynucleotide may comprise any transgene of interest (exogenous sequence). Exemplary exogenous sequences include, but are not limited to any polypeptide coding sequence (e.g., cDNAs or fragments thereof), promoter sequences, enhancer sequences, epitope tags, marker genes, cleavage enzyme recognition sites and various types of expression constructs. Marker genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence.

In some embodiments, the transgene comprises a polynucleotide encoding any polypeptide of which expression in the cell is desired, including, but not limited to antibodies, antigens, enzymes, receptors (cell surface or nuclear), hormones, lymphokines, cytokines, reporter polypeptides, growth factors, and functional fragments of any of the foregoing. In some embodiments, the exogenous sequence (transgene) comprises a polynucleotide encoding one or more recombinant receptor(s), e.g., functional non-TCR antigen receptors, chimeric antigen receptors (CARs), and T cell receptors (TCRs), such as transgenic TCRs, engineered TCRs or recombinant TCRs, and components of any of the foregoing.

In some embodiments, the coding sequences may be, for example, cDNAs. The exogenous sequences may also be a fragment of a transgene for linking with an endogenous gene sequence of interest. For example, a fragment of a transgene comprising sequence at the 3′ end of a gene of interest may be utilized to correct, via insertion or replacement, of a sequence encoding a mutation in the 3′ end of an endogenous gene sequence. Similarly, the fragment may comprise sequences similar to the 5′ end of the endogenous gene for insertion/replacement of the endogenous sequences to correct or modify such endogenous sequence. Additionally the fragment may encode a functional domain of interest (catalytic, secretory or the like) for linking in situ to an endogenous gene sequence to produce a fusion protein.

In some embodiments, the transgene further encodes one or more marker(s). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the polynucleotide, e.g., a polynucleotide encoding a recombinant receptor. In some embodiments, the transduction marker can indicate or confirm modification of a cell. In some embodiments, the surrogate marker is a protein that is made to be co-expressed on the cell surface with the recombinant receptor, e.g. TCR or CAR. In particular embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO:12 or 13) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR). In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, the nucleic acid encoding the marker is operably linked to a polynucleotide encoding for a linker sequence, such as a cleavable linker sequence, e.g., a T2A. For example, a marker, and optionally a linker sequence, can be any as disclosed in PCT Pub. No. WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 12 or 13 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 12 or 13.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some embodiments, such transgene further includes a T2A ribosomal skip element and/or a sequence encoding a marker such as a tEGFR sequence, e.g., downstream of a sequence encoding one chain of the TCR, such as set forth in SEQ ID NO: 12 or 13, respectively, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 12 or 13.

In some embodiments, the template polynucleotide encodes a recombinant receptor that serves to direct the function of a T cell. Exemplary of such encoded recombinant receptors include recombinant T cell receptors (TCRs). In some cases, a chimeric antigen receptor (CAR) is encoded. Chimeric Antigen Receptors (CARs) are molecules designed to target immune cells to specific molecular targets expressed on cell surfaces. In their most basic form, they are receptors introduced to a cell that couple a specificity domain expressed on the outside of the cell to signaling pathways on the inside of the cell such that when the specificity domain interacts with its target, the cell becomes activated. Often CARs are made from variants of T-cell receptors (TCRs) where a specificity domain such as an scFv or some type of receptor is fused to the signaling domain of a TCR. These constructs are then introduced into a T cell allowing the T cell to become activated in the presence of a cell expressing the target antigen, resulting in the attack on the targeted cell by the activated T cell in a non-MHC dependent manner (see Chicaybam et at (2011) Int Rev Immunol 30:294-311). Alternatively, CAR expression cassettes can be introduced into an immune cell for later engraftment such that the CAR cassette is under the control of a T cell specific promoter (e.g., the FOXP3 promoter, see Mantel et. al (2006) J. Immunol 176: 3593-3602).

In an exemplary embodiment, the template polynucleotide is included as an adeno-associated virus (AAV) vector construct, containing a nucleic acid sequence encoding a recombinant TCR α and TCR β chains under the control of a constitutive promoter, flanked by homology arms of 800 base pairs each on the 5′ and 3′ side of the nucleic acid sequence encoding the recombinant TCR for targeting at exon 1 of the endogenous TRAC gene.

Exemplary 5′ homology arm for targeting at TRAC include the sequence set forth in SEQ ID NO:124. Exemplary 3′ homology arm for targeting at TRAC include the sequence set forth in SEQ ID NO:125.

Construction of such expression cassettes, following the teachings of the present specification, utilizes methodologies that are known in molecular biology (see, for example, Ausubel or Maniatis). Before use of the expression cassette to generate a transgenic animal, the responsiveness of the expression cassette to the stress-inducer associated with selected control elements can be tested by introducing the expression cassette into a suitable cell line (e.g., primary cells, transformed cells, or immortalized cell lines).

Targeted insertion of non-coding nucleic acid sequence may also be achieved. Sequences encoding antisense RNAs, RNAi, shRNAs and micro RNAs (miRNAs) may also be used for targeted insertions. In additional embodiments, the template polynucleotide may comprise non-coding sequences that are specific target sites for additional nuclease designs. Subsequently, additional nucleases may be expressed in cells such that the original template polynucleotide is cleaved and modified by insertion of another template polynucleotide of interest. In this way, reiterative integrations of template polynucleotides may be generated allowing for trait stacking at a particular locus of interest, e.g., TRAC, TRBC1 and/or TRBC2 gene loci.

In some embodiments, the polynucleotide contains the structure: [5′ homology arm]-[transgene sequence]-[3′ homology arm]. In some embodiments, the polynucleotide contains the structure: [5′ homology arm]-[multicistronic element]-[transgene sequence]-[3′ homology arm].

In some embodiments, the polynucleotide contains the structure: [5′ homology arm]-[promoter]-[transgene sequence]-[3′ homology arm].

4. Delivery of Template Polynucleotides

In some embodiments, the polynucleotide, e.g., a polynucleotide such as a template polynucleotide encoding the chimeric receptor, are introduced into the cells in nucleotide form, e.g., as a polynucleotide or a vector. In particular embodiments, the polynucleotide contains a transgene that encodes the chimeric receptor or a portion thereof.

In some embodiments, the polynucleotide, e.g., template polynucleotide, is introduced into the cell for engineering, in addition to the agent(s) capable of inducing a targeted genetic disruption, e.g., nuclease and/or gRNAs. In some embodiments, the template polynucleotide(s) may be delivered prior to, simultaneously or after the agent(s) capable of inducing a targeted genetic disruption is introduced into a cell. In some embodiments, the template polynucleotide(s) are delivered simultaneously with the agents. In some embodiments, the template polynucleotides are delivered prior to the agents, for example, seconds to hours to days before the agents, including, but not limited to, 1 to 60 minutes (or any time there between) before the agents, 1 to 24 hours (or any time there between) before the agents or more than 24 hours before the agents. In some embodiments, the template polynucleotides are delivered after the agents, seconds to hours to days after the agents, including immediately after delivery of the agent, e.g., between 1 minute to 4 hours, such as about 10 minutes, 20 minutes, 30 minutes, 40 minutes, 50 minutes, 60 minutes, 90 minutes, 2 hours, 3 hours or 4 hours after delivery of the agents and/or preferably within 4 hours of delivery of the agents. In some embodiments, the template polynucleotide is delivered more than 4 hours after delivery of the agents. In some embodiments, the template polynucleotides are delivered after the agents, for example, including, but not limited to, within 1 second to 60 minutes (or any time there between) after the agents, 1 to 4 hours (or any time there between) after the agents or more than 4 hours after the agents.

In some embodiments, the template polynucleotides may be delivered using the same delivery systems as the agent(s) capable of inducing a targeted genetic disruption, e.g., nuclease and/or gRNAs. In some embodiments, the template polynucleotides may be delivered using different same delivery systems as the agent(s) capable of inducing a targeted genetic disruption, e.g., nuclease and/or gRNAs. In some embodiments, the template polynucleotide is delivered simultaneously with the agent(s). In other embodiments, the template polynucleotide is delivered at a different time, before or after delivery of the agent(s). Any of the delivery method described herein, e.g., in Section I.A.3 such as in Tables 6 and 7, for delivery of nucleic acids in the agent(s) capable of inducing a targeted genetic disruption, e.g., nuclease and/or gRNAs, can be used to deliver the template polynucleotide.

In some embodiments, the one or more agent(s) and the template polynucleotide are delivered in the same format or method. For example, in some embodiments, the one or more agent(s) and the template polynucleotide are both comprised in a vector, e.g., viral vector. In some embodiments, the template polynucleotide is encoded on the same vector backbone, e.g. AAV genome, plasmid DNA, as the Cas9 and gRNA. In some aspects, the one or more agent(s) and the template polynucleotide are in different formats, e.g., ribonucleic acid-protein complex (RNP) for the Cas9-gRNA agent and a linear DNA for the template polynucleotide, but they are delivered using the same method. In some aspects, the one or more agent(s) and the template polynucleotide are in different formats, e.g., ribonucleic acid-protein complex (RNP) for the Cas9-gRNA agent and the template polynucleotide is in contained in an AAV vector, and the RNP is delivered using a physical delivery method (e.g., electroporation) and the template polynucleotide is delivered via transduction of AAV viral preparations. In some aspects, the template polynucleotide is delivered immediately after, e.g., within about 1, 2, 3, 4, 5, 10, 20, 30, 40, 50 or 60 minutes after, the delivery of the one or more agent(s).

In some embodiments, the template polynucleotide is a linear or circular nucleic acid molecule, such as a linear or circular DNA or linear RNA, and can be delivered using any of the methods described in Section I.A.3 herein (e.g., Tables 6 and 7) for delivering nucleic acid molecules into the cell.

In particular embodiments, the polynucleotide, e.g., the template polynucleotide, are introduced into the cells in nucleotide form, e.g., as or within a non-viral vector. In some embodiments, the non-viral vector is or includes a polynucleotide, e.g., a DNA or RNA polynucleotide, that is suitable for transduction and/or transfection by any suitable and/or known non-viral method for gene delivery, such as but not limited to microinjection, electroporation, transient cell compression or squeezing (e.g., as described in Lee, et al. (2012) Nano Lett 12: 6322-27), lipid-mediated transfection, peptide-mediated delivery, e.g., cell-penetrating peptides, or a combination thereof. In some embodiments, the non-viral polynucleotide is delivered into the cell by a non-viral method described herein, such as a non-viral method listed in Table 7 herein.

In some embodiments, the template polynucleotide sequence can be comprised in a vector molecule containing sequences that are not homologous to the region of interest in the genomic DNA. In some embodiments, the virus is a DNA virus (e.g., dsDNA or ssDNA virus). In some embodiments, the virus is an RNA virus (e.g., ssRNA or dsRNA virus). Exemplary viral vectors/viruses include, e.g., retroviruses, lentiviruses, adenovirus, adeno-associated virus (AAV), vaccinia viruses, poxviruses, and herpes simplex viruses, or any of the viruses described elsewhere herein.

In some embodiments, the template polynucleotide can be transferred into cells using recombinant infectious virus particles, such as, e.g., vectors derived from simian virus 40 (SV40), adenoviruses, adeno-associated virus (AAV). In some embodiments, the template polynucleotide are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 November 29(11): 550-557 or HIV-1 derived lentiviral vectors.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109).

In some embodiments, the template polynucleotides and nucleases may be on the same vector, for example an AAV vector (e.g., AAV6). In some embodiments, the template polynucleotides are delivered using an AAV vector and the agent(s) capable of inducing a targeted genetic disruption, e.g., nuclease and/or gRNAs are delivered as a different form, e.g., as mRNAs encoding the nucleases and/or gRNAs. In some embodiments, the template polynucleotides and nucleases are delivered using the same type of method, e.g., a viral vector, but on separate vectors. In some embodiments, the template polynucleotides are delivered in a different delivery system as the agents capable of inducing a genetic disruption, e.g., nucleases and/or gRNAs. In some embodiments, the template polynucleotide is excised from a vector backbone in vivo, e.g., it is flanked by gRNA recognition sequences. In some embodiments, the template polynucleotide is on a separate polynucleotide molecule as the Cas9 and gRNA. In some embodiments, the Cas9 and the gRNA are introduced in the form of a ribonucleoprotein (RNP) complex, and the template polynucleotide is introduced as a polynucleotide molecule, e.g., in a vector or a linear nucleic acid molecule, e.g., linear DNA. Types or nucleic acids and vectors for delivery include any of those described in Section III herein.

5. Genetically Modified Locus

In some embodiments, the methods, compositions, articles of manufacture, and/or kits provided herein are useful to generate, manufacture, or produce genetically engineered cells, e.g., genetically engineered immune cells and/or T cells, that have or contain a modified gene locus. In particular embodiments, the methods provided herein result in genetically engineered cells that have or contain a modified gene locus. In particular embodiments, the modified locus is or contains a fusion of a transgene, e.g., a transgene described in Section I.B, and an open reading frame of an endogenous gene. In certain embodiments, the transgene encodes a portion of a recombinant protein and is inserted in-frame into the open reading frame of the endogenous gene, resulting in a modified locus that encodes the full recombinant protein. In some embodiments, the recombinant protein is a recombinant receptor. In some embodiments, the recombinant protein is a recombinant TCR.

In certain embodiments, the transgene encodes a portion of a recombinant TCR and is inserted in-frame within an endogenous open reading frame encoding a TCR constant domain. In some embodiments, the modified locus encodes the full recombinant TCR. In particular embodiments, a portion of the encoded recombinant TCR is encoded by a nucleic acid sequence present in the transgene, and the remaining portion of the recombinant TCR is encoded by a nucleic acid sequence present in the open reading frame of the endogenous gene. In particular embodiments, the transcription of the modified locus results in an mRNA that encodes the recombinant TCR. In particular embodiments, a portion of the mRNA is transcribed from a nucleic acid sequence present in the transgene, and the remaining portion of the mRNA is transcribed from a nucleic acid sequence present in the open reading frame of the endogenous gene. In some embodiments, the transgene is integrated at a target site immediately upstream of and in frame with of the region or portion of the open reading frame that encodes the remaining portion of the recombinant TCR.

In some embodiments, the mRNA transcribed from the modified locus contains a 3′UTR that is encoded by the endogenous gene and/or is identical to a 3′UTR of an mRNA that is transcribed from the endogenous gene, e.g., an endogenous and/or unmodified TRAC or TRBC gene. In some embodiments, the transgene contains a ribosomal skipping element upstream, e.g., immediately upstream, of the sequence of nucleic acids encoding the portion of the TCR. In certain embodiments, the transcription of the modified locus results in an mRNA that encodes the recombinant TCR. In some embodiments, the mRNA encoding the recombinant TCR contains a 5′UTR that is encoded by the endogenous gene and/or is identical to a 5′UTR of an mRNA that is transcribed from the endogenous gene, e.g., an endogenous and/or unmodified TRAC or TRBC gene.

In certain embodiments, the modified locus contains a nucleic acid sequence encoding a recombinant TCR having less introns than a locus, e.g., an endogenous or unmodified locus, encoding an endogenous and/or a native TCR. In particular embodiments, the nucleic acid sequence encoding the recombinant TCR has one, two, three, four, five, six, seven, eight, nine, ten, or more than ten fewer introns than a nucleic acid sequence of or within an unmodified locus, e.g., an endogenous locus, encoding a native and/or endogenous TCR. In certain embodiments, the nucleic acid sequence encoding the recombinant TCR has fewer introns than an unmodified TRAC locus. In particular embodiments, the nucleic acid sequence encoding the recombinant TCR has fewer introns than an unmodified TRBC locus, e.g., TRBC1 and/or TRBC2.

In certain embodiments, the modified locus contains a nucleic acid sequence that encodes a recombinant TCR, and the nucleic acid sequence contains at least one, at least two, at least three, at least four, at least five, at least six, at least seven, at least eight, at least nine, or at least ten introns. In particular embodiments, nucleic acid sequence contains one intron. In particular embodiments, the nucleic acid sequence contains two introns. In certain embodiments, the nucleic acid sequence contains three introns. In some embodiments, the introns are not within the nucleic acid sequence of the transgene.

In some embodiments, the modified locus is a modified TRAC locus. In some embodiments, the modified TRAC locus is or contains a fusion of a transgene and an open reading frame of the endogenous TRAC locus. In some embodiments, the modified locus encodes a complete, whole, and/or full length recombinant TCR receptor, a portion of which is encoded the by the transgene, e.g., by a nucleic acid sequence of or within the transgene, and the remaining portion of which is encoded by the open reading frame of the endogenous TRAC gene, e.g., a nucleic acid sequence at or within the open reading frame of the endogenous TRAC gene. In some embodiments, the transgene encodes a portion of a TCRα chain and the open reading frame encodes the remaining portion of the TCRα chain. In certain embodiments, the transgene encodes a TCRβ chain and a portion of a TCRα chain and the open reading frame encodes the remaining portion of the TCRα chain. In some embodiments, the transgene encodes a variable domain of the TCRα chain and the open reading frame encodes the constant region of the TCRα chain. In certain embodiments, transgene encodes a variable domain of the TCRα chain and a portion of the constant domain of the TCRα chain and the open reading frame encodes the remaining portion of the TCRα constant domain.

In some embodiments, the modified locus is a modified TRBC locus, e.g., TRBC1 or TRBC2. In some embodiments, the modified TRBC locus is or contains a fusion of a transgene and an open reading frame of the endogenous TRBC locus. In some embodiments, the modified locus encodes a complete, whole, and/or full length recombinant TCR receptor, a portion of which is encoded the by the transgene, e.g., by a nucleic acid sequence of or within the transgene, and the remaining portion of which is encoded by the open reading frame of the endogenous TRBC gene, e.g., a nucleic acid sequence at or within the open reading frame of the endogenous TRAC gene. In some embodiments, the transgene encodes a portion of a TCRβ chain and the open reading frame encodes the remaining portion of the TCRβ chain. In certain embodiments, the transgene encodes a TCRα chain and a portion of a TCRβ chain and the open reading frame encodes the remaining portion of the TCRβ chain. In some embodiments, the transgene encodes the variable domain of the TCRβ chain and the open reading frame encodes the constant region of the TCRβ chain. In certain embodiments, the transgene encodes a variable domain of the TCRβ and a portion of the constant domain of the TCRβ chain and the open reading frame encodes the remaining portion of the TCRβ constant domain.

In some embodiments, a recombinant TCR encoded by the modified locus is a functional TCR. In some embodiments, a recombinant TCR encoded by the modified locus binds to and/or is capable of binding to a target antigen. In some embodiments, the target antigen is associated with, specific to, and/or expressed on a cell or tissue that is associated with a disease, disorder, or condition. In some embodiments, disease, disorder, or condition is or includes an infectious disease or disorder, an autoimmune disease, an inflammatory disease, a tumor, or a cancer. In some embodiments, the target antigen is a tumor antigen or a pathogenic antigen. In particular embodiments, the pathogenic antigen is a bacterial antigen or viral antigen. In certain embodiments, a TCRα chain that is encoded by the modified TRAC locus binds and/or is capable of binding to a TCRβ chain. In some embodiments, a TCRβ chain that is encoded by the modified TRBC locus, e.g., a TRBC1 or TRBC2 locus, is capable of binding to a TCRα locus.

### II. NUCLEIC ACIDS, VECTORS AND DELIVERY

In some embodiments, the polynucleotide, e.g., a polynucleotide such as a template polynucleotide encoding the recombinant receptor and/or TCR, are introduced into the cells in nucleotide form, e.g., as a polynucleotide or a vector. In particular embodiments, the polynucleotide contains a transgene that encodes the recombinant receptor and/or TCR. In certain embodiments, the one or more agent(s) for genetic disruption are introduced into the cells in nucleic acid form, e.g., as polynucleotides and/or vectors. In some embodiments, the components for engineering can be delivered in various forms using various delivery methods, including as polynucleotides encoding the components, e.g., as described in Section I. Also provided are one or more polynucleotides (e.g., nucleic acid molecules) encoding one or more components of the one or more agent(s) capable of inducing a genetic disruption, and/or one or more template polynucleotides containing transgene, and vectors for genetically engineering cells for targeted integration of the transgene.

In some embodiments, provided are polynucleotides, e.g., template polynucleotides for targeting transgene at a specific genomic target location, e.g., at the TRAC, TRBC1 and/or TRBC2 locus. In some embodiments, provided are any template polynucleotides described herein, e.g., in Section I.B. In some embodiments, the template polynucleotide contains transgene that include nucleic acid sequences that encode a recombinant receptor or other polypeptides and/or factors, and homology arms for targeted integration. In some embodiments, the template polynucleotide can be contained in a vector.

In some embodiments, agents capable of inducing a genetic disruption can be encoded in one or more polynucleotides. In some embodiments, the component of the agents, e.g., Cas9 molecule and/or a gRNA molecule, can be encoded in one or more polynucleotides, and introduced into the cells. In some embodiments, the polynucleotide encoding one or more component of the agents can be included in a vector.

In some embodiments, a vector may comprise a sequence that encodes a Cas9 molecule and/or a gRNA molecule and/or template polynucleotides. A vector may also comprise a sequence encoding a signal peptide (e.g., for nuclear localization, nucleolar localization, mitochondrial localization), fused, e.g., to a Cas9 molecule sequence. For example, a vector may comprise a nuclear localization sequence (e.g., from SV40) fused to the sequence encoding the Cas9 molecule.

In particular embodiments, one or more regulatory/control elements, e.g., a promoter, an enhancer, an intron, a polyadenylation signal, a Kozak consensus sequence, internal ribosome entry sites (IRES), a 2A sequence, and splice acceptor or donor can be included in the vectors.

In some embodiments, the promoter is selected from among an RNA pol I, pol II or pol III promoter. In some embodiments, the promoter is recognized by RNA polymerase II (e.g., a CMV, SV40 early region or adenovirus major late promoter). In another embodiment, the promoter is recognized by RNA polymerase III (e.g., a U6 or H1 promoter).

In certain embodiments, the promoter is a regulated promoter (e.g., inducible promoter). In some embodiments, the promoter is an inducible promoter or a repressible promoter. In some embodiments, the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof.

In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1α promoter (EF1α), mouse phosphoglycerate kinase 1 promoter (PGK), and chicken β-Actin promoter coupled with CMV early enhancer (CAGG). In some embodiments, the constitutive promoter is a synthetic or modified promoter. In some embodiments, the promoter is or comprises an MND promoter, a synthetic promoter that contains the U3 region of a modified MoMuLV LTR with myeloproliferative sarcoma virus enhancer (sequence set forth in SEQ ID NO:18 or 126; see Challita et al. (1995) J. Virol. 69(2):748-755). In some embodiments, the promoter is a tissue-specific promoter. In another embodiment, the promoter is a viral promoter. In another embodiment, the promoter is a non-viral promoter. In some embodiments, exemplary promoters can include, but are not limited to, human elongation factor 1 alpha (EF1α) promoter (sequence set forth in SEQ ID NO: 4 or 5) or a modified form thereof (EF1α promoter with HTLV1 enhancer; sequence set forth in SEQ ID NO: 127) or the MND promoter (sequence set forth in SEQ ID NO:18 or 126). In some embodiments, the polynucleotide and/or vector does not include a regulatory element, e.g. promoter.

In particular embodiments, the polynucleotide, e.g., the polynucleotide encoding the recombinant receptor and/or TCR, are introduced into the cells in nucleotide form, e.g., as or within a non-viral vector. In some embodiments, the non-viral vector is or includes a polynucleotide, e.g., a DNA or RNA polynucleotide, that is suitable for transduction and/or transfection by any suitable and/or known non-viral method for gene delivery, such as but not limited to microinjection, electroporation, transient cell compression or squeezing (e.g., as described in Lee, et al. (2012) Nano Lett 12: 6322-27), lipid-mediated transfection, peptide-mediated delivery, or a combination thereof. In some embodiments, the non-viral polynucleotide is delivered into the cell by a non-viral method described herein, such as a non-viral method listed in Table 7.

In some embodiments, the vector or delivery vehicle is a viral vector (e.g., for generation of recombinant viruses). In some embodiments, the virus is a DNA virus (e.g., dsDNA or ssDNA virus). In some embodiments, the virus is an RNA virus (e.g., an ssRNA virus). Exemplary viral vectors/viruses include, e.g., retroviruses, lentiviruses, adenovirus, adeno-associated virus (AAV), vaccinia viruses, poxviruses, and herpes simplex viruses, or any of the viruses described elsewhere herein.

In some embodiments, the virus infects dividing cells. In another embodiment, the virus infects non-dividing cells. In another embodiment, the virus infects both dividing and non-dividing cells. In another embodiment, the virus can integrate into the host genome. In another embodiment, the virus is engineered to have reduced immunity, e.g., in human. In another embodiment, the virus is replication-competent. In another embodiment, the virus is replication-defective, e.g., having one or more coding regions for the genes necessary for additional rounds of virion replication and/or packaging replaced with other genes or deleted. In another embodiment, the virus causes transient expression of the Cas9 molecule and/or the gRNA molecule for the purposes of transient induction of genetic disruption. In another embodiment, the virus causes long-lasting, e.g., at least 1 week, 2 weeks, 1 month, 2 months, 3 months, 6 months, 9 months, 1 year, 2 years, or permanent expression, of the Cas9 molecule and/or the gRNA molecule. The packaging capacity of the viruses may vary, e.g., from at least about 4 kb to at least about 30 kb, e.g., at least about 5 kb, 10 kb, 15 kb, 20 kb, 25 kb, 30 kb, 35 kb, 40 kb, 45 kb, or 50 kb.

In some embodiments, the polynucleotide containing the agent(s) and/or template polynucleotide is delivered by a recombinant retrovirus. In another embodiment, the retrovirus (e.g., Moloney murine leukemia virus) comprises a reverse transcriptase, e.g., that allows integration into the host genome. In some embodiments, the retrovirus is replication-competent.

In another embodiment, the retrovirus is replication-defective, e.g., having one of more coding regions for the genes necessary for additional rounds of virion replication and packaging replaced with other genes, or deleted.

In some embodiments, the polynucleotide containing the agent(s) and/or template polynucleotide is delivered by a recombinant lentivirus. For example, the lentivirus is replication-defective, e.g., does not comprise one or more genes required for viral replication. In some embodiments, the virus is an HIV-derived lentivirus.

In some embodiments, the polynucleotide containing the agent(s) and/or template polynucleotide is delivered by a recombinant adenovirus. In another embodiment, the adenovirus is engineered to have reduced immunity in humans.

In some embodiments, the polynucleotide containing the agent(s) and/or template polynucleotide is delivered by a recombinant AAV. In some embodiments, the AAV can incorporate its genome into that of a host cell, e.g., a target cell as described herein. In another embodiment, the AAV is a self-complementary adeno-associated virus (scAAV), e.g., a scAAV that packages both strands which anneal together to form double stranded DNA. AAV serotypes that may be used in the disclosed methods, include AAV1, AAV2, modified AAV2 (e.g., modifications at Y444F, Y500F, Y730F and/or S662V), AAV3, modified AAV3 (e.g., modifications at Y705F, Y731F and/or T492V), AAV4, AAV5, AAV6, modified AAV6 (e.g., modifications at S663V and/or T492V), AAV7, AAV8, AAV 8.2, AAV9, AAV.rh10, modified AAV.rh10, AAV.rh32/33, modified AAV.rh32/33, AAV.rh43, modified AAV.rh43, AAV.rh64R1, modified AAV.rh64R1, and pseudotyped AAV, such as AAV2/8, AAV2/5 and AAV2/6 can also be used in the disclosed methods.

In some embodiments, the polynucleotide containing the agent(s) and/or template polynucleotide is delivered by a hybrid virus, e.g., a hybrid of one or more of the viruses described herein.

A packaging cell is used to form a virus particle that is capable of infecting a target cell. Such a cell includes a 293 cell, which can package adenovirus, and a ψ2 cell or a PA317 cell, which can package retrovirus. A viral vector used in gene therapy is usually generated by a producer cell line that packages a nucleic acid vector into a viral particle. The vector typically contains the minimal viral sequences required for packaging and subsequent integration into a host or target cell (if applicable), with other viral sequences being replaced by an expression cassette encoding the protein to be expressed, e.g., Cas9. For example, an AAV vector used in gene therapy typically only possesses inverted terminal repeat (ITR) sequences from the AAV genome which are required for packaging and gene expression in the host or target cell. The missing viral functions are supplied in trans by the packaging cell line. Henceforth, the viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, the viral vector has the ability of cell type recognition. For example, the viral vector can be pseudotyped with a different/alternative viral envelope glycoprotein; engineered with a cell type-specific receptor (e.g., genetic modification of the viral envelope glycoproteins to incorporate targeting ligands such as a peptide ligand, a single chain antibody, a growth factor); and/or engineered to have a molecular bridge with dual specificities with one end recognizing a viral glycoprotein and the other end recognizing a moiety of the target cell surface (e.g., ligand-receptor, monoclonal antibody, avidin-biotin and chemical conjugation).

In some embodiments, the viral vector achieves cell type specific expression. For example, a tissue-specific promoter can be constructed to restrict expression of the transgene (Cas9 and gRNA) in only a specific target cell. The specificity of the vector can also be mediated by microRNA-dependent control of transgene expression. In some embodiments, the viral vector has increased efficiency of fusion of the viral vector and a target cell membrane.

For example, a fusion protein such as fusion-competent hemagglutinin (HA) can be incorporated to increase viral uptake into cells. In some embodiments, the viral vector has the ability of nuclear localization. For example, a virus that requires the breakdown of the nuclear membrane (during cell division) and therefore will not infect a non-diving cell can be altered to incorporate a nuclear localization peptide in the matrix protein of the virus thereby enabling the transduction of non-proliferating cells.

### III. CELLS FOR GENETIC ENGINEERING

In some of the provided embodiments, the cells for engineering are immune cells, such as T cells. Provided are genetically engineered cells or cell populations wherein one or more of the cells contain a knock-out of one or more endogenous TCR genes and recombinant receptor-encoding nucleic acids and/or other transgene that are integrated into one or more of the endogenous TCR genes. Also provided are populations or compositions of such cells, compositions containing such cells and/or enriched for cells that are engineered using the provided methods.

In some embodiments, the cells for engineering include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4+ and/or of CD8+ T cells are naive T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In some embodiments, the cell is a regulatory T cell (Treg). In some embodiments, the cell further comprises a recombinant FOXP3 or variant thereof.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for engineering may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contain cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished in a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or subpopulation, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or ROR1, and positive selection based on a marker characteristic of central memory T cells, such as CD62L or CCR7, where the positive and negative selections are carried out in either order.

CD4+T helper cells are sorted into naive, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L- and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In vitro and In vivo, p 17-25 Edited by: S. A. Brooks and U. Schumacher © Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain aspects, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International PCT Publication No. WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood may be automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10:1567-1573; and Godin et al. (2008) J Biophoton. 1(5):355-376). In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° per minute and stored in the vapor phase of a liquid nitrogen storage tank.

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of nucleic acids encoding a recombinant receptor, e.g., a recombinant TCR.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling region of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In some embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

Various methods for the introduction of genetically engineered components, e.g., agents for inducing a genetic disruption and/or nucleic acids encoding recombinant receptors, e.g., CARs or TCRs, are known and may be used with the provided methods and compositions. Exemplary methods include those for transfer of nucleic acids encoding the polypeptides or receptors, including via viral vectors, e.g., retroviral or lentiviral, non-viral vectors or transposons, e.g. Sleeping Beauty transposon system. Methods of gene transfer can include transduction, electroporation or other method that results into gene transfer into the cell, or any delivery methods described in Section I.A. Other approaches and vectors for transfer of the nucleic acids encoding the recombinant products are those described, e.g., in WO2014055668 and U.S. Pat. No. 7,446,190.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, it may be desired to safeguard against the potential that overexpression of a stimulatory factor (for example, a lymphokine or a cytokine) could potentially result in an unwanted outcome or lower efficacy in a subject, such as a factor associated with toxicity in a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound.

Negative selectable genes include the Herpes simplex virus type I thymidine kinase (HSV-I TK) gene (Wigler et al., Cell 11:223, 1977) which confers ganciclovir sensitivity; the cellular hypoxanthine phosphribosyltransferase (HPRT) gene, the cellular adenine phosphoribosyltransferase (APRT) gene, bacterial cytosine deaminase (Mullen et al., Proc. Natl. Acad. Sci. USA. 89:33 (1992)).

In some embodiments, the cells, e.g., T cells, may be engineered either during or after expansion. This engineering for the introduction of the gene of the desired polypeptide or receptor can be carried out with any suitable retroviral vector, for example. The genetically modified cell population can then be liberated from the initial stimulus (the CD3/CD28 stimulus, for example) and subsequently be stimulated with a second type of stimulus (e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

As described herein, in some embodiments, the cells are incubated and/or cultured prior to or in connection with genetic engineering. The incubation steps can include culture, cultivation, stimulation, activation, propagation and/or freezing for preservation, e.g. cryopreservation.

### IV. RECOMBINANT T CELL RECEPTORS (TCRS)

In some embodiments, the recombinant receptor that is introduced into the cell is a T cell receptor (TCR) or an antigen-binding fragment thereof.

In some embodiments, the transgene for targeted integration encodes a recombinant receptor or an antigen-binding fragment thereof or a chain thereof. In some embodiments, the recombinant receptor is a recombinant antigen receptor, or a recombinant receptor that binds to an antigen. In some embodiments, the recombinant receptor is a recombinant or engineered T cell receptor (TCR). In some aspects, the recombinant TCR is different from the endogenous TCR encoded by the T cell. In some embodiments, the provided polynucleotides, vectors, compositions, methods, articles of manufacture, and/or kits are useful for engineering cells that express a recombinant TCR or an antigen-binding fragment thereof.

In some embodiments, the provided recombinant receptors, e.g., TCRs or CARs, are capable of binding to or recognizing, such as specifically binding to or recognizing, an antigen that is associated with, specific to, and/or expressed on a cell or tissue of a disease, disorder or condition, such as a cancer or a tumor. In some aspects, the antigen is in a form of a peptide, e.g., is a peptide antigen or a peptide epitope. In some embodiments, the provided TCRs bind to, such as specifically bind to, an antigen that is a peptide, in the context of a major histocompatibility (MHC) molecule.

The observation that recombinant receptor binds to an antigen, e.g., peptide antigen, or specifically binds to an antigen, e.g., peptide antigen, does not necessarily mean that it binds to an antigen of every species. For example, in some embodiments, features of binding to the antigen, e.g., peptide antigen in the context of an MHC, such as the ability to specifically bind thereto and/or to compete for binding thereto with a reference binding molecule or a receptor, and/or to bind with a particular affinity or compete to a particular degree, in some embodiments, refers to the ability with respect to a human antigen and the recombinant receptor may not have this feature with respect to the antigen from another species, such as mouse. In some aspects, the extent of binding of the recombinant receptor or an antigen-binding fragment thereof to an unrelated antigen or protein, such as an unrelated peptide antigen, is less than at or about 10% of the binding of the recombinant receptor or an antigen-binding fragment thereof to the antigen, e.g., cognate antigen as measured, e.g., by a radioimmunoassay (RIA), a peptide titration assay or a reporter assay.

A. T Cell Receptors (TCRs)

In some embodiments, a “T cell receptor” or “TCR” is a molecule that contains an a and β chains (also known as TCRα and TCRβ, respectively) or a variable γ and S chains (also known as TCRγ and TCRδ, respectively), or antigen-binding portions thereof, and which is capable of specifically binding to an antigen, e.g., a peptide antigen or peptide epitope, bound to an MHC molecule. In some embodiments, the TCR is in the a form. Typically, TCRs that exist in a and yS forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, the antigen that is bound or recognized, such as specifically bound or recognized, by the TCR is a peptide. In some aspects, the peptide that is bound or recognized, such as specifically bound or recognized, by the TCR is a peptide from an antigen. In some aspects, “antigen” bound or recognized by a TCR, includes a peptide.

Typically, specific binding of recombinant receptor, e.g. TCR, to a peptide epitope, e.g. in complex with an MHC, is governed by the presence of an antigen-binding site containing one or more complementarity determining regions (CDRs). In general, it is understood that specifically binds does not mean that the particular peptide epitope, e.g. in complex with an MHC, is the only thing to which the MHC-peptide molecule may bind, since non-specific binding interactions with other molecules may also occur. In some embodiments, binding of recombinant receptor to a peptide in the context of an MHC molecule is with a higher affinity than binding to such other molecules, e.g. another peptide in the context of an MHC molecule or an irrelevant (control) peptide in the context of an MHC molecule, such as at least about 2-fold, at least about 10-fold, at least about 20-fold, at least about 50-fold, or at least about 100-fold higher than binding affinity to such other molecules.

In some embodiments, the recombinant receptor, e.g., TCR, can be assessed for safety or off-target binding activity using any of a number of known screening assays. In some embodiments, generation of an immune response to a particular recombinant receptor, e.g., TCR, can be measured in the presence of cells that are known not to express the target peptide epitope, such as cells derived from normal tissue(s), allogenic cell lines that express one or more different MHC types or other tissue or cell sources. In some embodiments, the cells or tissues include normal cells or tissues. In some embodiments, the binding to cells can be tested in 2 dimensional cultures. In some embodiments, the binding to cells can be tested in 3 dimensional cultures. In some embodiments, as a control, the tissues or cells can be ones that are known to express the target epitope. The immune response can be assessed directly or indirectly, such as by assessing activation of immune cells such as T cells (e.g. cytotoxic activity), production of cytokine (e.g. interferon gamma), or activation of a signaling cascade, such as by reporter assays.

Unless otherwise stated, the term “TCR” should be understood to encompass full TCRs as well as antigen-binding portions or antigen-binding fragments thereof. In some embodiments, the TCR is an intact or full-length TCR, such as a TCR containing the alpha (a) chain and beta (β) chain. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds.

In some cases, an antigen-binding portion contains the variable domains of a TCR, such as variable α (V_{α}) chain and variable β (V_{β}) chain of a TCR, or antigen-binding fragments thereof sufficient to form a binding site for binding to a specific MHC-peptide complex.

In some embodiments, the variable domains of the TCR contain complementarity determining regions (CDRs), which generally are the primary contributors to antigen recognition and binding capabilities and specificity of the peptide, MHC and/or MHC-peptide complex. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the a chain can interact with the N-terminal part of certain antigenic peptides. In some contexts, CDR1 of the β chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the β-chain can contain a further hypervariable region (CDR4 or HVR4), which generally is involved in superantigen binding and not antigen recognition (Kotb (1995) Clinical Microbiology Reviews, 8:411-426).

In some embodiments, the TCRα chain and/or TCRβ chain of a TCR also can contain a constant domain, a transmembrane domain and/or a short cytoplasmic tail (see, e.g., Janeway et al., Immunobiology: The Immune System in Health and Disease, 3 Ed., Current Biology Publications, p. 4:33, 1997). In some aspects, each chain (e.g. alpha or beta) of the TCR can possess one N-terminal immunoglobulin variable domain, one immunoglobulin constant domain, a transmembrane region, and a short cytoplasmic tail at the C-terminal end. In some embodiments, a TCR, for example via the cytoplasmic tail, is associated with invariant proteins of the CD3 complex involved in mediating signal transduction. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3γ, CD3δ, CD3ε and CD3ζ chains) contain one or more immunoreceptor tyrosine-based activation motif or ITAM and generally are involved in the signaling capacity of the TCR complex.

In some embodiments, the various domains or regions of a TCR can be determined. In some cases, the exact locus of a domain or region can vary depending on the particular structural or homology modeling or other features used to describe a particular domain. It is understood that reference to amino acids, including to a specific sequence set forth as a SEQ ID NO used to describe domain organization of a recombinant receptor, e.g., TCR, are for illustrative purposes and are not meant to limit the scope of the embodiments provided. In some cases, the specific domain (e.g. variable or constant) can be several amino acids (such as one, two, three or four) longer or shorter. In some aspects, residues of a TCR are known or can be identified according to the International Immunogenetics Information System (IMGT) numbering system (see e.g. www.imgt.org; see also, Lefranc et al. (2003) Developmental and Comparative Immunology, 2&; 55-77; and The T Cell Factsbook 2nd Edition, Lefranc and LeFranc Academic Press 2001). Using this system, the CDR1 sequences within a TCR Vα chains and/or Vβ chain correspond to the amino acids present between residue numbers 27-38, inclusive, the CDR2 sequences within a TCR Vα chain and/or Vβ chain correspond to the amino acids present between residue numbers 56-65, inclusive, and the CDR3 sequences within a TCR Vα chain and/or Vβ chain correspond to the amino acids present between residue numbers 105-117, inclusive.

In some embodiments, the a chain and β chain of a TCR each further contain a constant domain. In some embodiments, the a chain constant domain (Ca) and β chain constant domain (Cβ) individually are mammalian, such as is a human or murine constant domain. In some embodiments, the constant domain is adjacent to the cell membrane. For example, in some cases, the extracellular portion of the TCR formed by the two chains contains two membrane-proximal constant domains, and two membrane-distal variable domains, which variable domains each contain CDRs.

In some embodiments, each of the TCRα and TCRβ constant domains is human. In some embodiments, the Cα is encoded by the TRAC gene (IMGT nomenclature) or is a variant thereof. In some embodiments, the Cα has or comprises the sequence of amino acids set forth in SEQ ID NO: 19 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 19. In some embodiments, the Cα has or comprises the sequence of amino acids set forth in any of SEQ ID NO:19. In some embodiments, the Cα has or comprises the sequence of amino acids, e.g., mature polypeptide, encoded by the nucleic acid sequence set forth in SEQ ID NO:1 or a sequence of amino acids that exhibits at least 85%, 86%, 87%, 88%, 89%, 90%, 91%, 92%, 93%, 94%, 95%, 96%, 97%, 98%, 99% or more sequence identity to the sequence of amino acids, e.g., mature polypeptide, encoded by the nucleic acid sequence set forth in SEQ ID NO:1. In some embodiments, the Cβ is encoded by TRBC1 or TRBC2 genes (IMGT nomenclature) or is a variant thereof. In some embodiments, the Cβ has or comprises the sequence of amino acids set forth in SEQ ID NO:20 or 21 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 20 or 21. In some embodiments, the Cβ has or comprises the sequence of amino acids set forth in SEQ ID NO: 20 or 21. In some embodiments, the Cβ has or comprises the sequence of amino acids, e.g., mature polypeptide, encoded by the nucleic acid sequence set forth in SEQ ID NO:2 or 3 or a sequence of amino acids that exhibits at least 85%, 86%, 87%, 88%, 89%, 90%, 91%, 92%, 93%, 94%, 95%, 96%, 97%, 98%, 99% or more sequence identity to the sequence of amino acids, e.g., mature polypeptide, encoded by the nucleic acid sequence set forth in SEQ ID NO:2 or 3.

In some embodiments, a portion of the recombinant TCR is encoded by the at least a portion of the open reading frame of a TRAC or TRBC gene. In certain embodiments, delivering, introducing, contacting, transfecting, and/or transducing the transgene, polynucleotide, or vector to a cell results in expression of the recombinant TCR. In some embodiments, a portion of the recombinant TCR is encoded by the transgene, polynucleotide, and/or vector, and the remaining portion is encoded by a nucleic acid sequence that is endogenous and/or native to the cell. In certain embodiments, the endogenous and/or native nucleic acid sequence is or is within an open reading frame of a TRAC or TRBC locus. In some embodiment, the TRAC or TRBC locus is human. In particular embodiments, the remaining portion of the TCR is a constant domain, e.g., a TCRα. or TCRβ constant domain. In certain embodiments, the remaining portion of the TCR is a portion of a constant domain, e.g., a portion of the TCRα, or TCRβ constant domain.

In some embodiments, any of the provided TCRs or antigen-binding fragments thereof can be a human/mouse chimeric TCR. In some cases, the TCR or antigen-binding fragment thereof have a chain and/or a chain comprising a mouse constant region. In some aspects, the Cα and/or Cβ regions are mouse constant regions. In some embodiments, the Cα is a mouse constant region that is or comprises the sequence of amino acids set forth in SEQ ID NO: 14, 15, 121 or 122 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 14, 15, 121 or 122. In some embodiments, the Cα is or comprises the sequence of amino acids set forth in SEQ ID NO: 14, 15, 121 or 122. In some embodiments, the Cβ is a mouse constant region that is or comprises the sequence of amino acids set forth in SEQ ID NO: 16, 17 or 123 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 16, 17 or 123. In some embodiments, the Cβ is or comprises the sequence of amino acids set forth in SEQ ID NO: 16, 17 or 123.

In some of any such embodiments, the TCR or antigen-binding fragment thereof containing one or more modifications in the a chain and/or β chain such that when the TCR or antigen-binding fragment thereof is expressed in a cell, the frequency of mispairing between the TCR α chain and β chain and an endogenous TCR α chain and β chain is reduced, the expression of the TCR α chain and β chain is increased and/or the stability of the TCR α chain and β chain is increased. In some embodiments, the one or more modifications is a replacement, deletion, or insertion of one or more amino acids in the Cα region and/or the Cβ region. In some aspects, the one or more modifications contain replacement(s) to introduce one or more cysteine residues that are capable of forming one or more non-native disulfide bridges between the a chain and 3 chain.

In some of any such embodiments, the TCR or antigen-binding fragment thereof containing a Cα region containing a cysteine at a position corresponding to position 48 with numbering as set forth in SEQ ID NO: 24 and/or a Cβ region containing a cysteine at a position corresponding to position 57 with numbering as set forth in SEQ ID NO: 20. In some embodiments, said Cα region contains the amino acid sequence set forth in any of SEQ ID NOS: 19 or 24, or a sequence of amino acids that has at least 90% sequence identity thereto containing one or more cysteine residues capable of forming a non-native disulfide bond with the chain; and/or said Cβ region contains the amino acid sequence set forth in any of SEQ ID NOS: 20, 21 or 25, or a sequence of amino acids that has at least 90% sequence identity thereto that contains one or more cysteine residues capable of forming a non-native disulfide bond with the a chain.

In some of any such embodiments, the TCR or antigen-binding fragment thereof is encoded by a nucleotide sequence that has been codon-optimized.

In some of any such embodiments, the binding molecule or TCR or antigen-binding fragment thereof is isolated or purified or is recombinant. In some of any such embodiments, the binding molecule or TCR or antigen-binding fragment thereof is human.

In some embodiments, the TCR may be a heterodimer of two chains a and that are linked, such as by a disulfide bond or disulfide bonds. In some embodiments, the constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains. In some embodiments, each of the constant and variable domains contains disulfide bonds formed by cysteine residues.

In some embodiments, the TCR can contain an introduced disulfide bond or bonds. In some embodiments, the native disulfide bonds are not present. In some embodiments, the one or more of the native cysteines (e.g. in the constant domain of the a chain and chain) that form a native interchain disulfide bond are substituted to another residue, such as to a serine or alanine. In some embodiments, an introduced disulfide bond can be formed by mutating non-cysteine residues on the alpha and β chains, such as in the constant domain of the a chain and β chain, to cysteine. In some embodiments, the presence of non-native cysteine residues (e.g. resulting in one or more non-native disulfide bonds) in a recombinant TCR can favor production of the desired recombinant TCR in a cell in which it is introduced over expression of a mismatched TCR pair containing a native TCR chain.

Exemplary non-native disulfide bonds of a TCR are described in published International PCT No. WO2006/000830, WO2006037960 and Kuball et al. (2007) Blood, 109:2331-2338. In some embodiments, cysteines can be introduced at residue Thr48 of the Cα chain and Ser57 of the Cβ chain, at residue Thr45 of the Cα chain and Ser77 of the Cβ chain, at residue Tyr10 of the Cα chain and Ser17 of the Cβ chain, at residue Thr45 of the Cα chain and Asp59 of the Cβ chain and/or at residue Serl5 of the Cα chain and Glu15 of the Cβ chain with reference to numbering of a Cα set forth in SEQ ID NO: 24 or C set forth in SEQ ID NO:20.

In some embodiments, any of the provided cysteine mutations can be made at a corresponding position in another sequence, for example, in the mouse Cα and C sequences described herein.

The term “corresponding” with reference to positions of a protein, such as recitation that amino acid positions “correspond to” amino acid positions in a disclosed sequence, such as set forth in the Sequence listing, refers to amino acid positions identified upon alignment with the disclosed sequence based on structural sequence alignment or using a standard alignment algorithm, such as the GAP algorithm. For example, corresponding residues can be determined by alignment of a reference sequence with the Cα sequence set forth in any of SEQ ID NO: 24 or the Cβ sequence set forth in SEQ ID NO: 20 by structural alignment methods as described herein. By aligning the sequences, corresponding residues can be identified, for example, using conserved and identical amino acid residues as guides.

Exemplary sequences (e.g. CDRs, V_{α} and/or V_{β} and constant region sequences) of provided TCRs are described herein.

In some embodiments, the recombinant TCR or antigen-binding portion thereof (or other MHC-peptide binding molecule, such as TCR-like antibody) is known to or likely or may recognize a peptide epitope or T cell epitope of a target polypeptide when presented by cells in the context of an MHC molecule, i.e. MHC-peptide complex of the target polypeptide. In some embodiments, the recombinant TCR (or other MHC-peptide binding molecule or TCR-like antibody) is known to or likely to exhibit specific binding for the T cell epitope of the target polypeptide, for example when displayed as an MHC-peptide complex. Methods of assessing binding or interaction of an MHC-peptide binding molecule (e.g. TCR or TCR-like antibody) are known, including any of the exemplary methods described herein.

In some embodiments, the MHC molecule is an MHC class I or an MHC class II molecule. In some embodiments, the MHC contains a polymorphic peptide binding site or binding groove that can, in some cases, complex with peptide epitopes of polypeptides, including peptide epitopes processed by the cell machinery. In some cases, MHC molecules can be displayed or expressed on the cell surface, including as a complex with peptide, i.e. MHC-peptide complex, for presentation of an antigen in a conformation recognizable by TCRs on T cells, or other MHC-peptide binding molecules. Generally, MHC class I molecules are heterodimers having a membrane spanning a chain, in some cases with three a domains, and a non-covalently associated 2 microglobulin. Generally, MHC class II molecules are composed of two transmembrane glycoproteins, α and β, both of which typically span the membrane. An MHC molecule can include an effective portion of an MHC that contains an antigen binding site or sites for binding a peptide and the sequences necessary for recognition by the appropriate binding molecule, such as TCR. In some embodiments, MHC class I molecules deliver peptides originating in the cytosol to the cell surface, where a peptide:MHC complex is recognized by T cells, such as generally CD8⁺ T cells, but in some cases CD4+ T cells. In some embodiments, MHC class II molecules deliver peptides originating in the vesicular system to the cell surface, where they are typically recognized by CD4⁺ T cells. Generally, MHC molecules are encoded by a group of linked loci, which are collectively termed H-2 in the mouse and human leukocyte antigen (HLA) in humans. In some aspects, human MHC can also be referred to as human leukocyte antigen (HLA).

In some embodiments, the peptide epitope or T cell epitope is a peptide that may be derived from or based on a fragment of a longer biological molecule, such as a polypeptide or protein, and which is capable of associating with or forming a complex with an MHC molecule.

In some embodiments, the peptide is about 8 to about 24 amino acids in length. In some embodiments, the peptide has a length of from or from about 9 to 22 amino acids for recognition in the MHC Class II complex. In some embodiments, the peptide has a length of from or from about 8 to 13 amino acids for recognition in the MHC Class I complex. In some embodiments, the MHC molecule and peptide epitope or T cell epitope are complexed or associated via non-covalent interactions of the peptide in the binding groove or cleft of the MHC molecule.

In some embodiments, the MHC-peptide complex is present or displayed on the surface of cells. In some embodiments, the MHC-peptide complex can be specifically recognized by a TCR or antigen-binding portion thereof, or other MHC-peptide binding molecule. In some embodiments, the T cell epitope or peptide epitope is capable of inducing an immune response in an animal by its binding characteristics to MHC molecules. In some embodiments, upon recognition of the T cell epitope, such as MHC-peptide complex, the TCR (or other MHC-peptide binding molecule) produces or triggers an activation signal to the T cell that induces a T cell response, such as T cell proliferation, cytokine production, a cytotoxic T cell response or other response.

In some embodiments, the TCR, or other MHC-peptide binding molecule, recognizes or potentially recognizes the T cell epitope in the context of an MHC class I molecule. MHC class I proteins are expressed in all nucleated cells of higher vertebrates. The MHC class I molecule is a heterodimer composed of a 46-kDa heavy chain which is non-covalently associated with the 12-kDa light chain 3-2 microglobulin. In humans, there are several MHC alleles, such as, for example, HLA-A2, HLA-A1, HLA-A3, HLA-A24, HLA-A28, HLA-A31, HLA-A33, HLA-A34, HLA-B7, HLA-B45 and HLA-Cw8. The sequences of MHC alleles are known and can be found, for example, at the IMGT/HLA database available at www.ebi.ac.uk/ipd/imgt/hla. In some embodiments, the MHC class I allele is an HLA-A2 allele, which in some populations is expressed by approximately 50% of the population. In some embodiments, the HLA-A2 allele can be an HLA-A*0201, *0202, *0203, *0206, or *0207 gene product. In some cases, there can be differences in the frequency of subtypes between different populations. For example, in some embodiments, more than 95% of the HLA-A2 positive Caucasian population is HLA-A*0201, whereas in the Chinese population the frequency has been reported to be approximately 23% HLA-A*0201, 45% HLA-A*0207, 8% HLA-A*0206 and 23% HLA-A*0203.

In some embodiments, MHC-class I restricted peptides are 8 to 15 amino acids in length, such as 8 to 10 amino acids in length. In some embodiments, MHC class I molecules bind peptides derived from endogenous antigens, such as tumor, viral or bacterial proteins produced within a diseased or infected cell, which have been processed within the cytoplasm of the cell via the cytosolic pathway. In some embodiments, MHC class I-peptide complexes displayed on the surface of the cell are typically recognized by TCRs expressed on CD8+ T cells, such as cytotoxic T cells. In some embodiments, MHC class I-peptide complexes can be recognized by TCRs expressed on CD4+ T cells, such as by TCRs exhibiting CD8− or partial CD8− independent binding.

In some embodiments, the TCR, or other MHC-peptide binding molecule, recognizes or potentially recognizes the T cell epitope in the context of an MHC class II molecule. MHC class II proteins are expressed in a subset of nucleated vertebrate cells, generally called antigen presenting cells (APCs). In humans, there are several MHC class II alleles, such as, for example, DR1, DR3, DR4, DR7, DR52, DQ1, DQ2, DQ4, DQ8 and DP1. In some embodiments, the MHC class II allele that is HLA-DRB1*0101, an HLA-DRB*0301, HLA-DRB*0701, HLA-DRB*0401 an HLA-DQB1*0201. The sequences of MHC alleles are known and can be found, for example, at the IMGT/HLA database available at www.ebi.ac.uk/ipd/imgt/hla.

In some embodiments, MHC-class II restricted peptides are generally between about 9 and 25 residues in length, such as between 15 and 25 residues or 13 and 18 residues in length, and, in some cases, contains a binding core region of about 9 amino acids or about 12 amino acids. In some embodiments, MHC class II molecules bind peptides derived from exogenous antigens, which are internalized by phagocytosis or endocytosis and processed within the endosomal/lysosomal pathway. In some embodiments, MHC class II-peptide complexes displayed on the surface of cells are typically recognized by CD4⁺ cells, such as helper T cells. In some embodiments, MHC class II-peptide complexes displayed can be recognized by TCRs expressed on CD8+ T cells

Typically, the peptide epitope or T cell epitope is a peptide portion of an antigen. In some embodiments, the antigen is known, and in some cases the peptide epitope recognized by the TCR or antigen-binding portion thereof (or other MHC-peptide binding molecules) also may be known, such a known prior to performing the provided method.

In some embodiments, the antigen is a tumor-associated antigen, an antigen expressed in a particular cell type associated with an autoimmune or inflammatory disease, or an antigen derived from a viral pathogen or a bacterial pathogen. In some embodiments, the antigen is an antigen involved in a disease. In some embodiments, the disease can be caused by malignancy or transformation of cells, such as a cancer. In some embodiments, the antigen can be an intracellular protein antigen from a tumor or cancer cell, such as a tumor-associated antigen. In some cases, because the majority of cancer antigens are derived from intracellular proteins that can only be targeted at the cell surface in the context of an MHC molecule, TCRs make the ideal candidate for therapeutics as they have evolved to recognize this class of antigen. In some embodiments, the disease can be caused by infection, such as by bacterial or viral infection. In some embodiments, the antigen is a viral-associated cancer antigen. In some cases, a recombinant TCR or antigen-binding portions thereof (and other MHC-peptide binding molecules) recognize or potentially recognize peptides derived from viral proteins that have been naturally processed in infected cells and displayed by an MHC molecule on the cell surface. In some embodiments, the disease can be an autoimmune disease. Other targets include those listed in The HLA Factsbook (Marsh et al. (2000)) and others that are known.

In some embodiments, the antigen is one that is associated with a tumor or cancer. In some embodiments, a tumor or cancer antigen is one that can be found on a malignant cell, found inside a malignant cell or is a mediator of tumor cell growth. In some embodiments, a tumor or cancer antigen is one that is predominantly expressed or over-expressed by a tumor cell or cancer cell. A number of tumor antigens have been identified and are known, including MHC-restricted, T cell-defined tumor antigens (see e.g. cancerimmunity.org/peptide/; Boon and Old (1997) Curr Opin Immunol, 9:681-3; Cheever et al. (2009) Clin Cancer Res, 15:5323-37). In some embodiments, tumor antigens include, but are not limited to, mutated peptides, differentiation antigens, and overexpressed antigens, all of which could serve as targets for therapies.

In some embodiments, the tumor or cancer antigen is a lymphoma antigen, (e.g., non-Hodgkin's lymphoma or Hodgkin's lymphoma), a B-cell lymphoma cancer antigen, a leukemia antigen, a myeloma (i.e., multiple myeloma or plasma cell myeloma) antigen, an acute lymphoblastic leukemia antigen, a chronic myeloid leukemia antigen, or an acute myelogenous leukemia antigen. In some embodiments, the cancer antigen is an antigen that is overexpressed in or associated with a cancer that is an adenocarcinomas, such as pancreas, colon, breast, ovarian, lung, prostate, head and neck, including multiple myelomas and some B cell lymphomas. In some embodiments, the antigen is associated with a cancer, such as prostate cancer, lung cancer, breast cancer, ovarian cancer, pancreatic cancer, skin cancer, liver cancer (e.g., hepatocellular adenocarcinoma), intestinal cancer, or bladder cancer.

In some embodiments, the antigen is a tumor antigen that can be a glioma-associated antigen, j-human chorionic gonadotropin, alphafetoprotein (AFP), B-cell maturation antigen (BCMA, BCM), B-cell activating factor receptor (BAFFR, BR3), and/or transmembrane activator and CAML interactor (TACI), Fc Receptor-like 5 (FCRL5, FcRH5), lectin-reactive AFP, thyroglobulin, RAGE-1, MN-CA IX, human telomerase reverse transcriptase, RUl, RU2 (AS), intestinal carboxyl esterase, muthsp70-2, M-CSF, Melanin-A/MART-1, WT-1, S-100, MBP, CD63, MUC1 (e.g. MUC1-8), p53, Ras, cyclin B1, HER-2/neu, carcinoembryonic antigen (CEA), gp100, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A11, MAGE-B1, MAGE-B2, MAGE-B3, MAGE-B4, MAGE-C1, BAGE, GAGE-1, GAGE-2, p15, tyrosinase (e.g. tyrosinase-related protein 1 (TRP-1) or tyrosinase-related protein 2 (TRP-2)), P-catenin, NY-ESO-1, LAGE-1a, PP1, MDM2, MDM4, EGVFvIII, Tax, SSX2, telomerase, TARP, pp65, CDK4, vimentin, S100, eIF-4A1, IFN-inducible p78, and melanotransferrin (p97), Uroplakin II, prostate specific antigen (PSA), human kallikrein (huK2), prostate specific membrane antigen (PSM), and prostatic acid phosphatase (PAP), neutrophil elastase, ephrin B2, BA-46, beta-catenin, Bcr-abl, E2A-PRL, H4-RET, IGH-IGK, MYL-RAR, Caspase 8 or a B-Raf antigen. Other tumor antigens can include any derived from FRa, CD24, CD44, CD133, CD 166, epCAM, CA-125, HE4, Oval, estrogen receptor, progesterone receptor, uPA, PAI-1, CD19, CD20, CD22, ROR1, mesothelin, CD33/IL3Ra, c-Met, PSMA, Glycolipid F77, GD-2, insulin growth factor (IGF)-I, IGF-II, IGF-I receptor and mesothelin. Specific tumor-associated antigens or T cell epitopes are known (see e.g. van der Bruggen et al. (2013) Cancer Immun, available at www.cancerimmunity.org/peptide/; Cheever et al. (2009) Clin Cancer Res, 15, 5323-37).

In some embodiments, the antigen is a viral antigen. Many viral antigen targets have been identified and are known, including peptides derived from viral genomes in HIV, HTLV and other viruses (see e.g., Addo et al. (2007) PLoS ONE, 2, e321; Tsomides et al. (1994) J Exp Med, 180, 1283-93; Utz et al. (1996) J Virol, 70, 843-51). Exemplary viral antigens include, but are not limited to, an antigen from hepatitis A, hepatitis B (e.g., HBV core and surface antigens (HBVc, HBVs)), hepatitis C (HCV), Epstein-Barr virus (e.g. EBVA), human papillomavirus (HPV; e.g. E6 and E7), human immunodeficiency type-1 virus (HIV1), Kaposi's sarcoma herpes virus (KSHV), human papilloma virus (HPV), influenza virus, Lassa virus, HTLN-1, HIN-1, HIN-II, CMN, EBN or HPN. In some embodiments, the target protein is a bacterial antigen or other pathogenic antigen, such as Mycobacterium tuberculosis (MT) antigens, trypanosome, e.g., Tiypansoma cruzi (T. cruzi), antigens such as surface antigen (TSA), or malaria antigens. Specific viral antigen or epitopes or other pathogenic antigens or T cell epitopes are known (see e.g., Addo et al. (2007) PLoS ONE, 2:e321; Anikeeva et al. (2009) Clin Immunol, 130:98-109).

In some embodiments, the antigen is an antigen derived from a virus associated with cancer, such as an oncogenic virus. For example, an oncogenic virus is one in which infection from certain viruses are known to lead to the development of different types of cancers, for example, hepatitis A, hepatitis B (e.g., HBV core and surface antigens (HBVc, HBVs)), hepatitis C (HCV), human papilloma virus (HPV), hepatitis viral infections, Epstein-Barr virus (EBV), human herpes virus 8 (HHV-8), human T-cell leukemia virus-1 (HTLV-1), human T-cell leukemia virus-2 (HTLV-2), or a cytomegalovirus (CMV) antigen.

In some embodiments, the viral antigen is an HPV antigen, which, in some cases, can lead to a greater risk of developing cervical cancer. In some embodiments, the antigen can be a HPV-16 antigen, and HPV-18 antigen, and HPV-31 antigen, an HPV-33 antigen or an HPV-35 antigen. In some embodiments, the viral antigen is an HPV-16 antigen (e.g., seroreactive regions of the E1, E2, E6 and/or E7 proteins of HPV-16, see e.g., U.S. Pat. No. 6,531,127) or an HPV-18 antigen (e.g., seroreactive regions of the L1 and/or L2 proteins of HPV-18, such as described in U.S. Pat. No. 5,840,306). In some embodiments, the viral antigen is an HPV-16 antigen that is from the E6 and/or E7 proteins of HPV-16. In some embodiments, the TCR is a TCR directed against an HPV-16 E6 or HPV-16 E7. In some embodiments, the TCR is a TCR described in, e.g., WO 2015/184228, WO 2015/009604 and WO 2015/009606.

In some embodiments, the viral antigen is a HBV or HCV antigen, which, in some cases, can lead to a greater risk of developing liver cancer than HBV or HCV negative subjects. For example, in some embodiments, the heterologous antigen is an HBV antigen, such as a hepatitis B core antigen or a hepatitis B envelope antigen (US2012/0308580).

In some embodiments, the viral antigen is an EBV antigen, which, in some cases, can lead to a greater risk for developing Burkitt's lymphoma, nasopharyngeal carcinoma and Hodgkin's disease than EBV negative subjects. For example, EBV is a human herpes virus that, in some cases, is found associated with numerous human tumors of diverse tissue origin. While primarily found as an asymptomatic infection, EBV-positive tumors can be characterized by active expression of viral gene products, such as EBNA-1, LMP-1 and LMP-2A. In some embodiments, the heterologous antigen is an EBV antigen that can include Epstein-Barr nuclear antigen (EBNA)-1, EBNA-2, EBNA-3A, EBNA-3B, EBNA-3C, EBNA-leader protein (EBNA-LP), latent membrane proteins LMP-1, LMP-2A and LMP-2B, EBV-EA, EBV-MA or EBV-VCA.

In some embodiments, the viral antigen is an HTLV-1 or HTLV-2 antigen, which, in some cases, can lead to a greater risk for developing T-cell leukemia than HTLV-1 or HTLV-2 negative subjects. For example, in some embodiments, the heterologous antigen is an HTLV-antigen, such as TAX.

In some embodiments, the viral antigen is a HHV-8 antigen, which, in some cases, can lead to a greater risk for developing Kaposi's sarcoma than HHV-8 negative subjects. In some embodiments, the heterologous antigen is a CMV antigen, such as pp65 or pp64 (see U.S. Pat. No. 8,361,473).

In some embodiments, the antigen is an autoantigen, such as an antigen of a polypeptide associated with an autoimmune disease or disorder. In some embodiments, the autoimmune disease or disorder can be multiple sclerosis (MS), rheumatoid arthritis (RA), Sjogren syndrome, scleroderma, polymyositis, dermatomyositis, systemic lupus erythematosus, juvenile rheumatoid arthritis, ankylosing spondylitis, myasthenia gravis (MG), bullous pemphigoid (antibodies to basement membrane at dermal-epidermal junction), pemphigus (antibodies to mucopolysaccharide protein complex or intracellular cement substance), glomerulonephritis (antibodies to glomerular basement membrane), Goodpasture's syndrome, autoimmune hemolytic anemia (antibodies to erythrocytes), Hashimoto's disease (antibodies to thyroid), pernicious anemia (antibodies to intrinsic factor), idiopathic thrombocytopenic purpura (antibodies to platelets), Grave's disease, or Addison's disease (antibodies to thyroglobulin). In some embodiments, the autoantigen, such as an autoantigen associated with an autoimmune disease described herein, can be collagen, such as type II collagen, mycobacterial heat shock protein, thyroglobulin, acetyl choline receptor (AcHR), myelin basic protein (MBP) or proteolipid protein (PLP). Specific autoimmune associated epitopes or antigens are known (see e.g., Bulek et al. (2012) Nat Immunol, 13:283-9; Harkiolaki et al. (2009) Immunity, 30:348-57; Skowera et al. (2008) J Clin Invest, 1(18): 3390-402).

In some embodiments, the identity of the peptide epitope of the target antigen is known, which, in some cases, can be used in producing or generating a TCR of interest or in assessing a functional activity or property, including in connection with the provided methods. In some embodiments, peptide epitopes can be determined or identified based on the presence of an HLA-restricted motif in a target antigen of interest. In some embodiments, peptides are identified using computer prediction models known. In some embodiments, for predicting MHC class I binding sites, such models include, but are not limited to, ProPred1 (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule. In some aspects, HLA-A*0201-binding motifs and the cleavage sites for proteasomes and immune-proteasomes using computer prediction models are known. For predicting MHC class I binding sites, such models include, but are not limited to, ProPredl (described in more detail in Singh and Raghava, ProPred: prediction of HLA-DR binding sites. BIOINFORMATICS 17(12):1236-1237 2001), and SYFPEITHI (see Schuler et al. SYFPEITHI, Database for Searching and T-Cell Epitope Prediction. in Immunoinformatics Methods in Molecular Biology, vol 409(1): 75-93 2007) Provided are methods of screening and cells employed in the methods of screening, such as T cells, that recognize an antigen or an epitope, in the context of a major histocompatibility complex (MHC) molecule.

In some embodiments, the MHC contains a polymorphic peptide binding site or binding groove that can, in some cases, complex with peptide epitopes of polypeptides, including peptide epitopes processed by the cell machinery. In some cases, MHC molecules can be displayed or expressed on the cell surface, including as a complex with peptide, i.e. MHC-peptide complex, for presentation of an antigen in a conformation recognizable by TCRs on T cells, or other MHC-peptide binding molecules. Generally, MHC class I molecules are heterodimers having a membrane spanning a chain, in some cases with three a domains, and a non-covalently associated 2 microglobulin. Generally, MHC class II molecules are composed of two transmembrane glycoproteins, α and β both of which typically span the membrane. An MHC molecule can include an effective portion of an MHC that contains an epitope binding site or sites for binding a peptide and the sequences necessary for recognition by the appropriate binding molecule, such as TCR. In some embodiments, MHC class I molecules deliver peptides originating in the cytosol to the cell surface, where a peptide:MHC complex is recognized by T cells, such as generally CD8 T cells, but in some cases CD4+ T cells. In some embodiments, MHC class II molecules deliver peptides originating in the vesicular system to the cell surface, where they are typically recognized by CD4⁺ T cells. Generally, MHC molecules are encoded by a group of linked loci, which are collectively termed H-2 in the mouse and human leukocyte antigen (HLA) in humans. In some aspects, human MHC can also be referred to as human leukocyte antigen (HLA).

In some embodiments, the peptide epitope or T cell epitope is a peptide that may be derived from or based on a fragment of a longer biological molecule, such as a polypeptide or protein, and which is capable of associating with or forming a complex with an MHC molecule. In some embodiments, the peptide is about 8 to about 24 amino acids in length. In some embodiments, the peptide has a length of from or from about 9 to 22 amino acids for recognition in the MHC Class II complex. In some embodiments, the peptide has a length of from or from about 8 to 13 amino acids for recognition in the MHC Class I complex. In some embodiments, the MHC molecule and peptide epitope or T cell epitope are complexed or associated via non-covalent interactions of the peptide in the binding groove or cleft of the MHC molecule.

In some embodiments, the MHC-peptide complex is present or displayed on the surface of cells. In some embodiments, the MHC-peptide complex can be specifically recognized by a TCR or antigen-binding portion thereof, or other MHC-peptide binding molecule. In some embodiments, the T cell epitope or peptide epitope is capable of inducing an immune response in an animal by its binding characteristics to MHC molecules. In some embodiments, upon recognition of the T cell epitope, such as MHC-peptide complex, the TCR (or other MHC-peptide binding molecule) produces or triggers an activation signal to the T cell that induces a T cell response, such as T cell proliferation, cytokine production, a cytotoxic T cell response or other response.

In some embodiments, the MHC-peptide binding molecule is a TCR or epitope binding fragment thereof. In some embodiments, the MHC-peptide binding molecule is a TCR-like CAR that contains an antibody or epitope binding fragment thereof, such as a TCR-like antibody, such as one that has been engineered to bind to MHC-peptide complexes. In some embodiments, such binding molecules bind to a binding sequence, such as a T cell epitope, containing an amino acid sequence or antigen of a target polypeptide. In some embodiments, the binding sequence of the target peptide or target polypeptide is known. In some embodiments, the MHC-peptide binding molecule can be derived from natural sources, or it may be partly or wholly synthetically or recombinantly produced.

In some embodiments, the MHC-peptide binding molecule is a molecule or portion thereof that possesses the ability to bind, e.g. specifically bind, to a peptide epitope that is presented or displayed in the context of an MHC molecule, i.e. an MHC-peptide complex, such as on the surface of a cell. In some embodiments, a binding molecule may include any naturally occurring, synthetic, semi-synthetic, or recombinantly produced molecule that can bind, e.g. specifically bind, to an MHC-peptide complex. Exemplary MHC-peptide binding molecules include T cell receptors or antibodies, or antigen-binding portions thereof, including single chain immunoglobulin variable regions (e.g., scTCR, scFv) thereof, that exhibit specific ability to bind to an MHC-peptide complex.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). A TCR may be cell-bound or in soluble form. In some embodiments, the TCR is in cell-bound form expressed on the surface of a cell.

In some embodiments a dTCR contains a first polypeptide wherein a sequence corresponding to a provided TCR α chain variable region sequence is fused to the N terminus of a sequence corresponding to a TCR α chain constant region extracellular sequence, and a second polypeptide wherein a sequence corresponding to a provided TCR β chain variable region sequence is fused to the N terminus a sequence corresponding to a TCR β chain constant region extracellular sequence, the first and second polypeptides being linked by a disulfide bond. In some embodiments, the bond can correspond to the native interchain disulfide bond present in native dimeric a TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a provided TCR α chain containing a variable α domain, a constant α domain and a first dimerization motif attached to the C-terminus of the constant α domain, and a provided TCR β chain comprising a variable β domain, a constant β domain and a first dimerization motif attached to the C-terminus of the constant R domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR, which is a single amino acid strand containing an a chain and a chain that is able to bind to MHC-peptide complexes. Typically, a scTCR can be generated using methods known, See e.g., International published PCT Nos. WO 96/13593, WO 96/18105, WO99/18129, WO 04/033685, WO2006/037960, WO2011/044186; U.S. Pat. No. 7,569,664; and Schlueter, C. J. et al. J. Mol. Biol. 256, 859 (1996).

In some embodiments, a scTCR contains a first segment constituted by an amino acid sequence corresponding to a sequence of a provided TCR α chain variable region, a second segment constituted by an amino acid sequence corresponding to a provided TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an amino acid sequence corresponding to a provided TCR β chain variable region, a second segment constituted by an amino acid sequence corresponding to a provided TCR α chain variable region sequence fused to the N terminus of an amino acid sequence corresponding to a TCR α chain constant domain extracellular sequence, and a linker sequence linking the C terminus of the first segment to the N terminus of the second segment.

In some embodiments, a scTCR contains a first segment constituted by a provided a chain variable region sequence fused to the N terminus of an a chain extracellular constant domain sequence, and a second segment constituted by a provided β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a provided TCR β chain variable region sequence fused to the N terminus of a chain extracellular constant domain sequence, and a second segment constituted by a provided a chain variable region sequence fused to the N terminus of a sequence a chain extracellular constant and transmembrane sequence, and, optionally, a linker sequence linking the C terminus of the first segment to the N terminus of the second segment.

In some embodiments, for the scTCR to bind an MHC-peptide complex, the a and R chains must be paired so that the variable region sequences thereof are orientated for such binding. Various methods of promoting pairing of an α and β in a scTCR are known. In some embodiments, a linker sequence is included that links the α and β chains to form the single polypeptide strand. In some embodiments, the linker should have sufficient length to span the distance between the C terminus of the a chain and the N terminus of the β chain, or vice versa, while also ensuring that the linker length is not so long so that it blocks or reduces bonding of the scTCR to the target peptide-MHC complex.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P-, wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)ₙ-P-, wherein n is 5 or 6 and P is proline, G is glycine and S is serine (SEQ ID NO: 22). In some embodiments, the linker has the sequence GSADDAKKDAAKKDGKS (SEQ ID NO: 23).

In some embodiments, a scTCR contains a disulfide bond between residues of the single amino acid strand, which, in some cases, can promote stability of the pairing between the a and β regions of the single chain molecule (see e.g. U.S. Pat. No. 7,569,664). In some embodiments, the scTCR contains a covalent disulfide bond linking a residue of the immunoglobulin region of the constant domain of the a chain to a residue of the immunoglobulin region of the constant domain of the β chain of the single chain molecule. In some embodiments, the disulfide bond corresponds to the native disulfide bond present in a native dTCR. In some embodiments, the disulfide bond in a native TCR is not present. In some embodiments, the disulfide bond is an introduced non-native disulfide bond, for example, by incorporating one or more cysteines into the constant region extracellular sequences of the first and second chain regions of the scTCR polypeptide. Exemplary cysteine mutations include any as described herein. In some cases, both a native and a non-native disulfide bond may be present.

In some embodiments, a scTCR is a non-disulfide linked truncated TCR in which heterologous leucine zippers fused to the C-termini thereof facilitate chain association (see e.g. International published PCT No. WO99/60120). In some embodiments, a scTCR contain a TCRα variable domain covalently linked to a TCRβ variable domain via a peptide linker (see e.g., International published PCT No. WO99/18129).

In some embodiments, any of the provided TCRs, including a dTCR or scTCR, can be linked to signaling domains that yield an active TCR on the surface of a T cell. In some embodiments, the TCR is expressed on the surface of cells. In some embodiments, the TCR does contain a sequence corresponding to a transmembrane sequence. In some embodiments, the transmembrane domain is positively charged. In some embodiments, the transmembrane domain can be a Cα or C transmembrane domain. In some embodiments, the transmembrane domain can be from a non-TCR origin, for example, a transmembrane region from CD3z, CD28 or B7.1. In some embodiments, the TCR does contain a sequence corresponding to cytoplasmic sequences. In some embodiments, the TCR contains a CD3z signaling domain. In some embodiments, the TCR is capable of forming a TCR complex with CD3.

In some embodiments, the TCR is a soluble TCR. In some embodiments, the soluble TCR has a structure as described in WO99/60120 or WO 03/020763. In some embodiments, the TCR does not contain a sequence corresponding to the transmembrane sequence, for example, to permit membrane anchoring into the cell in which it is expressed. In some embodiments, the TCR does not contain a sequence corresponding to cytoplasmic sequences.

In some embodiments, the recombinant receptor, e.g., TCR or antigen-binding fragment thereof, is or has been modified compared to a known recombinant receptor. In certain embodiments, the recombinant receptors, e.g., TCRs or antigen-binding fragments thereof, include one or more amino acid variations, e.g., substitutions, deletions, insertions, and/or mutations, compared to the sequence of a recombinant receptor, e.g., TCR, described herein or known. Exemplary variants include those designed to improve the binding affinity and/or other biological properties of the binding molecule. Amino acid sequence variants of a binding molecule may be prepared by introducing appropriate modifications into the nucleotide sequence encoding the binding molecule, or by peptide synthesis. Such modifications include, for example, deletions from, and/or insertions into and/or substitutions of residues within the amino acid sequences of the binding molecule. Any combination of deletion, insertion, and substitution can be made to arrive at the final construct, provided that the final construct possesses the desired characteristics, e.g., antigen-binding.

In some embodiments, directed evolution methods are used to generate TCRs with altered properties, such as with higher affinity for a specific peptide in the context of an MHC molecule. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a reference TCR, such as any provided herein, can be used as a template for producing mutagenized TCRs in which in one or more residues of the CDRs are mutated, and mutants with a desired altered property, such as higher affinity for peptide epitope in the context of an MHC molecule, are selected.

In certain embodiments, the recombinant receptors, e.g., TCRs or antigen-binding fragments thereof, include one or more amino acid substitutions, e.g., as compared to a recombinant receptor, e.g., TCR, sequence compared to a sequence of a natural repertoire, e.g., human repertoire. Sites of interest for substitutional mutagenesis include the CDRs, FRs and/or constant regions. Amino acid substitutions may be introduced into a binding molecule of interest and the products screened for a desired activity, e.g., retained/improved antigen affinity or avidity, decreased immunogenicity, improved half-life, CD8-independent binding or activity, surface expression, promotion of TCR chain pairing and/or other improved properties or functions.

In some embodiments, one or more residues within a CDR of a recombinant receptor, e.g., TCR, is/are substituted. In some embodiments, the substitution is made to revert a sequence or position in the sequence to a germline sequence, such as a binding molecule sequence found in the germline (e.g., human germline), for example, to reduce the likelihood of immunogenicity, e.g., upon administration to a human subject.

In certain embodiments, substitutions, insertions, or deletions may occur within one or more CDRs so long as such alterations do not substantially reduce the ability of the recombinant receptor, e.g., TCR or antigen-binding fragment thereof, to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variable sequences provided herein, each CDR either is unaltered, or contains no more than one, two or three amino acid substitutions.

Amino acid sequence insertions include amino- and/or carboxyl-terminal fusions ranging in length from one residue to polypeptides containing a hundred or more residues, as well as intrasequence insertions of single or multiple amino acid residues.

In some aspects, the TCR or antigen-binding fragment thereof may contain one or more modifications in the a chain and/or β chain such that when the TCR or antigen-binding fragment thereof is expressed in a cell, the frequency of mis-pairing between the TCR α chain and β chain and an endogenous TCR α chain and β chain is reduced, the expression of the TCR α chain and β chain is increased, and/or the stability of the TCR α chain and β chain is increased.

In some embodiments, the TCR contains one or more non-native cysteine residues to introduce a covalent disulfide bond linking a residue of the immunoglobulin region of the constant domain of the a chain to a residue of the immunoglobulin region of the constant domain of the β chain. In some embodiments, one or more cysteines can be incorporated into the constant region extracellular sequences of the first and second segments of the TCR polypeptide. Exemplary non-limiting modifications in a TCR to introduce a non-native cysteine residues are described herein (see also, International PCT No. WO2006/000830 and WO2006037960). In some cases, both a native and a non-native disulfide bond may be desirable. In some embodiments, the TCR or antigen-binding fragment is modified such that the interchain disulfide bond in a native TCR is not present.

In some embodiments, the transmembrane domain of the constant region of the TCR can be modified to contain a greater number of hydrophobic residues (see e.g. Haga-Friedman et al. (2012) Journal of Immunology, 188:5538-5546). In some embodiments, the transmembrane region of TCR α chain contains one or more mutations corresponding to S116L, G119V or F120L, with reference to numbering of a Cα set forth in SEQ ID NO: 24.

In some embodiments, the TCR or antigen-binding fragment thereof is encoded by a nucleotide sequence that is or has been codon-optimized. Exemplary codon-optimized variants are described elsewhere herein.

### V. COMPOSITIONS AND FORMULATIONS

Also provided are populations of engineered cells, compositions containing such cells and/or enriched for such cells. Among the compositions are pharmaceutical compositions and formulations for administration, such as for adoptive cell therapy. Also provided are therapeutic methods for administering the cells and compositions to subjects, e.g., patients.

In some embodiments, the provided cell population and/or compositions containing engineered cells include a cell population that exhibits more improved, uniform, homogeneous and/or stable expression and/or antigen binding by the recombinant receptor, e.g., exhibit reduced coefficient of variation, compared to the expression and/or antigen binding of cell populations and/or compositions generated using conventional methods. In some embodiments, the cell population and/or compositions exhibit at least 100%, 95%, 90%, 80%, 70%, 60%, 50%, 40%, 30%, 20% or 10% lower coefficient of variation of expression of the transgene and/or antigen binding by the recombinant receptor compared to a respective population generated using conventional methods, e.g., random integration of transgene. The coefficient of variation is defined as standard deviation of expression of the nucleic acid of interest (e.g., transgene encoding a recombinant receptor) within a population of cells, for example CD4+ and/or CD8+ T cells, divided by the mean of expression of the respective nucleic acid of interest in the respective population of cells. In some embodiments, the cell population and/or compositions exhibit a coefficient of variation that is lower than 0.70, 0.65, 0.60, 0.55, 0.50, 0.45, 0.40, 0.35 or 0.30 or less, when measured among CD4+ and/or CD8+ T cell populations that have been engineered using the methods provided herein.

In some embodiments, provided are cell population and/or compositions that include cells that have a targeted knock-in of the recombinant receptor-encoding transgene into one or more of the endogenous TCR gene loci, thereby having a knock-out of the one or more of the endogenous TCR gene loci, e.g., knock out of the target gene for integration, such as TRAC, TRBC1 and/or TRBC2. In some embodiments, all or substantially all of the cells in the cell population that have integration of the recombinant receptor-encoding transgene also have a knock-out of the one or more of the endogenous TCR gene loci. In some embodiments, at least 80%, 85%, 90%, 91%, 92%, 93%, 94%, 95%, 96%, 97%, 98%, 99%, or more of the cells in the cell population and/or composition that express the recombinant receptor, contain a knock-out of the one or more of the endogenous TCR gene loci, e.g., TRAC, TRBC1 and/or TRBC2. Thus, in the provided cell population and/or compositions, all or substantially all of the engineered cells that express the recombinant receptor, also contain a knock-out of the endogenous TCR, by virtue of targeted knock-in of the transgene into the endogenous TCR gene loci.

In some embodiments, provided are cell population and/or compositions that include a plurality of engineered immune cells comprising a recombinant receptor or an antigen-binding fragment thereof encoded by a transgene and a genetic disruption of at least one target site within a T cell receptor alpha constant (TRAC) gene and/or a T cell receptor beta constant (TRBC) gene, wherein at least or greater than 35%, 40%, 45%, 50%, 55%, 60%, 65%, 70%, 75%, 80%, or 90% of the cells in the composition comprise a genetic disruption at a target position within a T cell receptor alpha constant (TRAC) gene and/or a T cell receptor beta constant (TRBC) gene; and the transgene encoding the recombinant TCR or antigen-binding fragment thereof or a chain thereof is targeted at or near the target position via homology directed repair (HDR).

In some embodiments, expression and/or antigen binding by the recombinant receptor can be assessed using any reagents and/or assays described herein, e.g., in Section I.C. In some embodiments, expression is measured using a binding molecule that recognizes and/or specifically binds to the recombinant receptor or a portion thereof. For example, in some embodiments, expression of the recombinant receptor encoded by the transgene is assessed using an anti-TCR V 22 antibody, e.g., by flow cytometry. In some embodiments, antigen binding of a recombinant receptor that is a TCR, can be assessed using antigen that is isolated or purified or recombinant, cells expressing particular antigen, and/or using a TCR ligand (MHC-peptide complex).

In some embodiments, the provided compositions containing cells such as in which cells expressing the recombinant receptor and/or contain a knock-out of one or more of the endogenous TCR-encoding genes make up at least 30%, 40%, 50%, 60%, 70%, 80%, 90%, 91%, 92%, 93%, 94%, 95%, 96%, 97%, 98%, 99%, or more of the total cells in the composition or cells of a certain type such as T cells or CD8+ or CD4+ cells.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

The cells and compositions may be administered using standard administration techniques, formulations, and/or devices. Administration of the cells can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

### VI. METHODS OF ADMINISTRATION AND USES IN ADOPTIVE CELL THERAPY

Provided are methods of administering the cells, populations, and compositions, and uses of such cells, populations, and compositions to treat or prevent diseases, conditions, and disorders, including cancers. In some embodiments, the cells, populations, and compositions are administered to a subject or patient having the particular disease or condition to be treated, e.g., via adoptive cell therapy, such as adoptive T cell therapy. In some embodiments, cells and compositions prepared by the provided methods, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in a cancer expressing an antigen recognized by an engineered T cell.

As used herein, a “subject” is a mammal, such as a human or other animal, and typically is human. In some embodiments, the subject, e.g., patient, to whom the cells, cell populations, or compositions are administered is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. A sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject.

In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells can be administered by any suitable means. Dosing and administration may depend in part on whether the administration is brief or chronic. Various dosing schedules include but are not limited to single or multiple administrations over various time-points, bolus administration, and pulse infusion.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse.

In some aspects, the subject has not received prior treatment with another therapeutic agent.

The disease or condition that is treated in some aspects can be any in which expression of an antigen is associated with, specific to, and/or expressed on a cell or tissue of a disease, disorder or condition and/or involved in the etiology of a disease, condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by a bacterial, viral or other pathogen. Exemplary antigens, which include antigens associated with various diseases and conditions that can be treated, are described herein. In particular embodiments, the immunomodulatory polypeptide and/or recombinant receptor, e.g., the chimeric antigen receptor or TCR, specifically binds to an antigen associated with the disease or condition. In some embodiments, the subject has a disease, disorder or condition, optionally a cancer, a tumor, an autoimmune disease, disorder or condition, or an infectious disease.

In some embodiments, the disease, disorder or condition includes tumors associated with various cancers. The cancer can in some embodiments be any cancer located in the body of a subject, such as, but not limited to, cancers located at the head and neck, breast, liver, colon, ovary, prostate, pancreas, brain, cervix, bone, skin, eye, bladder, stomach, esophagus, peritoneum, or lung. For example, the anti-cancer agent can be used for the treatment of colon cancer, cervical cancer, cancer of the central nervous system, breast cancer, bladder cancer, anal carcinoma, head and neck cancer, ovarian cancer, endometrial cancer, small cell lung cancer, non-small cell lung carcinoma, neuroendocrine cancer, soft tissue carcinoma, penile cancer, prostate cancer, pancreatic cancer, gastric cancer, gall bladder cancer or esophageal cancer. In some cases, the cancer can be a cancer of the blood. In some embodiments, the disease, disorder or condition is a tumor, such as a solid tumor, lymphoma, leukemia, blood tumor, metastatic tumor, or other cancer or tumor type. In some embodiments, the disease, disorder or condition is selected from among cancers of the colon, lung, liver, breast, prostate, ovarian, skin, melanoma, bone, brain cancer, ovarian cancer, epithelial cancers, renal cell carcinoma, pancreatic adenocarcinoma, cervical carcinoma, colorectal cancer, glioblastoma, neuroblastoma, Ewing sarcoma, medulloblastoma, osteosarcoma, synovial sarcoma, and/or mesothelioma.

Among the diseases, conditions, and disorders are tumors, including solid tumors, hematologic malignancies, and melanomas, and including localized and metastatic tumors, infectious diseases, such as infection with a virus or other pathogen, e.g., HIV, HCV, HBV, CMV, HPV, and parasitic disease, and autoimmune and inflammatory diseases. In some embodiments, the disease, disorder or condition is a tumor, cancer, malignancy, neoplasm, or other proliferative disease or disorder. Such diseases include but are not limited to leukemia, lymphoma, e.g., acute myeloid (or myelogenous) leukemia (AML), chronic myeloid (or myelogenous) leukemia (CML), acute lymphocytic (or lymphoblastic) leukemia (ALL), chronic lymphocytic leukemia (CLL), hairy cell leukemia (HCL), small lymphocytic lymphoma (SLL), Mantle cell lymphoma (MCL), Marginal zone lymphoma, Burkitt lymphoma, Hodgkin lymphoma (HL), non-Hodgkin lymphoma (NHL), Anaplastic large cell lymphoma (ALCL), follicular lymphoma, refractory follicular lymphoma, diffuse large B-cell lymphoma (DLBCL) and multiple myeloma (MM), a B cell malignancy is selected from among acute lymphoblastic leukemia (ALL), adult ALL, chronic lymphoblastic leukemia (CLL), non-Hodgkin lymphoma (NHL), and Diffuse Large B-Cell Lymphoma (DLBCL).

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In some embodiments, antigen associated with the disease, disorder or condition is selected from ROR1, B cell maturation antigen (BCMA), carbonic anhydrase 9 (CAIX), tEGFR, Her2/neu (receptor tyrosine kinase erbB2), L1-CAM, CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRL5, FCRH5, fetal acetylcholine receptor, GD2, GD3, HMW-MAA, IL-22R-alpha, IL-13R-alpha2, kinase insert domain receptor (kdr), kappa light chain, Lewis Y, L1-cell adhesion molecule, (L1-CAM), Melanoma-associated antigen (MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-AI MAGE A1, HLA-A2 NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, ROR1, TAG72, VEGF-R2, carcinoembryonic antigen (CEA), Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, a pathogen-specific antigen.

In some embodiments, the antigen associated with the disease or disorder is selected from the group consisting of orphan tyrosine kinase receptor ROR1, tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, and MAGE A3 and/or biotinylated molecules, and/or molecules expressed by HIV, HCV, HBV or other pathogens.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 10⁴ and at or about 10⁹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight.

In some embodiments, the cells are administered at or within a certain range of error of between at or about 10⁴ and at or about 10⁹ CD4⁺ and/or CD8⁺ cells/kilograms (kg) body weight, such as between 10⁵ and 10⁶ CD4⁺ and/or CD8⁺ cells/kg body weight, for example, at or about 1×10⁵ CD4⁺ and/or CD8⁺ cells/kg, 1.5×10⁵ CD4⁺ and/or CD8⁺ cells/kg, 2×10⁵ CD4⁺ and/or CD8⁺ cells/kg, or 1×10⁶ CD4⁺ and/or CD8⁺ cells/kg body weight.

In some embodiments, the cells are administered at or within a certain range of error of, greater than, and/or at least about 1×10⁶, about 2.5×10⁶, about 5×10⁶, about 7.5×10⁶, or about 9×10⁶ CD4⁺ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4⁺ cells, and/or between about 10⁸ and 10¹² or between about 10¹⁰ and 10¹¹ CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4⁺ to CD8⁺ cells) is between at or about 1:5 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, for example, where the subject is a human, the dose includes fewer than about 1×10⁸ total recombinant receptor (e.g., recombinant TCR)-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the size of the dose is determined by the burden of the disease or condition in the subject. For example, in some aspects, the number of cells administered in the dose is determined based on the tumor burden that is present in the subject immediately prior to administration of the initiation of the dose of cells. In some embodiments, the size of the first and/or subsequent dose is inversely correlated with disease burden. In some aspects, as in the context of a large disease burden, the subject is administered a low number of cells. In other embodiments, as in the context of a lower disease burden, the subject is administered a larger number of cells.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD 107a, IFNγ, IL-2, and TNF. In some aspects the biological activity is measured by assessing clinical outcome, such as reduction in tumor burden or load.

In certain embodiments, the engineered cells are further modified in any number of ways, such that their therapeutic or prophylactic efficacy is increased. For example, the engineered CAR or TCR expressed by the population can be conjugated either directly or indirectly through a linker to a targeting moiety. The practice of conjugating compounds, e.g., the CAR or TCR, to targeting moieties is known. See, for instance, Wadwa et al., J. Drug Targeting 3: 1 1 1 (1995), and U.S. Pat. No. 5,087,616.

### VII. KITS AND ARTICLES OF MANUFACTURE

Also provided are articles of manufacture, systems, apparatuses, and kits useful in performing the provided embodiments. In some embodiments, the provided articles of manufacture or kits contain one or more components of the one or more agent(s) capable of inducing genetic disruption and/or template polynucleotide(s), e.g., template polynucleotides containing transgene encoding the recombinant receptor or antigen-binding fragment or chain thereof or the one or more second template polynucleotides. In some embodiments, the articles of manufacture or kits can be used in methods for engineering T cells to express a recombinant receptor and/or other polypeptides and assessing the produced cells and/or cell populations in accord with the provided methods. In some embodiments, the articles of manufacture or kits provided herein contain T cells and/or T cell compositions, such as any T cells and/or T cell compositions described herein.

In some embodiments, the articles of manufacture or kits include polypeptides, nucleic acids, vectors and/or polynucleotides useful in performing the provided methods. In some embodiments, the articles of manufacture or kits include one or more nucleic acid molecules, e.g., a plasmid or a DNA fragment, that comprises one or more components of the one or more agent(s) capable of inducing genetic disruption and/or template polynucleotide(s), e.g., template polynucleotides containing transgene encoding the recombinant receptor or antigen-binding fragment or chain thereof or the one or more second template polynucleotides. In some embodiments, the articles of manufacture or kits provided herein contain control vectors.

In some embodiments, the articles of manufacture or kits provided herein contain one or more agent, wherein each of the one or more agent is independently capable of inducing a genetic disruption of a target site within a T cell receptor alpha constant (TRAC) gene and/or a T cell receptor beta constant (TRBC) gene; and a template polynucleotide comprising a transgene encoding a recombinant TCR or an antigen-binding fragment or a chain thereof, wherein the transgene encoding the recombinant TCR or antigen-binding fragment or chain thereof is targeted for integration at or near the target site via homology directed repair (HDR).

In some embodiments, the articles of manufacture or kits provided herein contain T cells, and/or T cell compositions, such as any T cells, and/or T cell compositions described herein. In some embodiments, the T cells, and/or T cell compositions any of the modified T cells used the screening methods described herein. In some embodiments, the articles of manufacture or kits provided herein contain control T cells, and/or T cell compositions.

In some embodiments, the articles of manufacture or kits include one or more components used to assess the properties of the population and/or composition of engineered cells expressing a recombinant receptor. For example, the articles of manufacture or kits can include binding reagents, e.g., antibodies, MHC-peptide tetramers and/or probes, used to assess particular properties of the introduced recombinant TCRs, e.g., cell surface expression of the recombinant TCR, recognition of a peptide in the context of an MHC molecule and/or detectable signal produced by the reporter, e.g., a T cell activation reporter. In some embodiments, the articles of manufacture or kits can include components that are used for detection of particular properties, such as labeled components, e.g., fluorescently labeled components and/or components that can produce a detectable signal, e.g., substrates that can produce fluorescence or luminescence.

In some embodiments, the articles of manufacture or kits include one or more containers, typically a plurality of containers, packaging material, and a label or package insert on or associated with the container or containers and/or packaging, generally including instructions for use, e.g., instructions for introducing the components into the cells for engineering and/or for assessing the engineered cell populations and/or compositions. In some embodiments, the article of manufacture or kits include one or more instructions for administration of the engineered cells and/or cell compositions for therapy.

The articles of manufacture provided herein contain packaging materials. Packaging materials for use in packaging the provided materials are known. See, for example, U.S. Pat. Nos. 5,323,907, 5,052,558 and 5,033,252, each of which is incorporated herein in its entirety. Examples of packaging materials include, but are not limited to, blister packs, bottles, tubes, inhalers, pumps, bags, vials, containers, syringes, disposable laboratory supplies, e.g., pipette tips and/or plastic plates, or bottles. The articles of manufacture or kits can include a device so as to facilitate dispensing of the materials or to facilitate use in a high-throughput or large-scale manner, e.g., to facilitate use in robotic equipment. Typically, the packaging is non-reactive with the compositions contained therein.

In some embodiments, the agent capable of inducing genetic disruption and/or template polynucleotide(s) are packaged separately. In some embodiments, each container can have a single compartment. In some embodiments, other components of the articles of manufacture or kits are packaged separately, or together in a single compartment.

### VIII. DEFINITIONS

Unless defined otherwise, all terms of art, notations and other technical and scientific terms or terminology used herein are intended to have the same meaning as is commonly understood by one of ordinary skill to which the claimed subject matter pertains. In some cases, terms with commonly understood meanings are defined herein for clarity and/or for ready reference, and the inclusion of such definitions herein should not necessarily be construed to represent a substantial difference over what is generally understood.

The terms “polypeptide” and “protein” are used interchangeably to refer to a polymer of amino acid residues, and are not limited to a minimum length. Polypeptides, including the provided antibodies and antibody chains and other peptides, e.g., linkers, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding a TCR or an antibody” refers to one or more nucleic acid molecules encoding TCRα, or β chains (or fragments thereof) or antibody heavy and light chains (or fragments thereof), including such nucleic acid molecule(s) in a single vector or separate vectors, and such nucleic acid molecule(s) present at one or more locations in a host cell.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

In some embodiments, “operably linked” may include the association of components, such as a DNA sequence, e.g. a heterologous nucleic acid) and a regulatory sequence(s), in such a way as to permit gene expression when the appropriate molecules (e.g. transcriptional activator proteins) are bound to the regulatory sequence. Hence, it means that the components described are in a relationship permitting them to function in their intended manner.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill, for instance, using publicly available computer software such as BLAST, BLAST-2, ALIGN or Megalign (DNASTAR) software. Appropriate parameters for aligning sequences may be determined, including any algorithms needed to achieve maximal alignment over the full length of the sequences being compared.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”. In some embodiments, “about” may refer to ±25%, ±20%, ±15%, ±10%, ±5%, or ±1%.

As used herein, a composition refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### IX. EXEMPLARY EMBODIMENTS

Among the provided embodiments are:

1. A genetically engineered T cell, comprising a modified T cell receptor alpha constant (TRAC) locus, said modified TRAC locus comprising a nucleic acid encoding a recombinant TCR or portion thereof, said recombinant TCR or portion thereof comprising: (i) a TCR alpha (TCRα) chain comprising a variable alpha (Vα) domain and a constant domain, and (ii) a TCR beta (TCRβ) chain comprising a variable beta (Vβ) domain and a constant domain, wherein the nucleic acid sequence comprises of (a) a transgene sequence encoding the TCRβ and the Vα domain, and (b) an open reading frame of the endogenous TRAC locus or a partial sequence thereof, wherein the open reading frame encodes at least a portion of the Cα domain of the recombinant TCR.

2. The genetically engineered T cell of embodiment 1, wherein the transgene sequence is integrated via homology directed repair (HDR).

3. The genetically engineered T cell of embodiment 1 or embodiment 2, wherein the modified TRAC locus comprises an in-frame fusion of (i) a transgene sequence and (ii) an open reading frame or a partial sequence thereof of the endogenous TRAC locus.

4. The genetically engineered T cell of any of embodiments 1-3, wherein the transgene sequence does not comprise a sequence encoding a 3′ untranslated region (3′ UTR) or an intron.

5. The genetically engineered T cell of any of embodiments 1-4, wherein the open reading frame or a partial sequence thereof comprises a 3′ UTR of the endogenous TRAC locus.

6. The genetically engineered T cell of any of embodiments 1-5, wherein a portion of the Cα is encoded by the open reading frame of the endogenous TRAC locus or a partial sequence thereof, and a further portion of the Cα is encoded by the transgene sequence, wherein said further portion of Cα is less than the full length of a native Cα.

7. The genetically engineered T cell of any of embodiments 1-6, wherein the open reading frame or the partial sequence thereof comprises at least one intron and at least one exon of the endogenous TRAC locus.

8. The genetically engineered T cell of any of embodiments 1-7, wherein the transgene sequence is in-frame with one or more exons of the open reading frame or partial sequence thereof of the endogenous TRAC locus.

9. The genetically engineered T cell of any of embodiments 1-8, wherein the transgene sequence is integrated downstream of the most 5′ nucleotide of exon 1 and upstream of the most 3′ nucleotide of exon 1 of the open reading frame of the endogenous TRAC locus.

10. The genetically engineered T cell of any of embodiments 1-9, wherein the at least a portion of Cα is encoded by at least exons 2-4 of the open reading frame of the endogenous TRAC locus.

11. The genetically engineered T cell of any of embodiments 1-10, wherein the at least a portion Cα is encoded by at least a portion of exon 1 and exons 2-4 of the open reading frame of the endogenous TRAC locus.

12. The genetically engineered T cell of any of embodiments 1-11, wherein the at least a portion Cα is encoded by less than the full length of exon 1 of the open reading frame of the endogenous TRAC locus.

13. The genetically engineered T cell of any of embodiments 1-12, wherein the encoded TCRα chain is capable of dimerizing with a TCRβ chain.

14. The genetically engineered T cell of any of embodiments 1-13, wherein the encoded Cα comprises the sequence selected from any one of SEQ ID NOS: 14, 15, 19, or 24, or a sequence that exhibits at least 85%, 86%, 87%, 88%, 89%, 90%, 91%, 92%, 93%, 94%, 95%, 96%, 97%, 98%, 99% or more sequence identity to any one of SEQ ID NOS: 14, 15, 19, or 24, or a partial sequence thereof.

15. The genetically engineered T cell of any of embodiments 6-14, wherein the further portion of the Cα is encoded by a sequence of nucleotides starting from residue 3 and up to residue 3155 of the sequence set forth in SEQ ID NO:1 or one or more exons thereof or a sequence that exhibits at least 85%, 86%, 87%, 88%, 89%, 90%, 91%, 92%, 93%, 94%, 95%, 96%, 97%, 98%, 99% or more sequence identity to a sequence of nucleotides starting from residue 3 and up to residue 3155 of the sequence set forth in SEQ ID NO:1 or one or more exons thereof, or a partial sequence thereof.

16. The genetically engineered T cell of embodiment 6-15, wherein the further portion of the Cα is encoded by a sequence of nucleotides that is less than 400, less than 300, less than 250, less than 200, or less than 150 base pairs in length.

17. The genetically engineered T cell of embodiment 14 or embodiment 15, wherein the further portion of the Cα is encoded by a sequence of nucleotides that comprises less than four exons, less than three exons, less than two exons, one exon, or less than one full exon the open reading frame of the TRAC locus.

18. The genetically engineered T cell of any of embodiments 6-17, wherein the further portion of the Cα is encoded by a portion of exon 1 of the TRAC locus, wherein the portion of exon 1 is less than the full length of exon 1 the open reading frame of the TRAC locus.

19. The genetically engineered T cell of any of any of embodiments 6-18 wherein the further portion of the Cα is encoded by a portion of exon 1 of the TRAC locus, wherein the portion of exon 1 comprises a 5′ portion of exon 1.

20. The genetically engineered T cell of any of embodiments 6-19, wherein the further portion of the Cα comprises a sequence set forth in SEQ ID NO:142, or a sequence that exhibits at least 85%, 86%, 87%, 88%, 89%, 90%, 91%, 92%, 93%, 94%, 95%, 96%, 97%, 98%, 99% or more sequence identity to SEQ ID NO:142, or a partial sequence thereof.

21. The genetically engineered T cell of any of embodiments 6-20, wherein the further portion of the Cα and/or the Cβ region encoded by the nucleic acid sequence of (a) comprises one or more modifications, optionally a replacement, deletion, or insertion of one or more amino acids compared to a native Cα region and/or a native Cβ region, optionally said one or more modifications introduces one or more cysteine residues that are capable of forming one or more non-native disulfide bridges between the alpha chain and beta chain.

22. The genetically engineered T cell of any of embodiments 1-21, wherein the engineered T cell further comprises a genetic disruption at a TRBC locus.

23. The genetically engineered T cell of any of embodiments 1-22, wherein the engineered T cell further comprises a genetic disruption at a TRBC1 locus and/or a TRBC2 locus.

24. The genetically engineered T cell of any of embodiments 1-23, wherein the further portion of the Cα and/or the Cβ region encoded by the nucleic acid sequence of (a) comprises one or more modifications, optionally a replacement, deletion, or insertion of one or more amino acids compared to a native Cα region and/or a native Cβ region, optionally said one or more modifications introduces one or more cysteine residues that are capable of forming one or more non-native disulfide bridges between the alpha chain and beta chain.

25. A genetically engineered T cell, comprising a modified T cell receptor beta constant (TRBC) locus, said modified TRBC locus comprising a nucleic acid encoding a recombinant TCR or portion thereof, said recombinant TCR or portion thereof comprising: (i) a TCR beta (TCRβ) chain comprising a variable beta (Vβ) domain and a constant domain, and (ii) a TCR alpha (TCRα) chain comprising a variable alpha (Vα) domain and a constant domain, wherein the nucleic acid sequence comprises of (a) a transgene sequence encoding the TCRα and the Vβ domain, and (b) an open reading frame of the endogenous TRBC locus or a partial sequence thereof, wherein the open reading frame encodes at least a portion of the Cβ domain of the recombinant TCR.

26. The genetically engineered T cell of embodiment 25, wherein the transgene sequence is integrated via homology directed repair (HDR).

27. The genetically engineered T cell of embodiment 25 or embodiment 26, wherein the TRBC locus is a TRBC1 locus and/or a TRBC2 locus.

28. The genetically engineered T cell of embodiment 25 or embodiment 26, wherein the modified TRBC locus comprises an in-frame fusion of (i) a transgene sequence and (ii) an open reading frame or a partial sequence thereof of the endogenous TRBC locus.

29. The genetically engineered T cell of any of embodiments 25-28, wherein the transgene sequence does not comprise a sequence encoding a 3′ untranslated region (3′ UTR) or an intron.

30. The genetically engineered T cell of any of embodiments 25-29, wherein the open reading frame or a partial sequence thereof comprises a 3′ UTR of the endogenous TRBC locus.

31. The genetically engineered T cell of any of embodiments 25-30, wherein a portion of the Cβ is encoded by the open reading frame of the endogenous TRBC locus or a partial sequence thereof, and a further portion of the Cβ is encoded by the transgene sequence, wherein said further portion of Cβ is less than the full length of a native Cβ.

32. The genetically engineered T cell of any of embodiments 25-31, wherein the open reading frame or the partial sequence thereof comprises at least one intron and at least one exon of the endogenous TRBC locus.

33. The genetically engineered T cell of any of embodiments 25-32, wherein the transgene sequence is in-frame with one or more exons of the open reading frame or partial sequence thereof of the endogenous TRBC locus.

34. The genetically engineered T cell of any of embodiments 25-33, wherein the transgene sequence is integrated downstream of the most 5′ nucleotide of exon 1 and upstream of the most 3′ nucleotide of exon 1 of the open reading frame of the endogenous TRBC locus.

35. The genetically engineered T cell of any of embodiments 25-34, wherein the at least a portion of Cβ is encoded by at least exons 2-4 of the open reading frame of the endogenous TRBC locus.

36. The genetically engineered T cell of any of embodiments 25-35, wherein the at least a portion C is encoded by at least a portion of exon 1 and exons 2-4 of the open reading frame of the endogenous TRBC locus.

37. The genetically engineered T cell of any of embodiments 25-36, wherein the at least a portion C is encoded by less than the full length of exon 1 of the open reading frame of the endogenous TRBC locus.

38. The genetically engineered T cell of any of embodiments 25-37, wherein the encoded TCRβ chain is capable of dimerizing with a TCRα chain.

39. The genetically engineered T cell of any of embodiments 25-38, wherein the encoded Cβ comprises the sequence selected from any one of SEQ ID NO: 16, 17, 21, or 25, or a sequence that exhibits at least 85%, 86%, 87%, 88%, 89%, 90%, 91%, 92%, 93%, 94%, 95%, 96%, 97%, 98%, 99% or more sequence identity to SEQ ID NO: 16, 17, 21, or 25, or a partial sequence thereof.

40. The genetically engineered T cell of any of embodiments 31-39, wherein the further portion of the Cα is encoded by:

a sequence of nucleotides starting from residue 3 and up to residue 1445 of the sequence set forth in SEQ ID NO:2 or one or more exons thereof or a sequence that exhibits at least 85%, 86%, 87%, 88%, 89%, 90%, 91%, 92%, 93%, 94%, 95%, 96%, 97%, 98%, 99% or more sequence identity to a sequence of nucleotides starting from residue 3 and up to residue 1445 of the sequence set forth in SEQ ID NO:2 or one or more exons thereof, or a partial sequence thereof; or

a sequence of nucleotides starting from residue 3 and up to residue 1486 of the sequence set forth in SEQ ID NO:3 or one or more exons thereof or a sequence that exhibits at least 85%, 86%, 87%, 88%, 89%, 90%, 91%, 92%, 93%, 94%, 95%, 96%, 97%, 98%, 99% or more sequence identity to a sequence of nucleotides starting from residue 3 and up to residue 1486 of the sequence set forth in SEQ ID NO:3 or one or more exons thereof, or a partial sequence thereof.

41. The genetically engineered T cell of embodiment 31-40, wherein the further portion of the Cα is encoded by a sequence of nucleotides that is less than 400, less than 300, less than 250, less than 200, or less than 150 base pairs in length.

42. The genetically engineered T cell of embodiment 35 or embodiment 36, wherein the further portion of the Cα is encoded by a sequence of nucleotides that comprises less than four exons, less than three exons, less than two exons, one exon, or less than one full exon the open reading frame of a TRBC locus.

43. The genetically engineered T cell of any of embodiments 31-42, wherein the further portion of the Cα is encoded by a portion of exon 1 of a TRBC locus, wherein the portion of exon 1 is less than the full length of exon 1 the open reading frame of the TRBC locus.

44. The genetically engineered T cell of any of any of embodiments 31-43 wherein the further portion of the Cα is encoded by a portion of exon 1 of a TRBC locus, wherein the portion of exon 1 comprises a 5′ portion of exon 1.

45. The genetically engineered T cell of any of embodiments 25-44, wherein the further portion of the Cβ and/or the Cα region encoded by the nucleic acid sequence of (a) comprises one or more modifications, optionally a replacement, deletion, or insertion of one or more amino acids compared to a native Cβ region and/or a native Cα region, optionally said one or more modifications introduces one or more cysteine residues that are capable of forming one or more non-native disulfide bridges between the alpha chain and beta chain.

46. The genetically engineered T cell of any of embodiments 25-45, wherein the engineered T cell further comprises a genetic disruption at a TRAC locus.

47. The genetically engineered T cell of any of embodiments 1-46, wherein transgene sequence comprises one or more multicistronic element(s).

48. The genetically engineered T cell of embodiment 47, wherein the multicistronic element(s) is positioned between the nucleic acid sequence encoding the TCRα or a portion thereof and the nucleic acid sequence encoding the TCRβ or a portion thereof.

49. The genetically engineered T cell of embodiment 47 or embodiment 48, wherein the one or more multicistronic element(s) are upstream of the nucleic acid sequence encoding the TCR or a portion of the TCR or the nucleic acid molecule encoding the TCR.

50. The genetically engineered T cell of any of embodiments 47-49, wherein the multicistronic element is or comprises a ribosome skip sequence, optionally T2A, P2A, E2A, or F2A.

51. The genetically engineered T cell of any of embodiments 1-50, further comprising one or more heterologous regulatory or control element(s).

52. The genetically engineered T cell of any of embodiments 1-51, wherein transgene sequence comprises one or more heterologous or regulatory control element(s) operably linked to control expression of the TCR when expressed from a cell introduced with the genetically engineered T cell.

53. The genetically engineered T cell of embodiment 51 or embodiment 52, wherein the one or more heterologous regulatory or control element comprises a promoter, an enhancer, an intron, a polyadenylation signal, a Kozak consensus sequence, a splice acceptor sequence and/or a splice donor sequence.

54. The genetically engineered T cell of any of embodiments 51-53, wherein the heterologous regulatory or control element comprises a heterologous promoter.

55. The genetically engineered T cell of embodiment 54, wherein the heterologous promoter is selected from among a constitutive promoter, an inducible promoter, a repressible promoter, and/or a tissue-specific promoter.

56. The genetically engineered T cell of embodiment 54 or embodiment 55, wherein the heterologous promoter is or comprises a human elongation factor 1 alpha (EF1α) promoter or an MND promoter or a variant thereof.

57. The genetically engineered T cell of any of embodiment 1-56, wherein the Cα and/or the Cβ of the recombinant TCR comprises one or more non-native cysteines.

58. The genetically engineered T cell of any embodiments 1-57, wherein the recombinant TCR is capable of binding to an antigen that is associated with, specific to, and/or expressed on a cell or tissue that is associated with a disease, disorder, or condition.

59. The genetically engineered T cell of any of embodiments 1-58, wherein the disease, disorder, or condition is an infectious disease or disorder, an autoimmune disease, an inflammatory disease, a tumor, or a cancer.

60. The genetically engineered T cell of any of embodiments 1-59, wherein the antigen is a tumor antigen or a pathogenic antigen.

61. The genetically engineered T cell of embodiment 60, wherein the pathogenic antigen is a bacterial antigen or viral antigen.

62. The genetically engineered T cell of embodiment 61, wherein the antigen is a viral antigen, optionally a viral antigen from hepatitis A, hepatitis B, hepatitis C virus (HCV), human papilloma virus (HPV), hepatitis viral infections, Epstein-Barr virus (EBV), human herpes virus 8 (HHV-8), human T-cell leukemia virus-1 (HTLV-1), human T-cell leukemia virus-2 (HTLV-2), or a cytomegalovirus (CMV).

63. The genetically engineered T cell of embodiment 61 or embodiment 62, wherein the viral antigen is an antigen from an HPV selected from among HPV-16, HPV-18, HPV-31, HPV-33 and HPV-35.

64. The genetically engineered T cell of embodiment 61 or embodiment 62, wherein the viral antigen is an HPV-16 antigen that is an HPV-16 E6 or HPV-16 E7 antigen.

65. The genetically engineered T cell of embodiment 61 or embodiment 62, wherein the viral antigen is an EBV antigen selected from among Epstein-Barr nuclear antigen (EBNA)-1, EBNA-2, EBNA-3A, EBNA-3B, EBNA-3C, EBNA-leader protein (EBNA-LP), latent membrane proteins LMP-1, LMP-2A and LMP-2B, EBV-EA, EBV-MA and EBV-VCA.

66. The genetically engineered T cell of embodiment 61 or embodiment 62, wherein the viral antigen is an HTLV-antigen that is TAX.

67. The genetically engineered T cell of embodiment 61 or embodiment 62, wherein the viral antigen is an HBV antigen that is a hepatitis B core antigen or a hepatitis B envelope antigen.

68. The genetically engineered T cell of embodiment 61 or embodiment 62, wherein the antigen is a tumor antigen.

69. The genetically engineered T cell of embodiment 59 or 60, wherein the antigen is selected from among glioma-associated antigen, 3-human chorionic gonadotropin, alpha fetoprotein (AFP), lectin-reactive AFP, thyroglobulin, RAGE-1, MN-CA IX, human telomerase reverse transcriptase, RUl, RU2 (AS), intestinal carboxyl esterase, muthsp70-2, M-CSF, Melanin-A/MART-1, WT-1, S-100, MBP, CD63, MUC1 (e.g. MUC1-8), p53, Ras, cyclin B1, HER-2/neu, carcinoembryonic antigen (CEA), gp100, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A11, MAGE-B1, MAGE-B2, MAGE-B3, MAGE-B4, MAGE-C1, BAGE, GAGE-1, GAGE-2, p15, tyrosinase (e.g. tyrosinase-related protein 1 (TRP-1) or tyrosinase-related protein 2 (TRP-2)), P-catenin, NY-ESO-1, LAGE-1a, PP1, MDM2, MDM4, EGVFvIII, Tax, SSX2, telomerase, TARP, pp65, CDK4, vimentin, S100, eIF-4A1, IFN-inducible p78, melanotransferrin (p97), Uroplakin II, prostate specific antigen (PSA), human kallikrein (huK2), prostate specific membrane antigen (PSM), and prostatic acid phosphatase (PAP), neutrophil elastase, ephrin B2, BA-46, Bcr-abl, E2A-PRL, H4-RET, IGH-IGK, MYL-RAR, Caspase 8, FRa, CD24, CD44, CD133, CD 166, epCAM, CA-125, HE4, Oval, estrogen receptor, progesterone receptor, uPA, PAI-1, CD19, CD20, CD22, ROR1, CD33/IL3Ra, c-Met, PSMA, Glycolipid F77, GD-2, insulin growth factor (IGF)-I, IGF-II, IGF-I receptor and mesothelin.

70. The genetically engineered T cell of any of embodiments 1-69, wherein the T cell is a primary T cell derived from a subject, optionally wherein the subject is a human.

71. The genetically engineered T cell of any of embodiments 1-70, wherein the T cell is a CD8+ T cell or subtypes thereof.

72. The genetically engineered cell of any of embodiments 1-71, wherein the T cell is a CD4+ T cell or subtypes thereof.

73. The genetically engineered cell of any of embodiments 1-72, wherein the T cell is derived from a multipotent or pluripotent cell, which optionally is an iPSC.

74. A composition comprising a plurality of genetically engineered T cells of any of embodiments 1-73.

75. The composition of embodiment 74, comprising CD4+ and/or CD8+ T cells.

76. The composition of embodiment 74 or embodiment 75, wherein the composition comprises CD4+ and CD8+ T cells and the ratio of CD4+ to CD8+ T cells is from or from about 1:3 to 3:1, optionally 1:1.

77. The composition of any of embodiments 74-76, wherein:

at least 70%, 75%, 80%, 90%, 91%, 92%, 93%, 94%, 95%, 96%, 97%, 98%, or 99% of the engineered cells in the composition comprise a genetic disruption in or of an endogenous T cell receptor alpha constant region (TRAC) gene and/or a T cell receptor beta constant region (TRBC) gene; and/or

at least 80%, 90%, 91%, 92%, 93%, 94%, 95%, 96%, 97%, 98%, or 99% of the engineered cells in the composition do not express or do not express detectable levels of a gene product of an endogenous TRAC or TRBC gene.

78. The composition of any of embodiments 74-77, wherein at least or greater than 35%, 40%, 45%, 50%, 55%, 60%, 65%, 70%, 75%, 80%, or 90% of the cells in the composition express the recombinant TCR and/or exhibit antigen binding.

79. A polynucleotide, comprising:

(a) a nucleic acid sequence encoding a portion of a recombinant T cell receptor (TCR), said nucleic acid sequence encoding (i) a T cell receptor beta (TCRβ) chain comprising a variable beta (Vβ) domain and a constant beta (Cβ) domain; and (ii) a portion of a T cell receptor alpha (TCRα) chain, wherein the portion of the TCRα chain is less than a full-length native TCRα chain, and

(b) one or more homology arms linked to the nucleic acid sequence, wherein the one or more homology arms comprise a sequence homologous to one or more region(s) of an open reading frame of a TRAC locus.

80. The polynucleotide of embodiment 79, wherein the TCRα chain comprises a constant alpha region (Ca), wherein at least a portion of said Cα is encoded by the open reading frame of the endogenous TRAC locus or a partial sequence thereof when the TCR or antigen-binding fragment thereof is expressed from a cell introduced with the polynucleotide.

81. The polynucleotide of embodiment 79 or 80, wherein the nucleic acid sequence of (a) and the one of the one or more homology arms together comprise a sequence of nucleotides encoding the Cα that is less than the full length of a native Cα, wherein at least a portion of the Cα is encoded by the open reading frame of the endogenous TRAC locus or a partial sequence thereof when the TCR or antigen-binding fragment thereof is expressed from a cell introduced with the polynucleotide.

82. The polynucleotide of any of embodiments 79-81, wherein the polynucleotide is comprised in a viral vector.

83. The polynucleotide of any of embodiments 79-82, wherein the nucleic acid sequence encoding the TCRβ chain is upstream of the nucleic acid sequence encoding the portion of the TCRα chain.

84. The polynucleotide of any of embodiments 79-83, wherein the nucleic acid sequence of (a) does not comprise an intron.

85. The polynucleotide of any of embodiments 79-84, wherein the nucleic acid sequence of (a) is a sequence that is exogenous or heterologous to an open reading frame of an endogenous genomic TRAC locus of a T cell, optionally a human T cell.

86. The polynucleotide of any of embodiments 79-85, wherein the nucleic acid sequence of (a) is in-frame with one or more exons or a partial sequence thereof of the open reading frame of the TRAC locus comprised in the one or more homology arm(s).

87. The polynucleotide of any of embodiments 79-86, wherein the one or more exons or a partial sequence thereof of the open reading frame comprises a sequence within exon 1 of the open reading frame of the TRAC locus.

88. The polynucleotide of any of embodiments 79-87, wherein the TCRα chain is capable of dimerizing with a TCRβ chain, when produced from a cell introduced with the polynucleotide.

89. The polynucleotide of any of embodiments 79-88, wherein the portion of the TCRα chain comprises a variable alpha (Vα) domain.

90. The polynucleotide of any of embodiments 79-89, wherein a portion of the Cα is encoded by the open reading frame of the endogenous TRAC locus or a partial sequence thereof, and a further portion of the Cα is encoded by the nucleic acid sequence of (a), wherein said further portion of Cα is less than the full length of a native Cα.

91. The polynucleotide of embodiment 90, wherein the further portion of the Cα is encoded by a sequence of nucleotides starting from residue 3 and up to residue 3155 of the sequence set forth in SEQ ID NO:1 or one or more exons thereof or a sequence that exhibits at least 85%, 86%, 87%, 88%, 89%, 90%, 91%, 92%, 93%, 94%, 95%, 96%, 97%, 98%, 99% or more sequence identity to a sequence of nucleotides starting from residue 3 and up to residue 3155 of the sequence set forth in SEQ ID NO:1 or one or more exons thereof, or a partial sequence thereof.

92. The polynucleotide of embodiment 91, wherein the further portion of the Cα is encoded by a sequence of nucleotides that is less than 400, less than 300, less than 250, less than 200, or less than 150 base pairs in length.

93. The polynucleotide of embodiment 91 or 92, wherein the further portion of the Cα is encoded by a sequence of nucleotides that comprises less than four exons, less than three exons, less than two exons, one exon, or less than one full exon the open reading frame of the TRAC locus.

94. The polynucleotide of any of embodiments 91-93, wherein the further portion of the Cα is encoded by a portion of exon 1 of the TRAC locus, wherein the portion of exon 1 is less than the full length of exon 1 the open reading frame of the TRAC locus.

95. The polynucleotide of any of any of embodiments 90-94, wherein the further portion of the Cα is encoded by a portion of exon 1 of the TRAC locus, wherein the portion of exon 1 comprises a 5′ portion of exon 1.

96. The polynucleotide of any of embodiments 79-95, wherein the further portion of the Cα comprises a sequence set forth in SEQ ID NO:142, or a sequence that exhibits at least 85%, 86%, 87%, 88%, 89%, 90%, 91%, 92%, 93%, 94%, 95%, 96%, 97%, 98%, 99% or more sequence identity to SEQ ID NO:142, or a partial sequence thereof.

97. The polynucleotide of any of embodiments 79-96, wherein the further portion of the Cα and/or the Cβ region encoded by the nucleic acid sequence of (a) comprises one or more modifications, optionally a replacement, deletion, or insertion of one or more amino acids compared to a native Cα region and/or a native Cβ region, optionally said one or more modifications introduces one or more cysteine residues that are capable of forming one or more non-native disulfide bridges between the alpha chain and beta chain.

98. The polynucleotide of any of embodiments 79-97, wherein the one or more homology arm comprises a 5′ homology arm and/or a 3′ homology arm.

99. The polynucleotide of embodiment 98, wherein the 5′ homology arm and 3′ homology arm comprises nucleic acid sequences homologous to nucleic acid sequences surrounding a target site, wherein the target site is within the TRAC locus.

100. The polynucleotide of embodiment 99, wherein the target site is within exon 1 of the TRAC locus.

101. The polynucleotide of any of embodiments 98-100, wherein the 5′ homology arm comprises:

a) a sequence comprising at or at least at or at least 150, 200, 250, 300, 350, 400, 450, 500, 550, or 600 contiguous nucleotides to a sequence that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 124;

b) a sequence comprising at or at least at or at least 150, 200, 250, 300, 350, 400, 450, 500, 550, or 600 contiguous nucleotides of the sequence set forth in SEQ ID NO:124; or

c) the sequence set forth in SEQ ID NO: 124.

102. The polynucleotide of any of embodiments 98-101, wherein the 3′ homology arm comprises:

a) a sequence comprising at or at least at or at least 150, 200, 250, 300, 350, 400, 450, 500, 550, or 600 contiguous nucleotides to a sequence that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 125;

b) a sequence comprising at or at least at or at least 150, 200, 250, 300, 350, 400, 450, 500, 550, or 600 contiguous nucleotides of the sequence set forth in SEQ ID NO:125; or

c) the sequence set forth in SEQ ID NO: 125.

103. A polynucleotide, comprising:

(a) a nucleic acid sequence encoding a portion of a recombinant T cell receptor (TCR), said nucleic acid sequence encoding (i) a T cell receptor alpha (TCRα) chain comprising a variable alpha (Vα) domain and a constant alpha (Cα) domain; and (ii) a portion of a T cell receptor beta (TCRβ) chain, wherein the portion of the TCRβ chain is less than a full-length native TCRβ chain, and

(b) one or more homology arms linked to the nucleic acid sequence, wherein the one or more homology arms comprise a sequence homologous to one or more region(s) of an open reading frame of a TRBC locus.

104. The polynucleotide of embodiment 103, wherein the TCRβ chain comprises a constant beta (Cβ), wherein at least a portion of said C is encoded by the open reading frame of the endogenous TRAC locus or a partial sequence thereof when the TCR or antigen-binding fragment thereof is expressed from a cell introduced with the polynucleotide.

105. The polynucleotide of embodiment 103 or 104, wherein the nucleic acid sequence of (a) and the one of the one or more homology arms together comprise a sequence of nucleotides encoding the Cβ that is less than the full length of a native Cβ, wherein at least a portion of the Cβ is encoded by the open reading frame of the endogenous TRBC locus or a partial sequence thereof when the TCR or antigen-binding fragment thereof is expressed from a cell introduced with the polynucleotide.

106. The polynucleotide of any of embodiments 103-105, wherein the polynucleotide is comprised in a viral vector.

107. The polynucleotide of any of embodiments 103-106, wherein the TRBC locus is one or more of TRBC1 or TRBC2.

108. The polynucleotide of any of embodiments 103-107, wherein the nucleic acid sequence encoding the TCRα chain is upstream of nucleic acid sequence encoding the portion of the TCRβ chain.

109. The polynucleotide of any of embodiments 103-10⁸, wherein the nucleic acid sequence of (a) does not comprise an intron.

110. The polynucleotide of any of embodiments 103-109, wherein the nucleic acid sequence of (a) is a sequence that is exogenous or heterologous to an open reading frame of an endogenous genomic TRBC locus of a T cell, optionally a human T cell.

111. The polynucleotide of any of embodiments 103-110, wherein the nucleic acid sequence of (a) is in-frame with one or more exons or a partial sequence thereof of the open reading frame of the TRAC locus comprised in the one or more homology arm(s).

112. The polynucleotide of any of embodiments 103-111, wherein the one or more exons or a partial sequence thereof of the open reading frame is or comprises a sequence within exon 1 of the open reading frame of the TRBC locus.

113. The polynucleotide of any of embodiments 103-112, wherein the TCRβ chain is capable of dimerizing with a TCRα chain, when produced from a cell introduced with the polynucleotide.

114. The polynucleotide of any of embodiments 103-113, wherein the portion of the TCRβ chain comprises a variable beta (Vβ) domain.

115. The polynucleotide of any of embodiments 103-114, wherein a portion of the C is encoded by the open reading frame of the endogenous TRBC locus or a partial sequence thereof, and a further portion of the Cβ is encoded by the nucleic acid sequence of (a), wherein said further portion of Cβ is less than the full length of a native Cβ

116. The polynucleotide of embodiment 115, wherein the further portion of the Cβ is encoded by:

a sequence of nucleotides starting from residue 3 and up to residue 1445 of the sequence set forth in SEQ ID NO:2 or one or more exons thereof or a sequence that exhibits at least 85%, 86%, 87%, 88%, 89%, 90%, 91%, 92%, 93%, 94%, 95%, 96%, 97%, 98%, 99% or more sequence identity to a sequence of nucleotides starting from residue 3 and up to residue 1445 of the sequence set forth in SEQ ID NO:2 or one or more exons thereof, or a partial sequence thereof; or

a sequence of nucleotides starting from residue 3 and up to residue 1486 of the sequence set forth in SEQ ID NO:3 or one or more exons thereof or a sequence that exhibits at least 85%, 86%, 87%, 88%, 89%, 90%, 91%, 92%, 93%, 94%, 95%, 96%, 97%, 98%, 99% or more sequence identity to a sequence of nucleotides starting from residue 3 and up to residue 1486 of the sequence set forth in SEQ ID NO:3 or one or more exons thereof, or a partial sequence thereof.

117. The polynucleotide of embodiment 115 or 116, wherein the further portion of the Cβ is encoded by a sequence of nucleotides that is less than 400, less than 300, less than 250, less than 200, or less than 150 base pairs in length.

118. The polynucleotide of any of embodiments 115-117, wherein the further portion of the Cβ is encoded by a sequence of nucleotides that encodes less than four exons, less than three exons, less than two exons, one exon, or less than one full exon of the open reading frame of the TRBC locus.

119. The polynucleotide of any of embodiments 115-118, wherein the further portion of the Cβ is encoded by a portion of exon 1 of a TRBC locus, wherein the portion of exon 1 is less than the full length of exon 1 of the open reading frame of the TRBC locus.

120. The polynucleotide of embodiment 115-119, wherein the further portion of the TCRα constant domain is encoded by a portion of exon 1 of the TRAC locus, wherein the portion of exon 1 comprises a 5′ portion of exon 1.

121. The polynucleotide of any of embodiments 115-120, wherein the further portion of the Cβ and/or the Cα region encoded by the nucleic acid sequence of (a) comprises one or more modifications, optionally a replacement, deletion, or insertion of one or more amino acids compared to a native Cβ region and/or a native Cα region, optionally said one or more modifications introduces one or more cysteine residues that are capable of forming one or more non-native disulfide bridges between the alpha chain and beta chain.

122. The polynucleotide of any of embodiments 115-121, wherein the one or more homology arm comprises a 5′ homology arm and/or a 3′ homology arm.

123. The polynucleotide of embodiment 122, wherein the 5′ homology arm and 3′ homology arm comprises nucleic acid sequences homologous to nucleic acid sequences surrounding a target site, wherein the target site is within the open reading frame of the TRBC locus.

124. The polynucleotide of embodiment 123, wherein the target site is within exon 1 of the open reading frame of the TRBC locus.

125. The polynucleotide of any of embodiments 98-100 and 122-124, wherein the 5′ homology arm and 3′ homology arm independently are at least or at least about 10, 20, 30, 40, 50, 100, 200, 300, 400, 500, 600, 700, 800, 900, 1000, 1500, or 2000 nucleotides, or less than or less than about 10, 20, 30, 40, 50, 100, 200, 300, 400, 500, 600, 700, 800, 900, 1000, 1500, or 2000 nucleotides.

126. The polynucleotide of embodiment 125, wherein the 5′ homology arm and 3′ homology arm independently from or from about 100 to 1000 nucleotides, 100 to 750 nucleotides, 100 to 600 nucleotides, 100 to 400 nucleotides, 100 to 300 nucleotides, 100 to 200 nucleotides, 200 to 1000 nucleotides, 200 to 750 nucleotides, 200 to 600 nucleotides, 200 to 400 nucleotides, 200 to 300 nucleotides, 300 to 1000 nucleotides, 300 to 750 nucleotides, 300 to 600 nucleotides, 300 to 400 nucleotides, 400 to 1000 nucleotides, 400 to 750 nucleotides, 400 to 600 nucleotides, 600 to 1000 nucleotides, 600 to 750 nucleotides or 750 to 1000 nucleotides in length.

127. The polynucleotide of embodiment 125 or embodiment 126, wherein the 5′ homology arm and 3′ homology arm independently are, are about, or are less than about 600 nucleotides in length.

128. The polynucleotide of any of embodiments 79-127, wherein the nucleic acid sequence of (a) comprises one or more multicistronic element(s).

129. The polynucleotide of embodiment 128, wherein the multicistronic element(s) is positioned between the nucleic acid sequence encoding the TCRα or a portion thereof and the nucleic acid sequence encoding the TCRβ or a portion thereof.

130. The polynucleotide of embodiment 128 or embodiment 129, wherein the one or more multicistronic element(s) are upstream of the nucleic acid sequence encoding the TCR or a portion of the TCR or the nucleic acid molecule encoding the TCR.

131. The polynucleotide of any of embodiments 129 or embodiment 130, wherein the multicistronic element is or comprises a ribosome skip sequence, optionally T2A, P2A, E2A, or F2A.

132. The polynucleotide of any of embodiments 79-131, further comprising one or more heterologous regulatory or control element(s).

133. The polynucleotide of any of embodiments 79-132, wherein the nucleic acid sequence of (a) comprises one or more heterologous or regulatory control element(s) operably linked to control expression of the TCR when expressed from a cell introduced with the polynucleotide.

134. The polynucleotide of embodiment 132 or 133 wherein the one or more heterologous regulatory or control element comprises a promoter, an enhancer, an intron, a polyadenylation signal, a Kozak consensus sequence, a splice acceptor sequence and/or a splice donor sequence.

135. The polynucleotide of any of embodiments 132-134, wherein the heterologous regulatory or control element comprises a heterologous promoter.

136. The polynucleotide of embodiment 135, wherein the heterologous promoter is selected from among a constitutive promoter, an inducible promoter, a repressible promoter, and/or a tissue-specific promoter.

137. The polynucleotide of embodiment 135 or embodiment 136, wherein the heterologous promoter is or comprises a human elongation factor 1 alpha (EF1α) promoter or an MND promoter or a variant thereof.

138. The polynucleotide of any of embodiments 82-102 or 106-137, wherein the viral vector is an AAV vector.

139. The polynucleotide of embodiment 138, wherein the AAV vector is selected from among AAV1, AAV2, AAV3, AAV4, AAV5, AAV6, AAV7 or AAV8 vector.

140. The polynucleotide of embodiment 139, wherein the AAV vector is an AAV2 or AAV6 vector.

141. The polynucleotide of any of embodiments 82-102 or 106-137, wherein the viral vector is a retroviral vector.

142. The polynucleotide of any of embodiments 82-102 or 106-137, wherein the viral vector is a lentiviral vector.

143. The polynucleotide of any of embodiments 79-81, 84-105, or 107-137, wherein the polynucleotide is a linear polynucleotide.

144. The polynucleotide of embodiment 143, where in the linear polynucleotide is a double-stranded polynucleotide.

145. The polynucleotide of embodiment 144, where in the linear polynucleotide is a single-stranded polynucleotide.

146. The polynucleotide of any of embodiments 79-145, wherein the polynucleotide comprises the structure: [5′ homology arm]-[nucleic acid sequence of (a)]-[3′ homology arm].

147. The polynucleotide of any of embodiments 128-146, wherein the polynucleotide comprises the structure: [5′ homology arm]-[multicistronic element]-[nucleic acid sequence of (a)]-[3′ homology arm].

148. The polynucleotide of any of embodiments 134-147, wherein the polynucleotide comprises the structure: [5′ homology arm]-[promoter]-[nucleic acid sequence of (a)]-[3′ homology arm].

149. The polynucleotide of any of embodiments 79-148, wherein the recombinant TCR is capable of binding to an antigen that is associated with, specific to, and/or expressed on a cell or tissue that is associated with a disease, disorder, or condition.

150. The polynucleotide of embodiment 149, wherein the disease, disorder, or condition is an infectious disease or disorder, an autoimmune disease, an inflammatory disease, a tumor, or a cancer.

151. The polynucleotide of embodiment 149, wherein the antigen is a tumor antigen or a pathogenic antigen.

152. The polynucleotide of embodiment 151, wherein the pathogenic antigen is a bacterial antigen or viral antigen.

153. The polynucleotide of embodiment 152, wherein the antigen is a viral antigen, optionally a viral antigen from hepatitis A, hepatitis B, hepatitis C virus (HCV), human papilloma virus (HPV), hepatitis viral infections, Epstein-Barr virus (EBV), human herpes virus 8 (HHV-8), human T-cell leukemia virus-1 (HTLV-1), human T-cell leukemia virus-2 (HTLV-2), or a cytomegalovirus (CMV).

154. The polynucleotide of embodiment 152 or 153 wherein the antigen is an antigen from an HPV selected from among HPV-16, HPV-18, HPV-31, HPV-33 and HPV-35.

155. The polynucleotide of embodiment 152 or 153, wherein the antigen is an HPV-16 antigen that is an HPV-16 E6 or HPV-16 E7 antigen.

156 The polynucleotide of embodiment 152 or 153, wherein the viral antigen is an EBV antigen selected from among Epstein-Barr nuclear antigen (EBNA)-1, EBNA-2, EBNA-3A, EBNA-3B, EBNA-3C, EBNA-leader protein (EBNA-LP), latent membrane proteins LMP-1, LMP-2A and LMP-2B, EBV-EA, EBV-MA and EBV-VCA.

157. The polynucleotide of embodiment 152 or 153, wherein the viral antigen is an HTLV-antigen that is TAX.

158. The polynucleotide of embodiment 152 or 153, wherein the viral antigen is an HBV antigen that is a hepatitis B core antigen or a hepatitis B envelope antigen.

159. The polynucleotide of embodiment 151, wherein the antigen is a tumor antigen.

160. The polynucleotide of embodiment 159, wherein the antigen is selected from among glioma-associated antigen, 3-human chorionic gonadotropin, alpha fetoprotein (AFP), lectin-reactive AFP, thyroglobulin, RAGE-1, MN-CA IX, human telomerase reverse transcriptase, RUl, RU2 (AS), intestinal carboxyl esterase, mut hsp70-2, M-CSF, Melanin-A/MART-1, WT-1, S-100, MBP, CD63, MUC1 (e.g. MUC1-8), p53, Ras, cyclin B1, HER-2/neu, carcinoembryonic antigen (CEA), gp100, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A11, MAGE-B1, MAGE-B2, MAGE-B3, MAGE-B4, MAGE-C1, BAGE, GAGE-1, GAGE-2, p15, tyrosinase (e.g. tyrosinase-related protein 1 (TRP-1) or tyrosinase-related protein 2 (TRP-2)), β-catenin, NY-ESO-1, LAGE-1a, PP1, MDM2, MDM4, EGVFvIII, Tax, SSX2, telomerase, TARP, pp65, CDK4, vimentin, S100, eIF-4A1, IFN-inducible p78, melanotransferrin (p97), Uroplakin II, prostate specific antigen (PSA), human kallikrein (huK2), prostate specific membrane antigen (PSM), and prostatic acid phosphatase (PAP), neutrophil elastase, ephrin B2, BA-46, Bcr-abl, E2A-PRL, H4-RET, IGH-IGK, MYL-RAR, Caspase 8, FRa, CD24, CD44, CD133, CD 166, epCAM, CA-125, HE4, Oval, estrogen receptor, progesterone receptor, uPA, PAI-1, CD19, CD20, CD22, ROR1, CD33/IL3Ra, c-Met, PSMA, Glycolipid F77, GD-2, insulin growth factor (IGF)-I, IGF-II, IGF-I receptor and mesothelin.

161. A method of producing a genetically engineered T cell comprising a modified TRAC locus, comprising introducing the polynucleotide of any of embodiments 79-102 or 125-160 into a T cell comprising a genetic disruption at a TRAC locus.

162. A method of producing a genetically engineered T cell comprising a modified T cell receptor alpha constant (TRAC) locus, the method comprising:

(a) introducing, into a T cell, one or more agent(s) capable of inducing a genetic disruption at a target site within an endogenous TRAC locus of the T cell; and

(b) introducing into the T cell a polynucleotide comprising a transgene encoding a T cell receptor beta (TCRβ) chain and a portion of a T cell receptor alpha (TCRα) chain, wherein the portion is less than a full-length native TCRα chain, and wherein the transgene is targeted for integration within the endogenous TRAC locus via homology directed repair (HDR), thereby producing a genetically engineered cell comprising a modified TRAC locus.

163. A method of producing a genetically engineered T cell comprising a modified T cell receptor alpha constant (TRAC) locus, the method comprising introducing, into a T cell, a polynucleotide comprising a transgene encoding a T cell receptor beta (TCRβ) chain and a portion of a T cell receptor alpha (TCRα) chain, said T cell having a genetic disruption within the endogenous TRAC locus of the T cell, wherein the transgene is targeted for integration within the endogenous TRAC locus via homology directed repair (HDR), thereby producing a genetically engineered cell comprising a modified TRAC locus.

164. The method of embodiment 163, wherein the genetic disruption has been induced by one or more agent(s) capable of inducing a genetic disruption of one or more target site within the endogenous TRAC locus.

165. The method of any of embodiments 161-164, wherein the polynucleotide is from any of embodiments 79-102 or 125-160.

166. The method of any of embodiments 161-165, wherein the modified TRAC locus comprises a nucleic acid sequence encoding a recombinant TCR or portion thereof, wherein the nucleic acid sequence comprises an in-frame fusion of (i) a transgene sequence and (ii) an open reading frame or a partial sequence thereof of the endogenous TRAC locus.

167. The method of any of embodiments 161-166, wherein the transgene sequence does not comprise a sequence encoding a 3′ untranslated region (3′ UTR) or an intron.

168. The method of any of embodiments 161-167 wherein the open reading frame or a partial sequence thereof comprises a 3′ UTR of the endogenous TRAC locus.

169. The method of any of embodiments 161-168, wherein a portion of the Cα is encoded by the open reading frame of the endogenous TRAC locus or a partial sequence thereof, and a further portion of the Cα is encoded by the transgene sequence, wherein said further portion of Cα is less than the full length of a native Cα.

170. The method of any of embodiments 161-169, wherein the open reading frame or the partial sequence thereof comprises at least one intron and at least one exon of the endogenous TRAC locus.

171. The method of any of embodiments 161-170, wherein the transgene sequence is in-frame with one or more exons of the open reading frame or partial sequence thereof of the endogenous TRAC locus.

172. The method of any of embodiments 161-171, wherein the transgene sequence is integrated downstream of the most 5′ nucleotide of exon 1 and upstream of the most 3′ nucleotide of exon 1 of the open reading frame of the endogenous TRAC locus.

173. The method of any of embodiments 161-172, wherein the at least a portion of Cα is encoded by at least exons 2-4 of the open reading frame of the endogenous TRAC locus.

174. The method of any of embodiments 161-173, wherein the encoded Cα comprises the sequence selected from any one of SEQ ID NOS: 14, 15, 19, or 24, or a sequence that exhibits at least 85%, 86%, 87%, 88%, 89%, 90%, 91%, 92%, 93%, 94%, 95%, 96%, 97%, 98%, 99% or more sequence identity to any one of SEQ ID NOS: 14, 15, 19, or 24, or a partial sequence thereof.

175. The method of any of embodiments 169-174, wherein the further portion of the Cα comprises a sequence set forth in SEQ ID NO:142, or a sequence that exhibits at least 85%, 86%, 87%, 88%, 89%, 90%, 91%, 92%, 93%, 94%, 95%, 96%, 97%, 98%, 99% or more sequence identity to SEQ ID NO:142, or a partial sequence thereof.

176. The method of any of embodiments 161-175, wherein the engineered T cell further comprises inducing a genetic disruption at a TRBC locus.

177. The method of any of embodiments 161-174, wherein the engineered T cell comprises a genetic disruption at a TRBC1 locus and/or a TRBC2 locus.

178. A method of producing a genetically engineered T cell comprising a modified TRBC locus, comprising introducing the polynucleotide of any of embodiments 103-160 into a T cell comprising a genetic disruption at a TRBC locus.

179. A method of producing a genetically engineered T cell comprising a modified T cell receptor beta constant (TRBC) locus, the method comprising:

(a) introducing, into a T cell, one or more agent(s) capable of inducing a genetic disruption at a target site within an endogenous TRBC locus of the T cell; and

(b) introducing into the T cell a polynucleotide comprising a transgene encoding a T cell receptor alpha (TCRα) chain and a portion of a T cell receptor beta (TCRβ) chain, wherein the portion is less than a full-length native TCRβ chain, and wherein the transgene is targeted for integration within an endogenous TRBC locus via homology directed repair (HDR), thereby producing a genetically engineered cell comprising a modified TRBC locus.

180. A method of producing a genetically engineered T cell comprising a modified T cell receptor beta constant (TRBC) locus, the method comprising introducing, into a T cell, a polynucleotide comprising a transgene encoding a T cell receptor alpha (TCRα) chain and a portion of a T cell receptor beta (TCRβ) chain, said T cell having a genetic disruption within an endogenous TRBC locus of the T cell, wherein the transgene is targeted for integration within the endogenous TRBC locus via homology directed repair (HDR), thereby producing a genetically engineered cell comprising a modified TRBC locus.

181. The method of embodiment 180, wherein the genetic disruption has been induced by one or more agent(s) capable of inducing a genetic disruption of one or more target site within the endogenous TRBC locus.

182. The method of any of embodiments 178-181, wherein the TRBC locus is a TRBC1 locus and/or a TRBC2 locus.

183. The method of any of embodiments 178-182, wherein the polynucleotide is from any of embodiments 95-149.

184. The method of any of embodiments 178-183, wherein the modified TRBC locus comprises a nucleic acid sequence encoding a recombinant TCR or portion thereof, wherein the nucleic acid sequence comprises an in-frame fusion of (i) a transgene sequence and (ii) an open reading frame or a partial sequence thereof of the endogenous TRBC locus.

185. The method of any of embodiments 178-184, wherein the transgene sequence does not comprise a sequence encoding a 3′ untranslated region (3′ UTR) or an intron.

186. The method of any of embodiments 178-185, wherein the open reading frame or a partial sequence thereof comprises a 3′ UTR of the endogenous TRBC locus.

187. The method of any of embodiments 178-186, wherein a portion of the Cβ is encoded by the open reading frame of the endogenous TRBC locus or a partial sequence thereof, and a further portion of the Cβ is encoded by the transgene sequence, wherein said further portion of Cβ is less than the full length of a native Cβ.

188. The method of any of embodiments 178-187, wherein the open reading frame or the partial sequence thereof comprises at least one intron and at least one exon of the endogenous TRBC locus.

189. The method of any of embodiments 178-188, wherein the transgene sequence is in-frame with one or more exons of the open reading frame or partial sequence thereof of the endogenous TRBC locus.

190. The method of any of embodiments 178-189, wherein the transgene sequence is integrated downstream of the most 5′ nucleotide of exon 1 and upstream of the most 3′ nucleotide of exon 1 of the open reading frame of the endogenous TRBC locus.

191. The method of any of embodiments 178-190, wherein the at least a portion of Cβ is encoded by at least exons 2-4 of the open reading frame of the endogenous TRBC locus.

192. The method of any of embodiments 178-191, wherein the encoded Cβ comprises the sequence selected from any one of SEQ ID NO: 16, 17, 21, or 25, or a sequence that exhibits at least 85%, 86%, 87%, 88%, 89%, 90%, 91%, 92%, 93%, 94%, 95%, 96%, 97%, 98%, 99% or more sequence identity to SEQ ID NO: 16, 17, 21, or 25, or a partial sequence thereof.

193. The method of any of embodiments 177-192, wherein the engineered T cell further comprises inducing a genetic disruption at a TRAC locus.

194. The method of any of embodiments, wherein the one or more agent(s) capable of inducing a genetic disruption comprises a DNA binding protein or DNA-binding nucleic acid that specifically binds to or hybridizes to the target site.

195. The method of embodiment 194, wherein the one or more agent capable of inducing a genetic disruption comprises (a) a fusion protein comprising a DNA-targeting protein and a nuclease or (b) an RNA-guided nuclease.

196. The method of embodiment 195, wherein the DNA-targeting protein or RNA-guided nuclease comprises a zinc finger protein (ZFP), a TAL protein, or a clustered regularly interspaced short palindromic nucleic acid (CRISPR)-associated nuclease (Cas) specific for the target site.

197. The method of any of embodiments 194-196, wherein the one or more agent comprises a zinc finger nuclease (ZFN), a TAL-effector nuclease (TALEN), or and a CRISPR-Cas9 combination that specifically binds to, recognizes, or hybridizes to the target site.

198. The method of any of embodiments 162, 164-177, 179, or 181-197, wherein the each of the one or more agent comprises a guide RNA (gRNA) having a targeting domain that is complementary to the at least one target site.

199. The method of embodiment 198, wherein the one or more agent is introduced as a ribonucleoprotein (RNP) complex comprising the gRNA and a Cas9 protein.

200. The method of embodiment 199, wherein the RNP is introduced via electroporation, particle gun, calcium phosphate transfection, cell compression or squeezing.

201. The method of embodiment 199 or embodiment 200, wherein the RNP is introduced via electroporation.

202. The method of any of embodiments 162, 164-177, 179, or 181-197, wherein the one or more agent is introduced as one or more polynucleotide encoding the gRNA and/or a Cas9 protein.

203. The method of any of embodiments 198-202, wherein the gRNA has a targeting domain that is complementary to a target site in a TRAC locus and comprises a sequence selected from UCUCUCAGCUGGUACACGGC (SEQ ID NO:28), UGGAUUUAGAGUCUCUCAGC (SEQ ID NO:29), ACACGGCAGGGUCAGGGUUC (SEQ ID NO:30), GAGAAUCAAAAUCGGUGAAU (SEQ ID NO:31), GCUGGUACACGGCAGGGUCA (SEQ ID NO:32), CUCAGCUGGUACACGGC (SEQ ID NO:33), UGGUACACGGCAGGGUC (SEQ ID NO:34), GCUAGACAUGAGGUCUA (SEQ ID NO:35), GUCAGAUUUGUUGCUCC (SEQ ID NO:36), UCAGCUGGUACACGGCA (SEQ ID NO:37), GCAGACAGACUUGUCAC (SEQ ID NO:38), GGUACACGGCAGGGUCA (SEQ ID NO:39), CUUCAAGAGCAACAGUGCUG (SEQ ID NO:40), AGAGCAACAGUGCUGUGGCC (SEQ ID NO:41), AAAGUCAGAUUUGUUGCUCC (SEQ ID NO:42), ACAAAACUGUGCUAGACAUG (SEQ ID NO:43), AAACUGUGCUAGACAUG (SEQ ID NO:44), UGUGCUAGACAUGAGGUCUA (SEQ ID NO:45), GGCUGGGGAAGAAGGUGUCUUC (SEQ ID NO:46), GCUGGGGAAGAAGGUGUCUUC (SEQ ID NO:47), GGGGAAGAAGGUGUCUUC (SEQ ID NO:48), GUUUUGUCUGUGAUAUACACAU (SEQ ID NO:49), GGCAGACAGACUUGUCACUGGAUU (SEQ ID NO:50), GCAGACAGACUUGUCACUGGAUU (SEQ ID NO:51), GACAGACUUGUCACUGGAUU (SEQ ID NO:52), GUGAAUAGGCAGACAGACUUGUCA (SEQ ID NO:53), GAAUAGGCAGACAGACUUGUCA (SEQ ID NO:54), GAGUCUCUCAGCUGGUACACGG (SEQ ID NO:55), GUCUCUCAGCUGGUACACGG (SEQ ID NO:56), GGUACACGGCAGGGUCAGGGUU (SEQ ID NO:57) and GUACACGGCAGGGUCAGGGUU (SEQ ID NO:58).

204. The method of embodiment 203, wherein the gRNA has a targeting domain comprising the sequence GAGAAUCAAAAUCGGUGAAU (SEQ ID NO:31).

205. The method of any of embodiments 198-202, wherein the gRNA has a targeting domain that is complementary to a target site in one or both of a TRBC1 and a TRBC2 gene and comprises a sequence selected from CACCCAGAUCGUCAGCGCCG (SEQ ID NO:59), CAAACACAGCGACCUCGGGU (SEQ ID NO:60), UGACGAGUGGACCCAGGAUA (SEQ ID NO:61), GGCUCUCGGAGAAUGACGAG (SEQ ID NO:62), GGCCUCGGCGCUGACGAUCU (SEQ ID NO:63), GAAAAACGUGUUCCCACCCG (SEQ ID NO:64), AUGACGAGUGGACCCAGGAU (SEQ ID NO:65), AGUCCAGUUCUACGGGCUCU (SEQ ID NO:66), CGCUGUCAAGUCCAGUUCUA (SEQ ID NO:67), AUCGUCAGCGCCGAGGCCUG (SEQ ID NO:68), UCAAACACAGCGACCUCGGG (SEQ ID NO:69), CGUAGAACUGGACUUGACAG (SEQ ID NO:70), AGGCCUCGGCGCUGACGAUC (SEQ ID NO:71), UGACAGCGGAAGUGGUUGCG (SEQ ID NO:72), UUGACAGCGGAAGUGGUUGC (SEQ ID NO:73), UCUCCGAGAGCCCGUAGAAC (SEQ ID NO:74), CGGGUGGGAACACGUUUUUC (SEQ ID NO:75), GACAGGUUUGGCCCUAUCCU (SEQ ID NO:76), GAUCGUCAGCGCCGAGGCCU (SEQ ID NO:77), GGCUCAAACACAGCGACCUC (SEQ ID NO:78), UGAGGGUCUCGGCCACCUUC (SEQ ID NO:79), AGGCUUCUACCCCGACCACG (SEQ ID NO:80), CCGACCACGUGGAGCUGAGC (SEQ ID NO:81), UGACAGGUUUGGCCCUAUCC (SEQ ID NO:82), CUUGACAGCGGAAGUGGUUG (SEQ ID NO:83), AGAUCGUCAGCGCCGAGGCC (SEQ ID NO:84), GCGCUGACGAUCUGGGUGAC (SEQ ID NO:85), UGAGGGCGGGCUGCUCCUUG (SEQ ID NO:86), GUUGCGGGGGUUCUGCCAGA (SEQ ID NO:87), AGCUCAGCUCCACGUGGUCG (SEQ ID NO:88), GCGGCUGCUCAGGCAGUAUC (SEQ ID NO:89), GCGGGGGUUCUGCCAGAAGG (SEQ ID NO:90), UGGCUCAAACACAGCGACCU (SEQ ID NO:91), ACUGGACUUGACAGCGGAAG (SEQ ID NO:92), GACAGCGGAAGUGGUUGCGG (SEQ ID NO:93), GCUGUCAAGUCCAGUUCUAC (SEQ ID NO:94), GUAUCUGGAGUCAUUGAGGG (SEQ ID NO:95), CUCGGCGCUGACGAUCU (SEQ ID NO:96), CCUCGGCGCUGACGAUC (SEQ ID NO:97), CCGAGAGCCCGUAGAAC (SEQ ID NO:98), CCAGAUCGUCAGCGCCG (SEQ ID NO:99), GAAUGACGAGUGGACCC (SEQ ID NO:100), GGGUGACAGGUUUGGCCCUAUC (SEQ ID NO:101), GGUGACAGGUUUGGCCCUAUC (SEQ ID NO:102), GUGACAGGUUUGGCCCUAUC (SEQ ID NO:103), GACAGGUUUGGCCCUAUC (SEQ ID NO:104), GAUACUGCCUGAGCAGCCGCCU (SEQ ID NO:105), GACCACGUGGAGCUGAGCUGGUGG (SEQ ID NO:106), GUGGAGCUGAGCUGGUGG (SEQ ID NO:107), GGGCGGGCUGCUCCUUGAGGGGCU (SEQ ID NO:10⁸), GGCGGGCUGCUCCUUGAGGGGCU (SEQ ID NO:109), GCGGGCUGCUCCUUGAGGGGCU (SEQ ID NO:110), GGGCUGCUCCUUGAGGGGCU (SEQ ID NO:111), GGCUGCUCCUUGAGGGGCU (SEQ ID NO:112), GCUGCUCCUUGAGGGGCU (SEQ ID NO:113), GGUGAAUGGGAAGGAGGUGCACAG (SEQ ID NO:114), GUGAAUGGGAAGGAGGUGCACAG (SEQ ID NO:115) and GAAUGGGAAGGAGGUGCACAG (SEQ ID NO:116).

206. The method of embodiment 205, wherein the gRNA has a targeting domain comprising the sequence GGCCUCGGCGCUGACGAUCU (SEQ ID NO:63).

207. The method of embodiment of any of embodiments 161-206, wherein the T cell is a CD8+ T cell or subtypes thereof.

208. The method of embodiment 207, wherein the T cell is a CD4+ T cell or subtypes thereof.

209. The method of any of embodiments 161-206, wherein the T cell is derived from a multipotent or pluripotent cell, which optionally is an iPSC.

210. The method of any of embodiments 161-209, wherein the T cell comprises a T cell that is autologous to the subject.

211. The method of any of embodiments 161-209, wherein the T cell comprises a T cell that is allogeneic to the subject.

212. The method of any of embodiments 161-211, wherein the polynucleotide and/or the one or more polynucleotide encoding the gRNA and/or a Cas9 protein is comprised in one or more vector(s), which optionally are viral vector(s).

213. The method of embodiment 212, wherein the vector is an AAV vector.

214. The method of embodiment 213, wherein the AAV vector is selected from among AAV1, AAV2, AAV3, AAV4, AAV5, AAV6, AAV7 or AAV8 vector.

215. The method of embodiment 214, wherein the AAV vector is an AAV2 or AAV6 vector.

216. The method of embodiment 212, wherein the viral vector is a retroviral vector.

217. The method of embodiment 216, wherein the viral vector is a lentiviral vector.

218. The method of any of embodiments 161-211, wherein the polynucleoetide is a linear polynucleotide.

219. The method of embodiment 218, where in the linear polynucleotide is a double-stranded polynucleotide.

220. The method of embodiment 219, where in the linear polynucleotide is a single-stranded polynucleotide.

221. The method of any of embodiments 161-220, wherein the introduction of the one or more agent capable of inducing a genetic disruption and the introduction of the template polynucleotide are performed simultaneously or sequentially, in any order.

222. The method of any of embodiments 161-221, wherein the introduction of the template polynucleotide is performed after the introduction of the one or more agent capable of inducing a genetic disruption.

223. The method of embodiment 222, wherein the template polynucleotide is introduced immediately after, or within about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after the introduction of one or more agents 23 capable of inducing a genetic disruption.

224. An engineered T cell or a plurality of engineered T cells generated using the method of any of embodiments 161-2.

225. A composition, comprising the engineered T cell or plurality of engineered cells of embodiment 224.

226. The composition of embodiment 225, comprising CD4+ and/or CD8+ T cells.

227. The composition of embodiment 225 or embodiment 226, wherein the composition comprises CD4+ and CD8+ T cells and the ratio of CD4+ to CD8+ T cells is from or from about 1:3 to 3:1, optionally 1:1.

228. The composition of any of embodiments 225-227, wherein:

at least 70%, 75%, 80%, 90%, 91%, 92%, 93%, 94%, 95%, 96%, 97%, 98%, or 99% of the engineered cells in the composition comprise a genetic disruption in or of an endogenous T cell receptor alpha constant region (TRAC) gene and/or a T cell receptor beta constant region (TRBC) gene; and/or

at least 80%, 90%, 91%, 92%, 93%, 94%, 95%, 96%, 97%, 98%, or 99% of the engineered cells in the composition do not express or do not express detectable levels of a gene product of an endogenous TRAC or TRBC gene.

229. The composition of any of embodiments 225-228, wherein at least or greater than 35%, 40%, 45%, 50%, 55%, 60%, 65%, 70%, 75%, 80%, or 90% of the cells in the composition express the recombinant TCR and/or exhibit antigen binding.

230. A method of treatment comprising administering the engineered cell, plurality of engineered cells or composition of any of embodiments 224-229 to a subject.

231. Use of the engineered cell, plurality of engineered cells or composition of any of embodiments 224-229 for the treatment of a disease or disorder.

232. Use of the engineered cell, plurality of engineered cells or composition of any of embodiments 224-229 in the manufacture of a medicament for treating a disease or disorder.

233. The engineered cell, plurality of engineered cells or composition of any of embodiments 224-229 for use in treating a disease or disorder.

234. An article of manufacture comprising:

the polynucleotide of any of embodiments 79-102 or 116-149, and

one or more agent(s) capable of inducing a genetic disruption at a target site within a TRAC locus.

235. An article of manufacture comprising:

a polynucleotide comprising (a) a nucleic acid sequence encoding a T cell receptor beta (TCRβ) chain and a portion of a T cell receptor alpha (TCRα) chain, wherein the portion is less than a full-length native TCRα chain and (b) one or more homology arm(s) linked to the nucleic acid sequence, wherein the one or more homology arm(s) comprise a sequence homologous to one or more region(s) of an open reading frame of a TRAC locus, said open reading frame encoding a TCRα chain; and

one or more agent(s) capable of inducing a genetic disruption at a target site within a TRAC locus.

236. The article of manufacture of embodiment 201, wherein the polynucleotide is from any of embodiments 79-102 or 125-160.

237. An article of manufacture comprising:

the polynucleotide of any of embodiments 79-160, and

one or more agent(s) capable of inducing a genetic disruption at a target site within a TRBC locus.

238. An article of manufacture comprising:

a polynucleotide comprising (a) a nucleic acid sequence encoding a T cell receptor alpha (TCRα) chain and a portion of a T cell receptor beta (TCRβ) chain, wherein the portion is less than a full-length native TCRβ chain and (b) one or more homology arm(s) linked to the nucleic acid sequence, wherein the one or more homology arm(s) comprise a sequence homologous to one or more region(s) of an open reading frame of a TRBC locus, said open reading frame encoding a TCRβ chain; and

one or more agent(s) capable of inducing a genetic disruption at a target site within a TRAC locus.

239. The article of manufacture of embodiment 237 or 238, wherein the TRBC locus is TRBC1 and/or TRBC2.

240. The article of manufacture of embodiment 237 or 238, wherein the polynucleotide is from any of embodiments 103-160.

241. The article of manufacture of any of embodiments 234-241, wherein the one or more agent(s) capable of inducing a genetic disruption comprises a DNA binding protein or DNA-binding nucleic acid that specifically binds to or hybridizes to the target site.

242. The article of manufacture of embodiment 241, wherein the one or more agent capable of inducing a genetic disruption comprises (a) a fusion protein comprising a DNA-targeting protein and a nuclease or (b) an RNA-guided nuclease.

243. The article of manufacture of embodiment 242, wherein the DNA-targeting protein or RNA-guided nuclease comprises a zinc finger protein (ZFP), a TAL protein, or a clustered regularly interspaced short palindromic nucleic acid (CRISPR)-associated nuclease (Cas) specific for the target site.

244. The article of manufacture of any of embodiments 241-243, wherein the one or more agent comprises a zinc finger nuclease (ZFN), a TAL-effector nuclease (TALEN), or and a CRISPR-Cas9 combination that specifically binds to, recognizes, or hybridizes to the target site.

245. The article of manufacture of any of embodiments 241-244, wherein the each of the one or more agent comprises a guide RNA (gRNA) having a targeting domain that is complementary to the at least one target site.

246. The article of manufacture of embodiment 241-245, wherein the one or more agent is introduced as a ribonucleoprotein (RNP) complex comprising the gRNA and a Cas9 protein.

247. The article of manufacture of embodiment 246, wherein the RNP is introduced via electroporation, particle gun, calcium phosphate transfection, cell compression or squeezing.

248. The article of manufacture of embodiment 246 or embodiment 247, wherein the RNP is introduced via electroporation.

249. The article of manufacture of any of embodiments 245-248, wherein the one or more agent is introduced as one or more polynucleotide encoding the gRNA and/or a Cas9 protein.

250. The article of manufacture of any of embodiments 245-249, wherein the gRNA has a targeting domain that is complementary to a target site in a TRAC locus and comprises a sequence selected from UCUCUCAGCUGGUACACGGC (SEQ ID NO:28), UGGAUUUAGAGUCUCUCAGC (SEQ ID NO:29), ACACGGCAGGGUCAGGGUUC (SEQ ID NO:30), GAGAAUCAAAAUCGGUGAAU (SEQ ID NO:31), GCUGGUACACGGCAGGGUCA (SEQ ID NO:32), CUCAGCUGGUACACGGC (SEQ ID NO:33), UGGUACACGGCAGGGUC (SEQ ID NO:34), GCUAGACAUGAGGUCUA (SEQ ID NO:35), GUCAGAUUUGUUGCUCC (SEQ ID NO:36), UCAGCUGGUACACGGCA (SEQ ID NO:37), GCAGACAGACUUGUCAC (SEQ ID NO:38), GGUACACGGCAGGGUCA (SEQ ID NO:39), CUUCAAGAGCAACAGUGCUG (SEQ ID NO:40), AGAGCAACAGUGCUGUGGCC (SEQ ID NO:41), AAAGUCAGAUUUGUUGCUCC (SEQ ID NO:42), ACAAAACUGUGCUAGACAUG (SEQ ID NO:43), AAACUGUGCUAGACAUG (SEQ ID NO:44), UGUGCUAGACAUGAGGUCUA (SEQ ID NO:45), GGCUGGGGAAGAAGGUGUCUUC (SEQ ID NO:46), GCUGGGGAAGAAGGUGUCUUC (SEQ ID NO:47), GGGGAAGAAGGUGUCUUC (SEQ ID NO:48), GUUUUGUCUGUGAUAUACACAU (SEQ ID NO:49), GGCAGACAGACUUGUCACUGGAUU (SEQ ID NO:50), GCAGACAGACUUGUCACUGGAUU (SEQ ID NO:51), GACAGACUUGUCACUGGAUU (SEQ ID NO:52), GUGAAUAGGCAGACAGACUUGUCA (SEQ ID NO:53), GAAUAGGCAGACAGACUUGUCA (SEQ ID NO:54), GAGUCUCUCAGCUGGUACACGG (SEQ ID NO:55), GUCUCUCAGCUGGUACACGG (SEQ ID NO:56), GGUACACGGCAGGGUCAGGGUU (SEQ ID NO:57) and GUACACGGCAGGGUCAGGGUU (SEQ ID NO:58).

251. The article of manufacture of embodiment 250, wherein the gRNA has a targeting domain comprising the sequence GAGAAUCAAAAUCGGUGAAU (SEQ ID NO:31).

252. The article of manufacture of any of embodiments 245-251, wherein the gRNA has a targeting domain that is complementary to a target site in one or both of a TRBC1 and a TRBC2 gene and comprises a sequence selected from CACCCAGAUCGUCAGCGCCG (SEQ ID NO:59), CAAACACAGCGACCUCGGGU (SEQ ID NO:60), UGACGAGUGGACCCAGGAUA (SEQ ID NO:61), GGCUCUCGGAGAAUGACGAG (SEQ ID NO:62), GGCCUCGGCGCUGACGAUCU (SEQ ID NO:63), GAAAAACGUGUUCCCACCCG (SEQ ID NO:64), AUGACGAGUGGACCCAGGAU (SEQ ID NO:65), AGUCCAGUUCUACGGGCUCU (SEQ ID NO:66), CGCUGUCAAGUCCAGUUCUA (SEQ ID NO:67), AUCGUCAGCGCCGAGGCCUG (SEQ ID NO:68), UCAAACACAGCGACCUCGGG (SEQ ID NO:69), CGUAGAACUGGACUUGACAG (SEQ ID NO:70), AGGCCUCGGCGCUGACGAUC (SEQ ID NO:71), UGACAGCGGAAGUGGUUGCG (SEQ ID NO:72), UUGACAGCGGAAGUGGUUGC (SEQ ID NO:73), UCUCCGAGAGCCCGUAGAAC (SEQ ID NO:74), CGGGUGGGAACACGUUUUUC (SEQ ID NO:75), GACAGGUUUGGCCCUAUCCU (SEQ ID NO:76), GAUCGUCAGCGCCGAGGCCU (SEQ ID NO:77), GGCUCAAACACAGCGACCUC (SEQ ID NO:78), UGAGGGUCUCGGCCACCUUC (SEQ ID NO:79), AGGCUUCUACCCCGACCACG (SEQ ID NO:80), CCGACCACGUGGAGCUGAGC (SEQ ID NO:81), UGACAGGUUUGGCCCUAUCC (SEQ ID NO:82), CUUGACAGCGGAAGUGGUUG (SEQ ID NO:83), AGAUCGUCAGCGCCGAGGCC (SEQ ID NO:84), GCGCUGACGAUCUGGGUGAC (SEQ ID NO:85), UGAGGGCGGGCUGCUCCUUG (SEQ ID NO:86), GUUGCGGGGGUUCUGCCAGA (SEQ ID NO:87), AGCUCAGCUCCACGUGGUCG (SEQ ID NO:88), GCGGCUGCUCAGGCAGUAUC (SEQ ID NO:89), GCGGGGGUUCUGCCAGAAGG (SEQ ID NO:90), UGGCUCAAACACAGCGACCU (SEQ ID NO:91), ACUGGACUUGACAGCGGAAG (SEQ ID NO:92), GACAGCGGAAGUGGUUGCGG (SEQ ID NO:93), GCUGUCAAGUCCAGUUCUAC (SEQ ID NO:94), GUAUCUGGAGUCAUUGAGGG (SEQ ID NO:95), CUCGGCGCUGACGAUCU (SEQ ID NO:96), CCUCGGCGCUGACGAUC (SEQ ID NO:97), CCGAGAGCCCGUAGAAC (SEQ ID NO:98), CCAGAUCGUCAGCGCCG (SEQ ID NO:99), GAAUGACGAGUGGACCC (SEQ ID NO:100), GGGUGACAGGUUUGGCCCUAUC (SEQ ID NO:101), GGUGACAGGUUUGGCCCUAUC (SEQ ID NO:102), GUGACAGGUUUGGCCCUAUC (SEQ ID NO:103), GACAGGUUUGGCCCUAUC (SEQ ID NO:104), GAUACUGCCUGAGCAGCCGCCU (SEQ ID NO:105), GACCACGUGGAGCUGAGCUGGUGG (SEQ ID NO:106), GUGGAGCUGAGCUGGUGG (SEQ ID NO:107), GGGCGGGCUGCUCCUUGAGGGGCU (SEQ ID NO:108), GGCGGGCUGCUCCUUGAGGGGCU (SEQ ID NO:109), GCGGGCUGCUCCUUGAGGGGCU (SEQ ID NO:110), GGGCUGCUCCUUGAGGGGCU (SEQ ID NO:111), GGCUGCUCCUUGAGGGGCU (SEQ ID NO:112), GCUGCUCCUUGAGGGGCU (SEQ ID NO:113), GGUGAAUGGGAAGGAGGUGCACAG (SEQ ID NO:114), GUGAAUGGGAAGGAGGUGCACAG (SEQ ID NO:115) and GAAUGGGAAGGAGGUGCACAG (SEQ ID NO:116).

253. The article of manufacture of embodiment 252, wherein the gRNA has a targeting domain comprising the sequence GGCCUCGGCGCUGACGAUCU (SEQ ID NO:63).

254. A kit comprising an article of manufacture of any of embodiments 234-253, and instructions for use.

255. The kit of embodiment 254, wherein the instructions specify that the one or more agent(s) and the polynucleotide are introduced into the cell.

256. The kit of embodiment 254 or 255, wherein the instructions specify that the one or more agent(s) and the polynucleotide are introduced simultaneously or sequentially, in any order.

257. The kit of any of embodiments 254-257, wherein the instructions specify that the introduction of the polynucleotide is performed after the introduction of the one or more agent(s).

258 The kit of embodiment 257, wherein the instructions specify that the polynucleotide is introduced immediately after, or within about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after the introduction of one or more agents capable of inducing a genetic disruption.

### X. EXAMPLES

The following examples are included for illustrative purposes only and are not intended to limit the scope of the invention.

### Example 1: Generation of Engineered T Cells Expressing a Recombinant TCR

T cells were engineered to contain genetic disruptions of the endogenous TCR α constant (TRAC) and TCR β constant (TRBC) gene loci and engineered to express a recombinant T cell receptor (TCR) by targeted integration of the polynucleotide encoding the recombinant TCR into the endogenous TRAC locus.

A. Recombinant TCR Transgene Constructs

Polynucleotide constructs encoding an exemplary recombinant TCR that recognizes an epitope of the human papilloma virus (HPV) 16 oncoprotein E7 were generated containing a full TCRβ chain sequence and a partial TCRα chain sequence separated by a 2A ribosome skip sequence. The partial recombinant TCRα-encoding sequence included a full TCRα variable region sequence and a partial recombinant TCRα constant region sequence corresponding to a 5′ portion of exon 1 of the TRAC gene (set forth in SEQ ID NO: 142).

The sequences encoding the recombinant TCRs were also codon optimized and/or modified by mutation(s) to promote the formation of a non-native disulfide bond in the interface between the TCR constant domains to increase pairing and stability of the TCR. The non-native disulfide bond was promoted by modifying the TCR chains at residue 48 in exon 1 of the TCRα chain constant region (Ca) from Thr to Cys and residue 57 of the TCRβ chain constant region (Cβ) from Ser to Cys (see Kuball et al. (2007) Blood, 109:2331-2338). The construct was designed to produce an mRNA encoding full TCRβ and TCRα chains by incorporation of the partial recombinant TCRα exon 1 sequence, including the sequence encoding the modified Cys residue, into exon 1 of the endogenous TRAC gene, such that the endogenous TRAC gene exon 1 sequence is in frame with the partial recombinant TCRα-encoding sequence.

The sequences encoding the recombinant TCR were placed under the operable control of the human elongation factor 1 alpha (EF1α) promoter (sequence set forth in SEQ ID NO: 127), the MND promoter (sequence set forth in SEQ ID NO: 126), which is a synthetic promoter that contains the U3 region of a modified MoMuLV LTR with myeloproliferative sarcoma virus enhancer, or a P2A ribosome skipping element (sequence set forth in SEQ ID NO: 6) followed by the sequences encoding the recombinant TCR to drive expression of the recombinant TCR from the endogenous TCRα promoter upon HDR-mediated targeted integration in-frame into the human TRAC gene.

For targeted integration by HDR, an adeno-associated virus (AAV) vector construct was generated containing the exemplary recombinant TCR transgene flanked on the 5′ and 3′ ends by 600 bp homology arms that were homologous to the sequence surrounding the target integration site and for integration of the transgene to produce an in-frame fusion mRNA (5′ homology arm sequence set forth in SEQ ID NO: 124; 3′ homology arm sequence set forth in SEQ ID NO: 125).

Polynucleotides encoding the full TCRβ and TCRα chains of the exemplary TCR sequence that separated by P2A ribosomal skip sequences were also generated. These polynucleotides contained an SV40 poly A region placed 3′ of the sequence encoding the full TCRα chain and were flanked on each side by 600 bp homology arms that were homologous to sequences surrounding the genetic disruption at the TRAC gene.

AAV stocks were produced by triple transfection of an AAV vector that included the polynucleotides encoding the exemplary recombinant TCR, serotype-6 helper, and adenoviral helper plasmids into a 293T-17 cell line. Transfected cells were collected and lysed, and AAV stock was collected for transduction of cells.

B. Generation of Engineered T Cells

Primary human CD4+ and CD8+ T cells were isolated by immunoaffinity-based selection from human peripheral blood mononuclear cells (PBMCs) obtained from healthy donors. The resulting cells were stimulated for 72 hours by culturing with an anti-CD3/anti-CD28 reagent at 1:1 cell to bead ratio in media containing human serum, IL-2 (100 U/mL), IL-7 (10 ng/mL) and IL-15 (5 ng/mL) at 37° C. Following stimulation, CD3/CD28 beads were removed magnetically and the cells were washed with PBS prior to electroporation.

To knock out the endogenous TCR genes, cells were electroporated with ribonucleoprotein (RNP) complexes containing a guide RNA (gRNA) designed to target a target site within exon 1 of the TCR α constant regions gene (target site sequence for TRAC set forth in SEQ ID NO: 117 and a consensus target site sequence common to exon 1 of both TCR β constant regions 1 and 2 (target site sequence for TRBC set forth in SEQ ID NO: 118) and Streptococcus pyogenes Cas9 protein. The cells then were cultured in the media containing human serum and IL-2 (50 U/mL), IL-7 (5 ng/mL) and IL-15 (0.5 ng/mL).

For targeted integration of the recombinant TCR into the TRAC locus, cells were transduced with the appropriate AAV preparations described above, approximately 2 hours after RNP electroporation. The cells were subsequently expanded in culture media containing cytokines. Cells subject to mock transfection were used as control.

### Example 2: Assessment of Recombinant TCR Expression and Function

T cells were engineered to express the exemplary recombinant anti-HPV 16 E7 TCR as described in Example 1. On day 7 after transduction, the cells were assessed by flow cytometry for staining with antibodies for cell surface markers that included CD8, an anti-Vbeta22 antibody specific for the recombinant TCR, and with a peptide-MHC tetramer complexed with an antigen recognized by the recombinant TCR, HPV 16 E7(11-19) peptide (sequence set forth in SEQ ID NO: 143).

As shown in FIGS. 1A and 1B, HDR-mediated targeted knock-in of the recombinant TCR and knockout of endogenous TCRα and TCRβ resulted in expression of the recombinant TCR as indicated by the presence of CD8+ cells that express Vbeta22 (FIG. 1A) and bind to HPV16 E7(11-19)-MHC tetramer (FIG. 1B), as compared to mock transfection controls. Similar expression of the exemplary recombinant TCR was observed in cells following transduction with AAV encoding full recombinant TCRβ and TCRα chain sequences (designated SV40 pA), under control of the EF1α or MND promoter, as with AAV encoding the full recombinant TCRβ and fusion of recombinant and endogenous TCRα chain sequences (designated 3′ arm) under control of the endogenous TRAC promoter (designated P2A) or MND promoter.

Cytolytic activity was assessed by incubating recombinant TCR-expressing CD8+ effector cells with HPV-16 positive squamous cell carcinoma line, SCC-152, at an effector to target (E:T) ratio of 5:1. The target cells were labeled with NucLight Red (NLR) to permit tracking by fluorescent microscopy. As controls, co-cultures of target cells with CD8+ cells expressing a reference TCR capable of binding to HPV 16 E7 but containing mouse Cα and the Cβ regions were assessed (“E7 Ref mouse”) or mock transfected. As shown in FIG. 2, cytolytic activity of cells expressing the exemplary recombinant TCR, generated by transduction with AAV encoding full recombinant TCRβ and TCRα chain sequences (designated SV40 pA), under control of the MND promoter or the endogenous TRAC promoter (designated P2A); by with AAV encoding the full recombinant TCRβ and fusion of recombinant and endogenous TCRα chain sequences (designated 3′ arm) under control of the endogenous TRAC promoter or MND promoter were generally similar, and generally similar to the cytolytic activity of the reference TCR containing a mouse constant region.

### Example 3: Assessment of Homology Arm Lengths for Efficient HDR-Mediated Integration of Transgene Sequences

Polynucleotides containing an exemplary transgene, flanked by varying lengths of homology arms were introduced into T cells for targeted integration of the transgene via homology-dependent repair (HDR), and the efficiency of integration was assessed.

A. Recombinant Transgene Constructs and Generation of Engineered T Cells

Exemplary HDR template polynucleotides containing a transgene sequence encoding a green fluorescent protein (GFP), flanked by varying lengths of homology arms for targeted integration into the human TCRα constant region (TRAC) locus were introduced into T cells. Specifically, for integration by HDR, AAV preparations containing template nucleotide constructs encoding GFP were generated substantially as described in Example 1, except with the following differences: AAV constructs were generated containing a polynucleotide encoding GFP under the operable control of the MND promoter and linked to an SV40 poly(A) sequence, flanked by 50, 100, 200, 300, 400, 500, or 600 base pair of 5′ and 3′ homology arms (SEQ ID NOS: 229-235 and 236-242, respectively) homologous to sequences surrounding the target integration site in the human TCR α constant region (TRAC) gene. The total length of the AAV construct was made constant by using filler DNA sequences between the SV40 poly(A) sequence and the 3′ homology arm.

For targeted integration by HDR, primary human CD4+ and CD8+ T cells from four human donors (Donors 1-4) were combined at a 1:1 ratio, stimulated and subject to electroporation with ribonucleoprotein (RNP) complexes containing TRAC-targeting gRNA, generally as described in Examples 1-3. After electroporation, the cells were transduced with AAV preparations containing the HDR template polynucleotides containing various homology arm lengths described above. GFP expression was measured by flow cytometry at 24, 48, 72, 96 hours and 7 days after transduction with AAV, to determine an integration ratio (representing the percentage of total AAV inside of the cells that have integrated into the genome) based on the following formula:

$$Integration ratio = \frac{( \% high MFI GFP )}{( \% high MFI GFP + \% low MFI GFP )} .$$

The % high MFI and % low MFI indicate the percentage of cells that were above or below a threshold MFI by flow cytometry, representing cells containing integrated GFP transgene or non-integrated AAV construct, respectively. The changes in integration ratio with an increase of the length of the homology arm was assessed by subtracting the integration ratio of the next shortest arm length from the integration ratio of a particular arm length.

B. Assessment of Integration Ratio

As shown in FIGS. 3A-3B, a constant or increased integration ratio was observed at various time points assessed for each homology arm length, consistent with non-integrated AAV constructs diluting out over time. Assessing the changes in GFP expression patterns over time showed that the percentage of cells with integrated GFP transgene (high MFI) generally remained constant after 72 hours, but the percentage of cells containing non-integrated AAV only (low MFI) continued to decrease.

As shown in FIGS. 4A-4B, the most substantial gains in integration ratio appeared to occur at 200 bp of homology arm, with minor gains at 300 bp. No substantial gain was observed between 300 bp and 500 bp, and an increase in integration ratio was observed between 500 bp and 600 bp, in all donors. The results support using a minimum of 300 bp homology arms for high integration efficiency, with 600 bp homology arms providing even higher integration efficiency.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| # | SEQUENCE | ANNOTATION |
|---|---|---|
| 1 | atatccagaaccctgaccctgccgtgtaccagctgagagactctaaatccagtgac | Human TCR alpha |
| | aagtctgtctgcctattcaccgattttgattctcaaacaaatgtgtcacaaagtaa | constant (TRAC) |
| | ggattctgatgtgtatatcacagacaaaactgtgctagacatgaggtctatggact | NCBI Reference |
| | tcaagagcaacagtgctgtggcctggagcaacaaatctgactttgcatgtgcaaac | Sequence: |
| | gccttcaacaacagcattattccagaagacaccttcttccccagcccaggtaaggg | NG_001332.3, TRAC |
| | cagctttggtgccttcgcaggctgtttccttgcttcaggaatggccaggttctgcc | |
| | cagagctctggtcaatgatgtctaaaactcctctgattggtggtctcggccttatc | |
| | cattgccaccaaaaccctctttttactaagaaacagtgagccttgttctggcagtc | |
| | cagagaatgacacgggaaaaaagcagatgaagagaaggtggcaggagagggcacgt | |
| | ggcccagcctcagtctctccaactgagttcctgcctgcctgcctttgctcagactg | |
| | tttgccccttactgctcttctaggcctcattctaagccccttctccaagttgcctc | |
| | tccttatttctccctgtctgccaaaaaatctttcccagctcactaagtcagtctca | |
| | cgcagtcactcattaacccaccaatcactgattgtgccggcacatgaatgcaccag | |
| | gtgttgaagtggaggaattaaaaagtcagatgaggggtgtgcccagaggaagcacc | |
| | attctagttgggggagcccatctgtcagctgggaaaagtccaaataacttcagatt | |
| | ggaatgtgttttaactcagggttgagaaaacagctaccttcaggacaaaagtcagg | |
| | gaagggctctctgaagaaatgctacttgaagataccagccctaccaagggcaggga | |
| | gaggaccctatagaggcctgggacaggagctcaatgagaaaggagaagagcagcag | |
| | gcatgagttgaatgaaggaggcagggccgggtcacagggccttctaggccatgaga | |
| | gggtagacagtattctaaggacgccagaaagctgttgatcggcttcaagcagggga | |
| | gggacacctaatttgcttttcttttttttttttttttttttttttttttttgagat | |
| | ggagttttgctcttgttgcccaggctggagtgcaatggtgcatcttggctcactgc | |
| | aacctccgcctcccaggttcaagtgattctcctgcctcagcctcccgagtagctga | |
| | gattacaggcacccgccaccatgcctggctaattttttgtatttttagtagagaca | |
| | gggtttcactatgttggccaggctggtctcgaactcctgacctcaggtgatccacc | |
| | cgcttcagcctcccaaagtgctgggattacaggcgtgagccaccacacccggcctg | |
| | cttttcttaaagatcaatctgagtgctgtacggagagtgggttgtaagccaagagt | |
| | agaagcagaaagggagcagttgcagcagagagatgatggaggcctgggcagggtgg | |
| | tggcagggaggtaaccaacaccattcaggtttcaaaggtagaaccatgcagggatg | |
| | agaaagcaaagaggggatcaaggaaggcagctggattttggcctgagcagctgagt | |
| | caatgatagtgccgtttactaagaagaaaccaaggaaaaaatttggggtgcaggga | |
| | tcaaaactttttggaacatatgaaagtacgtgtttatactctttatggcccttgtc | |
| | actatgtatgcctcgctgcctccattggactctagaatgaagccaggcaagagcag | |
| | ggtctatgtgtgatggcacatgtggccagggtcatgcaacatgtactttgtacaaa | |
| | cagtgtatattgagtaaatagaaatggtgtccaggagccgaggtatcggtcctgcc | |
| | agggccaggggctctccctagcaggtgctcatatgctgtaagttccctccagatct | |
| | ctccacaaggaggcatggaaaggctgtagttgttcacctgcccaagaactaggagg | |
| | tctggggtgggagagtcagcctgctctggatgctgaaagaatgtctgtttttcctt | |
| | ttagaaagttcctgtgatgtcaagctggtcgagaaaagctttgaaacaggtaagac | |
| | aggggtctagcctgggtttgcacaggattgcggaagtgatgaacccgcaataaccc | |
| | tgcctggatgagggagtgggaagaaattagtagatgtgggaatgaatgatgaggaa | |
| | tggaaacagcggttcaagacctgcccagagctgggtggggtctctcctgaatccct | |
| | ctcaccatctctgactttccattctaagcactttgaggatgagtttctagcttcaa | |
| | tagaccaaggactctctcctaggcctctgtattcctttcaacagctccactgtcaa | |
| | gagagccagagagagcttctgggtggcccagctgtgaaatttctgagtcccttagg | |
| | gatagccctaaacgaaccagatcatcctgaggacagccaagaggttttgccttctt | |
| | tcaagacaagcaacagtactcacataggctgtgggcaatggtcctgtctctcaaga | |
| | atcccctgccactcctcacacccaccctgggcccatattcatttccatttgagttg | |
| | ttcttattgagtcatccttcctgtggtagcggaactcactaaggggcccatctgga | |
| | cccgaggtattgtgatgataaattctgagcacctaccccatccccagaagggctca | |
| | gaaataaaataagagccaagtctagtcggtgtttcctgtcttgaaacacaatactg | |
| | ttggccctggaagaatgcacagaatctgtttgtaaggggatatgcacagaagctgc | |
| | aagggacaggaggtgcaggagctgcaggcctcccccacccagcctgctctgccttg | |
| | gggaaaaccgtgggtgtgtcctgcaggccatgcaggcctgggacatgcaagcccat | |
| | aaccgctgtggcctcttggttttacagatacgaacctaaactttcaaaacctgtca | |
| | gtgattgggttccgaatcctcctcctgaaagtggccgggtttaatctgctcatgac | |
| | gctgcggctgtggtccagctgaggtgaggggccttgaagctgggagtggggtttag | |
| | ggacgcgggtctctgggtgcatcctaagctctgagagcaaacctccctgcagggtc | |
| | ttgcttttaagtccaaagcctgagcccaccaaactctcctacttcttcctgttaca | |
| | aattcctcttgtgcaataataatggcctgaaacgctgtaaaatatcctcatttcag | |
| | ccgcctcagttgcacttctcccctatgaggtaggaagaacagttgtttagaaacga | |
| | agaaactgaggccccacagctaatgagtggaggaagagagacacttgtgtacacca | |
| | catgccttgtgttgtacttctctcaccgtgtaacctcctcatgtcctctctcccca | |
| | gtacggctctcttagctcagtagaaagaagacattacactcatattacaccccaat | |
| | cctggctagagtctccgcaccctcctcccccagggtccccagtcgtcttgctgaca | |
| | actgcatcctgttccatcaccatcaaaaaaaaactccaggctgggtgcgggggctc | |
| | acacctgtaatcccagcactttgggaggcagaggcaggaggagcacaggagctgga | |
| | gaccagcctgggcaacacagggagaccccgcctctacaaaaagtgaaaaaattaac | |
| | caggtgtggtgctgcacacctgtagtcccagctacttaagaggctgagatgggagg | |
| | atcgcttgagccctggaatgttgaggctacaatgagctgtgattgcgtcactgcac | |
| | tccagcctggaagacaaagcaagatcctgtctcaaataataaaaaaaataagaact | |
| | ccagggtacatttgctcctagaactctaccacatagccccaaacagagccatcacc | |
| | atcacatccctaacagtcctgggtcttcctcagtgtccagcctgacttctgttctt | |
| | cctcattccagatctgcaagattgtaagacagcctgtgctccctcgctccttcctc | |
| | tgcattgcccctcttctccctctccaaacagagggaactctcctacccccaaggag | |
| | gtgaaagctgctaccacctctgtgcccccccggcaatgccaccaactggatcctac | |
| | ccgaatttatgattaagattgctgaagagctgccaaacactgctgccaccccctct | |
| | gttcccttattgctgcttgtcactgcctgacattcacggcagaggcaaggctgctg | |
| | cagcctcccctggctgtgcacattccctcctgctccccagagactgcctccgccat | |
| | cccacagatgatggatcttcagtgggttctcttgggctctaggtcctgcagaatgt | |
| | tgtgaggggtttatttttttttaatagtgttcataaagaaatacatagtattcttc | |
| | ttctcaagacgtggggggaaattatctcattatcgaggccctgctatgctgtgtat | |
| | ctgggcgtgttgtatgtcctgctgccgatgccttc | |
| 2 | aggacctgaacaaggtgttcccacccgaggtcgctgtgtttgagccatcagaagca | Human TCR beta |
| | gagatctcccacacccaaaaggccacactggtgtgcctggccacaggcttcttccc | constant 1 (TRBC1) |
| | cgaccacgtggagctgagctggtgggtgaatgggaaggaggtgcacagtggggtca | NCBI Reference |
| | gcacagacccgcagcccctcaaggagcagcccgccctcaatgactccagatactgc | Sequence: |
| | ctgagcagccgcctgagggtctcggccaccttctggcagaacccccgcaaccactt | NG_001333.2, |
| | ccgctgtcaagtccagttctacgggctctcggagaatgacgagtggacccaggata | TRBC1 |
| | gggccaaacccgtcacccagatcgtcagcgccgaggcctggggtagagcaggtgag | |
| | tggggcctggggagatgcctggaggagattaggtgagaccagctaccagggaaaat | |
| | ggaaagatccaggtagcagacaagactagatccaaaaagaaaggaaccagcgcaca | |
| | ccatgaaggagaattgggcacctgtggttcattcttctcccagattctcagcccaa | |
| | cagagccaagcagctgggtcccctttctatgtggcctgtgtaactctcatctgggt | |
| | ggtgccccccatccccctcagtgctgccacatgccatggattgcaaggacaatgtg | |
| | gctgacatctgcatggcagaagaaaggaggtgctgggctgtcagaggaagctggtc | |
| | tgggcctgggagtctgtgccaactgcaaatctgactttacttttaattgcctatga | |
| | aaataaggtctctcatttattttcctctccctgctttctttcagactgtggcttta | |
| | cctcgggtaagtaagcccttccttttcctctccctctctcatggttcttgacctag | |
| | aaccaaggcatgaagaactcacagacactggagggtggagggtgggagagaccaga | |
| | gctacctgtgcacaggtacccacctgtccttcctccgtgccaacagtgtcctacca | |
| | gcaaggggtcctgtctgccaccatcctctatgagatcctgctagggaaggccaccc | |
| | tgtatgctgtgctggtcagcgcccttgtgttgatggccatggtaagcaggagggca | |
| | ggatggggccagcaggctggaggtgacacactgacaccaagcacccagaagtatag | |
| | agtccctgccaggattggagctgggcagtagggagggaagagatttcattcaggtg | |
| | cctcagaagataacttgcacctctgtaggatcacagtggaagggtcatgctgggaa | |
| | ggagaagctggagtcaccagaaaacccaatggatgttgtgatgagccttactattt | |
| | gtgtggtcaatgggccctactactttctctcaatcctcacaactcctggctcttaa | |
| | taacccccaaaactttctcttctgcaggtcaagagaaaggatttctga | |
| 3 | aggacctgaaaaacgtgttcccacccgaggtcgctgtgtttgagccatcagaagca | Human TCR beta |
| | gagatctcccacacccaaaaggccacactggtatgcctggccacaggcttctaccc | constant 2 (TRBC2) |
| | cgaccacgtggagctgagctggtgggtgaatgggaaggaggtgcacagtggggtca | NCBI Reference |
| | gcacagacccgcagcccctcaaggagcagcccgccctcaatgactccagatactgc | Sequence: |
| | ctgagcagccgcctgagggtctcggccaccttctggcagaacccccgcaaccactt | NG_001333.2, |
| | ccgctgtcaagtccagttctacgggctctcggagaatgacgagtggacccaggata | TRBC2 |
| | gggccaaacccgtcacccagatcgtcagcgccgaggcctggggtagagcaggtgag | |
| | tggggcctggggagatgcctggaggagattaggtgagaccagctaccagggaaaat | |
| | ggaaagatccaggtagcggacaagactagatccagaagaaagccagagtggacaag | |
| | gtgggatgatcaaggttcacagggtcagcaaagcacggtgtgcacttcccccacca | |
| | agaagcatagaggctgaatggagcacctcaagctcattcttccttcagatcctgac | |
| | accttagagctaagctttcaagtctccctgaggaccagccatacagctcagcatct | |
| | gagtggtgtgcatcccattctcttctggggtcctggtttcctaagatcatagtgac | |
| | cacttcgctggcactggagcagcatgagggagacagaaccagggctatcaaaggag | |
| | gctgactttgtactatctgatatgcatgtgtttgtggcctgtgagtctgtgatgta | |
| | aggctcaatgtccttacaaagcagcattctctcatccatttttcttcccctgtttt | |
| | ctttcagactgtggcttcacctccggtaagtgagtctctcctttttctctctatct | |
| | ttcgccgtctctgctctcgaaccagggcatggagaatccacggacacaggggcgtg | |
| | agggaggccagagccacctgtgcacaggtgcctacatgctctgttcttgtcaacag | |
| | agtcttaccagcaaggggtcctgtctgccaccatcctctatgagatcttgctaggg | |
| | aaggccaccttgtatgccgtgctggtcagtgccctcgtgctgatggccatggtaag | |
| | gaggagggtgggatagggcagatgatgggggcaggggatggaacatcacacatggg | |
| | cataaaggaatctcagagccagagcacagcctaatatatcctatcacctcaatgaa | |
| | accataatgaagccagactggggagaaaatgcagggaatatcacagaatgcatcat | |
| | gggaggatggagacaaccagcgagccctactcaaattaggcctcagagcccgcctc | |
| | ccctgccctactcctgctgtgccatagcccctgaaaccctgaaaatgttctctctt | |
| | ccacaggtcaagagaaaggattccagaggctag | |
| 4 | cgtgaggctccggtgcccgtcagtgggcagagcgcacatcgcccacagtccccgag | EF1alpha promoter |
| | aagttggggggaggggtcggcaattgaaccggtgcctagagaaggtggcgcggggt | (GenBank: J04617.1) |
| | aaactgggaaagtgatgtcgtgtactggctccgcctttttcccgagggtgggggag | |
| | aaccgtatataagtgcagtagtcgccgtgaacgttctttttcgcaacgggtttgcc | |
| | gccagaacacaggtaagtgccgtgtgtggttcccgcgggcctggcctctttacggg | |
| | ttatggcccttgcgtgccttgaattacttccacgcccctggctgcagtacgtgatt | |
| | cttgatcccgagcttcgggttggaagtgggtgggagagttcgaggccttgcgctta | |
| | aggagccccttcgcctcgtgcttgagttgaggcctggcctgggcgctggggccgcc | |
| | gcgtgcgaatctggtggcaccttcgcgcctgtctcgctgctttcgataagtctcta | |
| | gccatttaaaatttttgatgacctgctgcgacgctttttttctggcaagatagtct | |
| | tgtaaatgcgggccaagatctgcacactggtatttcggtttttggggccgcgggcg | |
| | gcgacggggcccgtgcgtcccagcgcacatgttcggcgaggcggggcctgcgagcg | |
| | cggccaccgagaatcggacgggggtagtctcaagctggccggcctgctctggtgcc | |
| | tggcctcgcgccgccgtgtatcgccccgccctgggcggcaaggctggcccggtcgg | |
| | caccagttgcgtgagcggaaagatggccgcttcccggccctgctgcagggagctca | |
| | aaatggaggacgcggcgctcgggagagcgggcgggtgagtcacccacacaaaggaa | |
| | aagggcctttccgtcctcagccgtcgcttcatgtgactccacggagtaccgggcgc | |
| | cgtccaggcacctcgattagttctcgagcttttggagtacgtcgtctttaggttgg | |
| | ggggaggggttttatgcgatggagtttccccacactgagtgggtggagactgaagt | |
| | taggccagcttggcacttgatgtaattctccttggaatttgccctttttgagtttg | |
| | gatcttggttcattctcaagcctcagacagtggttcaaagtttttttcttccattt | |
| | caggtgtcgtgaa | |
| 5 | CGTGAGGCTCCGGTGCCCGTCAGTGGGCAGAGCGCACATCGCCCACAGTCCCCGAG | EFlalpha promoter |
| | AAGTTGGGGGGAGGGGTCGGCAATTGAACCGGTGCCTAGAGAAGGTGGCGCGGGGT | |
| | AAACTGGGAAAGTGATGTCGTGTACTGGCTCCGCCTTTTTCCCGAGGGTGGGGGAG | |
| | AACCGTATATAAGTGCACTAGTCGCCGTGAACGTTCTTTTTCGCAACGGGTTTGCC | |
| | GCCAGAACACAGGTAAGTGCCGTGTGTGGTTCCCGCGGGCCTGGCCTCTTTACGGG | |
| | TTATGGCCCTTGCGTGCCTTGAATTACTTCCACCTGGCTGCAGTACGTGATTCTTG | |
| | ATCCCGAGCTTCGGGTTGGAAGTGGGTGGGAGAGTTCGTGGCCTTGCGCTTAAGGA | |
| | GCCCCTTCGCCTCGTGCTTGAGTTGTGGCCTGGCCTGGGCGCTGGGGCCGCCGCGT | |
| | GCGAATCTGGTGGCACCTTCGCGCCTGTCTCGCTGCTTTCGATAAGTCTCTAGCCA | |
| | TTTAAAATTTTTGATGACCTGCTGCGACGCTTTTTTTCTGGCAAGATAGTCTTGTA | |
| | AATGCGGGCCAAGATCAGCACACTGGTATTTCGGTTTTTGGGGCCGCGGGCGGCGA | |
| | CGGGGCCCGTGCGTCCCAGCGCACATGTTCGGCGAGGCGGGGCCTGCGAGCGCGGC | |
| | CACCGAGAATCGGACGGGGGTAGTCTCAAGCTGCCCGGCCTGCTCTGGTGCCTGGC | |
| | CTCGCGCCGCCGTGTATCGCCCCGCCCTGGGCGGCAAGGCTGGCCCGGTCGGCACC | |
| | AGTTGCGTGAGCGGAAAGATGGCCGCTTCCCGGCCCTGCTGCAGGGAGCACAAAAT | |
| | GGAGGACGCGGCGCTCGGGAGAGCGGGCGGGTGAGTCACCCACACAAAGGAAAAGG | |
| | GCCTTTCCGTCCTCAGCCGTCGCTTCATGTGACTCCACGGAGTACCGGGCGCCGTC | |
| | CAGGCACCTCGATTAGTTCTCCAGCTTTTGGAGTACGTCGTCTTTAGGTTGGGGGG | |
| | AGGGGTTTTATGCGATGGAGTTTCCCCACACTGAGTGGGTGGAGACTGAAGTTAGG | |
| | CCAGCTTGGCACTTGATGTAATTCTCCTTGGAATTTGCCCTTTTTGAGTTTGGATC | |
| | TTGGTTCATTCTCAAGCCTCAGACAGTGGTTCAAAGTTTTTTTCTTCCATTTCAGG | |
| | TGTCGTGAAAACTACCCCTAAAAGCCAAA | |
| 6 | LEGGGEGRGSLLTCGDVEENPGPR | T2A |
| 7 | EGRGSLLTCGDVEENPGP | T2A |
| 8 | GSGATNFSLLKQAGDVEENPGP | P2A |
| 9 | ATNFSLLKQAGDVEENPGP | P2A |
| 10 | QCTNYALLKLAGDVESNPGP | E2A |
| 11 | VKQTLNFDLLKLAGDVESNPGP | F2A |
| 12 | MLLLVTSLLLCELPHPAFLLIPRKVCNGIGIGEFKDSLSINATNIKHFKNCTSISG | EGFRt |
| | DLHILPVAFRGDSFTHTPPLDPQELDILKTVKEITGFLLIQAWPENRTDLHAFENL | |
| | EIIRGRTKQHGQFSLAVVSLNITSLGLRSLKEISDGDVIISGNKNLCYANTINWKK | |
| | LFGTSGQKTKIISNRGENSCKATGQVCHALCSPEGCWGPEPRDCVSCRNVSRGREC | |
| | VDKCNLLEGEPREFVENSECIQCHPECLPQAMNITCTGRGPDNCIQCAHYIDGPHC | |
| | VKTCPAGVMGENNTLVWKYADAGHVCHLCHPNCTYGCTGPGLEGCPTNGPKIPSIA | |
| | TGMVGALLLLLVVALGIGLFM | |
| 13 | RKVCNGIGIGEFKDSLSINATNIKHFKNCTSISGDLHILPVAFRGDSFTHTPPLDP | EGFRt |
| | QELDILKTVKEITGFLLIQAWPENRTDLHAFENLEIIRGRTKQHGQFSLAVVSLNI | |
| | TSLGLRSLKEISDGDVIISGNKNLCYANTINWKKLFGTSGQKTKIISNRGENSCKA | |
| | TGQVCHALCSPEGCWGPEPRDCVSCRNVSRGRECVDKCNLLEGEPREFVENSECIQ | |
| | CHPECLPQAMNITCTGRGPDNCIQCAHYIDGPHCVKTCPAGVMGENNTLVWKYADA | |
| | GHVCHLCHPNCTYGCTGPGLEGCPTNGPKIPSIATGMVGALLLLLVVALGIGLFM | |
| 14 | DIQNPEPAVYQLKDPRSQDSTLCLFTDFDSQINVPKTMESGTFITDKTVLDMKAMD | Mouse TCR alpha |
| | SKSNGAIAWSNQTSFTCQDIFKETNATYPSSDVPCDATLTEKSFETDMNLNFQNLS | constant |
| | VMGLRILLLKVAGFNLLMTLRLWSS | |
| 15 | NIQNPEPAVYQLKDPRSQDSTLCLFTDFDSQINVPKTMESGTFITDKTVLDMKAMD | Mouse TCR alpha |
| | SKSNGAIAWSNQTSFTCQDIFKETNATYPSSDVPCDATLTEKSFETDMNLNFQNLS | constant |
| | VMGLRILLLKVAGFNLLMTLRLWSS | |
| 16 | EDLRNVTPPKVSLFEPSKAEIANKQKATLVCLARGFFPDHVELSWWVNGKEVHSGV | Mouse TCR beta |
| | STDPQAYKESNYSYCLSSRLRVSATFWHNPRNHFRCQVQFHGLSEEDKWPEGSPKP | constant (Uniprot |
| | VTQNISAEAWGRADCGITSASYQQGVLSATILYEILLGKATLYAVLVSTLVVMAMV | P01852) |
| | KRKNS | |
| 17 | DLRNVTPPKVSLFEPSKAEIANKQKATLVCLARGFFPDHVELSWWVNGKEVHSGVS | Mouse TCR beta |
| | TDPQAYKESNYSYCLSSRLRVSATFWHNPRNHFRCQVQFHGLSEEDKWPEGSPKPV | constant |
| | TQNISAEAWGRADCGITSASYHQGVLSATILYEILLGKATLYAVLVSGLVLMAMVK | |
| | RKNS | |
| 18 | gggtctctctggttagaccagatctgagcctgggagctctctggctaactagggaa | MND promoter |
| | cccactgcttaagcctcaataaagcttgccttgagtgcttcaagtagtgtgtgccc | |
| | gtctgttgtgtgactctggtaactagagatccctcagacccttttagtcagtgtgg | |
| | aaaatctctagca | |
| 19 | PNIQNPDPAVYQLRDSKSSDKSVCLFTDFDSQTNVSQSKDSDVYITDKTVLDMRSM | Human TCR alpha |
| | DFKSNSAVAWSNKSDFACANAFNNSIIPEDTFFPSPESSCDVKLVEKSFETDTNLN | constant (Uniprot |
| | FQNLSVIGFRILLLKVAGFNLLMTLRLWSS | P01848) |
| 20 | EDLNKVFPPEVAVFEPSEAEISHTQKATLVCLATGFFPDHVELSWWVNGKEVHSGV | Human TCR beta |
| | STDPQPLKEQPALNDSRYCLSSRLRVSATFWQNPRNHFRCQVQFYGLSENDEWTQD | constant 1 (Uniprot |
| | RAKPVTQIVSAEAWGRADCGFTSVSYQQGVLSATILYEILLGKATLYAVLVSALVL | P01850) |
| | MAMVKRKDF | |
| 21 | DLKNVFPPEVAVFEPSEAEISHTQKATLVCLATGFYPDHVELSWWVNGKEVHSGVS | Human TCR beta |
| | TDPQPLKEQPALNDSRYCLSSRLRVSATFWQNPRNHFRCQVQFYGLSENDEWTQDR | constant 2 (Uniprot |
| | AKPVTQIVSAEAWGRADCGFTSESYQQGVLSATILYEILLGKATLYAVLVSALVLM | A0A5B9) |
| | AMVKRKDSRG | |
| 22 | -PGGG-(SGGGG)n-P-wherein P is proline, G is glycine and | Linker |
| | S is serine | |
| 23 | GSADDAKKDAAKKDGKS | Linker |
| 24 | NIQNPDPAVYQLRDSKSSDKSVCLFTDFDSQTNVSQSKDSDVYITDKTVLDMRSMD | human TCR alpha |
| | FKSNSAVAWSNKSDFACANAFNNSIIPEDTFFPSPESSCDVKLVEKSFETDTNLNF | constant (Genbank |
| | QNLSVIGFRILLLKVAGFNLLMTLRLWSS | Accession No. |
| | | CAA26636.1) |
| 25 | EDLKNVFPPEVAVFEPSEAEISHTQKATLVCLATGFYPDHVELSWWVNGKEVHSGV | human TCR beta |
| | STDPQPLKEQPALNDSRYCLSSRLRVSATFWQNPRNHFRCQVQFYGLSENDEWTQD | constant (Uniprot |
| | RAKPVTQIVSAEAWGRADCGFTSESYQQGVLSATILYEILLGKATLYAVLVSALVL | Accession No. |
| | MAMVKRKDSRG | A0A0G2JNG9) |
| 26 | AGCGCTCTCGTACAGAGTTGGCATTATAATACGACTCACTATAGGGGAGAATCAAA | TRAC gRNA |
| | ATCGGTGAATGTTTTAGAGCTAGAAATAGCAAGTTAAAATAAGGCTAGTCCGTTAT | transcription sequence |
| | CAACTTGAAAAAGTGGCACCGAGTCGGTGCTTTTTTT | |
| 27 | GAGAAUCAAAAUCGGUGAAUGUUUUAGAGCUAGAAAUAGCAAGUUAAAAUAAGGCU | TRAC gRNA |
| | AGUCCGUUAUCAACUUGAAAAAGUGGCACCGAGUCGGUGCUUUU | sequence |
| 28 | UCUCUCAGCUGGUACACGGC | TRAC-10 gRNA |
| | | targeting domain |
| 29 | UGGAUUUAGAGUCUCUCAGC | TRAC-110 gRNA |
| | | targeting domain |
| 30 | ACACGGCAGGGUCAGGGUUC | TRAC-116 gRNA |
| | | targeting domain |
| 31 | GAGAAUCAAAAUCGGUGAAU | TRAC-16 gRNA |
| | | targeting domain |
| 32 | GCUGGUACACGGCAGGGUCA | TRAC-4 gRNA |
| | | targeting domain |
| 33 | CUCAGCUGGUACACGGC | TRAC-49 gRNA |
| | | targeting domain |
| 34 | UGGUACACGGCAGGGUC | TRAC-2 gRNA |
| | | targeting domain |
| 35 | GCUAGACAUGAGGUCUA | TRAC-30 gRNA |
| | | targeting domain |
| 36 | GUCAGAUUUGUUGCUCC | TRAC-43 gRNA |
| | | targeting domain |
| 37 | UCAGCUGGUACACGGCA | TRAC-23 gRNA |
| | | targeting domain |
| 38 | GCAGACAGACUUGUCAC | TRAC-34 gRNA |
| | | targeting domain |
| 39 | GGUACACGGCAGGGUCA | TRAC-25 gRNA |
| | | targeting domain |
| 40 | CUUCAAGAGCAACAGUGCUG | TRAC-128 gRNA |
| | | targeting domain |
| 41 | AGAGCAACAGUGCUGUGGCC | TRAC-105 gRNA |
| | | targeting domain |
| 42 | AAAGUCAGAUUUGUUGCUCC | TRAC-106 gRNA |
| | | targeting domain |
| 43 | ACAAAACUGUGCUAGACAUG | TRAC-123 gRNA |
| | | targeting domain |
| 44 | AAACUGUGCUAGACAUG | TRAC-64 gRNA |
| | | targeting domain |
| 45 | UGUGCUAGACAUGAGGUCUA | TRAC-97 gRNA |
| | | targeting domain |
| 46 | GGCUGGGGAAGAAGGUGUCUUC | TRAC-148 gRNA |
| | | targeting domain |
| 47 | GCUGGGGAAGAAGGUGUCUUC | TRAC-147 gRNA |
| | | targeting domain |
| 48 | GGGGAAGAAGGUGUCUUC | TRAC-234 gRNA |
| | | targeting domain |
| 49 | GUUUUGUCUGUGAUAUACACAU | TRAC-167 gRNA |
| | | targeting domain |
| 50 | GGCAGACAGACUUGUCACUGGAUU | TRAC-177 gRNA |
| | | targeting domain |
| 51 | GCAGACAGACUUGUCACUGGAUU | TRAC-176 gRNA |
| | | targeting domain |
| 52 | GACAGACUUGUCACUGGAUU | TRAC-257 gRNA |
| | | targeting domain |
| 53 | GUGAAUAGGCAGACAGACUUGUCA | TRAC-233 gRNA |
| | | targeting domain |
| 54 | GAAUAGGCAGACAGACUUGUCA | TRAC-231 gRNA |
| | | targeting domain |
| 55 | GAGUCUCUCAGCUGGUACACGG | TRAC-163 gRNA |
| | | targeting domain |
| 56 | GUCUCUCAGCUGGUACACGG | TRAC-241 gRNA |
| | | targeting domain |
| 57 | GGUACACGGCAGGGUCAGGGUU | TRAC-179 gRNA |
| | | targeting domain |
| 58 | GUACACGGCAGGGUCAGGGUU | TRAC-178 gRNA |
| | | targeting domain |
| 59 | CACCCAGAUCGUCAGCGCCG | TRBC-40 gRNA |
| | | targeting domain |
| 60 | CAAACACAGCGACCUCGGGU | TRBC-52 gRNA |
| | | targeting domain |
| 61 | UGACGAGUGGACCCAGGAUA | TRBC-25 gRNA |
| | | targeting domain |
| 62 | GGCUCUCGGAGAAUGACGAG | TRBC-35 gRNA |
| | | targeting domain |
| 63 | GGCCUCGGCGCUGACGAUCU | TRBC-50 gRNA |
| | | targeting domain |
| 64 | GAAAAACGUGUUCCCACCCG | TRBC-39 gRNA |
| | | targeting domain |
| 65 | AUGACGAGUGGACCCAGGAU | TRBC-49 gRNA |
| | | targeting domain |
| 66 | AGUCCAGUUCUACGGGCUCU | TRBC-51 gRNA |
| | | targeting domain |
| 67 | CGCUGUCAAGUCCAGUUCUA | TRBC-26 gRNA |
| | | targeting domain |
| 68 | AUCGUCAGCGCCGAGGCCUG | TRBC-47 gRNA |
| | | targeting domain |
| 69 | UCAAACACAGCGACCUCGGG | TRBC-45 gRNA |
| | | targeting domain |
| 70 | CGUAGAACUGGACUUGACAG | TRBC-34 gRNA |
| | | targeting domain |
| 71 | AGGCCUCGGCGCUGACGAUC | TRBC-227 gRNA |
| | | targeting domain |
| 72 | UGACAGCGGAAGUGGUUGCG | TRBC-41 gRNA |
| | | targeting domain |
| 73 | UUGACAGCGGAAGUGGUUGC | TRBC-30 gRNA |
| | | targeting domain |
| 74 | UCUCCGAGAGCCCGUAGAAC | TRBC-206 gRNA |
| | | targeting domain |
| 75 | CGGGUGGGAACACGUUUUUC | TRBC-32 gRNA |
| | | targeting domain |
| 76 | GACAGGUUUGGCCCUAUCCU | TRBC-276 gRNA |
| | | targeting domain |
| 77 | GAUCGUCAGCGCCGAGGCCU | TRBC-274 gRNA |
| | | targeting domain |
| 78 | GGCUCAAACACAGCGACCUC | TRBC-230 gRNA |
| | | targeting domain |
| 79 | UGAGGGUCUCGGCCACCUUC | TRBC-235 gRNA |
| | | targeting domain |
| 80 | AGGCUUCUACCCCGACCACG | TRBC-38 gRNA |
| | | targeting domain |
| 81 | CCGACCACGUGGAGCUGAGC | TRBC-223 gRNA |
| | | targeting domain |
| 82 | UGACAGGUUUGGCCCUAUCC | TRBC-221 gRNA |
| | | targeting domain |
| 83 | CUUGACAGCGGAAGUGGUUG | TRBC-48 gRNA |
| | | targeting domain |
| 84 | AGAUCGUCAGCGCCGAGGCC | TRBC-216 gRNA |
| | | targeting domain |
| 85 | GCGCUGACGAUCUGGGUGAC | TRBC-210 gRNA |
| | | targeting domain |
| 86 | UGAGGGCGGGCUGCUCCUUG | TRBC-268 gRNA |
| | | targeting domain |
| 87 | GUUGCGGGGGUUCUGCCAGA | TRBC-193 gRNA |
| | | targeting domain |
| 88 | AGCUCAGCUCCACGUGGUCG | TRBC-246 gRNA |
| | | targeting domain |
| 89 | GCGGCUGCUCAGGCAGUAUC | TRBC-228 gRNA |
| | | targeting domain |
| 90 | GCGGGGGUUCUGCCAGAAGG | TRBC-43 gRNA |
| | | targeting domain |
| 91 | UGGCUCAAACACAGCGACCU | TRBC-272 gRNA |
| | | targeting domain |
| 92 | ACUGGACUUGACAGCGGAAG | TRBC-33 gRNA |
| | | targeting domain |
| 93 | GACAGCGGAAGUGGUUGCGG | TRBC-44 gRNA |
| | | targeting domain |
| 94 | GCUGUCAAGUCCAGUUCUAC | TRBC-211 gRNA |
| | | targeting domain |
| 95 | GUAUCUGGAGUCAUUGAGGG | TRBC-253 gRNA |
| | | targeting domain |
| 96 | CUCGGCGCUGACGAUCU | TRBC-18 gRNA |
| | | targeting domain |
| 97 | CCUCGGCGCUGACGAUC | TRBC-6 gRNA |
| | | targeting domain |
| 98 | CCGAGAGCCCGUAGAAC | TRBC-85 gRNA |
| | | targeting domain |
| 99 | CCAGAUCGUCAGCGCCG | TRBC-129 gRNA |
| | | targeting domain |
| 100 | GAAUGACGAGUGGACCC | TRBC-93 gRNA |
| | | targeting domain |
| 101 | GGGUGACAGGUUUGGCCCUAUC | TRBC-415 gRNA |
| | | targeting domain |
| 102 | GGUGACAGGUUUGGCCCUAUC | TRBC-414 gRNA |
| | | targeting domain |
| 103 | GUGACAGGUUUGGCCCUAUC | TRBC-310 gRNA |
| | | targeting domain |
| 104 | GACAGGUUUGGCCCUAUC | TRBC-308 gRNA |
| | | targeting domain |
| 105 | GAUACUGCCUGAGCAGCCGCCU | TRBC-401 gRNA |
| | | targeting domain |
| 106 | GACCACGUGGAGCUGAGCUGGUGG | TRBC-468 gRNA |
| | | targeting domain |
| 107 | GUGGAGCUGAGCUGGUGG | TRBC-462 gRNA |
| | | targeting domain |
| 108 | GGGCGGGCUGCUCCUUGAGGGGCU | TRBC-424 gRNA |
| | | targeting domain |
| 109 | GGCGGGCUGCUCCUUGAGGGGCU | TRBC-423 gRNA |
| | | targeting domain |
| 110 | GCGGGCUGCUCCUUGAGGGGCU | TRBC-422 gRNA |
| | | targeting domain |
| 111 | GGGCUGCUCCUUGAGGGGCU | TRBC-420 gRNA |
| | | targeting domain |
| 112 | GGCUGCUCCUUGAGGGGCU | TRBC-419 gRNA |
| | | targeting domain |
| 113 | GCUGCUCCUUGAGGGGCU | TRBC-418 gRNA |
| | | targeting domain |
| 114 | GGUGAAUGGGAAGGAGGUGCACAG | TRBC-445 gRNA |
| | | targeting domain |
| 115 | GUGAAUGGGAAGGAGGUGCACAG | TRBC-444 gRNA |
| | | targeting domain |
| 116 | GAAUGGGAAGGAGGUGCACAG | TRBC-442 gRNA |
| | | targeting domain |
| 117 | ATTCACCGATTTTGATTCTC | TRAC target sequence |
| 118 | AGATCGTCAGCGCCGAGGCC | TRBC target sequence |
| 119 | CTGACCTCTTCTCTTCCTCCCACAG | HBB splice site |
| | | acceptor |
| 120 | TTTCTCTCCACAG | IgG splice site |
| | | acceptor |
| 121 | PYIQNPEPAVYQLKDPRSQDSTLCLFTDFDSQINVPKTMESGTFITDKTVLDMKAM | Mouse TCR alpha |
| | DSKSNGAIAWSNQTSFTCQDIFKETNATYPSSDVPCDATLTEKSFETDMNLNFQNL | constant (Uniprot |
| | SVMGLRILLLKVAGFNLLMTLRLWSS | P01849) |
| 122 | IQNPEPAVYQLKDPRSQDSTLCLFTDFDSQINVPKTMESGTFITDKCVLDMKAMDS | Modified mouse TCR |
| | KSNGAIAWSNQTSFTCQDIFKETNATYPSSDVPCDATLTEKSFETDMNLNFQNLLV | alpha constant |
| | IVLRILLLKVAGFNLLMTLRLWSS | |
| 123 | EDLRNVTPPKVSLFEPSKAEIANKQKATLVCLARGFFPDHVELSWWVNGKEVHSGV | Modified mouse TCR |
| | CTDPQAYKESNYSYCLSSRLRVSATFWHNPRNHFRCQVQFHGLSEEDKWPEGSPKP | beta constant |
| | VTQNISAEAWGRADCGITSASYQQGVLSATILYEILLGKATLYAVLVSTLVVMAMV | |
| | KRKNS | |
| 124 | CTCTATCAATGAGAGAGCAATCTCCTGGTAATGTGATAGATTTCCCAACTTAATGC | TRAC 5′ homology |
| | CAACATACCATAAACCTCCCATTCTGCTAATGCCCAGCCTAAGTTGGGGAGACCAC | arm |
| | TCCAGATTCCAAGATGTACAGTTTGCTTTGCTGGGCCTTTTTCCCATGCCTGCCTT | |
| | TACTCTGCCAGAGTTATATTGCTGGGGTTTTGAAGAAGATCCTATTAAATAAAAGA | |
| | ATAAGCAGTATTATTAAGTAGCCCTGCATTTCAGGTTTCCTTGAGTGGCAGGCCAG | |
| | GCCTGGCCGTGAACGTTCACTGAAATCATGGCCTCTTGGCCAAGATTGATAGCTTG | |
| | TGCCTGTCCCTGAGTCCCAGTCCATCACGAGCAGCTGGTTTCTAAGATGCTATTTC | |
| | CCGTATAAAGCATGAGACCGTGACTTGCCAGCCCCACAGAGCCCCGCCCTTGTCCA | |
| | TCACTGGCATCTGGACTCCAGCCTGGGTTGGGGCAAAGAGGGAAATGAGATCATGT | |
| | CCTAACCCTGATCCTCTTGTCCCACAGATATCCAGAACCCTGACCCTGCCGTGTAC | |
| | CAGCTGAGAGACTCTAAATCCAGTGACAAGTCTGTCTGCCTATTCACCGAT | |
| 125 | TTTGATTCTCAAACAAATGTGTCACAAAGTAAGGATTCTGATGTGTATATCACAGA | TRAC 3′ homology |
| | CAAAACTGTGCTAGACATGAGGTCTATGGACTTCAAGAGCAACAGTGCTGTGGCCT | arm |
| | GGAGCAACAAATCTGACTTTGCATGTGCAAACGCCTTCAACAACAGCATTATTCCA | |
| | GAAGACACCTTCTTCCCCAGCCCAGGTAAGGGCAGCTTTGGTGCCTTCGCAGGCTG | |
| | TTTCCTTGCTTCAGGAATGGCCAGGTTCTGCCCAGAGCTCTGGTCAATGATGTCTA | |
| | AAACTCCTCTGATTGGTGGTCTCGGCCTTATCCATTGCCACCAAAACCCTCTTTTT | |
| | ACTAAGAAACAGTGAGCCTTGTTCTGGCAGTCCAGAGAATGACACGGGAAAAAAGC | |
| | AGATGAAGAGAAGGTGGCAGGAGAGGGCACGTGGCCCAGCCTCAGTCTCTCCAACT | |
| | GAGTTCCTGCCTGCCTGCCTTTGCTCAGACTGTTTGCCCCTTACTGCTCTTCTAGG | |
| | CCTCATTCTAAGCCCCTTCTCCAAGTTGCCTCTCCTTATTTCTCCCTGTCTGCCAA | |
| | AAAATCTTTCCCAGCTCACTAAGTCAGTCTCACGCAGTCACTCATTAACCCACCAA | |
| | TCACTGATTGTG | |
| 126 | GAACAGAGAAACAGGAGAATATGGGCCAAACAGGATATCTGTGGTAAGCAGTTCCT | MND promoter |
| | GCCCCGGCTCAGGGCCAAGAACAGTTGGAACAGCAGAATATGGGCCAAACAGGATA | |
| | TCTGTGGTAAGCAGTTCCTGCCCCGGCTCAGGGCCAAGAACAGATGGTCCCCAGAT | |
| | GCGGTCCCGCCCTCAGCAGTTTCTAGAGAACCATCAGATGTTTCCAGGGTGCCCCA | |
| | AGGACCTGAAATGACCCTGTGCCTTATTTGAACTAACCAATCAGTTCGCTTCTCGC | |
| | TTCTGTTCGCGCGCTTCTGCTCCCCGAGCTCTATATAAGCAGAGCTCGTTTAGTGA | |
| | ACCGTCAGATC | |
| 127 | GGATCTGCGATCGCTCCGGTGCCCGTCAGTGGGCAGAGCGCACATCGCCCACAGTC | Ef1alpha promoter |
| | CCCGAGAAGTTGGGGGGAGGGGTCGGCAATTGAACCGGTGCCTAGAGAAGGTGGCG | with HTLV1 enhancer |
| | CGGGGTAAACTGGGAAAGTGATGTCGTGTACTGGCTCCGCCTTTTTCCCGAGGGTG | |
| | GGGGAGAACCGTATATAAGTGCAGTAGTCGCCGTGAACGTTCTTTTTCGCAACGGG | |
| | TTTGCCGCCAGAACACAGCTGAAGCTTCGAGGGGCTCGCATCTCTCCTTCACGCGC | |
| | CCGCCGCCCTACCTGAGGCCGCCATCCACGCCGGTTGAGTCGCGTTCTGCCGCCTC | |
| | CCGCCTGTGGTGCCTCCTGAACTGCGTCCGCCGTCTAGGTAAGTTTAAAGCTCAGG | |
| | TCGAGACCGGGCCTTTGTCCGGCGCTCCCTTGGAGCCTACCTAGACTCAGCCGGCT | |
| | CTCCACGCTTTGCCTGACCCTGCTTGCTCAACTCTACGTCTTTGTTTCGTTTTCTG | |
| | TTCTGCGCCGTTACAGATCCAAGCTGTGACCGGCGCCTAC | |
| 128 | GGATCTGGAGCGACGAATTTTAGTCTACTGAAACAAGCGGGAGACGTGGAGGAAAA | P2A nucleotide |
| | CCCTGGACCT | sequence |
| 129 | RVKFSRSAEPPAYQQGQNQLYNELNLGRREEYDVLDKRRGRDPEMGGKPRRKNPQE | CD3 zeta |
| | GLYNELQKDKMAEAYSEIGMKGERRRGKGHDGLYQGLSTATKDTYDALHMQALPPR | |
| 130 | RVKFSRSADAPAYKQGQNQLYNELNLGRREEYDVLDKRRGRDPEMGGKPRRKNPQE | CD3 zeta |
| | GLYNELQKDKMAEAYSEIGMKGERRRGKGHDGLYQGLSTATKDTYDALHMQALPPR | |
| 131 | ESKYGPPCPPCP | spacer (IgG4hinge) |
| 132 | GAATCTAAGTACGGACCGCCCTGCCCCCCTTGCCCT | spacer (IgG4hinge) |
| 133 | ESKYGPPCPPCPGQPREPQVYTLPPSQEEMTKNQVSLTCLVKGFYPSDIAVEWESN | Hinge-CH3 spacer |
| | GQPENNYKTTPPVLDSDGSFFLYSRLTVDKSRWQEGNVFSCSVMHEALHNHYTQKS | |
| | LSLSLGK | |
| 134 | ESKYGPPCPPCPAPEFLGGPSVFLFPPKPKDTLMISRTPEVTCVVVDVSQEDPEVQ | Hinge-CH2-CH3 |
| | FNWYVDGVEVHNAKTKPREEQFNSTYRVVSVLTVLHQDWLNGKEYKCKVSNKGLPS | spacer |
| | SIEKTISKAKGQPREPQVYTLPPSQEEMTKNQVSLTCLVKGFYPSDIAVEWESNGQ | |
| | PENNYKTTPPVLDSDGSFFLYSRLTVDKSRWQEGNVFSCSVMHEALHNHYTQKSLS | |
| | LSLGK | |
| 135 | RWPESPKAQASSVPTAQPQAEGSLAKATTAPATTRNTGRGGEEKKKEKEKEEQEER | IgD-hinge-Fc |
| | ETKTPECPSHTQPLGVYLLTPAVQDLWLRDKATFTCFVVGSDLKDAHLTWEVAGKV | |
| | PTGGVEEGLLERHSNGSQSQHSRLTLPRSLWNAGTSVTCTLNHPSLPPQRLMALRE | |
| | PAAQAPVKLSLNLLASSDPPEAASWLLCEVSGFSPPNILLMWLEDQREVNTSGFAP | |
| | ARPPPQPGSTTFWAWSVLRVPAPPSPQPATYTCVVSHEDSRTLLNASRSLEVSYVT | |
| | DH | |
| 136 | FWVLVVVGGVLACYSLLVTVAFIIFWV | CD28 (amino acids |
| | | 153-179 of Accession |
| | | No. P10747) |
| 137 | IEVMYPPPYLDNEKSNGTIIHVKGKHLCPSPLFPGPSKPFWVLVVVGGVLACYSLL | CD28 (amino acids |
| | VTVAFIIFWV | 114-179 of Accession |
| | | No. P10747) |
| 138 | RSKRSRLLHSDYMNMTPRRPGPTRKHYQPYAPPRDFAAYRS | CD28 (amino acids |
| | | 180-220 of P10747) |
| 139 | RSKRSRGGHSDYMNMTPRRPGPTRKHYQPYAPPRDFAAYRS | CD28 (LL to GG) |
| 140 | KRGRKKLLYIFKQPFMRPVQTTQEEDGCSCRFPEEEEGGCEL | 4-1BB (amino acids |
| | | 214-255 of Q07011.1) |
| 141 | RVKFSRSADAPAYQQGQNQLYNELNLGRREEYDVLDKRRGRDPEMGGKPRRKNPQE | CD3 zeta |
| | GLYNELQKDKMAEAYSEIGMKGERRRGKGHDGLYQGLSTATKDTYDALHMQALPPR | |
| 142 | CAGAACCCAGATCCCGCGGTATATCAACTGCGCGACTCAAAATCATCCGATAAGAG | Partial recombinant |
| | TGTCTGTTTGTTTACTGACTTCGACAGTCAAACTAATGTCTCTCAGAGCAAAGATT | TCR alpha constant |
| | CCGATGTCTACATCACTGACAAGTGC | region exon 1 |
| | | sequence |
| 143 | YMLDLQPET | HPV16 E7(11-19) |
| | | peptide |
| 144 | NNNNNNNNNNNNNNNNNNNNGUUUUAGAGCUAGAAAUAGCAAGUUAAAAUAAGGCU | TRAC gRNA |
| | AGUCCGUUAUCAACUUGAAAAAGUGGCACCGAGUCGGUGC | |
| 145 | NNNNNNNNNNNNNNNNNNNNGUUUUAGAGCUAUGCUGAAAAGCAUAGCAAGUUAAA | TRAC gRNA |
| | AUAAGGCUAGUCCGUUAUCAACUUGAAAAAGUGGCACCGAGUCGGUGC | |
| 146 | NNNNNNNNNNNNNNNNNNNNGUUUUAGAGCUAUGCUGGAAACAGCAUAGCAAGUUA | TRAC gRNA |
| | AAAUAAGGCUAGUCCGUUAUCAACUUGAAAAAGUGGCACCGAGUCGGUGC | |
| 147 | NNNNNNNNNNNNNNNNNNNNGUUUUAGAGCUAUGCUGUUUUGGAAACAAAACAGCA | TRAC gRNA |
| | UAGCAAGUUAAAAUAAGGCUAGUCCGUUAUCAACUUGAAAAAGUGGCACCGAGUCG | |
| | GUGC | |
| 148 | NNNNNNNNNNNNNNNNNNNNGUAUUAGAGCUAGAAAUAGCAAGUUAAUAUAAGGCU | TRAC gRNA |
| | AGUCCGUUAUCAACUUGAAAAAGUGGCACCGAGUCGGUGC | |
| 149 | NNNNNNNNNNNNNNNNNNNNGUUUAAGAGCUAGAAAUAGCAAGUUUAAAUAAGGCU | TRAC gRNA |
| | AGUCCGUUAUCAACUUGAAAAAGUGGCACCGAGUCGGUGC | |
| 150 | NNNNNNNNNNNNNNNNNNNNGUAUUAGAGCUAUGCUGUAUUGGAAACAAUACAGCA | TRAC gRNA |
| | UAGCAAGUUAAUAUAAGGCUAGUCCGUUAUCAACUUGAAAAAGUGGCACCGAGUCG | |
| | GUGC | |
| 151 | AAGGCUAGUCCGUUAUCAACUUGAAAAAGUGGCACCGAGUCGGUGCU | gRNA Proximal and |
| | | tail domain |
| 152 | AAGGCUAGUCCGUUAUCAACUUGAAAAAGUGGCACCGAGUCGGUGGUGC | gRNA Proximal and |
| | | tail domain |
| 153 | AAGGCUAGUCCGUUAUCAACUUGAAAAAGUGGCACCGAGUCGGUGCGGAUC | gRNA Proximal and |
| | | tail domain |
| 154 | AAGGCUAGUCCGUUAUCAACUUGAAAAAGUG | gRNA Proximal and |
| | | tail domain |
| 155 | AAGGCUAGUCCGUUAUCA | gRNA Proximal and |
| | | tail domain |
| 156 | AAGGCUAGUCCG | gRNA Proximal and |
| | | tail domain |
| 157 | NNNNNNNNNNNNNNNNNNNNGUUUUAGAGCUAGAAAUAGCAAGUUAAAAUAAGGCU | Exemplary chimeric |
| | AGUCCGUUAUCAACUUGAAAAAGUGGCACCGAGUCGGUGCUUUUUU | gRNA |
| 158 | NNNNNNNNNNNNNNNNNNNNGUUUUAGUACUCUGGAAACAGAAUCUACUAAAACAA | Exemplary chimeric |
| | GGCAAAAUGCCGUGUUUAUCUCGUCAACUUGUUGGCGAGAUUUUUU | gRNA |
| 159 | KKPYSIGLDIGTNSVGWAVVTDDYKVPAKKMKVLGNTDKSHIEKNLLGALLFDSGN | Streptococcus mutans |
| | TAEDRRLKRTARRRYTRRRNRILYLQEIFSEEMGKVDDSFFHRLEDSFLVTEDKRG | Cas9 |
| | ERHPIFGNLEEEVKYHENFPTIYHLRQYLADNPEKVDLRLVYLALAHIIKFRGHFL | |
| | IEGKFDTRNNDVQRLFQEFLAVYDNTFENSSLQEQNVQVEEILTDKISKSAKKDRV | |
| | LKLFPNEKSNGRFAEFLKLIVGNQADFKKHFELEEKAPLQFSKDTYEEELEVLLAQ | |
| | IGDNYAELFLSAKKLYDSILLSGILTVTDVGTKAPLSASMIQRYNEHQMDLAQLKQ | |
| | FIRQKLSDKYNEVFSDVSKDGYAGYIDGKTNQEAFYKYLKGLLNKIEGSGYFLDKI | |
| | EREDFLRKQRTFDNGSIPHQIHLQEMRAIIRRQAEFYPFLADNQDRIEKLLTFRIP | |
| | YYVGPLARGKSDFAWLSRKSADKITPWNFDEIVDKESSAEAFINRMTNYDLYLPNQ | |
| | KVLPKHSLLYEKFTVYNELTKVKYKTEQGKTAFFDANMKQEIFDGVFKVYRKVTKD | |
| | KLMDFLEKEFDEFRIVDLTGLDKENKVFNASYGTYHDLCKILDKDFLDNSKNEKIL | |
| | EDIVLTLTLFEDREMIRKRLENYSDLLTKEQVKKLERRHYTGWGRLSAELIHGIRN | |
| | KESRKTILDYLIDDGNSNRNFMQLINDDALSFKEEIAKAQVIGETDNLNQVVSDIA | |
| | GSPAIKKGILQSLKIVDELVKIMGHQPENIVVEMARENQFTNQGRRNSQQRLKGLT | |
| | DSIKEFGSQILKEHPVENSQLQNDRLFLYYLQNGRDMYTGEELDIDYLSQYDIDHI | |
| | IPQAFIKDNSIDNRVLTSSKENRGKSDDVPSKDVVRKMKSYWSKLLSAKLITQRKF | |
| | DNLTKAERGGLTDDDKAGFIKRQLVETRQITKHVARILDERFNTETDENNKKIRQV | |
| | KIVTLKSNLVSNFRKEFELYKVREINDYHHAHDAYLNAVIGKALLGVYPQLEPEFV | |
| | YGDYPHFHGHKENKATAKKFFYSNIMNFFKKDDVRTDKNGEIIWKKDEHISNIKKV | |
| | LSYPQVNIVKKVEEQTGGFSKESILPKGNSDKLIPRKTKKFYWDTKKYGGFDSPIV | |
| | AYSILVIADIEKGKSKKLKTVKALVGVTIMEKMTFERDPVAFLERKGYRNVQEENI | |
| | IKLPKYSLFKLENGRKRLLASARELQKGNEIVLPNHLGTLLYHAKNIHKVDEPKHL | |
| | DYVDKHKDEFKELLDVVSNFSKKYTLAEGNLEKIKELYAQNNGEDLKELASSFINL | |
| | LTFTAIGAPATFKFFDKNIDRKRYTSTTEILNATLIHQSITGLYETRIDLNKLGGD | |
| 160 | DKKYSIGLDIGTNSVGWAVITDEYKVPSKKFKVLGNTDRHSIKKNLIGALLFDSGE | Streptococcus |
| | TAEATRLKRTARRRYTRRKNRICYLQEIFSNEMAKVDDSFFHRLEESFLVEEDKKH | pyogenes Cas9 |
| | ERHPIFGNIVDEVAYHEKYPTIYHLRKKLVDSTDKADLRLIYLALAHMIKFRGHFL | |
| | IEGDLNPDNSDVDKLFIQLVQTYNQLFEENPINASGVDAKAILSARLSKSRRLENL | |
| | IAQLPGEKKNGLFGNLIALSLGLTPNFKSNFDLAEDAKLQLSKDTYDDDLDNLLAQ | |
| | IGDQYADLFLAAKNLSDAILLSDILRVNTEITKAPLSASMIKRYDEHHQDLTLLKA | |
| | LVRQQLPEKYKEIFFDQSKNGYAGYIDGGASQEEFYKFIKPILEKMDGTEELLVKL | |
| | NREDLLRKQRTFDNGSIPHQIHLGELHAILRRQEDFYPFLKDNREKIEKILTFRIP | |
| | YYVGPLARGNSRFAWMTRKSEETITPWNFEEVVDKGASAQSFIERMTNFDKNLPNE | |
| | KVLPKHSLLYEYFTVYNELTKVKYVTEGMRKPAFLSGEQKKAIVDLLFKTNRKVTV | |
| | KQLKEDYFKKIECFDSVEISGVEDRFNASLGTYHDLLKIIKDKDFLDNEENEDILE | |
| | DIVLTLTLFEDREMIEERLKTYAHLFDDKVMKQLKRRRYTGWGRLSRKLINGIRDK | |
| | QSGKTILDFLKSDGFANRNFMQLIHDDSLTFKEDIQKAQVSGQGDSLHEHIANLAG | |
| | SPAIKKGILQTVKVVDELVKVMGRHKPENIVIEMARENQTTQKGQKNSRERMKRIE | |
| | EGIKELGSQILKEHPVENTQLQNEKLYLYYLQNGRDMYVDQELDINRLSDYDVDHI | |
| | VPQSFLKDDSIDNKVLTRSDKNRGKSDNVPSEEVVKKMKNYWRQLLNAKLITQRKF | |
| | DNLTKAERGGLSELDKAGFIKRQLVETRQITKHVAQILDSRMNTKYDENDKLIREV | |
| | KVITLKSKLVSDFRKDFQFYKVREINNYHHAHDAYLNAVVGTALIKKYPKLESEFV | |
| | YGDYKVYDVRKMIAKSEQEIGKATAKYFFYSNIMNFFKTEITLANGEIRKRPLIET | |
| | NGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKN | |
| | PIDFLEAKGYKEVKKDLIIKLPKYSLFELENGRKRMLASAGELQKGNELALPSKYV | |
| | NFLYLASHYEKLKGSPEDNEQKQLFVEQHKHYLDEIIEQISEFSKRVILADANLDK | |
| | VLSAYNKHRDKPIREQAENIIHLFTLTNLGAPAAFKYFDTTIDRKRYTSTKEVLDA | |
| | TLIHQSITGLYETRIDLSQLGGD | |
| 161 | TKPYSIGLDIGTNSVGWAVTTDNYKVPSKKMKVLGNTSKKYIKKNLLGVLLFDSGI | Streptococcus |
| | TAEGRRLKRTARRRYTRRRNRILYLQEIFSTEMATLDDAFFQRLDDSFLVPDDKRD | thermophilus Cas9 |
| | SKYPIFGNLVEEKAYHDEFPTIYHLRKYLADSTKKADLRLVYLALAHMIKYRGHFL | |
| | IEGEFNSKNNDIQKNFQDFLDTYNAIFESDLSLENSKQLEEIVKDKISKLEKKDRI | |
| | LKLFPGEKNSGIFSEFLKLIVGNQADFRKCFNLDEKASLHFSKESYDEDLETLLGY | |
| | IGDDYSDVFLKAKKLYDAILLSGFLTVTDNETEAPLSSAMIKRYNEHKEDLALLKE | |
| | YIRNISLKTYNEVFKDDTKNGYAGYIDGKTNQEDFYVYLKKLLAEFEGADYFLEKI | |
| | DREDFLRKQRTFDNGSIPYQIHLQEMRAILDKQAKFYPFLAKNKERIEKILTFRIP | |
| | YYVGPLARGNSDFAWSIRKRNEKITPWNFEDVIDKESSAEAFINRMTSFDLYLPEE | |
| | KVLPKHSLLYETFNVYNELTKVRFIAESMRDYQFLDSKQKKDIVRLYFKDKRKVTD | |
| | KDIIEYLHAIYGYDGIELKGIEKQFNSSLSTYHDLLNIINDKEFLDDSSNEAIIEE | |
| | IIHTLTIFEDREMIKQRLSKFENIFDKSVLKKLSRRHYTGWGKLSAKLINGIRDEK | |
| | SGNTILDYLIDDGISNRNFMQLIHDDALSFKKKIQKAQIIGDEDKGNIKEVVKSLP | |
| | GSPAIKKGILQSIKIVDELVKVMGGRKPESIVVEMARENQYTNQGKSNSQQRLKRL | |
| | EKSLKELGSKILKENIPAKLSKIDNNALQNDRLYLYYLQNGKDMYTGDDLDIDRLS | |
| | NYDIDHIIPQAFLKDNSIDNKVLVSSASNRGKSDDVPSLEVVKKRKTFWYQLLKSK | |
| | LISQRKFDNLTKAERGGLSPEDKAGFIQRQLVETRQITKHVARLLDEKFNNKKDEN | |
| | NRAVRTVKIITLKSTLVSQFRKDFELYKVREINDFHHAHDAYLNAVVASALLKKYP | |
| | KLEPEFVYGDYPKYNSFRERKSATEKVYFYSNIMNIFKKSISLADGRVIERPLIEV | |
| | NEETGESVWNKESDLATVRRVLSYPQVNVVKKVEEQNHGLDRGKPKGLFNANLSSK | |
| | PKPNSNENLVGAKEYLDPKKYGGYAGISNSFTVLVKGTIEKGAKKKITNVLEFQGI | |
| | SILDRINYRKDKLNFLLEKGYKDIELIIELPKYSLFELSDGSRRMLASILSTNNKR | |
| | GEIHKGNQIFLSQKFVKLLYHAKRISNTINENHRKYVENHKKEFEELFYYILEFNE | |
| | NYVGAKKNGKLLNSAFQSWQNHSIDELCSSFIGPTGSERKGLFELTSRGSAADFEF | |
| | LGVKIPRYRDYTPSSLLKDATLIHQSVTGLYETRIDLAKLGEG | |
| 162 | KKPYTIGLDIGTNSVGWAVLTDQYDLVKRKMKIAGDSEKKQIKKNFWGVRLFDEGQ | Listeria innocua Cas9 |
| | TAADRRMARTARRRIERRRNRISYLQGIFAEEMSKTDANFFCRLSDSFYVDNEKRN | |
| | SRHPFFATIEEEVEYHKNYPTIYHLREELVNSSEKADLRLVYLALAHIIKYRGNFL | |
| | IEGALDTQNTSVDGIYKQFIQTYNQVFASGIEDGSLKKLEDNKDVAKILVEKVTRK | |
| | EKLERILKLYPGEKSAGMFAQFISLIVGSKGNFQKPFDLIEKSDIECAKDSYEEDL | |
| | ESLLALIGDEYAELFVAAKNAYSAVVLSSIITVAETETNAKLSASMIERFDTHEED | |
| | LGELKAFIKLHLPKHYEEIFSNTEKHGYAGYIDGKTKQADFYKYMKMTLENIEGAD | |
| | YFIAKIEKENFLRKQRTFDNGAIPHQLHLEELEAILHQQAKYYPFLKENYDKIKSL | |
| | VTFRIPYFVGPLANGQSEFAWLTRKADGEIRPWNIEEKVDFGKSAVDFIEKMTNKD | |
| | TYLPKENVLPKHSLCYQKYLVYNELTKVRYINDQGKTSYFSGQEKEQIFNDLFKQK | |
| | RKVKKKDLELFLRNMSHVESPTIEGLEDSFNSSYSTYHDLLKVGIKQEILDNPVNT | |
| | EMLENIVKILTVFEDKRMIKEQLQQFSDVLDGVVLKKLERRHYTGWGRLSAKLLMG | |
| | IRDKQSHLTILDYLMNDDGLNRNLMQLINDSNLSFKSIIEKEQVTTADKDIQSIVA | |
| | DLAGSPAIKKGILQSLKIVDELVSVMGYPPQTIVVEMARENQTTGKGKNNSRPRYK | |
| | SLEKAIKEFGSQILKEHPTDNQELRNNRLYLYYLQNGKDMYTGQDLDIHNLSNYDI | |
| | DHIVPQSFITDNSIDNLVLTSSAGNREKGDDVPPLEIVRKRKVFWEKLYQGNLMSK | |
| | RKFDYLTKAERGGLTEADKARFIHRQLVETRQITKNVANILHQRFNYEKDDHGNTM | |
| | KQVRIVTLKSALVSQFRKQFQLYKVRDVNDYHHAHDAYLNGVVANTLLKVYPQLEP | |
| | EFVYGDYHQFDWFKANKATAKKQFYTNIMLFFAQKDRIIDENGEILWDKKYLDTVK | |
| | KVMSYRQMNIVKKTEIQKGEFSKATIKPKGNSSKLIPRKTNWDPMKYGGLDSPNMA | |
| | YAVVIEYAKGKNKLVFEKKIIRVTIMERKAFEKDEKAFLEEQGYRQPKVLAKLPKY | |
| | TLYECEEGRRRMLASANEAQKGNQQVLPNHLVTLLHHAANCEVSDGKSLDYIESNR | |
| | EMFAELLAHVSEFAKRYTLAEANLNKINQLFEQNKEGDIKAIAQSFVDLMAFNAMG | |
| | APASFKFFETTIERKRYNNLKELLNSTIIYQSITGLYESRKRLDD | |
| 163 | GAGAATCAAAATCGGTGAAT | TRAC target sequence |
| | | 2 |
| 164 | GGCCTCGGCGCTGACGATCT | TRBC target sequence |
| | | 2 |
| 165 | GAGAATCAAAATCGGTGAATAGG | TRAC target sequence |
| | | with PAM 3 |
| 166 | TTCAAAACCTGTCAGTGATTGGG | TRAC target sequence |
| | | with PAM 4 |
| 167 | TGTGCTAGACATGAGGTCTATGG | TRAC target sequence |
| | | with PAM 5 |
| 168 | CGTCATGAGCAGATTAAACCCGG | TRAC target sequence |
| | | with PAM 6 |
| 169 | TCAGGGTTCTGGATATCTGTGGG | TRAC target sequence |
| | | with PAM 7 |
| 170 | GTCAGGGTTCTGGATATCTGTGG | TRAC target sequence |
| | | with PAM 8 |
| 171 | TTCGGAACCCAATCACTGACAGG | TRAC target sequence |
| | | with PAM 9 |
| 172 | TAAACCCGGCCACTTTCAGGAGG | TRAC target sequence |
| | | with PAM 10 |
| 173 | AAAGTCAGATTTGTTGCTCCAGG | TRAC target sequence |
| | | with PAM 11 |
| 174 | AACAAATGTGTCACAAAGTAAGG | TRAC target sequence |
| | | with PAM 12 |
| 175 | TGGATTTAGAGTCTCTCAGCTGG | TRAC target sequence |
| | | with PAM 13 |
| 176 | TAGGCAGACAGACTTGTCACTGG | TRAC target sequence |
| | | with PAM 14 |
| 177 | AGCTGGTACACGGCAGGGTCAGG | TRAC target sequence |
| | | with PAM 15 |
| 178 | GC TGGTACACGGCAGGGTCAGGG | TRAC target sequence |
| | | with PAM 16 |
| 179 | TCTCTCAGCTGGTACACGGCAGG | TRAC target sequence |
| | | with PAM 17 |
| 180 | TTTCAAAACCTGTCAGTGATTGG | TRAC target sequence |
| | | with PAM 18 |
| 181 | GATTAAACCCGGCCACTTTCAGG | TRAC target sequence |
| | | with PAM 19 |
| 182 | CTCGACCAGCTTGACATCACAGG | TRAC target sequence |
| | | with PAM 20 |
| 183 | AGAGTCTCTCAGCTGGTACACGG | TRAC target sequence |
| | | with PAM 21 |
| 184 | CTCTCAGCTGGTACACGGCAGGG | TRAC target sequence |
| | | with PAM 22 |
| 185 | AAGTTCCTGTGATGTCAAGCTGG | TRAC target sequence |
| | | with PAM 23 |
| 186 | ATCCTCCTCCTGAAAGTGGCCGG | TRAC target sequence |
| | | with PAM 24 |
| 187 | TGCTCATGACGCTGCGGCTGTGG | TRAC target sequence |
| | | with PAM 25 |
| 188 | ACAAAACTGTGCTAGACATGAGG | TRAC target sequence |
| | | with PAM 26 |
| 189 | ATTTGTTTGAGAATCAAAATCGG | TRAC target sequence |
| | | with PAM 27 |
| 190 | CATCACAGGAACTTTCTAAAAGG | TRAC target sequence |
| | | with PAM 28 |
| 191 | GTCGAGAAAAGCTTTGAAACAGG | TRAC target sequence |
| | | with PAM 29 |
| 192 | CCACTTTCAGGAGGAGGATTCGG | TRAC target sequence |
| | | with PAM 30 |
| 193 | CTGACAGGTTTTGAAAGTTTAGG | TRAC target sequence |
| | | with PAM 31 |
| 194 | AGCTTTGAAACAGGTAAGACAGG | TRAC target sequence |
| | | with PAM 32 |
| 195 | TGGAATAATGCTGTTGTTGAAGG | TRAC target sequence |
| | | with PAM 33 |
| 196 | AGAGCAACAGTGCTGTGGCCTGG | TRAC target sequence |
| | | with PAM 34 |
| 197 | CTGTGGTCCAGCTGAGGTGAGGG | TRAC target sequence |
| | | with PAM 35 |
| 198 | CTGCGGCTGTGGTCCAGCTGAGG | TRAC target sequence |
| | | with PAM 36 |
| 199 | TGTGGTCCAGCTGAGGTGAGGGG | TRAC target sequence |
| | | with PAM 37 |
| 200 | CTTCTTCCCCAGCCCAGGTAAGG | TRAC target sequence |
| | | with PAM 38 |
| 201 | ACACGGCAGGGTCAGGGTTCTGG | TRAC target sequence |
| | | with PAM 39 |
| 202 | CTTCAAGAGCAACAGTGCTGTGG | TRAC target sequence |
| | | with PAM 40 |
| 203 | CTGGGGAAGAAGGTGTCTTCTGG | TRAC target sequence |
| | | with PAM 41 |
| 204 | TCCTCCTCCTGAAAGTGGCCGGG | TRAC target sequence |
| | | with PAM 42 |
| 205 | TTAATCTGCTCATGACGCTGCGG | TRAC target sequence |
| | | with PAM 43 |
| 206 | ACCCGGCCACTTTCAGGAGGAGG | TRAC target sequence |
| | | with PAM 44 |
| 207 | TTCTTCCCCAGCCCAGGTAAGGG | TRAC target sequence |
| | | with PAM 45 |
| 208 | CTTACCTGGGCTGGGGAAGAAGG | TRAC target sequence |
| | | with PAM 46 |
| 209 | GACACCTTCTTCCCCAGCCCAGG | TRAC target sequence |
| | | with PAM 47 |
| 210 | GCTGTGGTCCAGCTGAGGTGAGG | TRAC target sequence |
| | | with PAM 48 |
| 211 | CCGAATCCTCCTCCTGAAAGTGG | TRAC target sequence |
| | | with PAM 49 |
| 212 | GCTGTCAAGTCCAGTTCTACGGG | TRBC target sequence |
| | | with PAM 3 |
| 213 | CTATGGACTTCAAGAGCAACAGTGCTGT | TRAC ZFN target |
| | | sequence 1 |
| 214 | CTCATGTCTAGCACAGTTTTGTCTGTGA | TRAC ZFN target |
| | | sequence 2 |
| 215 | GTGCTGTGGCCTGGAGCAACAAATCTGA | TRAC ZFN target |
| | | sequence 3 |
| 216 | TTGCTCTTGAAGTCCATAGACCTCATGT | TRAC ZFN target |
| | | sequence 4 |
| 217 | GCTGTGGCCTGGAGCAACAAATCTGACT | TRAC ZFN target |
| | | sequence 5 |
| 218 | CTGTTGCTCTTGAAGTCCATAGACCTCA | TRAC ZFN target |
| | | sequence 6 |
| 219 | CTGTGGCCTGGAGCAACAAATCTGACTT | TRAC ZFN target |
| | | sequence 7 |
| 220 | CTGACTTTGCATGTGCAAACGCCTTCAA | TRAC ZFN target |
| | | sequence 8 |
| 221 | TTGTTGCTCCAGGCCACAGCACTGTTGC | TRAC ZFN target |
| | | sequence 9 |
| 222 | TGAAAGTGGCCGGGTTTAATCTGCTCAT | TRAC ZFN target |
| | | sequence 10 |
| 223 | AGGAGGATTCGGAACCCAATCACTGACA | TRAC ZFN target |
| | | sequence 11 |
| 224 | GAGGAGGATTCGGAACCCAATCACTGAC | TRAC ZFN target |
| | | sequence 12 |
| 225 | CCGTAGAACTGGACTTGACAGCGGAAGT | TRBC ZFN target |
| | | sequence 1 |
| 226 | TCTCGGAGAATGACGAGTGGACCCAGGA | TRBC ZFN target |
| | | sequence 2 |
| 227 | MAAFKPNSINYILGLDIGIASVGWAMVEIDEEENPIRLIDLGVRVFERAEVPKTGD | Neisseria meningitidis |
| | SLAMARRLARSVRRLTRRRAHRLLRTRRLLKREGVLQAANFDENGLIKSLPNTPWQ | Cas9 |
| | LRAAALDRKLTPLEWSAVLLHLIKHRGYLSQRKNEGETADKELGALLKGVAGNAHA | |
| | LQTGDFRTPAELALNKFEKESGHIRNQRSDYSHTFSRKDLQAELILLFEKQKEFGN | |
| | PHVSGGLKEGIETLLMTQRPALSGDAVQKMLGHCTFEPAEPKAAKNTYTAERFIWL | |
| | TKLNNLRILEQGSERPLTDTERATLMDEPYRKSKLTYAQARKLLGLEDTAFFKGLR | |
| | YGKDNAEASTLMEMKAYHAISRALEKEGLKDKKSPLNLSPELQDEIGTAFSLFKTD | |
| | EDITGRLKDRIQPEILEALLKHISFDKFVQISLKALRRIVPLMEQGKRYDEACAEI | |
| | YGDHYGKKNTEEKIYLPPIPADEIRNPVVLRALSQARKVINGVVRRYGSPARIHIE | |
| | TAREVGKSFKDRKEIEKRQEENRKDREKAAAKFREYFPNFVGEPKSKDILKLRLYE | |
| | QQHGKCLYSGKEINLGRLNEKGYVEIDHALPFSRTWDDSFNNKVLVLGSENQNKGN | |
| | QTPYEYFNGKDNSREWQEFKARVETSRFPRSKKQRILLQKFDEDGFKERNLNDTRY | |
| | VNRFLCQFVADRMRLTGKGKKRVFASNGQITNLLRGFWGLRKVRAENDRHHALDAV | |
| | VVACSTVAMQQKITRFVRYKEMNAFDGKTIDKETGEVLHQKTHFPQPWEFFAQEVM | |
| | IRVFGKPDGKPEFEEADTLEKLRTLLAEKLSSRPEAVHEYVTPLFVSRAPNRKMSG | |
| | QGHMETVKSAKRLDEGVSVLRVPLTQLKLKDLEKMVNREREPKLYEALKARLEAHK | |
| | DDPAKAFAEPFYKYDKAGNRTQQVKAVRVEQVQKTGVWVRNHNGIADNATMVRVDV | |
| | FEKGDKYYLVPIYSWQVAKGILPDRAVVQGKDEEDWQLIDDSFNFKFSLHPNDLVE | |
| | VITKKARMFGYFASCHRGTGNINIRIHDLDHKIGKNGILEGIGVKTALSFQKYQID | |
| | ELGKEIRPCRLKKRPPVR | |
| 228 | MDKKYSIGLDIGTNSVGWAVITDEYKVPSKKFKVLGNTDRHSIKKNLIGALLFDSG | Streptococcus |
| | ETAEATRLKRTARRRYTRRKNRICYLQEIFSNEMAKVDDSFFHRLEESFLVEEDKK | pyogenes Cas9 |
| | HERHPIFGNIVDEVAYHEKYPTIYHLRKKLVDSTDKADLRLIYLALAHMIKFRGHF | |
| | LIEGDLNPDNSDVDKLFIQLVQTYNQLFEENPINASGVDAKAILSARLSKSRRLEN | |
| | LIAQLPGEKKNGLFGNLIALSLGLTPNFKSNFDLAEDAKLQLSKDTYDDDLDNLLA | |
| | QIGDQYADLFLAAKNLSDAILLSDILRVNTEITKAPLSASMIKRYDEHHQDLTLLK | |
| | ALVRQQLPEKYKEIFFDQSKNGYAGYIDGGASQEEFYKFIKPILEKMDGTEELLVK | |
| | LNREDLLRKQRTFDNGSIPHQIHLGELHAILRRQEDFYPFLKDNREKIEKILTFRI | |
| | PYYVGPLARGNSRFAWMTRKSEETITPWNFEEVVDKGASAQSFIERMTNFDKNLPN | |
| | EKVLPKHSLLYEYFTVYNELTKVKYVTEGMRKPAFLSGEQKKAIVDLLFKTNRKVT | |
| | VKQLKEDYFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGRLSRKLINGIRD | |
| | KQSGKTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRERMKRI | |
| | EEGIKELGSQILKEHPVENTQLQNEKLYLYYLQNGRDMYVDQELDINRLSDYDVDH | |
| | IVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEF | |
| | VYGDYKVYDVRKMIAKSEQEIGKATAKYFFYSNIMNFFKTEITLANGEIRKRPLIE | |
| | TNGETGEIVWDKGRDFATVRKVLSMPQVNIVKKTEVQTGGFSKESILPKRNSDKLI | |
| | ARKKDWDPKKYGGFDSPTVAYSVLVVAKVEKGKSKKLKSVKELLGITIMERSSFEK | |
| | NPIDFLEAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVILADANLD | |
| | KVLSAYNKHRDKPIREQAENIIHLFTLTNLGAPAAFKYFDTTIDRKRYTSTKEVLD | |
| | ATLIHQSITGLYETRIDLSQLGGD | |
| 229 | AGCTGAGAGACTCTAAATCCAGTGACAAGTCTGTCTGCCTATTCACCGAT | TRAC 50 bp 5′ |
| | | Homology Arm |
| 230 | CTGATCCTCTTGTCCCACAGATATCCAGAACCCTGACCCTGCCGTGTACCAGCTGA | TRAC 100 bp 5′ |
| | GAGACTCTAAATCCAGTGACAAGTCTGTCTGCCTATTCACCGAT | Homology Arm |
| 231 | GTGACTTGCCAGCCCCACAGAGCCCCGCCCTTGTCCATCACTGGCATCTGGACTCC | TRAC 200 bp 5′ |
| | AGCCTGGGTTGGGGCAAAGAGGGAAATGAGATCATGTCCTAACCCTGATCCTCTTG | Homology Arm |
| | TCCCACAGATATCCAGAACCCTGACCCTGCCGTGTACCAGCTGAGAGACTCTAAAT | |
| | CCAGTGACAAGTCTGTCTGCCTATTCACCGAT | |
| 232 | CCTCTTGGCCAAGATTGATAGCTTGTGCCTGTCCCTGAGTCCCAGTCCATCACGAG | TRAC 300 bp 5′ |
| | CAGCTGGTTTCTAAGATGCTATTTCCCGTATAAAGCATGAGACCGTGACTTGCCAG | Homology Arm |
| | CCCCACAGAGCCCCGCCCTTGTCCATCACTGGCATCTGGACTCCAGCCTGGGTTGG | |
| | GGCAAAGAGGGAAATGAGATCATGTCCTAACCCTGATCCTCTTGTCCCACAGATAT | |
| | CCAGAACCCTGACCCTGCCGTGTACCAGCTGAGAGACTCTAAATCCAGTGACAAGT | |
| | CTGTCTGCCTATTCACCGAT | |
| 233 | ATTAAATAAAAGAATAAGCAGTATTATTAAGTAGCCCTGCATTTCAGGTTTCCTTG | TRAC 400 bp 5′ |
| | AGTGGCAGGCCAGGCCTGGCCGTGAACGTTCACTGAAATCATGGCCTCTTGGCCAA | Homology Arm |
| | GATTGATAGCTTGTGCCTGTCCCTGAGTCCCAGTCCATCACGAGCAGCTGGTTTCT | |
| | AAGATGCTATTTCCCGTATAAAGCATGAGACCGTGACTTGCCAGCCCCACAGAGCC | |
| | CCGCCCTTGTCCATCACTGGCATCTGGACTCCAGCCTGGGTTGGGGCAAAGAGGGA | |
| | AATGAGATCATGTCCTAACCCTGATCCTCTTGTCCCACAGATATCCAGAACCCTGA | |
| | CCCTGCCGTGTACCAGCTGAGAGACTCTAAATCCAGTGACAAGTCTGTCTGCCTAT | |
| | TCACCGAT | |
| 234 | CTCCAGATTCCAAGATGTACAGTTTGCTTTGCTGGGCCTTTTTCCCATGCC | TGCCT TRAC 500 bp 5′ |
| | TTACTCTGCCAGAGTTATATTGCTGGGGTTTTGAAGAAGATCCTATTAAATAAAAG | Homology Arm |
| | AATAAGCAGTATTATTAAGTAGCCCTGCATTTCAGGTTTCCTTGAGTGGCAGGCCA | |
| | GGCCTGGCCGTGAACGTTCACTGAAATCATGGCCTCTTGGCCAAGATTGATAGCTT | |
| | GTGCCTGTCCCTGAGTCCCAGTCCATCACGAGCAGCTGGTTTCTAAGATGCTATTT | |
| | CCCGTATAAAGCATGAGACCGTGACTTGCCAGCCCCACAGAGCCCCGCCCTTGTCC | |
| | ATCACTGGCATCTGGACTCCAGCCTGGGTTGGGGCAAAGAGGGAAATGAGATCATG | |
| | TCCTAACCCTGATCCTCTTGTCCCACAGATATCCAGAACCCTGACCCTGCCGTGTA | |
| | CCAGCTGAGAGACTCTAAATCCAGTGACAAGTCTGTCTGCCTATTCACCGAT | |
| 235 | AGAGAGCAATCTCCTGGTAATGTGATAGATTTCCCAACTTAATGCCAACATACCAT | TRAC 600 bp 5′ |
| | AAACCTCCCATTCTGCTAATGCCCAGCCTAAGTTGGGGAGACCACTCCAGATTCCA | Homology Arm |
| | AGATGTACAGTTTGCTTTGCTGGGCCTTTTTCCCATGCCTGCCTTTACTCTGCCAG | |
| | AGTTATATTGCTGGGGTTTTGAAGAAGATCCTATTAAATAAAAGAATAAGCAGTAT | |
| | TATTAAGTAGCCCTGCATTTCAGGTTTCCTTGAGTGGCAGGCCAGGCCTGGCCGTG | |
| | AACGTTCACTGAAATCATGGCCTCTTGGCCAAGATTGATAGCTTGTGCCTGTCCCT | |
| | GAGTCCCAGTCCATCACGAGCAGCTGGTTTCTAAGATGCTATTTCCCGTATAAAGC | |
| | ATGAGACCGTGACTTGCCAGCCCCACAGAGCCCCGCCCTTGTCCATCACTGGCATC | |
| | TGGACTCCAGCCTGGGTTGGGGCAAAGAGGGAAATGAGATCATGTCCTAACCCTGA | |
| | TCCTCTTGTCCCACAGATATCCAGAACCCTGACCCTGCCGTGTACCAGCTGAGAGA | |
| | CTCTAAATCCAGTGACAAGTCTGTCTGCCTATTCACCGAT | |
| 236 | TTTGATTCTCAAACAAATGTGTCACAAAGTAAGGATTCTGATGTGTATAT | TRAC 50 bp 3′ |
| | | Homology Arm |
| 237 | TTTGATTCTCAAACAAATGTGTCACAAAGTAAGGATTCTGATGTGTATATCACAGA | TRAC 100 bp 3′ |
| | CAAAACTGTGCTAGACATGAGGTCTATGGACTTCAAGAGCAACA | Homology Arm |
| 238 | TTTGATTCTCAAACAAATGTGTCACAAAGTAAGGATTCTGATGTGTATATCACAGA | TRAC 200 bp 3′ |
| | CAAAACTGTGCTAGACATGAGGTCTATGGACTTCAAGAGCAACAGTGCTGTGGCCT | Homology Arm |
| | GGAGCAACAAATCTGACTTTGCATGTGCAAACGCCTTCAACAACAGCATTATTCCA | |
| | GAAGACACCTTCTTCCCCAGCCCAGGTAAGGG | |
| 239 | TTTGATTCTCAAACAAATGTGTCACAAAGTAAGGATTCTGATGTGTATATCACAGA | TRAC 300 bp 3′ |
| | CAAAACTGTGCTAGACATGAGGTCTATGGACTTCAAGAGCAACAGTGCTGTGGCCT | Homology Arm |
| | GGAGCAACAAATCTGACTTTGCATGTGCAAACGCCTTCAACAACAGCATTATTCCA | |
| | GAAGACACCTTCTTCCCCAGCCCAGGTAAGGGCAGCTTTGGTGCCTTCGCAGGCTG | |
| | TTTCCTTGCTTCAGGAATGGCCAGGTTCTGCCCAGAGCTCTGGTCAATGATGTCTA | |
| | AAACTCCTCTGATTGGTGGT | |
| 240 | TTTGATTCTCAAACAAATGTGTCACAAAGTAAGGATTCTGATGTGTATATCACAGA | TRAC 400 bp 3′ |
| | CAAAACTGTGCTAGACATGAGGTCTATGGACTTCAAGAGCAACAGTGCTGTGGCCT | Homology Arm |
| | GGAGCAACAAATCTGACTTTGCATGTGCAAACGCCTTCAACAACAGCATTATTCCA | |
| | GAAGACACCTTCTTCCCCAGCCCAGGTAAGGGCAGCTTTGGTGCCTTCGCAGGCTG | |
| | TTTCCTTGCTTCAGGAATGGCCAGGTTCTGCCCAGAGCTCTGGTCAATGATGTCTA | |
| | AAACTCCTCTGATTGGTGGTCTCGGCCTTATCCATTGCCACCAAAACCCTCTTTTT | |
| | ACTAAGAAACAGTGAGCCTTGTTCTGGCAGTCCAGAGAATGACACGGGAAAAAAGC | |
| | AGATGAAG | |
| 241 | TTTGATTCTCAAACAAATGTGTCACAAAGTAAGGATTCTGATGTGTATATCACAGA | TRAC 500 bp 3′ |
| | CAAAACTGTGCTAGACATGAGGTCTATGGACTTCAAGAGCAACAGTGCTGTGGCCT | Homology Arm |
| | GGAGCAACAAATCTGACTTTGCATGTGCAAACGCCTTCAACAACAGCATTATTCCA | |
| | GAAGACACCTTCTTCCCCAGCCCAGGTAAGGGCAGCTTTGGTGCCTTCGCAGGCTG | |
| | TTTCCTTGCTTCAGGAATGGCCAGGTTCTGCCCAGAGCTCTGGTCAATGATGTCTA | |
| | AAACTCCTCTGATTGGTGGTCTCGGCCTTATCCATTGCCACCAAAACCCTCTTTTT | |
| | ACTAAGAAACAGTGAGCCTTGTTCTGGCAGTCCAGAGAATGACACGGGAAAAAAGC | |
| | AGATGAAGAGAAGGTGGCAGGAGAGGGCACGTGGCCCAGCCTCAGTCTCTCCAACT | |
| | GAGTTCCTGCCTGCCTGCCTTTGCTCAGACTGTTTGCCCCTTACTGCTCTTC | |
| 242 | TTTGATTCTCAAACAAATGTGTCACAAAGTAAGGATTCTGATGTGTATATCACAGA | TRAC 600 bp 3′ |
| | CAAAACTGTGCTAGACATGAGGTCTATGGACTTCAAGAGCAACAGTGCTGTGGCCT | Homology Arm |
| | GGAGCAACAAATCTGACTTTGCATGTGCAAACGCCTTCAACAACAGCATTATTCCA | |
| | GAAGACACCTTCTTCCCCAGCCCAGGTAAGGGCAGCTTTGGTGCCTTCGCAGGCTG | |
| | TTTCCTTGCTTCAGGAATGGCCAGGTTCTGCCCAGAGCTCTGGTCAATGATGTCTA | |
| | AAACTCCTCTGATTGGTGGTCTCGGCCTTATCCATTGCCACCAAAACCCTCTTTTT | |
| | ACTAAGAAACAGTGAGCCTTGTTCTGGCAGTCCAGAGAATGACACGGGAAAAAAGC | |
| | AGATGAAGAGAAGGTGGCAGGAGAGGGCACGTGGCCCAGCCTCAGTCTCTCCAACT | |
| | GAGTTCCTGCCTGCCTGCCTTTGCTCAGACTGTTTGCCCCTTACTGCTCTTCTAGG | |
| | CCTCATTCTAAGCCCCTTCTCCAAGTTGCCTCTCCTTATTTCTCCCTGTCTGCCAA | |
| | AAAATCTTTCCCAGCTCACTAAGTCAGTCTCACGCAGTCA | |
| 243 | NIQKPDPAVYQLRDSKSSDKSVCLFTDFDSQTNVSQSKDSDVYITDKTVLDMRSMD | Human TCR alpha |
| | FKSNSAVAWSNKSDFACANAFNNSIIPADTFFPSPESSCDVKLVEKSFETDTNLNF | constant |
| | QNLSVIGFRILLLKVAGFNLLMTLRLWSS | |
| 244 | HIQNPDPAVYQLRDSKSSDKSVCLFTDFDSQTNVSQSKDSDVYITDKTVLDMRSMD | Human TCR alpha |
| | FKSNSAVAWSNKSDFACANAFNNSIIPEDTFFPSPESSCDVKLVEKSFETDTNLNF | constant |
| | QNLSVIGFRILLLKVAGFNLLMTLRLWSS | |
| 245 | YIQNPDPAVYQLRDSKSSDKSVCLFTDFDSQTNVSQSKDSDVYITDKTVLDMRSMD | Human TCR alpha |
| | FKSNSAVAWSNKSDFACANAFNNSIIPEDTFFPSPESSCDVKLVEKSFETDTNLNF | constant |
| | QNLSVIGFRILLLKVAGFNLLMTLRLWSS | |
| 246 | DIQNPDPAVYQLRDSKSSDKSVCLFTDFDSQTNVSQSKDSDVYITDKTVLDMRSMD | Human TCR alpha |
| | FKSNSAVAWSNKSDFACANAFNNSIIPEDTFFPSPESSCDVKLVEKSFETDTNLNF | constant |
| | QNLSVIGFRILLLKVAGFNLLMTLRLWSS | |
| 247 | PNIQKPDPAVYQLRDSKSSDKSVCLFTDFDSQTNVSQSKDSDVYITDKTVLDMRSM | Human TCR alpha |
| | DFKSNSAVAWSNKSDFACANAFNNSIIPADTFFPSPESSCDVKLVEKSFETDTNLN | constant |
| | FQNLSVIGFRILLLKVAGFNLLMTLRLWSS | |
| 248 | NIQKPDPAVYQLRDSKSSDKSVCLFTDFDSQTNVSQSKDSDVYITDKCVLDMRSMD | Human TCR alpha |
| | FKSNSAVAWSNKSDFACANAFNNSIIPADTFFPSPESSCDVKLVEKSFETDTNLNF | constant |
| | QNLSVIGFRILLLKVAGFNLLMTLRLWSS | |
| 249 | HIQNPDPAVYQLRDSKSSDKSVCLFTDFDSQTNVSQSKDSDVYITDKCVLDMRSMD | Human TCR alpha |
| | FKSNSAVAWSNKSDFACANAFNNSIIPEDTFFPSPESSCDVKLVEKSFETDTNLNF | constant |
| | QNLSVIGFRILLLKVAGFNLLMTLRLWSS | |
| 250 | YIQNPDPAVYQLRDSKSSDKSVCLFTDFDSQTNVSQSKDSDVYITDKCVLDMRSMD | Human TCR alpha |
| | FKSNSAVAWSNKSDFACANAFNNSIIPEDTFFPSPESSCDVKLVEKSFETDTNLNF | constant |
| | QNLSVIGFRILLLKVAGFNLLMTLRLWSS | |
| 251 | NIQNPDPAVYQLRDSKSSDKSVCLFTDFDSQTNVSQSKDSDVYITDKCVLDMRSMD | Human TCR alpha |
| | FKSNSAVAWSNKSDFACANAFNNSIIPEDTFFPSPESSCDVKLVEKSFETDTNLNF | constant |
| | QNLSVIGFRILLLKVAGFNLLMTLRLWSS | |
| 252 | DIQNPDPAVYQLRDSKSSDKSVCLFTDFDSQTNVSQSKDSDVYITDKCVLDMRSMD | Human TCR alpha |
| | FKSNSAVAWSNKSDFACANAFNNSIIPEDTFFPSPESSCDVKLVEKSFETDTNLNF | constant |
| | QNLSVIGFRILLLKVAGFNLLMTLRLWSS | |
| 253 | EDLNKVFPPEVAVFEPSEAEISHTQKATLVCLATGFFPDHVELSWWVNGKEVHSGV | Human TCR beta |
| | CTDPQPLKEQPALNDSRYCLSSRLRVSATFWQNPRNHFRCQVQFYGLSENDEWTQD | constant |
| | RAKPVTQIVSAEAWGRADCGFTSVSYQQGVLSATILYEILLGKATLYAVLVSALVL | |
| | MAMVKRKDF | |
| 254 | TEDLKNVFPPEVAVFEPSEAEISHTQKATLVCLATGFYPDHVELSWWVNGKEVHSG | Human TCR beta |
| | VSTDPQPLKEQPALNDSRYCLSSRLRVSATFWQNPRNHFRCQVQFYGLSENDEWTQ | constant |
| | DRAKPVTQIVSAEAWGRADCGFTSESYQQGVLSATILYEILLGKATLYAVLVSALV | |
| | LMAMVKRKDSRG | |
| 255 | LEDLKNVFPPEVAVFEPSEAEISHTQKATLVCLATGFYPDHVELSWWVNGKEVHSG | Human TCR beta |
| | VSTDPQPLKEQPALNDSRYCLSSRLRVSATFWQNPRNHFRCQVQFYGLSENDEWTQ | constant |
| | DRAKPVTQIVSAEAWGRADCGFTSESYQQGVLSATILYEILLGKATLYAVLVSALV | |
| | LMAMVKRKDSRG | |
| 256 | EDLKNVFPPEVAVFEPSEAEISHTQKATLVCLATGFYPDHVELSWWVNGKEVHSGV | Human TCR beta |
| | CTDPQPLKEQPALNDSRYCLSSRLRVSATFWQNPRNHFRCQVQFYGLSENDEWTQD | constant |
| | RAKPVTQIVSAEAWGRADCGFTSESYQQGVLSATILYEILLGKATLYAVLVSALVL | |
| | MAMVKRKDSRG | |
| 257 | TEDLKNVFPPEVAVFEPSEAEISHTQKATLVCLATGFYPDHVELSWWVNGKEVHSG | Human TCR beta |
| | VCTDPQPLKEQPALNDSRYCLSSRLRVSATFWQNPRNHFRCQVQFYGLSENDEWTQ | constant |
| | DRAKPVTQIVSAEAWGRADCGFTSESYQQGVLSATILYEILLGKATLYAVLVSALV | |
| | LMAMVKRKDSRG | |
| 258 | LEDLKNVFPPEVAVFEPSEAEISHTQKATLVCLATGFYPDHVELSWWVNGKEVHSG | Human TCR beta |
| | VCTDPQPLKEQPALNDSRYCLSSRLRVSATFWQNPRNHFRCQVQFYGLSENDEWTQ | constant |
| | DRAKPVTQIVSAEAWGRADCGFTSESYQQGVLSATILYEILLGKATLYAVLVSALV | |
| | LMAMVKRKDSRG | |
| 259 | QNPDPAVYQLRDSKSSDKSVCLFTDFDSQTNVSQSKDSDVYITDKC | Partial TCR alpha |
| | | constant region |
| 260 | GTGCTAGACATGAGGTCTATGGACTTCAAGAGCAACAGTGCTGTGGCCTGGAGCAA | Partial endogenous |
| | CAAATCTGACTTTGCATGTGCAAACGCCTTCAACAACAGCATTATTCCAGAAGACA | TCR alpha constant |
| | CCTTCTTCCCCAGCCCAGG | region |
| 261 | VLDMRSMDFKSNSAVAWSNKSDFACANAFNNSIIPEDTFFPSP | Partial TCR alpha |
| | | constant region |

1. A genetically engineered T cell, comprising a modified T cell receptor alpha constant (TRAC) locus, said modified TRAC locus comprising a nucleic acid encoding a recombinant TCR or portion thereof, said recombinant TCR or portion thereof comprising: (i) a TCR alpha (TCRα) chain comprising a variable alpha (Vα) domain and a constant alpha (Cα) domain, and (ii) a TCR beta (TCRβ) chain comprising a variable beta (Vβ) domain and a constant beta (Cβ) domain, wherein the nucleic acid sequence comprises of (a) a transgene sequence encoding the TCRβ and the Vα domain, and (b) an open reading frame of the endogenous TRAC locus or a partial sequence thereof, wherein the open reading frame encodes at least a portion of the Cα domain of the recombinant TCR.

2. A genetically engineered T cell, comprising a modified T cell receptor alpha constant (TRAC) locus, said modified TRAC locus comprising a nucleic acid encoding a recombinant TCR or portion thereof, said recombinant TCR or portion thereof comprising: (i) a TCR alpha (TCRα) chain comprising a variable alpha (Vα) domain and a constant alpha (Cα) domain, and (ii) a TCR beta (TCRβ) chain comprising a variable beta (Vβ) domain and a constant beta (Cβ) domain, wherein the nucleic acid sequence comprises of (a) a transgene sequence encoding the TCRβ and the Vα domain, and (b) an open reading frame of the endogenous TRAC locus or a partial sequence thereof, wherein the open reading frame encodes at least a portion of the Cα domain of the recombinant TCR, wherein:
a portion of the Cα is encoded by the open reading frame of the endogenous TRAC locus or a partial sequence thereof, and a further portion of the Cα is encoded by the transgene sequence, wherein said further portion of Cα is less than the full length of a native Cα; and
the further portion of the Cα and/or the Cβ region encoded by the nucleic acid sequence of (a) comprises one or more modifications compared to a native Cα region and/or a native Cβ region, said one or more modifications introduces one or more cysteine residues that are capable of forming one or more non-native disulfide bridges between the alpha chain and beta chain and/or wherein the Cα and/or the Cβ of the recombinant TCR comprises one or more non-native cysteines.

3. A genetically engineered T cell, comprising a modified T cell receptor alpha constant (TRAC) locus, said modified TRAC locus comprising a nucleic acid encoding a recombinant TCR or portion thereof, said recombinant TCR or portion thereof comprising: (i) a TCR alpha (TCRα) chain comprising a variable alpha (Vα) domain and a constant alpha (Cα) domain, and (ii) a TCR beta (TCRβ) chain comprising a variable beta (Vβ) domain and a constant beta (Cβ) domain, wherein the nucleic acid sequence comprises of (a) a transgene sequence encoding the TCRβ and the Vα domain, and (b) an open reading frame of the endogenous TRAC locus or a partial sequence thereof, wherein the open reading frame encodes at least a portion of the Cα domain of the recombinant TCR, wherein the transgene sequence comprises one or more heterologous or regulatory control element(s) comprising a heterologous promoter, operably linked to control expression of the TCR when expressed from a cell introduced with the genetically engineered T cell.

4. The genetically engineered T cell of any of claims 1-3, wherein the transgene sequence has been integrated via homology directed repair (HDR).

5. The genetically engineered T cell of any of claims 1-4, wherein the modified TRAC locus comprises an in-frame fusion of (i) a transgene sequence and (ii) an open reading frame or a partial sequence thereof of the endogenous TRAC locus, optionally wherein the transgene sequence is in-frame with one or more exons of the open reading frame or partial sequence thereof of the endogenous TRAC locus.

6. The genetically engineered T cell of any of claims 1-5, wherein the transgene sequence does not comprise a sequence encoding a 3′ untranslated region (3′ UTR) or an intron.

7. The genetically engineered T cell of any of claims 1 and 3-6, wherein a portion of the Cα is encoded by the open reading frame of the endogenous TRAC locus or a partial sequence thereof, and a further portion of the Cα is encoded by the transgene sequence, wherein said further portion of Cα is less than the full length of a native Cα.

8. The genetically engineered T cell of any of claims 1-7, wherein the open reading frame or the partial sequence thereof comprises at least one intron and at least one exon of the endogenous TRAC locus.

9. The genetically engineered T cell of any of claims 2, 7 and 8, wherein the further portion of the Cα is encoded by a sequence of nucleotides that comprises less than four exons, less than three exons, less than two exons, one exon, or less than one full exon the open reading frame of the TRAC locus.

10. The genetically engineered T cell of any of claims 2 and 7-9, wherein the further portion of the Cα is encoded by a sequence of nucleotides that is less than 400, less than 300, less than 250, less than 200, or less than 150 base pairs in length.

11. The genetically engineered T cell of any of claims 2 and 7-10, wherein the further portion of the Cα is encoded by a portion of exon 1 of the TRAC locus, wherein the portion of exon 1 is less than the full length of exon 1 the open reading frame of the TRAC locus.

12. The genetically engineered T cell of any of claims 1-11, wherein the transgene sequence has been integrated downstream of the most 5′ nucleotide of exon 1 and upstream of the most 3′ nucleotide of exon 1 of the open reading frame of the endogenous TRAC locus.

13. The genetically engineered T cell of any of claims 1-2, wherein the at least a portion of Cα is encoded by at least a portion of exon 1 and exons 2-4 of the open reading frame of the endogenous TRAC locus.

14. The genetically engineered T cell of any of claims 1-13, wherein the encoded TCRα chain is capable of dimerizing with a TCRβ chain.

15. The genetically engineered T cell of any of claims 1-14, wherein the encoded Cα comprises the sequence selected from any one of SEQ ID NOS: 14, 15, 19, and 24, or a sequence that exhibits at least 85%, 86%, 87%, 88%, 89%, 90%, 91%, 92%, 93%, 94%, 95%, 96%, 97%, 98%, 99% or more sequence identity to any one of SEQ ID NOS: 14, 15, 19, and 24, or a partial sequence thereof.

16. The genetically engineered T cell of any of claims 1-14, wherein the encoded Cα comprises the sequence selected from any one of SEQ ID NOS: 19, 24 and 243-252, or a sequence that exhibits at least 85%, 86%, 87%, 88%, 89%, 90%, 91%, 92%, 93%, 94%, 95%, 96%, 97%, 98%, 99% or more sequence identity to any one of SEQ ID NOS: 19, 24 and 243-252, or a partial sequence thereof.

17. The genetically engineered T cell of any of claims 2 and 7-16, wherein the at least a portion of Cα is encoded by a sequence of nucleotides starting from residue 3 and up to residue 3155 of the sequence set forth in SEQ ID NO:1 or one or more exons thereof or a sequence that exhibits at least 85%, 86%, 87%, 88%, 89%, 90%, 91%, 92%, 93%, 94%, 95%, 96%, 97%, 98%, 99% or more sequence identity to a sequence of nucleotides starting from residue 3 and up to residue 3155 of the sequence set forth in SEQ ID NO:1 or one or more exons thereof, or a partial sequence thereof.

18. The genetically engineered T cell of any of claims 2 and 7-17, wherein the further portion of the Cα comprises a sequence set forth in SEQ ID NO:142, or a sequence that exhibits at least 85%, 86%, 87%, 88%, 89%, 90%, 91%, 92%, 93%, 94%, 95%, 96%, 97%, 98%, 99% or more sequence identity to SEQ ID NO:142, or a partial sequence thereof.

19. The genetically engineered T cell of any of claims 7-18, wherein the further portion of the Cα and/or the Cβ region encoded by the nucleic acid sequence of (a) comprises one or more modifications, optionally a replacement, deletion, or insertion of one or more amino acids compared to a native Cα region and/or a native Cβ region, optionally said one or more modifications introduces one or more cysteine residues that are capable of forming one or more non-native disulfide bridges between the alpha chain and beta chain and/or wherein the Cα and/or the Cβ of the recombinant TCR comprises one or more non-native cysteines.

20. The genetically engineered T cell of claim 2 or claim 19, wherein the introduction of one or more cysteine residues comprises replacement of a non-cysteine residue with a cysteine residue.

21. The genetically engineered T cell of any of claims 2, 19 and 20, wherein the encoded Cα region comprises a cysteine at a position corresponding to position 48 with numbering as set forth in any of SEQ ID NO: 24; and/or the encoded Cβ region comprises a cysteine at a position corresponding to position 57 with numbering as set forth in SEQ ID NO: 20.

22. The genetically engineered T cell of any of claims 2 and 19-21, wherein the encoded Cα comprises the sequence selected from any one of SEQ ID NOS: 248-252, or a partial sequence thereof.

23. The genetically engineered T cell of any of claims 1-22, wherein the engineered T cell further comprises a genetic disruption at a TRBC locus.

24. A genetically engineered T cell, comprising a modified T cell receptor beta constant (TRBC) locus, said modified TRBC locus comprising a nucleic acid encoding a recombinant TCR or portion thereof, said recombinant TCR or portion thereof comprising: (i) a TCR beta (TCRβ) chain comprising a variable beta (Vβ) domain and a constant beta (Cβ) domain, and (ii) a TCR alpha (TCRα) chain comprising a variable alpha (Vα) domain and a constant alpha (Cα) domain, wherein the nucleic acid sequence comprises of (a) a transgene sequence encoding the TCRα and the Vβ domain, and (b) an open reading frame of the endogenous TRBC locus or a partial sequence thereof, wherein the open reading frame encodes at least a portion of the Cβ domain of the recombinant TCR.

25. A genetically engineered T cell, comprising a modified T cell receptor beta constant (TRBC) locus, said modified TRBC locus comprising a nucleic acid encoding a recombinant TCR or portion thereof, said recombinant TCR or portion thereof comprising: (i) a TCR beta (TCRβ) chain comprising a variable beta (Vβ) domain and a constant beta (Cβ) domain, and (ii) a TCR alpha (TCRα) chain comprising a variable alpha (Vα) domain and a constant alpha (Cα) domain, wherein the nucleic acid sequence comprises of (a) a transgene sequence encoding the TCRα and the Vβ domain, and (b) an open reading frame of the endogenous TRBC locus or a partial sequence thereof, wherein the open reading frame encodes at least a portion of the Cβ domain of the recombinant TCR, wherein:
a portion of the Cβ is encoded by the open reading frame of the endogenous TRBC locus or a partial sequence thereof, and a further portion of the Cβ is encoded by the transgene sequence, wherein said further portion of Cβ is less than the full length of a native Cβ; and
the further portion of the Cβ and/or the Cα region encoded by the nucleic acid sequence of (a) comprises one or more modifications, optionally a replacement, deletion, or insertion of one or more amino acids compared to a native Cβ region and/or a native Cα region, optionally said one or more modifications introduces one or more cysteine residues that are capable of forming one or more non-native disulfide bridges between the alpha chain and beta chain.

26. The genetically engineered T cell of claim 24 or claim 25, wherein the transgene sequence has been integrated via homology directed repair (HDR).

27. The genetically engineered T cell of any of claims 24-26, wherein the TRBC locus is a TRBC1 locus and/or a TRBC2 locus.

28. The genetically engineered T cell of any of claims 24-27, wherein the modified TRBC locus comprises an in-frame fusion of (i) a transgene sequence and (ii) an open reading frame or a partial sequence thereof of the endogenous TRBC locus, optionally wherein the transgene sequence is in-frame with one or more exons of the open reading frame or partial sequence thereof of the endogenous TRBC locus.

29. The genetically engineered T cell of any of claims 24-28, wherein the transgene sequence does not comprise a sequence encoding a 3′ untranslated region (3′ UTR) or an intron.

30. The genetically engineered T cell of any of claims 24 and 26-29, wherein a portion of the Cβ is encoded by the open reading frame of the endogenous TRBC locus or a partial sequence thereof, and a further portion of the Cβ is encoded by the transgene sequence, wherein said further portion of Cβ is less than the full length of a native Cβ.

31. The genetically engineered T cell of any of claims 24-30, wherein the open reading frame or the partial sequence thereof comprises at least one intron and at least one exon of the endogenous TRBC locus.

32. The genetically engineered T cell of any of claims 25, 30 and 31, wherein the further portion of the Cβ is encoded by a sequence of nucleotides that comprises less than four exons, less than three exons, less than two exons, one exon, or less than one full exon the open reading frame of a TRBC locus.

33. The genetically engineered T cell of any of claims 25 and 30-32, wherein the further portion of the Cβ is encoded by a sequence of nucleotides that is less than 400, less than 300, less than 250, less than 200, or less than 150 base pairs in length.

34. The genetically engineered T cell of any of claims 25 and 30-33, wherein the further portion of the Cβ is encoded by a portion of exon 1 of a TRBC locus, wherein the portion of exon 1 is less than the full length of exon 1 the open reading frame of the TRBC locus.

35. The genetically engineered T cell of any of claims 24-34, wherein the transgene sequence has been integrated downstream of the most 5′ nucleotide of exon 1 and upstream of the most 3′ nucleotide of exon 1 of the open reading frame of the endogenous TRBC locus.

36. The genetically engineered T cell of any of claims 24-35, wherein the at least a portion of Cβ is encoded by at least a portion of exon 1 and exons 2-4 of the open reading frame of the endogenous TRBC locus.

37. The genetically engineered T cell of any of claims 24-36, wherein the encoded TCRβ chain is capable of dimerizing with a TCRα chain.

38. The genetically engineered T cell of any of claims 24-37, wherein the encoded Cβ comprises the sequence selected from any one of SEQ ID NO: 16, 17, 21, and 25, or a sequence that exhibits at least 85%, 86%, 87%, 88%, 89%, 90%, 91%, 92%, 93%, 94%, 95%, 96%, 97%, 98%, 99% or more sequence identity to any one of SEQ ID NO: 16, 17, 21, and 25, or a partial sequence thereof.

39. The genetically engineered T cell of any of claims 24-38, wherein the encoded Cβ comprises the sequence selected from any one of SEQ ID NO: 20, 21, 25 and 253-258 or a sequence that exhibits at least 85%, 86%, 87%, 88%, 89%, 90%, 91%, 92%, 93%, 94%, 95%, 96%, 97%, 98%, 99% or more sequence identity to any one of SEQ ID NO: 20, 21, 25 and 253-258, or a partial sequence thereof.

40. The genetically engineered T cell of any of claims 25 and 30-39, wherein the at least a portion of Cβ is encoded by:
a sequence of nucleotides starting from residue 3 and up to residue 1445 of the sequence set forth in SEQ ID NO:2 or one or more exons thereof or a sequence that exhibits at least 85%, 86%, 87%, 88%, 89%, 90%, 91%, 92%, 93%, 94%, 95%, 96%, 97%, 98%, 99% or more sequence identity to a sequence of nucleotides starting from residue 3 and up to residue 1445 of the sequence set forth in SEQ ID NO:2 or one or more exons thereof, or a partial sequence thereof; or
a sequence of nucleotides starting from residue 3 and up to residue 1486 of the sequence set forth in SEQ ID NO:3 or one or more exons thereof or a sequence that exhibits at least 85%, 86%, 87%, 88%, 89%, 90%, 91%, 92%, 93%, 94%, 95%, 96%, 97%, 98%, 99% or more sequence identity to a sequence of nucleotides starting from residue 3 and up to residue 1486 of the sequence set forth in SEQ ID NO:3 or one or more exons thereof, or a partial sequence thereof.

41. The genetically engineered T cell of any of claims 25 and 30-39, wherein the further portion of the Cβ and/or the Cα region encoded by the nucleic acid sequence of (a) comprises one or more modifications, optionally a replacement, deletion, or insertion of one or more amino acids compared to a native Cβ region and/or a native Cα region, optionally said one or more modifications introduces one or more cysteine residues that are capable of forming one or more non-native disulfide bridges between the alpha chain and beta chain.

42. The genetically engineered T cell of claim 25 and claim 41, wherein the introduction of one or more cysteine residues comprises replacement of a non-cysteine residue with a cysteine residue.

43. The genetically engineered T cell of any of claims 25, 41 and 42, wherein the encoded Cα region comprises a cysteine at a position corresponding to position 48 with numbering as set forth in any of SEQ ID NO: 24; and/or the encoded Cβ region comprises a cysteine at a position corresponding to position 57 with numbering as set forth in SEQ ID NO: 20.

44. The genetically engineered T cell of any of claims 25 and 41-43, wherein the encoded Cβ comprises the sequence selected from any one of SEQ ID NOS: 253 and 256-258, or a partial sequence thereof.

45. The genetically engineered T cell of any of claims 24-44, wherein the engineered T cell further comprises a genetic disruption at a TRAC locus.

46. The genetically engineered T cell of any of claims 1-45, wherein the transgene sequence comprises one or more multicistronic element(s).

47. The genetically engineered T cell of claim 46, wherein the multicistronic element(s) is positioned between the nucleic acid sequence encoding the TCRα or a portion thereof and the nucleic acid sequence encoding the TCRβ or a portion thereof and/or upstream of the nucleic acid sequence encoding the TCR or a portion of the TCR.

48. The genetically engineered T cell of claim 46 or claim 47, wherein the multicistronic element is or comprises a ribosome skip sequence, optionally T2A, P2A, E2A, or F2A.

49. The genetically engineered T cell of any of claims 1-48, wherein the transgene sequence comprises one or more heterologous or regulatory control element(s) operably linked to control expression of the TCR when expressed from a cell introduced with the genetically engineered T cell.

50. The genetically engineered T cell of claim 49, wherein the heterologous regulatory or control element comprises a heterologous promoter.

51. The genetically engineered T cell of claim 50, wherein the heterologous promoter is selected from among a constitutive promoter, an inducible promoter, a repressible promoter, and/or a tissue-specific promoter.

52. The genetically engineered T cell of claim 50 or claim 51, wherein the heterologous promoter is or comprises a human elongation factor 1 alpha (EF1α) promoter or an MND promoter or a variant thereof.

53. The genetically engineered T cell of any claims 1-52, wherein the recombinant TCR is capable of binding to an antigen that is associated with, specific to, and/or expressed on a cell or tissue that is associated with a disease, disorder, or condition.

54. The genetically engineered T cell of claim 53, wherein the disease, disorder, or condition is an infectious disease or disorder, an autoimmune disease, an inflammatory disease, a tumor, or a cancer.

55. The genetically engineered T cell of any of claims 1-54, wherein the T cell is a primary T cell derived from a subject, optionally wherein the subject is a human.

56. The genetically engineered T cell of any of claims 1-55, wherein the T cell is a CD8+ T cell or subtypes thereof.

57. The genetically engineered cell of any of claims 1-55, wherein the T cell is a CD4+ T cell or subtypes thereof.

58. The genetically engineered cell of any of claims 1-57, wherein the T cell is derived from a multipotent or pluripotent cell, which optionally is an iPSC.

59. A composition comprising a plurality of genetically engineered T cells of any of claims 1-58.

60. A composition comprising a plurality of genetically engineered T cells comprising a modified T cell receptor alpha constant (TRAC) locus, said modified TRAC locus comprising a nucleic acid encoding a recombinant TCR or portion thereof, said recombinant TCR or portion thereof comprising: (i) a TCR alpha (TCRα) chain comprising a variable alpha (Vα) domain and a constant alpha (Cα) domain, and (ii) a TCR beta (TCRβ) chain comprising a variable beta (Vβ) domain and a constant beta (Cβ) domain, wherein the nucleic acid sequence comprises of (a) a transgene sequence encoding the TCRβ and the Vα domain, and (b) an open reading frame of the endogenous TRAC locus or a partial sequence thereof, wherein the open reading frame encodes at least a portion of the Cα domain of the recombinant TCR, and said recombinant TCR is capable of binding to an antigen that is associated with, specific to, and/or expressed on a cell or tissue that is associated with a disease, disorder, or condition; wherein:
at least 70%, 75%, 80%, 90%, 91%, 92%, 93%, 94%, 95%, 96%, 97%, 98%, or 99% of the engineered cells in the composition comprise a genetic disruption in or of an endogenous T cell receptor alpha constant region (TRAC) gene and/or a T cell receptor beta constant region (TRBC) gene;
at least 80%, 90%, 91%, 92%, 93%, 94%, 95%, 96%, 97%, 98%, or 99% of the engineered cells in the composition do not express or do not express detectable levels of a gene product of an endogenous TRAC or TRBC gene; and/or
at least or greater than 35%, 40%, 45%, 50%, 55%, 60%, 65%, 70%, 75%, 80%, or 90% of the cells in the composition express the recombinant TCR and/or exhibits binding to the antigen.

61. A composition comprising a plurality of genetically engineered T cells comprising a modified T cell receptor alpha constant (TRAC) locus, said modified TRAC locus comprising a nucleic acid encoding a recombinant TCR or portion thereof, said recombinant TCR or portion thereof comprising: (i) a TCR alpha (TCRα) chain comprising a variable alpha (Vα) domain and a constant alpha (Cα) domain, and (ii) a TCR beta (TCRβ) chain comprising a variable beta (Vβ) domain and a constant beta (Cβ) domain, wherein the nucleic acid sequence comprises of (a) a transgene sequence encoding the TCRβ and the Vα domain, and (b) an open reading frame of the endogenous TRAC locus or a partial sequence thereof, wherein the open reading frame encodes at least a portion of the Cα domain of the recombinant TCR;
a portion of the Cα is encoded by the open reading frame of the endogenous TRAC locus or a partial sequence thereof, and a further portion of the Cα is encoded by the transgene sequence, wherein said further portion of Cα is less than the full length of a native Cα; and
the further portion of the Cα and/or the Cβ region encoded by the nucleic acid sequence of (a) comprises one or more modifications compared to a native Cα region and/or a native Cβ region, said one or more modifications introduces one or more cysteine residues that are capable of forming one or more non-native disulfide bridges between the alpha chain and beta chain.

62. A composition comprising a plurality of genetically engineered T cells comprising a modified T cell receptor beta constant (TRBC) locus, said modified TRBC locus comprising a nucleic acid encoding a recombinant TCR or portion thereof, said recombinant TCR or portion thereof comprising: (i) a TCR beta (TCRβ) chain comprising a variable beta (Vβ) domain and a constant beta (Cβ) domain, and (ii) a TCR alpha (TCRα) chain comprising a variable alpha (Vα) domain and a constant alpha (Cα) domain, wherein the nucleic acid sequence comprises of (a) a transgene sequence encoding the TCRα and the Vβ domain, and (b) an open reading frame of the endogenous TRBC locus or a partial sequence thereof, wherein the open reading frame encodes at least a portion of the Cβ domain of the recombinant TCR, and said recombinant TCR is capable of binding to an antigen that is associated with, specific to, and/or expressed on a cell or tissue that is associated with a disease, disorder, or condition; wherein:
at least 70%, 75%, 80%, 90%, 91%, 92%, 93%, 94%, 95%, 96%, 97%, 98%, or 99% of the engineered cells in the composition comprise a genetic disruption in or of an endogenous T cell receptor alpha constant region (TRAC) gene and/or a T cell receptor beta constant region (TRBC) gene;
at least 80%, 90%, 91%, 92%, 93%, 94%, 95%, 96%, 97%, 98%, or 99% of the engineered cells in the composition do not express or do not express detectable levels of a gene product of an endogenous TRAC or TRBC gene; and/or
at least or greater than 35%, 40%, 45%, 50%, 55%, 60%, 65%, 70%, 75%, 80%, or 90% of the cells in the composition express the recombinant TCR and/or exhibits binding to the antigen.

63. A composition comprising a plurality of genetically engineered T cells comprising a modified T cell receptor beta constant (TRBC) locus, said modified TRBC locus comprising a nucleic acid encoding a recombinant TCR or portion thereof, said recombinant TCR or portion thereof comprising: (i) a TCR beta (TCRβ) chain comprising a variable beta (Vβ) domain and a constant beta (Cβ) domain, and (ii) a TCR alpha (TCRα) chain comprising a variable alpha (Vα) domain and a constant alpha (Cα) domain, wherein the nucleic acid sequence comprises of (a) a transgene sequence encoding the TCRα and the Vβ domain, and (b) an open reading frame of the endogenous TRBC locus or a partial sequence thereof, wherein the open reading frame encodes at least a portion of the Cβ domain of the recombinant TCR; wherein:
a portion of the Cβ is encoded by the open reading frame of the endogenous TRBC locus or a partial sequence thereof, and a further portion of the Cβ is encoded by the transgene sequence, wherein said further portion of Cβ is less than the full length of a native Cβ; and
the further portion of the Cβ and/or the Cα region encoded by the nucleic acid sequence of (a) comprises one or more modifications, optionally a replacement, deletion, or insertion of one or more amino acids compared to a native Cβ region and/or a native Cα region, optionally said one or more modifications introduces one or more cysteine residues that are capable of forming one or more non-native disulfide bridges between the alpha chain and beta chain.

64. The composition of claim 61 or claim 63, wherein the recombinant TCR is capable of binding to an antigen that is associated with, specific to, and/or expressed on a cell or tissue that is associated with a disease, disorder, or condition.

65. The composition of any of claims 59-64, wherein the composition comprises CD4+ and/or CD8+ T cells.

66. The composition of any of claims 59-65, wherein the composition comprises CD4+ and CD8+ T cells and the ratio of CD4+ to CD8+ T cells is from or from about 1:3 to 3:1, optionally 1:1.

67. The composition of any of claims 59, 61 and 63-66, wherein:
at least 70%, 75%, 80%, 90%, 91%, 92%, 93%, 94%, 95%, 96%, 97%, 98%, or 99% of the engineered cells in the composition comprise a genetic disruption in or of an endogenous T cell receptor alpha constant region (TRAC) gene and/or a T cell receptor beta constant region (TRBC) gene; and/or
at least 80%, 90%, 91%, 92%, 93%, 94%, 95%, 96%, 97%, 98%, or 99% of the engineered cells in the composition do not express or do not express detectable levels of a gene product of an endogenous TRAC or TRBC gene.

68. The composition of any of claims 59, 61 and 63-67, wherein at least or greater than 35%, 40%, 45%, 50%, 55%, 60%, 65%, 70%, 75%, 80%, or 90% of the cells in the composition express the recombinant TCR and/or exhibits binding to the antigen.

69. A polynucleotide, comprising:
(a) a nucleic acid sequence encoding a portion of a recombinant T cell receptor (TCR), said nucleic acid sequence encoding (i) a T cell receptor beta (TCRβ) chain comprising a variable beta (Vβ) domain and a constant beta (Cβ) domain; and (ii) a portion of a T cell receptor alpha (TCRα) chain, wherein the portion of the TCRα chain is less than a full-length of a native TCRα chain, and
(b) one or more homology arms linked to the nucleic acid sequence, wherein the one or more homology arms comprise a sequence homologous to one or more region(s) of an open reading frame of a TRAC locus; wherein the polynucleotide is comprised in a viral vector.

70. A polynucleotide, comprising:
(a) a nucleic acid sequence encoding a portion of a recombinant T cell receptor (TCR), said nucleic acid sequence encoding (i) a T cell receptor beta (TCRβ) chain comprising a variable beta (Vβ) domain and a constant beta (Cβ) domain; and (ii) a portion of a T cell receptor alpha (TCRα) chain, wherein the portion of the TCRα chain is less than a full-length of a native TCRα chain, and
(b) one or more homology arms linked to the nucleic acid sequence, wherein the one or more homology arms comprise a sequence homologous to one or more region(s) of an open reading frame of a TRAC locus; wherein, when the TCR or antigen-binding fragment thereof is expressed from a cell introduced with the polynucleotide:
a portion of the Cα is encoded by the open reading frame of the endogenous TRAC locus or a partial sequence thereof, and a further portion of the Cα is encoded by the transgene sequence, wherein said further portion of Cα is less than the full length of a native Cα; and
the further portion of the Cα and/or the Cβ region encoded by the nucleic acid sequence of (a) comprises one or more modifications compared to a native Cα region and/or a native Cβ region, said one or more modifications introduces one or more cysteine residues that are capable of forming one or more non-native disulfide bridges between the alpha chain and beta chain and/or wherein the Cα and/or the Cβ of the recombinant TCR comprises one or more non-native cysteines.

71. A polynucleotide, comprising:
(a) a nucleic acid sequence encoding a portion of a recombinant T cell receptor (TCR), said nucleic acid sequence encoding (i) a T cell receptor beta (TCRβ) chain comprising a variable beta (Vβ) domain and a constant beta (Cβ) domain; and (ii) a portion of a T cell receptor alpha (TCRα) chain, wherein the portion of the TCRα chain is less than a full-length of a native TCRα chain, and
(b) one or more homology arms linked to the nucleic acid sequence, wherein the one or more homology arms comprise a sequence homologous to one or more region(s) of an open reading frame of a TRAC locus; wherein the transgene sequence comprises one or more heterologous or regulatory control element(s) comprising a heterologous promoter, operably linked to control expression of the TCR when expressed from a cell introduced with the genetically engineered T cell.

72. The polynucleotide of any of claims 69-71, wherein the TCRα chain comprises a constant alpha region (Ca), wherein at least a portion of said Cα is encoded by the open reading frame of the endogenous TRAC locus or a partial sequence thereof when the TCR or antigen-binding fragment thereof is expressed from a cell introduced with the polynucleotide; and/or
the nucleic acid sequence of (a) and the one of the one or more homology arms together comprise a sequence of nucleotides encoding the Cα that is less than the full length of a native Cα, wherein at least a portion of the Cα is encoded by the open reading frame of the endogenous TRAC locus or a partial sequence thereof when the TCR or antigen-binding fragment thereof is expressed from a cell introduced with the polynucleotide.

73. The polynucleotide of any of claims 69-72, wherein the nucleic acid sequence encoding the TCRβ chain is upstream of the nucleic acid sequence encoding the portion of the TCRα chain.

74. The polynucleotide of any of claims 69-73, wherein the nucleic acid sequence of (a) does not comprise a sequence encoding a 3′ untranslated region (3′ UTR) or an intron.

75. The polynucleotide of any of claims 69-74, wherein the nucleic acid sequence of (a) is in-frame with one or more exons or a partial sequence thereof of the open reading frame of the TRAC locus comprised in the one or more homology arm(s).

76. The polynucleotide of any of claims 69 and 71-75, wherein a portion of the Cα is encoded by the open reading frame of the endogenous TRAC locus or a partial sequence thereof, and a further portion of the Cα is encoded by the nucleic acid sequence of (a), wherein said further portion of Cα is less than the full length of a native Cα.

77. The polynucleotide of any of claims 69-76, wherein the open reading frame or the partial sequence thereof comprises at least one intron and at least one exon of the endogenous TRAC locus.

78. The polynucleotide of any of claims 70, 76 and 77, wherein the further portion of the Cα is encoded by a sequence of nucleotides that comprises less than four exons, less than three exons, less than two exons, one exon, or less than one full exon the open reading frame of the TRAC locus.

79. The polynucleotide of any of claims 70 and 76-78 wherein the further portion of the Cα is encoded by a sequence of nucleotides that is less than 400, less than 300, less than 250, less than 200, or less than 150 base pairs in length.

80. The polynucleotide of any of claims 70 and 76-79, wherein the further portion of the Cα is encoded by a portion of exon 1 of the TRAC locus, wherein the portion of exon 1 is less than the full length of exon 1 the open reading frame of the TRAC locus.

81. The polynucleotide of any of claims 69-80, wherein the TCRα chain is capable of dimerizing with a TCRβ chain, when produced from a cell introduced with the polynucleotide.

82. The polynucleotide of any of claims 69-81, wherein the encoded Cα comprises the sequence selected from any one of SEQ ID NOS: 19, 24 and 243-252, or a sequence that exhibits at least 85%, 86%, 87%, 88%, 89%, 90%, 91%, 92%, 93%, 94%, 95%, 96%, 97%, 98%, 99% or more sequence identity to any one of SEQ ID NOS: 19, 24 and 243-252, or a partial sequence thereof, when produced from a cell introduced with the polynucleotide.

83. The polynucleotide of any of claims 70 and 76-82, wherein the at least a portion of Cα is encoded by a sequence of nucleotides starting from residue 3 and up to residue 3155 of the sequence set forth in SEQ ID NO:1 or one or more exons thereof or a sequence that exhibits at least 85%, 86%, 87%, 88%, 89%, 90%, 91%, 92%, 93%, 94%, 95%, 96%, 97%, 98%, 99% or more sequence identity to a sequence of nucleotides starting from residue 3 and up to residue 3155 of the sequence set forth in SEQ ID NO:1 or one or more exons thereof, or a partial sequence thereof.

84. The polynucleotide of any of claims 70 and 76-83, wherein the further portion of the Cα comprises a sequence set forth in SEQ ID NO:142, or a sequence that exhibits at least 85%, 86%, 87%, 88%, 89%, 90%, 91%, 92%, 93%, 94%, 95%, 96%, 97%, 98%, 99% or more sequence identity to SEQ ID NO:142, or a partial sequence thereof.

85. The polynucleotide of any of claims 76-84, wherein the further portion of the Cα and/or the Cβ region encoded by the nucleic acid sequence of (a) comprises one or more modifications, optionally a replacement, deletion, or insertion of one or more amino acids compared to a native Cα region and/or a native Cβ region, optionally said one or more modifications introduces one or more cysteine residues that are capable of forming one or more non-native disulfide bridges between the alpha chain and beta chain.

86. The polynucleotide of claim 70 and claim 85, wherein the introduction of one or more cysteine residues comprises replacement of a non-cysteine residue with a cysteine residue.

87. The polynucleotide of any of claims 70, 85 and 86, wherein the encoded Cα region comprises a cysteine at a position corresponding to position 48 with numbering as set forth in any of SEQ ID NO: 24; and/or the encoded Cβ region comprises a cysteine at a position corresponding to position 57 with numbering as set forth in SEQ ID NO: 20, when produced from a cell introduced with the polynucleotide.

88. The polynucleotide of any of claims 70 and 85-87, wherein the encoded Cα comprises the sequence selected from any one of SEQ ID NOS: 248-252, or a partial sequence thereof, when produced from a cell introduced with the polynucleotide.

89. The polynucleotide of any of claims 69-88, wherein the portion of the TCRα chain comprises a variable alpha (Vα) domain.

90. The polynucleotide of any of claims 69-89, wherein the one or more homology arm comprises a 5′ homology arm and/or a 3′ homology arm.

91. The polynucleotide of claim 90, wherein the 5′ homology arm and 3′ homology arm comprises nucleic acid sequences homologous to nucleic acid sequences surrounding a target site, wherein the target site is within the TRAC locus.

92. The polynucleotide of claim 91 wherein the target site is within exon 1 of the TRAC locus.

93. The polynucleotide of any of claims 90-92, wherein the 5′ homology arm comprises:
a) a sequence comprising at or at least at or at least 150, 200, 250, 300, 350, 400, 450, 500, 550, or 600 contiguous nucleotides to a sequence that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 124;
b) a sequence comprising at or at least at or at least 150, 200, 250, 300, 350, 400, 450, 500, 550, or 600 contiguous nucleotides of the sequence set forth in SEQ ID NO:124; or
c) the sequence set forth in SEQ ID NO: 124.

94. The polynucleotide of any of claims 90-93, wherein the 3′ homology arm comprises:
a) a sequence comprising at or at least at or at least 150, 200, 250, 300, 350, 400, 450, 500, 550, or 600 contiguous nucleotides to a sequence that exhibits at least 85%, 86%, 87%, 88%, 89%, 90%, 91%, 92%, 93%, 94%, 95%, 96%, 97%, 98%, 99% or more sequence identity to the sequence set forth in SEQ ID NO: 125;
b) a sequence comprising at or at least at or at least 150, 200, 250, 300, 350, 400, 450, 500, 550, or 600 contiguous nucleotides of the sequence set forth in SEQ ID NO:125; or
c) the sequence set forth in SEQ ID NO: 125.

95. A polynucleotide, comprising:
(a) a nucleic acid sequence encoding a portion of a recombinant T cell receptor (TCR), said nucleic acid sequence encoding (i) a T cell receptor alpha (TCRα) chain comprising a variable alpha (Vα) domain and a constant alpha (Cα) domain; and (ii) a portion of a T cell receptor beta (TCRβ) chain, wherein the portion of the TCRβ chain is less than a full-length native TCRβ chain, and
(b) one or more homology arms linked to the nucleic acid sequence, wherein the one or more homology arms comprise a sequence homologous to one or more region(s) of an open reading frame of a TRBC locus.

96. The polynucleotide of claim 95, wherein the TCRβ chain comprises a constant beta (Cβ), wherein at least a portion of said C is encoded by the open reading frame of the endogenous TRAC locus or a partial sequence thereof when the TCR or antigen-binding fragment thereof is expressed from a cell introduced with the polynucleotide and/or the nucleic acid sequence of (a) and the one of the one or more homology arms together comprise a sequence of nucleotides encoding the Cβ that is less than the full length of a native Cβ, wherein at least a portion of the Cβ is encoded by the open reading frame of the endogenous TRBC locus or a partial sequence thereof when the TCR or antigen-binding fragment thereof is expressed from a cell introduced with the polynucleotide.

97. The polynucleotide of claim 95 or claim 96, wherein the TRBC locus is one or more of TRBC1 or TRBC2.

98. The polynucleotide of any of claims 95-97, wherein the nucleic acid sequence encoding the TCRα chain is upstream of nucleic acid sequence encoding the portion of the TCRβ chain.

99. The polynucleotide of any of claims 95-98, wherein the nucleic acid sequence of (a) does not comprise a sequence encoding a 3′ untranslated region (3′ UTR) or an intron.

100. The polynucleotide of any of claims 95-99, wherein the nucleic acid sequence of (a) is in-frame with one or more exons or a partial sequence thereof of the open reading frame of the TRAC locus comprised in the one or more homology arm(s).

101. The polynucleotide of any of claims 95-100, wherein a portion of the Cβ is encoded by the open reading frame of the endogenous TRBC locus or a partial sequence thereof, and a further portion of the Cβ is encoded by the nucleic acid sequence of (a), wherein said further portion of Cβ is less than the full length of a native Cβ

102. The polynucleotide of any of claims 95-101 wherein the open reading frame or the partial sequence thereof comprises at least one intron and at least one exon of the endogenous TRBC locus.

103. The polynucleotide of claim 101 or claim 102, wherein the further portion of the Cβ is encoded by a sequence of nucleotides that encodes less than four exons, less than three exons, less than two exons, one exon, or less than one full exon of the open reading frame of the TRBC locus.

104. The polynucleotide of any of claims 101-103, wherein the further portion of the Cβ is encoded by a sequence of nucleotides that is less than 400, less than 300, less than 250, less than 200, or less than 150 base pairs in length.

105. The polynucleotide of any of claims 101-104, wherein the further portion of the Cβ is encoded by a portion of exon 1 of a TRBC locus, wherein the portion of exon 1 is less than the full length of exon 1 of the open reading frame of the TRBC locus.

106. The polynucleotide of any of claims 95-105, wherein the TCRβ chain is capable of dimerizing with a TCRα chain, when produced from a cell introduced with the polynucleotide.

107. The polynucleotide of any of claims 69-106, wherein the encoded Cβ comprises the sequence selected from any one of SEQ ID NO: 20, 21, 25 and 253-258 or a sequence that exhibits at least 85%, 86%, 87%, 88%, 89%, 90%, 91%, 92%, 93%, 94%, 95%, 96%, 97%, 98%, 99% or more sequence identity to any one of SEQ ID NO: 20, 21, 25 and 253-258, or a partial sequence thereof, when produced from a cell introduced with the polynucleotide.

108. The polynucleotide of any of claims 101-107, wherein the at least a portion of Cβ is encoded by:
a sequence of nucleotides starting from residue 3 and up to residue 1445 of the sequence set forth in SEQ ID NO:2 or one or more exons thereof or a sequence that exhibits at least 85%, 86%, 87%, 88%, 89%, 90%, 91%, 92%, 93%, 94%, 95%, 96%, 97%, 98%, 99% or more sequence identity to a sequence of nucleotides starting from residue 3 and up to residue 1445 of the sequence set forth in SEQ ID NO:2 or one or more exons thereof, or a partial sequence thereof; or
a sequence of nucleotides starting from residue 3 and up to residue 1486 of the sequence set forth in SEQ ID NO:3 or one or more exons thereof or a sequence that exhibits at least 85%, 86%, 87%, 88%, 89%, 90%, 91%, 92%, 93%, 94%, 95%, 96%, 97%, 98%, 99% or more sequence identity to a sequence of nucleotides starting from residue 3 and up to residue 1486 of the sequence set forth in SEQ ID NO:3 or one or more exons thereof, or a partial sequence thereof.

109. The polynucleotide of any of claims 101-108, wherein the further portion of the Cβ and/or the Cα region encoded by the nucleic acid sequence of (a) comprises one or more modifications, optionally a replacement, deletion, or insertion of one or more amino acids compared to a native Cβ region and/or a native Cα region, optionally said one or more modifications introduces one or more cysteine residues that are capable of forming one or more non-native disulfide bridges between the alpha chain and beta chain.

110. The polynucleotide of claim 109, wherein the introduction of one or more cysteine residues comprises replacement of a non-cysteine residue with a cysteine residue.

111. The polynucleotide of claim 109 or claim 110, wherein the encoded Cα region comprises a cysteine at a position corresponding to position 48 with numbering as set forth in any of SEQ ID NO: 24; and/or the encoded Cβ region comprises a cysteine at a position corresponding to position 57 with numbering as set forth in SEQ ID NO: 20.

112. The polynucleotide of any of claims 109-111, wherein the encoded Cβ comprises the sequence selected from any one of SEQ ID NOS: 253 and 256-258, or a partial sequence thereof.

113. The polynucleotide of any of claims 95-112, wherein the portion of the TCRβ chain comprises a variable beta (Vβ) domain.

114. The polynucleotide of any of claims 95-113, wherein the one or more homology arm comprises a 5′ homology arm and/or a 3′ homology arm.

115. The polynucleotide of claim 114, wherein the 5′ homology arm and 3′ homology arm comprises nucleic acid sequences homologous to nucleic acid sequences surrounding a target site, wherein the target site is within the open reading frame of the TRBC locus.

116. The polynucleotide of claim 115, wherein the target site is within exon 1 of the open reading frame of the TRBC locus.

117. The polynucleotide of any of claims 69-116, wherein the nucleic acid sequence of (a) is a sequence that is exogenous or heterologous to an open reading frame of an endogenous genomic TRAC locus of a T cell, optionally a human T cell.

118. The polynucleotide of any of claims 90-94 and 114-117, wherein the 5′ homology arm and 3′ homology arm independently are between at or about 50 and at or about 100 nucleotides in length, at or about 100 and at or about 250 nucleotides in length, at or about 250 and at or about 500 nucleotides in length, at or about 500 and at or about 750 nucleotides in length, at or about 750 and at or about 1000 nucleotides in length, or at or about 1000 and at or about 2000 nucleotides in length.

119. The polynucleotide of any of claims 90-94 and 114-118, the 5′ homology arm and 3′ homology arm independently are from at or about 100 to at or about 1000 nucleotides, 100 to 750 nucleotides, 100 to 600 nucleotides, 100 to 400 nucleotides, 100 to 300 nucleotides, 100 to 200 nucleotides, 200 to 1000 nucleotides, 200 to 750 nucleotides, 200 to 600 nucleotides, 200 to 400 nucleotides, 200 to 300 nucleotides, 300 to 1000 nucleotides, 300 to 750 nucleotides, 300 to 600 nucleotides, 300 to 400 nucleotides, 400 to 1000 nucleotides, 400 to 750 nucleotides, 400 to 600 nucleotides, 600 to 1000 nucleotides, 600 to 750 nucleotides or 750 to 1000 nucleotides in length.

120. The polynucleotide of any of claims 90-94 and 114-119, wherein the 5′ homology arm and 3′ homology arm independently are at or about 200, 300, 400, 500, 600, 700 or 800 nucleotides in length, or any value between any of the foregoing.

121. The polynucleotide of any of claims 90-94 and 114-120, wherein the 5′ homology arm and 3′ homology arm independently are greater than at or about 300 nucleotides in length, optionally wherein the 5′ homology arm and 3′ homology arm independently are at or about 400, 500 or 600 nucleotides in length or any value between any of the foregoing.

122. The polynucleotide of any of claims 90-94 and 114-121, wherein the 5′ homology arm and 3′ homology arm independently are, are about, or are less than about 600 nucleotides in length.

123. The polynucleotide of any of claims 90-94 and 114-122, wherein the 5′ homology arm and 3′ homology arm independently are greater than at or about 300 nucleotides in length.

124. The polynucleotide of any of claims 69-123, wherein the nucleic acid sequence of (a) comprises one or more multicistronic element(s).

125. The polynucleotide of claim 124, wherein the multicistronic element(s) is positioned between the nucleic acid sequence encoding the TCRα or a portion thereof and the nucleic acid sequence encoding the TCRβ or a portion thereof and/or are upstream of the nucleic acid sequence encoding the TCR or a portion of the TCR.

126. The polynucleotide of claim 124 or claim 125, wherein the multicistronic element is or comprises a ribosome skip sequence, optionally T2A, P2A, E2A, or F2A.

127. The polynucleotide of any of claims 69-126, wherein the nucleic acid sequence of (a) comprises one or more heterologous or regulatory control element(s) operably linked to control expression of the TCR when expressed from a cell introduced with the polynucleotide.

128. The polynucleotide of claim 127, wherein the heterologous regulatory or control element comprises a heterologous promoter.

129. The polynucleotide of claim 128, wherein the heterologous promoter is selected from among a constitutive promoter, an inducible promoter, a repressible promoter, and/or a tissue-specific promoter.

130. The polynucleotide of claim 128 or claim 129, wherein the heterologous promoter is or comprises a human elongation factor 1 alpha (EF1α) promoter or an MND promoter or a variant thereof.

131. The polynucleotide of any of claims 69-130, wherein the polynucleotide is comprised in a viral vector.

132. The polynucleotide of claim 131, wherein the viral vector is an AAV vector, optionally selected from among AAV1, AAV2, AAV3, AAV4, AAV5, AAV6, AAV7 or AAV8 vector.

133. The polynucleotide of claim 132, wherein the AAV vector is an AAV2 or AAV6 vector.

134. The polynucleotide of any of claims 69-133, wherein the polynucleotide comprises the structure: [5′ homology arm]-[nucleic acid sequence of (a)]-[3′ homology arm].

135. The polynucleotide of any of claims 124-134, wherein the polynucleotide comprises the structure: [5′ homology arm]-[multicistronic element]-[nucleic acid sequence of (a)]-[3′ homology arm].

136. The polynucleotide of any of claims 128-134, wherein the polynucleotide comprises the structure: [5′ homology arm]-[heterologous promoter]-[nucleic acid sequence of (a)]-[3′ homology arm].

137. The polynucleotide of any of claims 69-136, wherein the encoded recombinant TCR is capable of binding to an antigen that is associated with, specific to, and/or expressed on a cell or tissue that is associated with a disease, disorder, or condition.

138. The polynucleotide of claim 137, wherein the disease, disorder, or condition is an infectious disease or disorder, an autoimmune disease, an inflammatory disease, a tumor, or a cancer.

139. The polynucleotide of any of claims 69-138, wherein the polynucleotide is at least at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length, or any value between any of the foregoing.

140. The polynucleotide of any of claims 69-139, wherein the polynucleotide is between at or about 2500 and at or about 5000 nucleotides, at or about 3500 and at or about 4500 nucleotides, or at or about 3750 nucleotides and at or about 4250 nucleotides in length.

141. A method of producing a genetically engineered T cell comprising a modified TRAC locus, comprising introducing the polynucleotide of any of claims 69-94 or 117-140 into a T cell comprising a genetic disruption at a TRAC locus.

142. A method of producing a genetically engineered T cell comprising a modified T cell receptor alpha constant (TRAC) locus, the method comprising:
(a) introducing into a T cell, one or more agent(s) capable of inducing a genetic disruption at a target site within an endogenous TRAC locus of the T cell; and
(b) introducing into the T cell a polynucleotide comprising a transgene sequence encoding a T cell receptor beta (TCRβ) chain and a portion of a T cell receptor alpha (TCRα) chain, wherein the portion is less than a full-length native TCRα chain, and wherein:
the introduction of the template polynucleotide is performed after the introduction of the one or more agent(s) capable of inducing a genetic disruption; and
the transgene is targeted for integration within the endogenous TRAC locus via homology directed repair (HDR), thereby producing a genetically engineered cell comprising a modified TRAC locus.

143. A method of producing a genetically engineered T cell comprising a modified T cell receptor alpha constant (TRAC) locus, the method comprising introducing, into a T cell, a polynucleotide comprising a transgene sequence encoding a T cell receptor beta (TCRβ) chain and a portion of a T cell receptor alpha (TCRα) chain, said T cell having a genetic disruption within the endogenous TRAC locus of the T cell, wherein the transgene is targeted for integration within the endogenous TRAC locus via homology directed repair (HDR), thereby producing a genetically engineered cell comprising a modified TRAC locus.

144. A method of producing a genetically engineered T cell comprising a modified T cell receptor alpha constant (TRAC) locus, the method comprising:
(a) introducing into a T cell, one or more agent(s) capable of inducing a genetic disruption at a target site within an endogenous TRAC locus of the T cell; and
(b) introducing into the T cell a polynucleotide comprising a transgene sequence encoding a T cell receptor beta (TCRβ) chain and a portion of a T cell receptor alpha (TCRα) chain, wherein the portion is less than a full-length native TCRα chain; and the transgene is targeted for integration within the endogenous TRAC locus via homology directed repair (HDR);
thereby producing a genetically engineered cell comprising a modified TRAC locus, wherein, upon targeted integration of the transgene:
a portion of the Cα is encoded by the open reading frame of the endogenous TRAC locus or a partial sequence thereof, and a further portion of the Cα is encoded by the transgene sequence, wherein said further portion of Cα is less than the full length of a native Cα; and
the further portion of the Cα and/or the Cβ region encoded by the nucleic acid sequence of (a) comprises one or more modifications compared to a native Cα region and/or a native Cβ region, said one or more modifications introduces one or more cysteine residues that are capable of forming one or more non-native disulfide bridges between the alpha chain and beta chain and/or wherein the Cα and/or the Cβ of the recombinant TCR comprises one or more non-native cysteines.

145. A method of producing a genetically engineered T cell comprising a modified T cell receptor alpha constant (TRAC) locus, the method comprising introducing, into a T cell, a polynucleotide comprising a transgene sequence encoding a T cell receptor beta (TCRβ) chain and a portion of a T cell receptor alpha (TCRα) chain, said T cell having a genetic disruption within the endogenous TRAC locus of the T cell; and the transgene is targeted for integration within the endogenous TRAC locus via homology directed repair (HDR); thereby producing a genetically engineered cell comprising a modified TRAC locus, wherein, upon targeted integration of the transgene:
a portion of the Cα is encoded by the open reading frame of the endogenous TRAC locus or a partial sequence thereof, and a further portion of the Cα is encoded by the transgene sequence, wherein said further portion of Cα is less than the full length of a native Cα; and
the further portion of the Cα and/or the Cβ region encoded by the nucleic acid sequence of (a) comprises one or more modifications compared to a native Cα region and/or a native Cβ region, said one or more modifications introduces one or more cysteine residues that are capable of forming one or more non-native disulfide bridges between the alpha chain and beta chain and/or wherein the Cα and/or the Cβ of the recombinant TCR comprises one or more non-native cysteines.

146. The method of any of claims 141, 143 and 145, wherein the genetic disruption has been induced by one or more agent(s) capable of inducing a genetic disruption of one or more target site within the endogenous TRAC locus.

147. The method of any of claims 141-146, wherein the polynucleotide is the polynucleotide of any of claims 69-94 or 117-140.

148. The method of any of claims 141-147, wherein the modified TRAC locus comprises a nucleic acid sequence encoding a recombinant TCR or portion thereof, wherein the nucleic acid sequence comprises an in-frame fusion of (i) a transgene sequence and (ii) an open reading frame or a partial sequence thereof of the endogenous TRAC locus, optionally wherein the transgene sequence is in-frame with one or more exons of the open reading frame or partial sequence thereof of the endogenous TRAC locus.

149. The method of any of claims 141-148, wherein the engineered T cell further comprises inducing a genetic disruption at a TRBC locus.

150. A method of producing a genetically engineered T cell comprising a modified TRBC locus, comprising introducing the polynucleotide of any of claims 95-140 into a T cell comprising a genetic disruption at a TRBC locus.

151. A method of producing a genetically engineered T cell comprising a modified T cell receptor beta constant (TRBC) locus, the method comprising:
(a) introducing, into a T cell, one or more agent(s) capable of inducing a genetic disruption at a target site within an endogenous TRBC locus of the T cell; and
(b) introducing into the T cell a polynucleotide comprising a transgene sequence encoding a T cell receptor alpha (TCRα) chain and a portion of a T cell receptor beta (TCRβ) chain, wherein the portion is less than a full-length native TCRβ chain, and wherein the transgene is targeted for integration within an endogenous TRBC locus via homology directed repair (HDR), thereby producing a genetically engineered cell comprising a modified TRBC locus.

152. A method of producing a genetically engineered T cell comprising a modified T cell receptor beta constant (TRBC) locus, the method comprising introducing, into a T cell, a polynucleotide comprising a transgene sequence encoding a T cell receptor alpha (TCRα) chain and a portion of a T cell receptor beta (TCRβ) chain, said T cell having a genetic disruption within an endogenous TRBC locus of the T cell, wherein the transgene is targeted for integration within the endogenous TRBC locus via homology directed repair (HDR), thereby producing a genetically engineered cell comprising a modified TRBC locus.

153. The method of claim 150 and claim 152, wherein the genetic disruption has been induced by one or more agent(s) capable of inducing a genetic disruption of one or more target site within the endogenous TRBC locus.

154. The method of any of claims 150-153, wherein the TRBC locus is a TRBC1 locus and/or a TRBC2 locus.

155. The method of any of claims 151-154, wherein the polynucleotide is the polynucleotide of any of claims 95-140.

156. The method of any of claims 151-155, wherein the modified TRBC locus comprises a nucleic acid sequence encoding a recombinant TCR or portion thereof, wherein the nucleic acid sequence comprises an in-frame fusion of (i) a transgene sequence and (ii) an open reading frame or a partial sequence thereof of the endogenous TRBC locus, optionally wherein the transgene sequence is in-frame with one or more exons of the open reading frame or partial sequence thereof of the endogenous TRBC locus.

157. The method of any of claims 151-156, wherein the engineered T cell further comprises inducing a genetic disruption at a TRAC locus.

158. The method of any of claims 142 and 144-157, wherein the one or more agent(s) capable of inducing a genetic disruption comprises a DNA binding protein or DNA-binding nucleic acid that specifically binds to or hybridizes to the target site.

159. The method of any of claims 142 and 144-158, wherein the one or more agent(s) comprises a zinc finger protein (ZFP), a TAL protein, or a clustered regularly interspaced short palindromic nucleic acid (CRISPR)-associated nuclease (Cas) specific for the target site.

160. The method of any of claims 142 and 144-159, wherein the one or more agent comprises a CRISPR-Cas9 combination and the CRISPR-Cas9 combination a guide RNA (gRNA) having a targeting domain that is complementary to the at least one target site.

161. The method of claim 160, wherein the one or more agent is introduced as a ribonucleoprotein (RNP) complex comprising the gRNA and a Cas9 protein.

162. The method of claim 161, wherein the concentration of the RNP is or is about 1 μM to at or about 5 μM, optionally wherein the concentration of the RNP is or is about 2 PM.

163. The method of claim 161 or claim 162, wherein the RNP is introduced via electroporation.

164. The method of any of claims 160-163, wherein the gRNA has a targeting domain that is complementary to a target site in a TRAC locus and comprises a sequence selected from the group consisting of UCUCUCAGCUGGUACACGGC (SEQ ID NO:28), UGGAUUUAGAGUCUCUCAGC (SEQ ID NO:29), ACACGGCAGGGUCAGGGUUC (SEQ ID NO:30), GAGAAUCAAAAUCGGUGAAU (SEQ ID NO:31), GCUGGUACACGGCAGGGUCA (SEQ ID NO:32), CUCAGCUGGUACACGGC (SEQ ID NO:33), UGGUACACGGCAGGGUC (SEQ ID NO:34), GCUAGACAUGAGGUCUA (SEQ ID NO:35), GUCAGAUUUGUUGCUCC (SEQ ID NO:36), UCAGCUGGUACACGGCA (SEQ ID NO:37), GCAGACAGACUUGUCAC (SEQ ID NO:38), GGUACACGGCAGGGUCA (SEQ ID NO:39), CUUCAAGAGCAACAGUGCUG (SEQ ID NO:40), AGAGCAACAGUGCUGUGGCC (SEQ ID NO:41), AAAGUCAGAUUUGUUGCUCC (SEQ ID NO:42), ACAAAACUGUGCUAGACAUG (SEQ ID NO:43), AAACUGUGCUAGACAUG (SEQ ID NO:44), UGUGCUAGACAUGAGGUCUA (SEQ ID NO:45), GGCUGGGGAAGAAGGUGUCUUC (SEQ ID NO:46), GCUGGGGAAGAAGGUGUCUUC (SEQ ID NO:47), GGGGAAGAAGGUGUCUUC (SEQ ID NO:48), GUUUUGUCUGUGAUAUACACAU (SEQ ID NO:49), GGCAGACAGACUUGUCACUGGAUU (SEQ ID NO:50), GCAGACAGACUUGUCACUGGAUU (SEQ ID NO:51), GACAGACUUGUCACUGGAUU (SEQ ID NO:52), GUGAAUAGGCAGACAGACUUGUCA (SEQ ID NO:53), GAAUAGGCAGACAGACUUGUCA (SEQ ID NO:54), GAGUCUCUCAGCUGGUACACGG (SEQ ID NO:55), GUCUCUCAGCUGGUACACGG (SEQ ID NO:56), GGUACACGGCAGGGUCAGGGUU (SEQ ID NO:57) and GUACACGGCAGGGUCAGGGUU (SEQ ID NO:58).

165. The method of claim 164, wherein the gRNA has a targeting domain comprising the sequence GAGAAUCAAAAUCGGUGAAU (SEQ ID NO:31).

166. The method of any of claims 160-163, wherein the gRNA has a targeting domain that is complementary to a target site in one or both of a TRBC1 and a TRBC2 gene and comprises a sequence selected from the group consisting of CACCCAGAUCGUCAGCGCCG (SEQ ID NO:59), CAAACACAGCGACCUCGGGU (SEQ ID NO:60), UGACGAGUGGACCCAGGAUA (SEQ ID NO:61), GGCUCUCGGAGAAUGACGAG (SEQ ID NO:62), GGCCUCGGCGCUGACGAUCU (SEQ ID NO:63), GAAAAACGUGUUCCCACCCG (SEQ ID NO:64), AUGACGAGUGGACCCAGGAU (SEQ ID NO:65), AGUCCAGUUCUACGGGCUCU (SEQ ID NO:66), CGCUGUCAAGUCCAGUUCUA (SEQ ID NO:67), AUCGUCAGCGCCGAGGCCUG (SEQ ID NO:68), UCAAACACAGCGACCUCGGG (SEQ ID NO:69), CGUAGAACUGGACUUGACAG (SEQ ID NO:70), AGGCCUCGGCGCUGACGAUC (SEQ ID NO:71), UGACAGCGGAAGUGGUUGCG (SEQ ID NO:72), UUGACAGCGGAAGUGGUUGC (SEQ ID NO:73), UCUCCGAGAGCCCGUAGAAC (SEQ ID NO:74), CGGGUGGGAACACGUUUUUC (SEQ ID NO:75), GACAGGUUUGGCCCUAUCCU (SEQ ID NO:76), GAUCGUCAGCGCCGAGGCCU (SEQ ID NO:77), GGCUCAAACACAGCGACCUC (SEQ ID NO:78), UGAGGGUCUCGGCCACCUUC (SEQ ID NO:79), AGGCUUCUACCCCGACCACG (SEQ ID NO:80), CCGACCACGUGGAGCUGAGC (SEQ ID NO:81), UGACAGGUUUGGCCCUAUCC (SEQ ID NO:82), CUUGACAGCGGAAGUGGUUG (SEQ ID NO:83), AGAUCGUCAGCGCCGAGGCC (SEQ ID NO:84), GCGCUGACGAUCUGGGUGAC (SEQ ID NO:85), UGAGGGCGGGCUGCUCCUUG (SEQ ID NO:86), GUUGCGGGGGUUCUGCCAGA (SEQ ID NO:87), AGCUCAGCUCCACGUGGUCG (SEQ ID NO:88), GCGGCUGCUCAGGCAGUAUC (SEQ ID NO:89), GCGGGGGUUCUGCCAGAAGG (SEQ ID NO:90), UGGCUCAAACACAGCGACCU (SEQ ID NO:91), ACUGGACUUGACAGCGGAAG (SEQ ID NO:92), GACAGCGGAAGUGGUUGCGG (SEQ ID NO:93), GCUGUCAAGUCCAGUUCUAC (SEQ ID NO:94), GUAUCUGGAGUCAUUGAGGG (SEQ ID NO:95), CUCGGCGCUGACGAUCU (SEQ ID NO:96), CCUCGGCGCUGACGAUC (SEQ ID NO:97), CCGAGAGCCCGUAGAAC (SEQ ID NO:98), CCAGAUCGUCAGCGCCG (SEQ ID NO:99), GAAUGACGAGUGGACCC (SEQ ID NO:100), GGGUGACAGGUUUGGCCCUAUC (SEQ ID NO:101), GGUGACAGGUUUGGCCCUAUC (SEQ ID NO:102), GUGACAGGUUUGGCCCUAUC (SEQ ID NO:103), GACAGGUUUGGCCCUAUC (SEQ ID NO:104), GAUACUGCCUGAGCAGCCGCCU (SEQ ID NO:105), GACCACGUGGAGCUGAGCUGGUGG (SEQ ID NO:106), GUGGAGCUGAGCUGGUGG (SEQ ID NO:107), GGGCGGGCUGCUCCUUGAGGGGCU (SEQ ID NO:108), GGCGGGCUGCUCCUUGAGGGGCU (SEQ ID NO:109), GCGGGCUGCUCCUUGAGGGGCU (SEQ ID NO:110), GGGCUGCUCCUUGAGGGGCU (SEQ ID NO:111), GGCUGCUCCUUGAGGGGCU (SEQ ID NO:112), GCUGCUCCUUGAGGGGCU (SEQ ID NO:113), GGUGAAUGGGAAGGAGGUGCACAG (SEQ ID NO:114), GUGAAUGGGAAGGAGGUGCACAG (SEQ ID NO:115) and GAAUGGGAAGGAGGUGCACAG (SEQ ID NO:116).

167. The method of claim 166, wherein the gRNA has a targeting domain comprising the sequence GGCCUCGGCGCUGACGAUCU (SEQ ID NO:63).

168. The method of any of claims 141-167, wherein the T cell is a primary T cell from a subject.

169. The method of claim 168, wherein the subject has or is suspected of having the disease, or disorder condition.

170. The method of claim 168, wherein the subject is or is suspected of being healthy.

171. The method of claim of any of claims 141-170, wherein the T cell is a CD8+ T cell or subtypes thereof.

172. The method of any of claims 141-170, wherein the T cell is a CD4+ T cell or subtypes thereof.

173. The method of any of claims 141-167, wherein the T cell is derived from a multipotent or pluripotent cell, which optionally is an iPSC.

174. The method of any of claims 168-173, wherein the T cell comprises a T cell that is autologous to the subject.

175. The method of any of claims 168-173, wherein the T cell comprises a T cell that is allogeneic to the subject.

176. The method of any of claims 141-175, wherein the polynucleotide is comprised in a viral vector.

177. The method of claim 176, wherein the vector is an AAV vector, optionally selected from among AAV1, AAV2, AAV3, AAV4, AAV5, AAV6, AAV7 or AAV8 vector.

178. The method of claim 177, wherein the AAV vector is an AAV2 or AAV6 vector.

179. The method of any of claims 141-178, wherein the recombinant TCR is capable of binding to an antigen that is associated with, specific to, and/or expressed on a cell or tissue that is associated with a disease, disorder, or condition.

180. The method of any of claims 144-179, wherein the introduction of the one or more agent capable of inducing a genetic disruption and the introduction of the template polynucleotide are performed simultaneously or sequentially, in any order.

181. The method of any of claims 144-180, wherein the introduction of the template polynucleotide is performed after the introduction of the one or more agent capable of inducing a genetic disruption.

182. The method of claim 181, wherein the template polynucleotide is introduced immediately after, or within at or about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after the introduction of one or more agents capable of inducing a genetic disruption, optionally at or about 2 hours after the introduction of the one or more agents.

183. The method of any of claims 141-182, wherein the method is performed in a plurality of T cells.

184. The method of claim 183, wherein the plurality T cells comprise CD4+ T cells, CD8+ T cells or CD4+ and CD8+ T cells.

185. The method of claim 183 or claim 184, wherein the plurality of T cells comprise CD4+ and CD8+ T cells and the ratio of CD4+ to CD8+ T cells is at or about 1:3 to at or about 3:1, optionally at or about 1:2 to at or about 2:1, optionally at or about 1:1.

186. The method of any of claims 142 and 144-185, wherein prior to the introducing of the one or more agent, the method comprises incubating the cells, in vitro with a stimulatory agent(s) under conditions to stimulate or activate the one or more T cells.

187. The method of claim 186, wherein the stimulatory agent (s) comprises and anti-CD3 and/or anti-CD28 antibodies, optionally anti-CD3/anti-CD28 beads, optionally wherein the bead to cell ratio is or is about 1:1.

188. The method of claim 186 or claim 187, comprising removing the stimulatory agent(s) from the one or more immune cells prior to the introducing with the one or more agents.

189. The method of any of claims 142 and 144-185, wherein the method further comprises incubating the cells prior to, during or subsequent to the introducing of the one or more agents and/or the introducing of the template polynucleotide with one or more recombinant cytokines, optionally wherein the one or more recombinant cytokines are selected from the group consisting of IL-2, IL-7, and IL-15.

190. The method of claim 189, wherein the one or more recombinant cytokine is added at a concentration selected from a concentration of IL-2 from at or about 10 U/mL to at or about 200 U/mL, optionally at or about 50 IU/mL to at or about 100 U/mL; IL-7 at a concentration of 0.5 ng/mL to 50 ng/mL, optionally at or about 5 ng/mL to at or about 10 ng/mL and/or IL-15 at a concentration of 0.1 ng/mL to 20 ng/mL, optionally at or about 0.5 ng/mL to at or about 5 ng/mL.

191. The method of claim 189 or claim 190, wherein the incubation is carried out subsequent to the introducing of the one or more agents and the introducing of the template polynucleotide for up to or approximately 24 hours, 36 hours, 48 hours, 3, 4, 5, 6, 7, 8, 9, 10, 11, 12, 13, 14, 15, 16, 17, 18, 19, 20 or 21 days, optionally up to or about 7 days.

192. The method of any of claims 141-191, wherein the polynucleotide is at least at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length, or any value between any of the foregoing.

193. The method of any of claims 141-192, wherein the polynucleotide is between at or about 2500 and at or about 5000 nucleotides, at or about 3500 and at or about 4500 nucleotides, or at or about 3750 nucleotides and at or about 4250 nucleotides in length.

194. An engineered T cell or a plurality of engineered T cells generated using the method of any of claims 141-193.

195. A composition, comprising the engineered T cell or plurality of engineered cells of claim 194.

196. The composition of claim 195, comprising CD4+ and/or CD8+ T cells.

197. The composition of claim 195 or claim 196, wherein the composition comprises CD4+ and CD8+ T cells and the ratio of CD4+ to CD8+ T cells is from or from about 1:3 to 3:1, optionally 1:1.

198. The composition of any of claims 195-197, wherein:
at least 70%, 75%, 80%, 90%, 91%, 92%, 93%, 94%, 95%, 96%, 97%, 98%, or 99% of the engineered cells in the composition comprise a genetic disruption in or of an endogenous T cell receptor alpha constant region (TRAC) gene and/or a T cell receptor beta constant region (TRBC) gene; and/or
at least 80%, 90%, 91%, 92%, 93%, 94%, 95%, 96%, 97%, 98%, or 99% of the engineered cells in the composition do not express or do not express detectable levels of a gene product of an endogenous TRAC or TRBC gene.

199. The composition of any of claims 195-198, wherein at least or greater than 35%, 40%, 45%, 50%, 55%, 60%, 65%, 70%, 75%, 80%, or 90% of the cells in the composition express the recombinant TCR and/or exhibits binding to the antigen.

200. A method of treatment comprising administering the engineered cell, plurality of engineered cells or composition of any of claims 194-199 to a subject.

201. Use of the engineered cell, plurality of engineered cells or composition of any of claims 194-199 for the treatment of a disease or disorder.

202. Use of the engineered cell, plurality of engineered cells or composition of any of claims 194-199 in the manufacture of a medicament for treating a disease or disorder.

203. The engineered cell, plurality of engineered cells or composition of any of claims 194-199 for use in treating a disease or disorder.

204. An article of manufacture comprising:
the polynucleotide of any of claims 69-94 or 117-140, and
one or more agent(s) capable of inducing a genetic disruption at a target site within a TRAC locus.

205. An article of manufacture comprising:
a polynucleotide comprising (a) a nucleic acid sequence encoding a T cell receptor beta (TCRβ) chain and a portion of a T cell receptor alpha (TCRα) chain, wherein the portion is less than a full-length native TCRα chain and (b) one or more homology arm(s) linked to the nucleic acid sequence, wherein the one or more homology arm(s) comprise a sequence homologous to one or more region(s) of an open reading frame of a TRAC locus, said open reading frame encoding a TCRα chain; and
one or more agent(s) capable of inducing a genetic disruption at a target site within a TRAC locus.

206. The article of manufacture of claim 205, wherein the polynucleotide comprises the polynucleotide of any of claims 69-94 or 117-140.

207. An article of manufacture comprising:
the polynucleotide of any of claims 95-140, and
one or more agent(s) capable of inducing a genetic disruption at a target site within a TRBC locus.

208. An article of manufacture comprising:
a polynucleotide comprising (a) a nucleic acid sequence encoding a T cell receptor alpha (TCRα) chain and a portion of a T cell receptor beta (TCRβ) chain, wherein the portion is less than a full-length native TCRβ chain and (b) one or more homology arm(s) linked to the nucleic acid sequence, wherein the one or more homology arm(s) comprise a sequence homologous to one or more region(s) of an open reading frame of a TRBC locus, said open reading frame encoding a TCRβ chain; and
one or more agent(s) capable of inducing a genetic disruption at a target site within a TRAC locus.

209. The article of manufacture of claim 207 or 208, wherein the polynucleotide comprises the polynucleotide of any of claims 95-140.

210. A kit comprising an article of manufacture of any of claims 204-209, and instructions for use.


SEQUENCE LISTING (261 sequences)

SEQ ID NO: 1 - DNA, 4627 bp, Homo sapiens
  Human TCR alpha constant (TRAC)
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttc

SEQ ID NO: 2 - DNA, 1448 bp, Homo sapiens
  Human TCR beta constant 1 (TRBC1)
aggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcagacaagactagatccaaaaagaaaggaaccagcgcacaccatgaaggagaattgggcacctgtggttcattcttctcccagattctcagcccaacagagccaagcagctgggtcccctttctatgtggcctgtgtaactctcatctgggtggtgccccccatccccctcagtgctgccacatgccatggattgcaaggacaatgtggctgacatctgcatggcagaagaaaggaggtgctgggctgtcagaggaagctggtctgggcctgggagtctgtgccaactgcaaatctgactttacttttaattgcctatgaaaataaggtctctcatttattttcctctccctgctttctttcagactgtggctttacctcgggtaagtaagcccttccttttcctctccctctctcatggttcttgacctagaaccaaggcatgaagaactcacagacactggagggtggagggtgggagagaccagagctacctgtgcacaggtacccacctgtccttcctccgtgccaacagtgtcctaccagcaaggggtcctgtctgccaccatcctctatgagatcctgctagggaaggccaccctgtatgctgtgctggtcagcgcccttgtgttgatggccatggtaagcaggagggcaggatggggccagcaggctggaggtgacacactgacaccaagcacccagaagtatagagtccctgccaggattggagctgggcagtagggagggaagagatttcattcaggtgcctcagaagataacttgcacctctgtaggatcacagtggaagggtcatgctgggaaggagaagctggagtcaccagaaaacccaatggatgttgtgatgagccttactatttgtgtggtcaatgggccctactactttctctcaatcctcacaactcctggctcttaataacccccaaaactttctcttctgcaggtcaagagaaaggatttctga

SEQ ID NO: 3 - DNA, 1489 bp, Homo sapiens
  Human TCR beta constant 2 (TRBC2)
aggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcggacaagactagatccagaagaaagccagagtggacaaggtgggatgatcaaggttcacagggtcagcaaagcacggtgtgcacttcccccaccaagaagcatagaggctgaatggagcacctcaagctcattcttccttcagatcctgacaccttagagctaagctttcaagtctccctgaggaccagccatacagctcagcatctgagtggtgtgcatcccattctcttctggggtcctggtttcctaagatcatagtgaccacttcgctggcactggagcagcatgagggagacagaaccagggctatcaaaggaggctgactttgtactatctgatatgcatgtgtttgtggcctgtgagtctgtgatgtaaggctcaatgtccttacaaagcagcattctctcatccatttttcttcccctgttttctttcagactgtggcttcacctccggtaagtgagtctctcctttttctctctatctttcgccgtctctgctctcgaaccagggcatggagaatccacggacacaggggcgtgagggaggccagagccacctgtgcacaggtgcctacatgctctgttcttgtcaacagagtcttaccagcaaggggtcctgtctgccaccatcctctatgagatcttgctagggaaggccaccttgtatgccgtgctggtcagtgccctcgtgctgatggccatggtaaggaggagggtgggatagggcagatgatgggggcaggggatggaacatcacacatgggcataaaggaatctcagagccagagcacagcctaatatatcctatcacctcaatgaaaccataatgaagccagactggggagaaaatgcagggaatatcacagaatgcatcatgggaggatggagacaaccagcgagccctactcaaattaggcctcagagcccgcctcccctgccctactcctgctgtgccatagcccctgaaaccctgaaaatgttctctcttccacaggtcaagagaaaggattccagaggctag

SEQ ID NO: 4 - DNA, 1189 bp, Artificial Sequence
  EF1alpha promoter
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaa

SEQ ID NO: 5 - DNA, 1205 bp, Artificial Sequence
  EF1alpha promoter
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcactagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaaa

SEQ ID NO: 6 - protein, 24 aa, Artificial Sequence
  T2A
LEGGGEGRGSLLTCGDVEENPGPR

SEQ ID NO: 7 - protein, 18 aa, Artificial Sequence
  T2A
EGRGSLLTCGDVEENPGP

SEQ ID NO: 8 - protein, 22 aa, Artificial Sequence
  P2A
GSGATNFSLLKQAGDVEENPGP

SEQ ID NO: 9 - protein, 19 aa, Artificial Sequence
  P2A
ATNFSLLKQAGDVEENPGP

SEQ ID NO: 10 - protein, 20 aa, Artificial Sequence
  E2A
QCTNYALLKLAGDVESNPGP

SEQ ID NO: 11 - protein, 22 aa, Artificial Sequence
  F2A
VKQTLNFDLLKLAGDVESNPGP

SEQ ID NO: 12 - protein, 357 aa, Artificial Sequence
  EGFRt
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 13 - protein, 335 aa, Artificial Sequence
  EGFRt
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 14 - protein, 137 aa, Mus musculus
  Mouse TCR alpha constant
DIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 15 - protein, 137 aa, Mus musculus
  Mouse TCR alpha constant
NIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 16 - protein, 173 aa, Mus musculus
  Mouse TCR beta constant
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 17 - protein, 172 aa, Mus musculus
  Mouse TCR beta constant
DLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKRKNS

SEQ ID NO: 18 - DNA, 181 bp, Artificial Sequence
  MND promoter
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 19 - protein, 142 aa, Homo sapiens
  Human TCR alpha constant
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 20 - protein, 177 aa, Homo sapiens
  Human TCR beta constant 1
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 21 - protein, 178 aa, Homo sapiens
  Human TCR beta constant 2
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 22 - protein, 10 aa, Artificial Sequence
  Linker
  REPEAT (0)...(0): SGGGG is repeated 5 or 6 times
PGGGSGGGGP

SEQ ID NO: 23 - protein, 17 aa, Artificial Sequence
  Linker
GSADDAKKDAAKKDGKS

SEQ ID NO: 24 - protein, 141 aa, Homo sapiens
  human TCR alpha constant
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 25 - protein, 179 aa, Homo sapiens
  human TCR beta constant
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 26 - DNA, 149 bp, Artificial Sequence
  TRAC gRNA transcription sequence
agcgctctcgtacagagttggcattataatacgactcactataggggagaatcaaaatcggtgaatgttttagagctagaaatagcaagttaaaataaggctagtccgttatcaacttgaaaaagtggcaccgagtcggtgcttttttt

SEQ ID NO: 27 - RNA, 100 nt, Artificial Sequence
  TRAC gRNA sequence
gagaaucaaaaucggugaauguuuuagagcuagaaauagcaaguuaaaauaaggcuaguccguuaucaacuugaaaaaguggcaccgagucggugcuuuu

SEQ ID NO: 28 - RNA, 20 nt, Artificial Sequence
  TRAC-10 gRNA targeting domain
ucucucagcugguacacggc

SEQ ID NO: 29 - RNA, 20 nt, Artificial Sequence
  TRAC-110 gRNA targeting domain
uggauuuagagucucucagc

SEQ ID NO: 30 - RNA, 20 nt, Artificial Sequence
  TRAC-116 gRNA targeting domain
acacggcagggucaggguuc

SEQ ID NO: 31 - RNA, 20 nt, Artificial Sequence
  TRAC-16 gRNA targeting domain
gagaaucaaaaucggugaau

SEQ ID NO: 32 - RNA, 20 nt, Artificial Sequence
  TRAC-4 gRNA targeting domain
gcugguacacggcaggguca

SEQ ID NO: 33 - RNA, 17 nt, Artificial Sequence
  TRAC-49 gRNA targeting domain
cucagcugguacacggc

SEQ ID NO: 34 - RNA, 17 nt, Artificial Sequence
  TRAC-2 gRNA targeting domain
ugguacacggcaggguc

SEQ ID NO: 35 - RNA, 17 nt, Artificial Sequence
  TRAC-30 gRNA targeting domain
gcuagacaugaggucua

SEQ ID NO: 36 - RNA, 17 nt, Artificial Sequence
  TRAC-43 gRNA targeting domain
gucagauuuguugcucc

SEQ ID NO: 37 - RNA, 17 nt, Artificial Sequence
  TRAC-23 gRNA targeting domain
ucagcugguacacggca

SEQ ID NO: 38 - RNA, 17 nt, Artificial Sequence
  TRAC-34 gRNA targeting domain
gcagacagacuugucac

SEQ ID NO: 39 - RNA, 17 nt, Artificial Sequence
  TRAC-25 gRNA targeting domain
gguacacggcaggguca

SEQ ID NO: 40 - RNA, 20 nt, Artificial Sequence
  TRAC-128 gRNA targeting domain
cuucaagagcaacagugcug

SEQ ID NO: 41 - RNA, 20 nt, Artificial Sequence
  TRAC-105 gRNA targeting domain
agagcaacagugcuguggcc

SEQ ID NO: 42 - RNA, 20 nt, Artificial Sequence
  TRAC-106 gRNA targeting domain
aaagucagauuuguugcucc

SEQ ID NO: 43 - RNA, 20 nt, Artificial Sequence
  TRAC-123 gRNA targeting domain
acaaaacugugcuagacaug

SEQ ID NO: 44 - RNA, 17 nt, Artificial Sequence
  TRAC-64 gRNA targeting domain
aaacugugcuagacaug

SEQ ID NO: 45 - RNA, 20 nt, Artificial Sequence
  TRAC-97 gRNA targeting domain
ugugcuagacaugaggucua

SEQ ID NO: 46 - RNA, 22 nt, Artificial Sequence
  TRAC-148 gRNA targeting domain
ggcuggggaagaaggugucuuc

SEQ ID NO: 47 - RNA, 21 nt, Artificial Sequence
  TRAC-147 gRNA targeting domain
gcuggggaagaaggugucuuc

SEQ ID NO: 48 - RNA, 18 nt, Artificial Sequence
  TRAC-234 gRNA targeting domain
ggggaagaaggugucuuc

SEQ ID NO: 49 - RNA, 22 nt, Artificial Sequence
  TRAC-167 gRNA targeting domain
guuuugucugugauauacacau

SEQ ID NO: 50 - RNA, 24 nt, Artificial Sequence
  TRAC-177 gRNA targeting domain
ggcagacagacuugucacuggauu

SEQ ID NO: 51 - RNA, 23 nt, Artificial Sequence
  TRAC-176 gRNA targeting domain
gcagacagacuugucacuggauu

SEQ ID NO: 52 - RNA, 20 nt, Artificial Sequence
  TRAC-257 gRNA targeting domain
gacagacuugucacuggauu

SEQ ID NO: 53 - RNA, 24 nt, Artificial Sequence
  TRAC-233 gRNA targeting domain
gugaauaggcagacagacuuguca

SEQ ID NO: 54 - RNA, 22 nt, Artificial Sequence
  TRAC-231 gRNA targeting domain
gaauaggcagacagacuuguca

SEQ ID NO: 55 - RNA, 22 nt, Artificial Sequence
  TRAC-163 gRNA targeting domain
gagucucucagcugguacacgg

SEQ ID NO: 56 - RNA, 20 nt, Artificial Sequence
  TRAC-241 gRNA targeting domain
gucucucagcugguacacgg

SEQ ID NO: 57 - RNA, 22 nt, Artificial Sequence
  TRAC-179 gRNA targeting domain
gguacacggcagggucaggguu

SEQ ID NO: 58 - RNA, 21 nt, Artificial Sequence
  TRAC-178 gRNA targeting domain
guacacggcagggucaggguu

SEQ ID NO: 59 - RNA, 20 nt, Artificial Sequence
  TRBC-40 gRNA targeting domain
cacccagaucgucagcgccg

SEQ ID NO: 60 - RNA, 20 nt, Artificial Sequence
  TRBC-52 gRNA targeting domain
caaacacagcgaccucgggu

SEQ ID NO: 61 - RNA, 20 nt, Artificial Sequence
  TRBC-25 gRNA targeting domain
ugacgaguggacccaggaua

SEQ ID NO: 62 - RNA, 20 nt, Artificial Sequence
  TRBC-35 gRNA targeting domain
ggcucucggagaaugacgag

SEQ ID NO: 63 - RNA, 20 nt, Artificial Sequence
  TRBC-50 gRNA targeting domain
ggccucggcgcugacgaucu

SEQ ID NO: 64 - RNA, 20 nt, Artificial Sequence
  TRBC-39 gRNA targeting domain
gaaaaacguguucccacccg

SEQ ID NO: 65 - RNA, 20 nt, Artificial Sequence
  TRBC-49 gRNA targeting domain
augacgaguggacccaggau

SEQ ID NO: 66 - RNA, 20 nt, Artificial Sequence
  TRBC-51 gRNA targeting domain
aguccaguucuacgggcucu

SEQ ID NO: 67 - RNA, 20 nt, Artificial Sequence
  TRBC-26 gRNA targeting domain
cgcugucaaguccaguucua

SEQ ID NO: 68 - RNA, 20 nt, Artificial Sequence
  TRBC-47 gRNA targeting domain
aucgucagcgccgaggccug

SEQ ID NO: 69 - RNA, 20 nt, Artificial Sequence
  TRBC-45 gRNA targeting domain
ucaaacacagcgaccucggg

SEQ ID NO: 70 - RNA, 20 nt, Artificial Sequence
  TRBC-34 gRNA targeting domain
cguagaacuggacuugacag

SEQ ID NO: 71 - RNA, 20 nt, Artificial Sequence
  TRBC-227 gRNA targeting domain
aggccucggcgcugacgauc

SEQ ID NO: 72 - RNA, 20 nt, Artificial Sequence
  TRBC-41 gRNA targeting domain
ugacagcggaagugguugcg

SEQ ID NO: 73 - RNA, 20 nt, Artificial Sequence
  TRBC-30 gRNA targeting domain
uugacagcggaagugguugc

SEQ ID NO: 74 - RNA, 20 nt, Artificial Sequence
  TRBC-206 gRNA targeting domain
ucuccgagagcccguagaac

SEQ ID NO: 75 - RNA, 20 nt, Artificial Sequence
  TRBC-32 gRNA targeting domain
cgggugggaacacguuuuuc

SEQ ID NO: 76 - RNA, 20 nt, Artificial Sequence
  TRBC-276 gRNA targeting domain
gacagguuuggcccuauccu

SEQ ID NO: 77 - RNA, 20 nt, Artificial Sequence
  TRBC-274 gRNA targeting domain
gaucgucagcgccgaggccu

SEQ ID NO: 78 - RNA, 20 nt, Artificial Sequence
  TRBC-230 gRNA targeting domain
ggcucaaacacagcgaccuc

SEQ ID NO: 79 - RNA, 20 nt, Artificial Sequence
  TRBC-235 gRNA targeting domain
ugagggucucggccaccuuc

SEQ ID NO: 80 - RNA, 20 nt, Artificial Sequence
  TRBC-38 gRNA targeting domain
aggcuucuaccccgaccacg

SEQ ID NO: 81 - RNA, 20 nt, Artificial Sequence
  TRBC-223 gRNA targeting domain
ccgaccacguggagcugagc

SEQ ID NO: 82 - RNA, 20 nt, Artificial Sequence
  TRBC-221 gRNA targeting domain
ugacagguuuggcccuaucc

SEQ ID NO: 83 - RNA, 20 nt, Artificial Sequence
  TRBC-48 gRNA targeting domain
cuugacagcggaagugguug

SEQ ID NO: 84 - RNA, 20 nt, Artificial Sequence
  TRBC-216 gRNA targeting domain
agaucgucagcgccgaggcc

SEQ ID NO: 85 - RNA, 20 nt, Artificial Sequence
  TRBC-210 gRNA targeting domain
gcgcugacgaucugggugac

SEQ ID NO: 86 - RNA, 20 nt, Artificial Sequence
  TRBC-268 gRNA targeting domain
ugagggcgggcugcuccuug

SEQ ID NO: 87 - RNA, 20 nt, Artificial Sequence
  TRBC-193 gRNA targeting domain
guugcggggguucugccaga

SEQ ID NO: 88 - RNA, 20 nt, Artificial Sequence
  TRBC-246 gRNA targeting domain
agcucagcuccacguggucg

SEQ ID NO: 89 - RNA, 20 nt, Artificial Sequence
  TRBC-228 gRNA targeting domain
gcggcugcucaggcaguauc

SEQ ID NO: 90 - RNA, 20 nt, Artificial Sequence
  TRBC-43 gRNA targeting domain
gcggggguucugccagaagg

SEQ ID NO: 91 - RNA, 20 nt, Artificial Sequence
  TRBC-272 gRNA targeting domain
uggcucaaacacagcgaccu

SEQ ID NO: 92 - RNA, 20 nt, Artificial Sequence
  TRBC-33 gRNA targeting domain
acuggacuugacagcggaag

SEQ ID NO: 93 - RNA, 20 nt, Artificial Sequence
  TRBC-44 gRNA targeting domain
gacagcggaagugguugcgg

SEQ ID NO: 94 - RNA, 20 nt, Artificial Sequence
  TRBC-211 gRNA targeting domain
gcugucaaguccaguucuac

SEQ ID NO: 95 - RNA, 20 nt, Artificial Sequence
  TRBC-253 gRNA targeting domain
guaucuggagucauugaggg

SEQ ID NO: 96 - RNA, 17 nt, Artificial Sequence
  TRBC-18 gRNA targeting domain
cucggcgcugacgaucu

SEQ ID NO: 97 - RNA, 17 nt, Artificial Sequence
  TRBC-6 gRNA targeting domain
ccucggcgcugacgauc

SEQ ID NO: 98 - RNA, 17 nt, Artificial Sequence
  TRBC-85 gRNA targeting domain
ccgagagcccguagaac

SEQ ID NO: 99 - RNA, 17 nt, Artificial Sequence
  TRBC-129 gRNA targeting domain
ccagaucgucagcgccg

SEQ ID NO: 100 - RNA, 17 nt, Artificial Sequence
  TRBC-93 gRNA targeting domain
gaaugacgaguggaccc

SEQ ID NO: 101 - RNA, 22 nt, Artificial Sequence
  TRBC-415 gRNA targeting domain
gggugacagguuuggcccuauc

SEQ ID NO: 102 - RNA, 21 nt, Artificial Sequence
  TRBC-414 gRNA targeting domain
ggugacagguuuggcccuauc

SEQ ID NO: 103 - RNA, 20 nt, Artificial Sequence
  TRBC-310 gRNA targeting domain
gugacagguuuggcccuauc

SEQ ID NO: 104 - RNA, 18 nt, Artificial Sequence
  TRBC-308 gRNA targeting domain
gacagguuuggcccuauc

SEQ ID NO: 105 - RNA, 22 nt, Artificial Sequence
  TRBC-401 gRNA targeting domain
gauacugccugagcagccgccu

SEQ ID NO: 106 - RNA, 24 nt, Artificial Sequence
  TRBC-468 gRNA targeting domain
gaccacguggagcugagcuggugg

SEQ ID NO: 107 - RNA, 18 nt, Artificial Sequence
  TRBC-462 gRNA targeting domain
guggagcugagcuggugg

SEQ ID NO: 108 - RNA, 24 nt, Artificial Sequence
  TRBC-424 gRNA targeting domain
gggcgggcugcuccuugaggggcu

SEQ ID NO: 109 - RNA, 23 nt, Artificial Sequence
  TRBC-423 gRNA targeting domain
ggcgggcugcuccuugaggggcu

SEQ ID NO: 110 - RNA, 22 nt, Artificial Sequence
  TRBC-422 gRNA targeting domain
gcgggcugcuccuugaggggcu

SEQ ID NO: 111 - RNA, 20 nt, Artificial Sequence
  TRBC-420 gRNA targeting domain
gggcugcuccuugaggggcu

SEQ ID NO: 112 - RNA, 19 nt, Artificial Sequence
  TRBC-419 gRNA targeting domain
ggcugcuccuugaggggcu

SEQ ID NO: 113 - RNA, 18 nt, Artificial Sequence
  TRBC-418 gRNA targeting domain
gcugcuccuugaggggcu

SEQ ID NO: 114 - RNA, 24 nt, Artificial Sequence
  TRBC-445 gRNA targeting domain
ggugaaugggaaggaggugcacag

SEQ ID NO: 115 - RNA, 23 nt, Artificial Sequence
  TRBC-444 gRNA targeting domain
gugaaugggaaggaggugcacag

SEQ ID NO: 116 - RNA, 21 nt, Artificial Sequence
  TRBC-442 gRNA targeting domain
gaaugggaaggaggugcacag

SEQ ID NO: 117 - DNA, 20 bp, Artificial Sequence
  TRAC target sequence
attcaccgattttgattctc

SEQ ID NO: 118 - DNA, 20 bp, Artificial Sequence
  TRBC target sequence
agatcgtcagcgccgaggcc

SEQ ID NO: 119 - DNA, 25 bp, Artificial Sequence
  HBB splice site acceptor
ctgacctcttctcttcctcccacag

SEQ ID NO: 120 - DNA, 13 bp, Artificial Sequence
  IgG splice site acceptor
tttctctccacag

SEQ ID NO: 121 - protein, 138 aa, mus musculus
  Mouse TCR alpha constant
PYIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 122 - protein, 136 aa, Artificial Sequence
  Modified mouse TCR alpha constant
IQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 123 - protein, 173 aa, Artificial Sequence
  Modified mouse TCR beta constant
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 124 - DNA, 611 bp, Artificial Sequence
  TRAC 5′ homology arm
ctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgat

SEQ ID NO: 125 - DNA, 628 bp, Artificial Sequence
  TRAC 3′ homology arm
tttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtg

SEQ ID NO: 126 - DNA, 347 bp, Artificial Sequence
  MND promoter
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatc

SEQ ID NO: 127 - DNA, 544 bp, Artificial Sequence
  Ef1alpha promoter with HTLV1 enhancer
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 128 - DNA, 66 bp, Artificial Sequence
  P2A nucleotide sequence
ggatctggagcgacgaattttagtctactgaaacaagcgggagacgtggaggaaaaccctggacct

SEQ ID NO: 129 - protein, 112 aa, homo sapiens
  CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 112 aa, homo sapiens
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 12 aa, homo sapiens
  spacer (IgG4hinge)
ESKYGPPCPPCP

SEQ ID NO: 132 - DNA, 36 bp, homo sapiens
  spacer (IgG4hinge)
gaatctaagtacggaccgccctgccccccttgccct

SEQ ID NO: 133 - protein, 119 aa, homo sapiens
  Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 134 - protein, 229 aa, homo sapiens
  Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 135 - protein, 282 aa, homo sapiens
  IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 136 - protein, 27 aa, homo sapiens
  CD28
FWVLVVVGGVLACYSLLVTVAFIIFWV

SEQ ID NO: 137 - protein, 66 aa, homo sapiens
  CD28
IEVMYPPPYLDNEKSNGTIIHVKGKHLCPSPLFPGPSKPFWVLVVVGGVLACYSLLVTVAFIIFWV

SEQ ID NO: 138 - protein, 41 aa, homo sapiens
  CD28
RSKRSRLLHSDYMNMTPRRPGPTRKHYQPYAPPRDFAAYRS

SEQ ID NO: 139 - protein, 41 aa, homo sapiens
  CD28 (LL to GG)
RSKRSRGGHSDYMNMTPRRPGPTRKHYQPYAPPRDFAAYRS

SEQ ID NO: 140 - protein, 42 aa, homo sapiens
  4-1BB
KRGRKKLLYIFKQPFMRPVQTTQEEDGCSCRFPEEEEGGCEL

SEQ ID NO: 141 - protein, 112 aa, homo sapiens
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - DNA, 138 bp, Artificial Sequence
  Partial recombinant TCRalpha constant region exon 1 sequence
cagaacccagatcccgcggtatatcaactgcgcgactcaaaatcatccgataagagtgtctgtttgtttactgacttcgacagtcaaactaatgtctctcagagcaaagattccgatgtctacatcactgacaagtgc

SEQ ID NO: 143 - protein, 9 aa, Artificial Sequence
  HPV16 E7(11-19) peptide
YMLDLQPET

SEQ ID NO: 144 - RNA, 96 nt, Artificial Sequence
  TRAC gRNA
  modified_base (1)...(20): a, c, u, g, unknown or other
  misc_feature (1)...(20): n is a, c, g, or u
nnnnnnnnnnnnnnnnnnnnguuuuagagcuagaaauagcaaguuaaaauaaggcuaguccguuaucaacuugaaaaaguggcaccgagucggugc

SEQ ID NO: 145 - RNA, 104 nt, Artificial Sequence
  exemplary gRNA complementary domain
  modified_base (1)...(20): a, c, u, g, unknown or other
  misc_feature (1)...(20): n is a, c, g, or u
nnnnnnnnnnnnnnnnnnnnguuuuagagcuaugcugaaaagcauagcaaguuaaaauaaggcuaguccguuaucaacuugaaaaaguggcaccgagucggugc

SEQ ID NO: 146 - RNA, 106 nt, Artificial Sequence
  TRAC gRNA
  modified_base (1)...(20): a, c, u, g, unknown or other
  misc_feature (1)...(20): n is a, c, g, or u
nnnnnnnnnnnnnnnnnnnnguuuuagagcuaugcuggaaacagcauagcaaguuaaaauaaggcuaguccguuaucaacuugaaaaaguggcaccgagucggugc

SEQ ID NO: 147 - RNA, 116 nt, Artificial Sequence
  TRAC gRNA
  modified_base (1)...(20): a, c, u, g, unknown or other
  misc_feature (1)...(20): n is a, c, g, or u
nnnnnnnnnnnnnnnnnnnnguuuuagagcuaugcuguuuuggaaacaaaacagcauagcaaguuaaaauaaggcuaguccguuaucaacuugaaaaaguggcaccgagucggugc

SEQ ID NO: 148 - RNA, 96 nt, Artificial Sequence
  TRAC gRNA
  modified_base (1)...(20): a, c, u, g, unknown or other
  misc_feature (1)...(20): n is a, c, g, or u
nnnnnnnnnnnnnnnnnnnnguauuagagcuagaaauagcaaguuaauauaaggcuaguccguuaucaacuugaaaaaguggcaccgagucggugc

SEQ ID NO: 149 - RNA, 96 nt, Artificial Sequence
  TRAC gRNA
  modified_base (1)...(20): a, c, u, g, unknown or other
  misc_feature (1)...(20): n is a, c, g, or u
nnnnnnnnnnnnnnnnnnnnguuuaagagcuagaaauagcaaguuuaaauaaggcuaguccguuaucaacuugaaaaaguggcaccgagucggugc

SEQ ID NO: 150 - RNA, 116 nt, Artificial Sequence
  TRAC gRNA
  modified_base (1)...(20): a, c, u, g, unknown or other
  misc_feature (1)...(20): n is a, c, g, or u
nnnnnnnnnnnnnnnnnnnnguauuagagcuaugcuguauuggaaacaauacagcauagcaaguuaauauaaggcuaguccguuaucaacuugaaaaaguggcaccgagucggugc

SEQ ID NO: 151 - RNA, 47 nt, Artificial Sequence
  gRNA Proximal and tail domain
aaggcuaguccguuaucaacuugaaaaaguggcaccgagucggugcu

SEQ ID NO: 152 - RNA, 49 nt, Artificial Sequence
  gRNA Proximal and tail domain
aaggcuaguccguuaucaacuugaaaaaguggcaccgagucgguggugc

SEQ ID NO: 153 - RNA, 51 nt, Artificial Sequence
  gRNA Proximal and tail domain
aaggcuaguccguuaucaacuugaaaaaguggcaccgagucggugcggauc

SEQ ID NO: 154 - RNA, 31 nt, Artificial Sequence
  gRNA Proximal and tail domain
aaggcuaguccguuaucaacuugaaaaagug

SEQ ID NO: 155 - RNA, 18 nt, Artificial Sequence
  gRNA Proximal and tail domain
aaggcuaguccguuauca

SEQ ID NO: 156 - RNA, 12 nt, Artificial Sequence
  gRNA Proximal and tail domain
aaggcuaguccg

SEQ ID NO: 157 - RNA, 102 nt, Artificial Sequence
  Exemplary chimeric gRNA
  modified_base (1)...(20): a, c, u, g, unknown or other
  misc_feature (1)...(20): n is a, c, g, or u
nnnnnnnnnnnnnnnnnnnnguuuuagagcuagaaauagcaaguuaaaauaaggcuaguccguuaucaacuugaaaaaguggcaccgagucggugcuuuuuu

SEQ ID NO: 158 - RNA, 102 nt, Artificial Sequence
  Exemplary chimeric gRNA
  modified_base (1)...(20): a, c, u, g, unknown or other
  misc_feature (1)...(20): n is a, c, g, or u
nnnnnnnnnnnnnnnnnnnnguuuuaguacucuggaaacagaaucuacuaaaacaaggcaaaaugccguguuuaucucgucaacuuguuggcgagauuuuuu

SEQ ID NO: 159 - protein, 1344 aa, Artificial Sequence
  Streptococcus mutans Cas9
KKPYSIGLDIGTNSVGWAVVTDDYKVPAKKMKVLGNTDKSHIEKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEELEVLLAQIGDNYAELFLSAKKLYDSILLSGILTVTDVGTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKLLTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKSYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHKENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLNKLGGD

SEQ ID NO: 160 - protein, 1367 aa, Artificial Sequence
  Streptococcus pyogenes Cas9
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 161 - protein, 1387 aa, Artificial Sequence
  Streptococcus thermophilus Cas9
TKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 162 - protein, 1333 aa, Artificial Sequence
  Listeria innocua Cas9
KKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 163 - DNA, 20 bp, Artificial Sequence
  TRAC target sequence 2
gagaatcaaaatcggtgaat

SEQ ID NO: 164 - DNA, 20 bp, Artificial Sequence
  TRBC target sequence 2
ggcctcggcgctgacgatct

SEQ ID NO: 165 - DNA, 23 bp, Artificial Sequence
  TRAC target sequence with PAM 3
gagaatcaaaatcggtgaatagg

SEQ ID NO: 166 - DNA, 23 bp, Artificial Sequence
  TRAC target sequence with PAM 4
ttcaaaacctgtcagtgattggg

SEQ ID NO: 167 - DNA, 23 bp, Artificial Sequence
  TRAC target sequence with PAM 5
tgtgctagacatgaggtctatgg

SEQ ID NO: 168 - DNA, 23 bp, Artificial Sequence
  TRAC target sequence with PAM 6
cgtcatgagcagattaaacccgg

SEQ ID NO: 169 - DNA, 23 bp, Artificial Sequence
  TRAC target sequence with PAM 7
tcagggttctggatatctgtggg

SEQ ID NO: 170 - DNA, 23 bp, Artificial Sequence
  TRAC target sequence with PAM 8
gtcagggttctggatatctgtgg

SEQ ID NO: 171 - DNA, 23 bp, Artificial Sequence
  TRAC target sequence with PAM 9
ttcggaacccaatcactgacagg

SEQ ID NO: 172 - DNA, 23 bp, Artificial Sequence
  TRAC target sequence with PAM 10
taaacccggccactttcaggagg

SEQ ID NO: 173 - DNA, 23 bp, Artificial Sequence
  TRAC target sequence with PAM 11
aaagtcagatttgttgctccagg

SEQ ID NO: 174 - DNA, 23 bp, Artificial Sequence
  TRAC target sequence with PAM 12
aacaaatgtgtcacaaagtaagg

SEQ ID NO: 175 - DNA, 23 bp, Artificial Sequence
  TRAC target sequence with PAM 13
tggatttagagtctctcagctgg

SEQ ID NO: 176 - DNA, 23 bp, Artificial Sequence
  TRAC target sequence with PAM 14
taggcagacagacttgtcactgg

SEQ ID NO: 177 - DNA, 23 bp, Artificial Sequence
  TRAC target sequence with PAM 15
agctggtacacggcagggtcagg

SEQ ID NO: 178 - DNA, 23 bp, Artificial Sequence
  TRAC target sequence with PAM 16
gctggtacacggcagggtcaggg

SEQ ID NO: 179 - DNA, 23 bp, Artificial Sequence
  TRAC target sequence with PAM 17
tctctcagctggtacacggcagg

SEQ ID NO: 180 - DNA, 23 bp, Artificial Sequence
  TRAC target sequence with PAM 18
tttcaaaacctgtcagtgattgg

SEQ ID NO: 181 - DNA, 23 bp, Artificial Sequence
  TRAC target sequence with PAM 19
gattaaacccggccactttcagg

SEQ ID NO: 182 - DNA, 23 bp, Artificial Sequence
  TRAC target sequence with PAM 20
ctcgaccagcttgacatcacagg

SEQ ID NO: 183 - DNA, 23 bp, Artificial Sequence
  TRAC target sequence with PAM 21
agagtctctcagctggtacacgg

SEQ ID NO: 184 - DNA, 23 bp, Artificial Sequence
  TRAC target sequence with PAM 22
ctctcagctggtacacggcaggg

SEQ ID NO: 185 - DNA, 23 bp, Artificial Sequence
  TRAC target sequence with PAM 23
aagttcctgtgatgtcaagctgg

SEQ ID NO: 186 - DNA, 23 bp, Artificial Sequence
  TRAC target sequence with PAM 24
atcctcctcctgaaagtggccgg

SEQ ID NO: 187 - DNA, 23 bp, Artificial Sequence
  TRAC target sequence with PAM 25
tgctcatgacgctgcggctgtgg

SEQ ID NO: 188 - DNA, 23 bp, Artificial Sequence
  TRAC target sequence with PAM 26
acaaaactgtgctagacatgagg

SEQ ID NO: 189 - DNA, 23 bp, Artificial Sequence
  TRAC target sequence with PAM 27
atttgtttgagaatcaaaatcgg

SEQ ID NO: 190 - DNA, 23 bp, Artificial Sequence
  TRAC target sequence with PAM 28
catcacaggaactttctaaaagg

SEQ ID NO: 191 - DNA, 23 bp, Artificial Sequence
  TRAC target sequence with PAM 29
gtcgagaaaagctttgaaacagg

SEQ ID NO: 192 - DNA, 23 bp, Artificial Sequence
  TRAC target sequence with PAM 30
ccactttcaggaggaggattcgg

SEQ ID NO: 193 - DNA, 23 bp, Artificial Sequence
  TRAC target sequence with PAM 31
ctgacaggttttgaaagtttagg

SEQ ID NO: 194 - DNA, 23 bp, Artificial Sequence
  TRAC target sequence with PAM 32
agctttgaaacaggtaagacagg

SEQ ID NO: 195 - DNA, 23 bp, Artificial Sequence
  TRAC target sequence with PAM 33
tggaataatgctgttgttgaagg

SEQ ID NO: 196 - DNA, 23 bp, Artificial Sequence
  TRAC target sequence with PAM 34
agagcaacagtgctgtggcctgg

SEQ ID NO: 197 - DNA, 23 bp, Artificial Sequence
  TRAC target sequence with PAM 35
ctgtggtccagctgaggtgaggg

SEQ ID NO: 198 - DNA, 23 bp, Artificial Sequence
  TRAC target sequence with PAM 36
ctgcggctgtggtccagctgagg

SEQ ID NO: 199 - DNA, 23 bp, Artificial Sequence
  TRAC target sequence with PAM 37
tgtggtccagctgaggtgagggg

SEQ ID NO: 200 - DNA, 23 bp, Artificial Sequence
  TRAC target sequence with PAM 38
cttcttccccagcccaggtaagg

SEQ ID NO: 201 - DNA, 23 bp, Artificial Sequence
  TRAC target sequence with PAM 39
acacggcagggtcagggttctgg

SEQ ID NO: 202 - DNA, 23 bp, Artificial Sequence
  TRAC target sequence with PAM 40
cttcaagagcaacagtgctgtgg

SEQ ID NO: 203 - DNA, 23 bp, Artificial Sequence
  TRAC target sequence with PAM 41
ctggggaagaaggtgtcttctgg

SEQ ID NO: 204 - DNA, 23 bp, Artificial Sequence
  TRAC target sequence with PAM 42
tcctcctcctgaaagtggccggg

SEQ ID NO: 205 - DNA, 23 bp, Artificial Sequence
  TRAC target sequence with PAM 43
ttaatctgctcatgacgctgcgg

SEQ ID NO: 206 - DNA, 23 bp, Artificial Sequence
  TRAC target sequence with PAM 44
acccggccactttcaggaggagg

SEQ ID NO: 207 - DNA, 23 bp, Artificial Sequence
  TRAC target sequence with PAM 45
ttcttccccagcccaggtaaggg

SEQ ID NO: 208 - DNA, 23 bp, Artificial Sequence
  TRAC target sequence with PAM 46
cttacctgggctggggaagaagg

SEQ ID NO: 209 - DNA, 23 bp, Artificial Sequence
  TRAC target sequence with PAM 47
gacaccttcttccccagcccagg

SEQ ID NO: 210 - DNA, 23 bp, Artificial Sequence
  TRAC target sequence with PAM 48
gctgtggtccagctgaggtgagg

SEQ ID NO: 211 - DNA, 23 bp, Artificial Sequence
  TRAC target sequence with PAM 49
ccgaatcctcctcctgaaagtgg

SEQ ID NO: 212 - DNA, 23 bp, Artificial Sequence
  TRBC target sequence with PAM 3
gctgtcaagtccagttctacggg

SEQ ID NO: 213 - DNA, 28 bp, Artificial Sequence
  TRAC ZFN target sequence 1
ctatggacttcaagagcaacagtgctgt

SEQ ID NO: 214 - DNA, 28 bp, Artificial Sequence
  TRAC ZFN target sequence 2
ctcatgtctagcacagttttgtctgtga

SEQ ID NO: 215 - DNA, 28 bp, Artificial Sequence
  TRAC ZFN target sequence 3
gtgctgtggcctggagcaacaaatctga

SEQ ID NO: 216 - DNA, 28 bp, Artificial Sequence
  TRAC ZFN target sequence 4
ttgctcttgaagtccatagacctcatgt

SEQ ID NO: 217 - DNA, 27 bp, Artificial Sequence
  TRAC ZFN target sequence 5
ctgtggcctggagcaacaaatctgact

SEQ ID NO: 218 - DNA, 28 bp, Artificial Sequence
  TRAC ZFN target sequence 6
ctgttgctcttgaagtccatagacctca

SEQ ID NO: 219 - DNA, 28 bp, Artificial Sequence
  TRAC ZFN target sequence 7
ctgtggcctggagcaacaaatctgactt

SEQ ID NO: 220 - DNA, 28 bp, Artificial Sequence
  TRAC ZFN target sequence 8
ctgactttgcatgtgcaaacgccttcaa

SEQ ID NO: 221 - DNA, 28 bp, Artificial Sequence
  TRAC ZFN target sequence 9
ttgttgctccaggccacagcactgttgc

SEQ ID NO: 222 - DNA, 28 bp, Artificial Sequence
  TRAC ZFN target sequence 10
tgaaagtggccgggtttaatctgctcat

SEQ ID NO: 223 - DNA, 28 bp, Artificial Sequence
  TRAC ZFN target sequence 11
aggaggattcggaacccaatcactgaca

SEQ ID NO: 224 - DNA, 28 bp, Artificial Sequence
  TRAC ZFN target sequence 12
gaggaggattcggaacccaatcactgac

SEQ ID NO: 225 - DNA, 28 bp, Artificial Sequence
  TRBC ZFN target sequence 1
ccgtagaactggacttgacagcggaagt

SEQ ID NO: 226 - DNA, 28 bp, Artificial Sequence
  TRBC ZFN target sequence 2
tctcggagaatgacgagtggacccagga

SEQ ID NO: 227 - protein, 1082 aa, Artificial Sequence
  Neisseria meningitidis Cas9
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 228 - protein, 1368 aa, Artificial Sequence [sequence omitted]

SEQ ID NO: 229 - DNA, 50 bp, Artificial Sequence
  TRAC 50 bp 5′ Homology Arm
agctgagagactctaaatccagtgacaagtctgtctgcctattcaccgat

SEQ ID NO: 230 - DNA, 100 bp, Artificial Sequence
  TRAC 100 bp 5′ Homology Arm
ctgatcctcttgtcccacagatatccagaaccctgaccctgccgtgtaccagctgagagactctaaatccagtgacaagtctgtctgcctattcaccgat

SEQ ID NO: 231 - DNA, 200 bp, Artificial Sequence
  TRAC 200 bp 5′ Homology Arm
gtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgat

SEQ ID NO: 232 - DNA, 300 bp, Artificial Sequence
  TRAC 300 bp 5′ Homology Arm
cctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgat

SEQ ID NO: 233 - DNA, 400 bp, Artificial Sequence
  TRAC 400 bp 5′ Homology Arm
attaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgat

SEQ ID NO: 234 - DNA, 500 bp, Artificial Sequence
  TRAC 500 bp 5′ Homology Arm
ctccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgat

SEQ ID NO: 235 - DNA, 600 bp, Artificial Sequence
  TRAC 600 bp 5′ Homology Arm
agagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgat

SEQ ID NO: 236 - DNA, 50 bp, Artificial Sequence
  TRAC 50 bp 3′ Homology Arm
tttgattctcaaacaaatgtgtcacaaagtaaggattctgatgtgtatat

SEQ ID NO: 237 - DNA, 100 bp, Artificial Sequence
  TRAC 100 bp 3′ Homology Arm
tttgattctcaaacaaatgtgtcacaaagtaaggattctgatgtgtatatcacagacaaaactgtgctagacatgaggtctatggacttcaagagcaaca

SEQ ID NO: 238 - DNA, 200 bp, Artificial Sequence
  TRAC 200 bp 3′ Homology Arm
tttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaaggg

SEQ ID NO: 239 - DNA, 300 bp, Artificial Sequence
  TRAC 300 bp 3′ Homology Arm
tttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggt

SEQ ID NO: 240 - DNA, 400 bp, Artificial Sequence
  TRAC 400 bp 3′ Homology Arm
tttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaag

SEQ ID NO: 241 - DNA, 500 bp, Artificial Sequence
  TRAC 500 bp 3′ Homology Arm
tttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttc

SEQ ID NO: 242 - DNA, 600 bp, Artificial Sequence
  TRAC 600 bp 3′ Homology Arm
tttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtca

SEQ ID NO: 243 - protein, 141 aa, Artificial Sequence
  Human TCR alpha constant
NIQKPDPAVYQLRDSKSSDKSVCLFTDFDSQTNVSQSKDSDVYITDKTVLDMRSMDFKSNSAVAWSNKSDFACANAFNNSIIPADTFFPSPESSCDVKLVEKSFETDTNLNFQNLSVIGFRILLLKVAGFNLLMTLRLWSS

SEQ ID NO: 244 - protein, 141 aa, Artificial Sequence
  Human TCR alpha constant
HIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 245 - protein, 141 aa, Artificial Sequence
  Human TCR alpha constant
YIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 246 - protein, 141 aa, Artificial Sequence
  Human TCR alpha constant
DIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 247 - protein, 142 aa, Artificial Sequence
  Human TCR alpha constant
PNIQKPDPAVYQLRDSKSSDKSVCLFTDFDSQTNVSQSKDSDVYITDKTVLDMRSMDFKSNSAVAWSNKSDFACANAFNNSIIPADTFFPSPESSCDVKLVEKSFETDTNLNFQNLSVIGFRILLLKVAGFNLLMTLRLWSS

SEQ ID NO: 248 - protein, 141 aa, Artificial Sequence
  Human TCR alpha constant
NIQKPDPAVYQLRDSKSSDKSVCLFTDFDSQTNVSQSKDSDVYITDKCVLDMRSMDFKSNSAVAWSNKSDFACANAFNNSIIPADTFFPSPESSCDVKLVEKSFETDTNLNFQNLSVIGFRILLLKVAGFNLLMTLRLWSS

SEQ ID NO: 249 - protein, 141 aa, Artificial Sequence
  Human TCR alpha constant
HIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 250 - protein, 141 aa, Artificial Sequence
  Human TCR alpha constant
YIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 251 - protein, 141 aa, Artificial Sequence
  Human TCR alpha constant
NIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 252 - protein, 141 aa, Artificial Sequence
  Human TCR alpha constant
DIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 253 - protein, 177 aa, Artificial Sequence
  Human TCR beta constant
EDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 254 - protein, 180 aa, Artificial Sequence
  Human TCR beta constant
TEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 255 - protein, 180 aa, Artificial Sequence
  Human TCR beta constant
LEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 256 - protein, 179 aa, Artificial Sequence
  Human TCR beta constant
EDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 257 - protein, 180 aa, Artificial Sequence
  Human TCR beta constant
TEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 258 - protein, 180 aa, Artificial Sequence
  Human TCR beta constant
LEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 259 - protein, 46 aa, Artificial Sequence
  Partial TCR alpha constant region
QNPDPAVYQLRDSKSSDKSVCLFTDFDSQTNVSQSKDSDVYITDKC

SEQ ID NO: 260 - DNA, 131 bp, Artificial Sequence
  Partial endogenous TCR alpha constant region
gtgctagacatgaggtctatggacttcaagagcaacagtgctgtggcctggagcaacaaatctgactttgcatgtgcaaacgccttcaacaacagcattattccagaagacaccttcttccccagcccagg

SEQ ID NO: 261 - protein, 43 aa, Artificial Sequence
  Partial TCR alpha constant region
VLDMRSMDFKSNSAVAWSNKSDFACANAFNNSIIPEDTFFPSP